(19) United States
(12) Patent Application Publication
(10) Pub. No.: US 2017/0298119 A1
(43) Pub. Date: Oct. 19, 2017

(54) ANTIBODY MOLECULES TO ZIKA VIRUS AND USES THEREOF

(71) Applicant: VISTERRA, INC., Cambridge, MA (US)

(72) Inventors: Andrew M. Wollacott, Boston, MA (US); Luke Robinson, Quincy, MA (US); Gregory Babcock, Marlborough, MA (US); Zachary Holmes Shriver, Winchester, MA (US)

(21) Appl. No.: 15/488,153

(22) Filed: Apr. 14, 2017

Related U.S. Application Data
(60) Provisional application No. 62/325,776, filed on Apr. 21, 2016.
(60) Provisional application No. 62/323,376, filed on Apr. 15, 2016.

(51) Int. Cl. C07K 16/10; A61K 39/12; C12N 7/00; A61K 39/00

(52) U.S. Cl. CPC C07K 16/10; C12N 7/00; A61K 39/12; C07K 2317/76; C07K 2317/33; C07K 2317/24; A61K 2039/505; C07K 2317/92; C12N 2770/24134; C07K 2317/565

(57) ABSTRACT

Antibody molecules that specifically bind to Zika virus are disclosed. The antibody molecules can be used to treat, prevent, and/or diagnose Zika virus infection and disorders associated with Zika virus infection.

### CROSS REFERENCE TO RELATED APPLICATIONS

This application claims the benefit of U.S. Provisional Application No. 62/323,376, filed Apr. 15, 2016, and U.S. Provisional Application No. 62/325,776, filed Apr. 21, 2016. The contents of the aforesaid applications are hereby incorporated by reference in their entirety.

### BACKGROUND

Zika virus (ZIKV) is an arthropod-borne Flavivirus that, since 2015, has rapidly spread through South and Central America and the Caribbean (Fauci and Morens. N Engl J Med. 2016; 374(7): 601-4). ZIKV was first discovered in Uganda in 1947 in the course of mosquito and primate surveillance. The infection, known as Zika fever, often causes no or only mild symptoms, and had until now remained a rare disorder confined within a narrow equatorial belt from Africa to Asia. Recent studies suggest that this infection is associated with microcephaly and neuroinvasiveness, including onset of Guillain-Barre Syndrome (Mlakar et al. N Engl J Med. 2016; 374(10):951-8; Rubin et al. N Engl J Med. 2016; 374(10):984-5; de Paula Freitas et al. JAMA Ophthalmol. 2016 Feb. 9. doi: 10.1001/jamaophthalmol.2016.0267). The World Health Organization (WHO) has declared the Zika virus an international public health emergency, prompted by growing concern that an increased incidence of microcephaly in fetuses born to mothers infected with ZIKV is linked to the recent outbreaks.

There is a need for developing new approaches for treating, preventing, and diagnosing Zika virus infection and other disorders that share similar disease mechanisms.

### SUMMARY

This disclosure provides, at least in part, antibody molecules that bind to Zika virus, e.g., the envelop (E) protein of Zika virus, e.g., domain III of the E protein; and that comprise one or more functional and/or structural properties disclosed herein. In an embodiment, the antibody molecule binds to and/or reduces (e.g., inhibits or neutralizes) one or more activities of Zika virus. In an embodiment, the antibody molecule binds to an epitope that includes one or more amino acid residues in domain III of the Zika virus E protein. In an embodiment, the antibody molecule is not cross-reactive with a heterologous virus, e.g., another flavivirus (e.g., a dengue virus). While not wishing to be bound by theory, it is believed that in an embodiment, cross-reactive antibodies having low avidity or neutralizing ability can lead to enhanced viral infection, e.g., through the mechanism of antibody-dependent enhancement (ADE). For example, non-neutralizing antibodies can bind to the virus and be taken up by monocytes through Fc-FcγR interaction, leading to enhanced viral replication and/or increased in viral load. In an embodiment, the antibody molecules described herein can achieve effective or optimal inhibition of Zika virus, while reducing the risk of antibody-dependent enhancement of infection, by targeting certain region(s) on the E protein (e.g., domain III of the E protein). In an embodiment, the antibody molecule is selected from Table 1, or competes for binding to Zika virus with an antibody molecule selected from Table 1. In an embodiment, the antibody molecule binds to the same or overlapping epitope recognized by an antibody molecule selected from Table 1. In an embodiment, the antibody molecule comprises one or more heavy chain variable regions and/or one or more light chain variable regions described in Table 1. In an embodiment, the antibody molecule comprises one or more heavy chain CDRs and/or one or more light chain CDRs described in Table 1. Further provided herein are vaccines and immunogens that can induce an immune response against, or confers protection against, Zika virus. In an embodiment, the vaccine or immunogen comprises an epitope described herein. Nucleic acid molecules encoding the antibody molecules, vaccines or immunogens, expression vectors, host cells, compositions (e.g., pharmaceutical compositions), kits, containers, and methods for making the antibody molecules or vaccines, are also provided. The antibody molecules, compositions, and vaccines disclosed herein can be used (alone or in combination with other agents or therapeutic modalities) to treat, prevent, and/or diagnose Zika virus infection or disorders associated with Zika virus infection.

In an aspect, the disclosure features an anti-Zika virus (ZIKV) antibody molecule, which has one or more (e.g., 2, 3, 4, 5, 6, 7, 8, 9, 10, 11, 12, 13, 14, 15 16, 17, 18, 19, 20, 21, 22, or all) of the following properties:

a) binds, or substantially binds, to an E protein of Zika virus, e.g., domain III of the E protein (e.g., the lateral ridge of domain III of the E protein);

b) binds, or substantially binds, to an epitope on Zika virus, e.g., an E protein of Zika virus, that is not cross-reactive to one or more different flaviviruses, e.g., dengue virus, Japanese encephalitis virus, West Nile virus, yellow fever virus, or combination thereof;

c) binds, or substantially binds, to an epitope on Zika virus comprising one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) amino acid residues in domain III of an E protein;

d) binds, or substantially binds, to an epitope on Zika virus, e.g., the same, similar, or overlapping epitope as the epitope recognized by a monoclonal antibody described in Table 1;

e) binds, or substantially binds, to a Zika virus (e.g., an E protein of a Zika virus, e.g., domain III of the E protein) with high affinity, e.g., with a dissociation constant (K_{D}) of about 100 nM or less, e.g., about 10 nM or less, about 5 nM or less, about 1 nM or less, about 0.5 nM or less, about 0.1 nM or less, about 0.05 nM or less, or about 0.01 nM or less, e.g., about 10 nM to about 0.01 nM, about 5 nM to about 0.01 nM, about 3 to about 0.05 nM, or about 1 nM and about 0.1 nM, e.g., about 50 nM or less, about 40 nM or less, about 30 nM or less, about 20 nM or less, about 10 nM or less, about 8 nM or less, about 6 nM or less, about 4 nM or less, about 2 nM or less, about 1 nM or less, about 0.5 nM or less, about 0.2 nM or less, about 0.1 nM or less, about 0.05 nM or less, about 0.02 nM or less, or about 0.01 nM or less, e.g., as determined by ELISA or SPR;

f) shows the same or similar binding affinity or specificity, or both, as a monoclonal antibody described in Table 1;

g) shows the same or similar binding affinity or specificity, or both, as an antibody molecule comprising a heavy chain variable region and/or a light chain variable region described in Table 1;

h) shows the same or similar binding affinity or specificity, or both, as an antibody molecule comprising one or more (e.g., two or three) heavy chain CDRs and/or one or more (e.g., two or three) light chain CDRs described in Table 1;

i) shows the same or similar binding affinity or specificity, or both, as an antibody molecule comprising an amino acid sequence shown in Table 1;

j) shows the same or similar binding affinity or specificity, or both, as an antibody molecule comprising an amino acid sequence encoded by a nucleotide sequence shown in Table 2;

k) inhibits, e.g., competitively inhibits, the binding of a second antibody molecule to Zika virus, wherein the second antibody molecule is an antibody molecule chosen from Table 1;

l) competes for binding with a second antibody molecule to Zika virus, wherein the second antibody molecule is an antibody molecule chosen from Table 1;

m) has one or more biological properties of a monoclonal antibody chosen from Table 1;

n) has one or more structural properties of a monoclonal antibody chosen from Table 1;

o) has one or more pharmacokinetic properties of a monoclonal antibody chosen from Table 1;

p) reduces (e.g., inhibits) one or more biological activities of Zika virus, in vitro, ex vivo, or in vivo;

q) reduces (e.g., inhibits) binding of Zika virus to a cell surface, fusion between a Zika virus E protein and an endosomal membrane, or both;

r) inhibits (e.g., neutralizes) Zika virus e.g., in a microneutralization assay;

s) inhibits (e.g., neutralizes) Zika virus with an EC50 of about 10 μg/mL or less, e.g., about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a method described herein, e.g., a microneutralization assay;

t) inhibits (e.g., neutralizes) Zika virus with an EC90 of about 20 μg/mL or less, e.g., about 10 μg/mL or less, about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a method described herein, e.g., a microneutralization assay;

u) does not bind, or does not substantially bind, to domain II of a Zika virus E protein, or binds to domain II of a Zika virus E protein with low affinity, e.g., with a dissociation constant (K_{D}) of about 500 nM or more, e.g., about 750 nM or more, about 1 μM or more, about 2 μM or more, about 3 μM or more, about 4 μM or more, or about 5 μM or more, e.g., as determined by a method described herein, e.g., ELISA or SPR;

v) binds, or substantially binds, to the same or overlapping epitope as an anti-ZIKV antibody molecule that binds to an E protein of Zika virus, e.g., domain III of the E protein; or

w) competes for binding to Zika virus with an anti-ZIKV antibody molecule that binds to an E protein of Zika virus, e.g., domain III of the E protein.

In an embodiment, the antibody molecule binds, or substantially binds, to the E protein of Zika virus, e.g., domain III of the E protein (e.g., the lateral ridge of domain III of the E protein).

In an embodiment, the antibody molecule binds, or substantially binds, to an epitope on Zika virus, e.g., an E protein of Zika virus, that is not cross-reactive to one or more different flaviviruses, e.g., dengue virus, Japanese encephalitis virus, West Nile virus, yellow fever virus, or combination thereof.

In an embodiment, the antibody molecule binds, or substantially binds, to an epitope on Zika virus comprising one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) amino acid residues in domain III of the E protein.

In an embodiment, the antibody molecule binds, or substantially binds, to an epitope on Zika virus, e.g., the same, similar, or overlapping epitope as the epitope recognized by a monoclonal antibody described in Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule binds, or substantially binds, to Zika virus (e.g., the E protein of Zika virus, e.g., domain III of the E protein) with high affinity, e.g., with a dissociation constant (K_{D}) of about 100 nM or less, e.g., about 10 nM or less, about 5 nM or less, about 1 nM or less, about 0.5 nM or less, about 0.1 nM or less, about 0.05 nM or less, or about 0.01 nM or less, e.g., about 10 nM to about 0.01 nM, about 5 nM to about 0.01 nM, about 3 to about 0.05 nM, or about 1 nM and about 0.1 nM, e.g., about 50 nM or less, about 40 nM or less, about 30 nM or less, about 20 nM or less, about 10 nM or less, about 8 nM or less, about 6 nM or less, about 4 nM or less, about 2 nM or less, about 1 nM or less, about 0.5 nM or less, about 0.2 nM or less, about 0.1 nM or less, about 0.05 nM or less, about 0.02 nM or less, or about 0.01 nM or less, e.g., as determined by a method described herein, e.g., ELISA or SPR.

In an embodiment, the antibody molecule shows the same or similar binding affinity or specificity, or both, as a monoclonal antibody described in Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule shows the same or similar binding affinity or specificity, or both, as an antibody molecule comprising a heavy chain variable region (VH) and/or a light chain variable region (VL) described in Table 1, e.g., a heavy chain variable region and/or a light chain variable region of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule shows the same or similar binding affinity or specificity, or both, as an antibody molecule comprising one or more (e.g., two or three) heavy chain CDRs (HCDR1, HCDR2, or HCDR3) and/or one or more (e.g., two or three) light chain CDRs (LCDR1, LCDR2, or LCDR3) described in Table 1, e.g., one or more (e.g., two or three) heavy chain CDRs and/or one or more (e.g., two or three) light chain CDRs of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule shows the same or similar binding affinity or specificity, or both, as an antibody molecule comprising an amino acid sequence shown in Table 1.

In an embodiment, the antibody shows the same or similar binding affinity or specificity, or both, as an antibody molecule comprising an amino acid sequence encoded by a nucleotide sequence shown in Table 2.

In an embodiment, the antibody molecule inhibits, e.g., competitively inhibits, the binding of a second antibody molecule to Zika virus, wherein the second antibody molecule is an antibody molecule chosen from Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule competes for binding with a second antibody molecule to Zika virus, wherein the second antibody molecule is an antibody molecule chosen from Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule has one or more biological properties of a monoclonal antibody chosen from Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule has one or more structural properties of a monoclonal antibody chosen from Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule has one or more pharmacokinetic properties of a monoclonal antibody chosen from Table 1, e.g., any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule reduces (e.g., inhibits) one or more biological activities of Zika virus, in vitro, ex vivo, or in vivo.

In an embodiment, the antibody molecule reduces (e.g., inhibits) binding of Zika virus to a cell surface, fusion between a Zika virus E protein and an endosomal membrane, or both.

In an embodiment, the antibody molecule inhibits (e.g., neutralizes) Zika virus e.g., in a microneutralization assay.

In an embodiment, the antibody molecule inhibits (e.g., neutralizes) Zika virus with an EC50 of about 10 μg/mL or less, e.g., about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a method described herein, e.g., a microneutralization assay.

In an embodiment, the antibody molecule inhibits (e.g., neutralizes) Zika virus with an EC90 of about 20 μg/mL or less, e.g., about 10 μg/mL or less, about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a method described herein, e.g., a microneutralization assay.

In an embodiment, the antibody molecule does not bind, or does not substantially bind, to domain II of the E protein, or binds to domain II of the E protein with low affinity, e.g., with a dissociation constant (K_{D}) of about 500 nM or more, e.g., about 750 nM or more, about 1 μM or more, about 2 μM or more, about 3 μM or more, about 4 μM or more, or about 5 μM or more, e.g., as determined by a method described herein, e.g., ELISA or SPR.

In an embodiment, the antibody molecule binds, or substantially binds, to the same or overlapping epitope as an anti-ZIKV antibody molecule that binds to the E protein of a Zika virus, e.g., domain III of the E protein.

In an embodiment, the antibody molecule competes for binding to Zika virus with an anti-ZIKV antibody molecule that binds to the E protein of a Zika virus, e.g., domain III of the E protein.

In an embodiment, the antibody molecule binds, or substantially binds, to one or more ZIKV strains chosen from Zika virus isolate Brazil-ZKV2015 (KU497555.1), Zika virus isolate SSABR1 (KU707826.1), Zika virus strain Natal RGN (KU527068.1), Zika virus strain BeH815744 (KU365780.1), Zika virus strain BeH819966 (KU365779.1), Zika virus strain BeH819015 (KU365778.1), Zika virus strain BeH818995 (KU365777.1), Zika virus strain ZikaSPH2015 (KU321639.1), Zika virus isolate Si322 (KU646828.1), Zika virus isolate Si323 (KU646827.1), Zika virus strain 103344 (KU501216.1), Zika virus strain 8375 (KU501217.1), Zika virus strain MRS_OPY_Martinique_PaRi_2015 (KU647676.1), Zika virus isolate Z1106027 (KU312315.1), Zika virus strain PRVABC59 (KU501215.1), Zika virus isolate Z1106032 (KU312313.1), Zika virus isolate Z1106031 (KU312314.1), Zika virus isolate Z1106033 (KU312312), Zika virus strain Haiti/1225/2014 (KU509998.1), Zika virus strain CK-ISL 2014 (KJ634273.1), Zika virus isolate Zika virus/H.sapiens-tc/THA/2014/SV0127-14 (KU681081.3), Zika virus strain H/PF/2013 (KJ776791.1), Zika virus strain PLCal_ZV (KF993678.1), Zika virus isolate Zika virus/H.sapiens-tc/PHL/2012/CPC-0740 (KU681082.3), Zika virus associated with an epidemic, Yap State, Micronesia, 2007 (EU545988.1), Zika virus isolate ARB13565 (KF268948.1), Zika virus isolate ArD_41519 (HQ234501.1), Zika virus isolate IbH_30656 (HQ234500.1), Zika virus isolate P6-740 (HQ234499.1), Zika virus strain (YP002790881.1), Zika virus strain (NC_012532.1), Zika virus isolate MR766-NIID (LC002520.1), Zika virus isolate MR_766 (HQ234498.1), Zika virus strain MR 766 (AY632535.2; DQ859059.1), Zika virus strain ArD158095 (KF383121.1), Zika virus isolate ARB15076 (KF268949.1), Zika virus isolate ARB7701 (KF268950.1), Zika virus strain ArD158084 (KF383119.1), Zika virus strain ArD157995 (KF383118.1), or Zika virus strain ArD7117 (KF383116.1).

In an embodiment, the antibody molecule binds, or substantially binds, to a Zika virus strain that comprises a glycosylation site at N154. In an embodiment, the antibody molecule binds, or substantially binds, to a Zika virus strain that confers neurovirulence.

In an embodiment, the antibody molecule comprises one or more (e.g., two or three) heavy chain CDRs (HCDR1, HCDR2, or HCDR3) described in Table 1, e.g., one or more (e.g., two or three) heavy chain CDRs of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule comprises one or more (e.g., two or three) light chain CDRs (LCDR1, LCDR2, or LCDR3) described in Table 1, e.g., one or more (e.g., two or three) light chain CDRs of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule comprises one or more (e.g., two or three) heavy chain CDRs (HCDR1, HCDR2, or HCDR3) and one or more (e.g., two or three) light chain CDRs (LCDR1, LCDR2, or LCDR3) described in Table 1, e.g., one or more (e.g., two or three) heavy chain CDRs and/or one or more (e.g., two or three) light chain CDRs of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule comprises a VH described in Table 1, e.g., a VH of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12, or a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the aforesaid VH amino acid sequence.

In an embodiment, the antibody molecule comprises a VL described in Table 1, e.g., a VL of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12, or a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the aforesaid VL amino acid sequence.

In an embodiment, the antibody molecule comprises a VH and a VL described in Table 1, e.g., a VH of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12, or a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the aforesaid VH amino acid sequence; and a VL of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12, or a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the aforesaid VL amino acid sequence.

In an embodiment, the antibody molecule is a Fab, F(ab′)2, Fv, or a single chain Fv fragment (scFv). In an embodiment, the antibody molecule comprises a heavy chain constant region chosen from IgG1, IgG2, IgG3, or IgG4. In an embodiment, the antibody molecule comprises a light chain constant region chosen from the light chain constant region of kappa or lambda.

In an embodiment, the antibody molecule is an isolated antibody molecule. In an embodiment, the antibody molecule is a synthetic antibody molecule. In an embodiment, the antibody molecule is a humanized antibody molecule. In an embodiment, the antibody molecule comprises one or more framework regions derived from a human framework germline sequence.

In an embodiment, the antibody molecule comprises an Fc region. In an embodiment, the Fc region comprises a half-life enhancing mutation, a mutation that is capable of disrupting an Fc effector function, or both. In an embodiment, the Fc region comprises one or more mutations or combinations of mutations described herein, e.g., chosen from M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof. Alternatively, or additionally, in an embodiment, the Fc region comprises (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V; (b) a mutation or a combination of mutations capable of disrupting an Fc effector function, e.g., L234A/L235A (also known as “LALA” mutation), or (c) both (a) and (b).

In an embodiment, the Fc region comprises mutations T256D/Q311V/A378V and a mutation or a combination of mutations capable of disrupting an Fc effector function, e.g., L234A/L235A. In an embodiment, the Fc region comprises mutations H285N/T307Q/N315D and a mutation or a combination of mutations capable of disrupting an Fc effector function, e.g., L234A/L235A. In an embodiment, the Fc region comprises mutations H285D/T307Q/A378V and a mutation or a combination of mutations capable of disrupting an Fc effector function, e.g., L234A/L235A. In an embodiment, the Fc region comprises mutations T307Q/Q311V/A378V and a mutation or a combination of mutations capable of disrupting an Fc effector function, e.g., L234A/L235A. In an embodiment, the Fc region comprises mutations T256D/N286D/T307R/Q311V/A378V and a mutation or a combination of mutations capable of disrupting an Fc effector function, e.g., L234A/L235A. In an embodiment, the Fc region comprises mutations T256D/T307R/Q311V and a mutation or a combination of mutations capable of disrupting an Fc effector function, e.g., L234A/L235A.

In another aspect, the disclosure features an anti-ZIKV antibody molecule, comprising a heavy chain variable region (VH) and a light chain variable region (VL), wherein the VH comprises three heavy chain complementarity determining regions (HCDR1, HCDR2, and HCDR3), and the VL comprises three light chain complementarity determining regions (LCDR1, LCDR2, and LCDR3), and wherein:

(a) the VH comprises: (i) an HCDR1 comprising the amino acid sequence G-Y-S/T-T-T/S-Y (SEQ ID NO: 351); (ii) an HCDR2 comprising the amino acid sequence Y-P-R-S-N/G-N/H (SEQ ID NO: 352); and (iii) an HCDR3 comprising the amino acid sequence E-N/D-Y-G-S-V/T/D-Y (SEQ ID NO: 353); and the VL comprises: (i) an LCDR1 comprising the amino acid sequence K/R-A-S-Q-S/N-V-G-T/E-A-V-A (SEQ ID NO: 354); (ii) an LCDR2 comprising the amino acid sequence S-A-S-N/D-R/L-Y-T (SEQ ID NO: 355); and (iii) an LCDR3 comprising the amino acid sequence Q-Q-F-S/Y-N/S-Y-P-F/Y-T (SEQ ID NO: 356); or

(b) the VH comprises: (i) an HCDR1 comprising the amino acid sequence S/T-Y-G-I-S(SEQ ID NO: 357); (ii) an HCDR2 comprising the amino acid sequence V-I-Y-P-R-S-N/G-N/H-T-Y-Y-N/A-E/Q-R/K-F/L-R/Q-G (SEQ ID NO: 358); and (iii) an HCDR3 comprising the amino acid sequence E-N/D-Y-G-S-V/T/D-Y (SEQ ID NO: 353); and the VL comprises: (i) an LCDR1 comprising the amino acid sequence K/R-A-S-Q-S/N-V-G-T/E-A-V-A (SEQ ID NO: 354); (ii) an LCDR2 comprising the amino acid sequence S-A-S-N/D-R/L-Y-T (SEQ ID NO: 355); and (iii) an LCDR3 comprising the amino acid sequence Q-Q-F-S/Y-N/S-Y-P-F/Y-T (SEQ ID NO: 356).

In an embodiment, the VH comprises: (i) an HCDR1 comprising the amino acid sequence of SEQ ID NO: 101 or 107; (ii) an HCDR2 comprising the amino acid sequence of any of SEQ ID NOs: 102, 108, or 114; or (iii) an HCDR3 comprising the amino acid sequence of any of SEQ ID NOs: 103, 113, 115, or 116; and the VL comprises: (i) an LCDR1 comprising the amino acid of any of SEQ ID NOs: 104, 109, 111, or 117; (ii) an LCDR2 comprising the amino acid of any of SEQ ID NOs: 105, 112, or 118; or (iii) an LCDR3 comprising the amino acid sequence of any of SEQ ID NOs: 106, 110, or 119.

In an embodiment, the VH comprises: (i) an HCDR1 comprising the amino acid sequence of SEQ ID NO: 201 or 203; (ii) an HCDR2 comprising the amino acid sequence of any of SEQ ID NOs: 202, 204, 205, 206, 207, 208, or 209; or (iii) an HCDR3 comprising the amino acid sequence of any of SEQ ID NOs: 103, 113, 115, or 116; and the VL comprises: (i) an LCDR1 comprising the amino acid of any of SEQ ID NOs: 104, 109, 111, or 117; (ii) an LCDR2 comprising the amino acid of any of SEQ ID NOs: 105, 112, or 118; or (iii) an LCDR3 comprising the amino acid sequence of any of SEQ ID NOs: 106, 110, or 119.

In an embodiment, the antibody molecule comprises the HCDR1, HCDR2, HCDR3, LCDR1, LCDR2, and LCDR3 of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the antibody molecule comprises a VH comprising the amino acid sequence of any of SEQ ID NOs: 1, 3, 5, 9, 10, 11, 12, 13, 14, 15, 16, 17, or 22, or a an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith. In an embodiment, the antibody molecule comprises a VH comprising the amino acid sequence of any of SEQ ID NOs: 1, 3, 5, 9, 10, 11, 12, 13, 14, 15, 16, 17, or 22.

In an embodiment, the antibody molecule comprises a VL comprising the amino acid sequence of any of SEQ ID NOs: 2, 4, 6, 7, 8, 18, 19, 20, or 21, or a an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith. In an embodiment, the antibody molecule comprises a VL comprising the amino acid sequence of any of SEQ ID NOs: 2, 4, 6, 7, 8, 18, 19, 20, or 21.

In an embodiment, the antibody molecule comprises:

(a) a VH comprising the amino acid sequence of SEQ ID NO: 1 and a VL comprising the amino acid sequence of SEQ ID NO: 2;

(b) a VH comprising the amino acid sequence of SEQ ID NO: 3 and a VL comprising the amino acid sequence of SEQ ID NO: 4;

(c) a VH comprising the amino acid sequence of SEQ ID NO: 5 and a VL comprising the amino acid sequence of SEQ ID NO: 6;

(d) a VH comprising the amino acid sequence of SEQ ID NO: 5 and a VL comprising the amino acid sequence of SEQ ID NO: 7;

(e) a VH comprising the amino acid sequence of SEQ ID NO: 5 and a VL comprising the amino acid sequence of SEQ ID NO: 8;

(f) a VH comprising the amino acid sequence of SEQ ID NO: 9 and a VL comprising the amino acid sequence of SEQ ID NO: 6;

(g) a VH comprising the amino acid sequence of SEQ ID NO: 9 and a VL comprising the amino acid sequence of SEQ ID NO: 7;

(h) a VH comprising the amino acid sequence of SEQ ID NO: 9 and a VL comprising the amino acid sequence of SEQ ID NO: 8;

(i) a VH comprising the amino acid sequence of SEQ ID NO: 10 and a VL comprising the amino acid sequence of SEQ ID NO: 6;

(j) a VH comprising the amino acid sequence of SEQ ID NO: 10 and a VL comprising the amino acid sequence of SEQ ID NO: 7;

(k) a VH comprising the amino acid sequence of SEQ ID NO: 10 and a VL comprising the amino acid sequence of SEQ ID NO: 8;

(l) a VH comprising the amino acid sequence of SEQ ID NO: 11 and a VL comprising the amino acid sequence of SEQ ID NO: 6;

(m) a VH comprising the amino acid sequence of SEQ ID NO: 11 and a VL comprising the amino acid sequence of SEQ ID NO: 7;

(n) a VH comprising the amino acid sequence of SEQ ID NO: 11 and a VL comprising the amino acid sequence of SEQ ID NO: 8;

(o) a VH comprising the amino acid sequence of SEQ ID NO: 12 and a VL comprising the amino acid sequence of SEQ ID NO: 6;

(p) a VH comprising the amino acid sequence of SEQ ID NO: 12 and a VL comprising the amino acid sequence of SEQ ID NO: 7;

(q) a VH comprising the amino acid sequence of SEQ ID NO: 12 and a VL comprising the amino acid sequence of SEQ ID NO: 8;

(r) a VH comprising the amino acid sequence of SEQ ID NO: 13 and a VL comprising the amino acid sequence of SEQ ID NO: 6;

(s) a VH comprising the amino acid sequence of SEQ ID NO: 13 and a VL comprising the amino acid sequence of SEQ ID NO: 18;

(t) a VH comprising the amino acid sequence of SEQ ID NO: 13 and a VL comprising the amino acid sequence of SEQ ID NO: 19;

(u) a VH comprising the amino acid sequence of SEQ ID NO: 13 and a VL comprising the amino acid sequence of SEQ ID NO: 20;

(v) a VH comprising the amino acid sequence of SEQ ID NO: 14 and a VL comprising the amino acid sequence of SEQ ID NO: 6;

(w) a VH comprising the amino acid sequence of SEQ ID NO: 14 and a VL comprising the amino acid sequence of SEQ ID NO: 18;

(x) a VH comprising the amino acid sequence of SEQ ID NO: 14 and a VL comprising the amino acid sequence of SEQ ID NO: 19;

(y) a VH comprising the amino acid sequence of SEQ ID NO: 14 and a VL comprising the amino acid sequence of SEQ ID NO: 20;

(z) a VH comprising the amino acid sequence of SEQ ID NO: 15 and a VL comprising the amino acid sequence of SEQ ID NO: 6;

(aa) a VH comprising the amino acid sequence of SEQ ID NO: 15 and a VL comprising the amino acid sequence of SEQ ID NO: 18;

(bb) a VH comprising the amino acid sequence of SEQ ID NO: 15 and a VL comprising the amino acid sequence of SEQ ID NO: 19;

(cc) a VH comprising the amino acid sequence of SEQ ID NO: 15 and a VL comprising the amino acid sequence of SEQ ID NO: 20;

(dd) a VH comprising the amino acid sequence of SEQ ID NO: 16 and a VL comprising the amino acid sequence of SEQ ID NO: 6;

(ee) a VH comprising the amino acid sequence of SEQ ID NO: 16 and a VL comprising the amino acid sequence of SEQ ID NO: 18;

(ff) a VH comprising the amino acid sequence of SEQ ID NO: 16 and a VL comprising the amino acid sequence of SEQ ID NO: 19;

(gg) a VH comprising the amino acid sequence of SEQ ID NO: 16 and a VL comprising the amino acid sequence of SEQ ID NO: 20;

(hh) a VH comprising the amino acid sequence of SEQ ID NO: 17 and a VL comprising the amino acid sequence of SEQ ID NO: 6;

(ii) a VH comprising the amino acid sequence of SEQ ID NO: 17 and a VL comprising the amino acid sequence of SEQ ID NO: 18;

(jj) a VH comprising the amino acid sequence of SEQ ID NO: 17 and a VL comprising the amino acid sequence of SEQ ID NO: 19;

(kk) a VH comprising the amino acid sequence of SEQ ID NO: 17 and a VL comprising the amino acid sequence of SEQ ID NO: 20;

(ll) a VH comprising the amino acid sequence of SEQ ID NO: 13 and a VL comprising the amino acid sequence of SEQ ID NO: 20;

(mm) a VH comprising the amino acid sequence of SEQ ID NO: 13 and a VL comprising the amino acid sequence of SEQ ID NO: 7;

(nn) a VH comprising the amino acid sequence of SEQ ID NO: 13 and a VL comprising the amino acid sequence of SEQ ID NO: 6;

(oo) a VH comprising the amino acid sequence of SEQ ID NO: 13 and a VL comprising the amino acid sequence of SEQ ID NO: 21;

(pp) a VH comprising the amino acid sequence of SEQ ID NO: 10 and a VL comprising the amino acid sequence of SEQ ID NO: 20;

(qq) a VH comprising the amino acid sequence of SEQ ID NO: 10 and a VL comprising the amino acid sequence of SEQ ID NO: 7;

(rr) a VH comprising the amino acid sequence of SEQ ID NO: 10 and a VL comprising the amino acid sequence of SEQ ID NO: 6;

(ss) a VH comprising the amino acid sequence of SEQ ID NO: 10 and a VL comprising the amino acid sequence of SEQ ID NO: 21;

(tt) a VH comprising the amino acid sequence of SEQ ID NO: 22 and a VL comprising the amino acid sequence of SEQ ID NO: 20;

(uu) a VH comprising the amino acid sequence of SEQ ID NO: 22 and a VL comprising the amino acid sequence of SEQ ID NO: 7;

(vv) a VH comprising the amino acid sequence of SEQ ID NO: 22 and a VL comprising the amino acid sequence of SEQ ID NO: 6; or

(ww) a VH comprising the amino acid sequence of SEQ ID NO: 22 and a VL comprising the amino acid sequence of SEQ ID NO: 21.

In an embodiment, the antibody molecule comprises an Fc region. In an embodiment, the Fc region comprises one or more mutations or one or more combinations of mutations described herein, e.g., chosen from M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof. Alternatively, or additionally, in an embodiment, the Fc region comprises (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V; (b) a mutation or a combination of mutations capable of disrupting an Fc effector function, e.g., L234A/L235A (also known as “LALA” mutation), or (c) both (a) and (b).

In an embodiment, the antibody molecule binds to the E protein of Zika virus, e.g., domain III of the E protein (e.g., the lateral ridge of domain III of the E protein).

In an embodiment, the antibody molecule binds to an epitope on Zika virus, e.g., an E protein of Zika virus, that is not cross-reactive to one or more different flaviviruses, e.g., dengue virus, Japanese encephalitis virus, West Nile virus, yellow fever virus, or combination thereof.

In an embodiment, the antibody molecule binds to an epitope on Zika virus comprising one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) amino acid residues in domain III of the E protein.

In an embodiment, the antibody molecule binds to Zika virus (e.g., the E protein of Zika virus, e.g., domain III of the E protein) with high affinity, e.g., with a dissociation constant (K_{D}) of about 100 nM or less, e.g., about 10 nM or less, about 5 nM or less, about 1 nM or less, about 0.5 nM or less, about 0.1 nM or less, about 0.05 nM or less, or about 0.01 nM or less, e.g., about 10 nM to about 0.01 nM, about 5 nM to about 0.01 nM, about 3 to about 0.05 nM, or about 1 nM and about 0.1 nM, e.g., about 50 nM or less, about 40 nM or less, about 30 nM or less, about 20 nM or less, about 10 nM or less, about 8 nM or less, about 6 nM or less, about 4 nM or less, about 2 nM or less, about 1 nM or less, about 0.5 nM or less, about 0.2 nM or less, about 0.1 nM or less, about 0.05 nM or less, about 0.02 nM or less, or about 0.01 nM or less, e.g., as determined by ELISA or SPR.

In an embodiment, the antibody molecule reduces (e.g., inhibits) one or more biological activities of Zika virus, in vitro, ex vivo, or in vivo.

In an embodiment, the antibody molecule reduces (e.g., inhibits) binding of Zika virus to a cell surface, fusion between a Zika virus E protein and an endosomal membrane, or both.

In an embodiment, the antibody molecule inhibits (e.g., neutralizes) Zika virus e.g., in a microneutralization assay.

In an embodiment, the antibody molecule inhibits (e.g., neutralizes) Zika virus with an EC50 of about 10 μg/mL or less, e.g., about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a microneutralization assay.

In an embodiment, the antibody molecule inhibits (e.g., neutralizes) Zika virus with an EC90 of about 20 μg/mL or less, e.g., about 10 μg/mL or less, about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a microneutralization assay.

In an embodiment, the antibody molecule does not bind to domain II of the E protein, or binds to domain II of the E protein with low affinity, e.g., with a dissociation constant (K_{D}) of about 500 nM or more, e.g., about 750 nM or more, about 1 μM or more, about 2 μM or more, about 3 μM or more, about 4 μM or more, or about 5 μM or more, e.g., as determined by ELISA or SPR.

In an embodiment, the antibody molecule binds to one or more ZIKV strains chosen from Zika virus isolate Brazil-ZKV2015 (KU497555.1), Zika virus isolate SSABR1 (KU707826.1), Zika virus strain Natal RGN (KU527068.1), Zika virus strain BeH815744 (KU365780.1), Zika virus strain BeH819966 (KU365779.1), Zika virus strain BeH819015 (KU365778.1), Zika virus strain BeH818995 (KU365777.1), Zika virus strain ZikaSPH2015 (KU321639.1), Zika virus isolate Si322 (KU646828.1), Zika virus isolate Si323 (KU646827.1), Zika virus strain 103344 (KU501216.1), Zika virus strain 8375 (KU501217.1), Zika virus strain MRS_OPY_Martinique_PaRi_2015 (KU647676.1), Zika virus isolate Z1106027 (KU312315.1), Zika virus strain PRVABC59 (KU501215.1), Zika virus isolate Z1106032 (KU312313.1), Zika virus isolate Z1106031 (KU312314.1), Zika virus isolate Z1106033 (KU312312), Zika virus strain Haiti/1225/2014 (KU509998.1), Zika virus strain CK-ISL 2014 (KJ634273.1), Zika virus isolate Zika virus/H. sapiens-tc/THA/2014/SV0127-14 (KU681081.3), Zika virus strain H/PF/2013 (KJ776791.1), Zika virus strain PLCal_ZV (KF993678.1), Zika virus isolate Zika virus/H.sapiens-tc/PHL/2012/CPC-0740 (KU681082.3), Zika virus associated with an epidemic, Yap State, Micronesia, 2007 (EU545988.1), Zika virus isolate ARB13565 (KF268948.1), Zika virus isolate ArD_41519 (HQ234501.1), Zika virus isolate IbH_30656 (HQ234500.1), Zika virus isolate P6-740 (HQ234499.1), Zika virus strain (YP002790881.1), Zika virus strain (NC_012532.1), Zika virus isolate MR766-NIID (LC002520.1), Zika virus isolate MR_766 (HQ234498.1), Zika virus strain MR 766 (AY632535.2; DQ859059.1), Zika virus strain ArD158095 (KF383121.1), Zika virus isolate ARB15076 (KF268949.1), Zika virus isolate ARB7701 (KF268950.1), Zika virus strain ArD158084 (KF383119.1), Zika virus strain ArD157995 (KF383118.1), or Zika virus strain ArD7117 (KF383116.1).

In an embodiment, the antibody molecule binds to a Zika virus strain comprising a glycosylation site at N154. In an embodiment, the antibody molecule binds to a Zika virus strain that confers neurovirulence.

In an embodiment, the antibody molecule is a Fab, F(ab′)2, Fv, or a single chain Fv fragment (scFv). In an embodiment, the antibody molecule comprises a heavy chain constant region chosen from IgG1, IgG2, IgG3, or IgG4. In an embodiment, the antibody molecule comprises a light chain constant region chosen from the light chain constant region of kappa or lambda.

In an embodiment, the antibody molecule is an isolated antibody molecule. In an embodiment, the antibody molecule is a synthetic antibody molecule. In an embodiment, the antibody molecule is a humanized antibody molecule. In an embodiment, the antibody molecule comprises one or more framework regions derived from a human framework germline sequence. In an embodiment, the antibody molecule is affinity matured antibody molecule.

In another aspect, the disclosure features an anti-ZIKV antibody molecule comprising a VH and a VL, wherein the VH comprises three heavy chain complementarity determining regions (HCDR1, HCDR2, and HCDR3), and the VL comprises three light chain complementarity determining regions (LCDR1, LCDR2, and LCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of an monoclonal antibody described in Table 1; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the monoclonal antibody; or (iii) an HCDR3 comprising an amino acid sequence that differs by no more than 1, 2, or 3 amino acid residues from, or has at least 85, 90, 95, 99 or 100% homology with, the amino acid sequence of the HCDR3 of the monoclonal antibody; and wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of the monoclonal antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the monoclonal antibody; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of the monoclonal antibody.

In an embodiment, the monoclonal antibody is chosen from ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In an embodiment, the VH comprises: (i) an HCDR1 comprising an amino acid sequence that differs by no more than 1, 2, or 3 amino acid residues from, or has at least 85, 90, 95, 99 or 100% homology with, the amino acid sequence of the HCDR1 of the monoclonal antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the monoclonal antibody; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the monoclonal antibody.

In an embodiment, the VL comprises: (i) an LCDR1 comprising an amino acid sequence that differs by no more than 1, 2, or 3 amino acid residues from, or has at least 85, 90, 95, 99 or 100% homology with, the amino acid sequence of the LCDR1 of the monoclonal antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the monoclonal antibody; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the monoclonal antibody.

In an embodiment, the VH comprises: an HCDR1 comprising the amino acid sequence of the HCDR1 of the monoclonal antibody; an HCDR2 comprising the amino acid sequence of the HCDR2 of the monoclonal antibody; and an HCDR3 comprising the amino acid sequence of the HCDR3 of the monoclonal antibody.

In an embodiment, the VL comprises: an LCDR1 comprising the amino acid sequence of the LCDR1 of the monoclonal antibody; an LCDR2 comprising the amino acid sequence of the LCDR2 of the monoclonal antibody; and an LCDR3 comprising the amino acid sequence of the LCDR3 of the monoclonal antibody.

In an embodiment, the antibody molecule comprises a VH comprising: an HCDR1 comprising the amino acid sequence of the HCDR1 of the monoclonal antibody; an HCDR2 comprising the amino acid sequence of the HCDR2 of the monoclonal antibody; and an HCDR3 comprising the amino acid sequence of the HCDR3 of the monoclonal antibody, and a VL comprising: an LCDR1 comprising the amino acid sequence of the LCDR1 of the monoclonal antibody; an LCDR2 comprising the amino acid sequence of the LCDR2 of the monoclonal antibody; and an LCDR3 comprising the amino acid sequence of the LCDR3 of the monoclonal antibody.

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of the monoclonal antibody; or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of the monoclonal antibody.

In an embodiment, the antibody molecule comprises a VH comprising the amino acid sequence of the VH of the monoclonal antibody. In an embodiment, the antibody molecule comprises a VL comprising the amino acid sequence of the VL of the monoclonal antibody. In an embodiment, the antibody molecule comprises a VH comprising the amino acid sequence of the VH of the monoclonal antibody, and a VL comprising the amino acid sequence of the VL of the monoclonal antibody.

In an embodiment, the antibody molecule comprises a VH encoded by a nucleotide sequence of the monoclonal antibody, e.g., as described in Table 2. In an embodiment, the antibody molecule comprises a VL encoded by a nucleotide sequence of the monoclonal antibody, e.g., as described in Table 2. In an embodiment, the antibody molecule comprises a VH encoded by a nucleotide sequence of the monoclonal antibody, e.g., as described in Table 2, and a VL encoded by a nucleotide sequence of the monoclonal antibody, e.g., as described in Table 2.

In an embodiment, the antibody molecule is a humanized monoclonal antibody. In an embodiment, the antibody molecule comprises one or more framework regions derived from a human framework germline sequence. In an embodiment, the antibody molecule is an affinity matured antibody.

In an embodiment, the antibody molecule comprises a heavy chain constant region chosen from IgG1, IgG2, IgG3, or IgG4. In an embodiment, the antibody molecule comprises a light chain constant region chosen from the light chain constant region of kappa or lambda. In an embodiment, the antibody molecule is a Fab, F(ab′)2, Fv, or a single chain Fv fragment (scFv).

In an embodiment, the antibody molecule is an isolated antibody molecule. In an embodiment, the antibody molecule is a synthetic antibody molecule.

In an embodiment, the antibody molecule comprises an Fc region. In an embodiment, the Fc region comprises one or more mutations or one or more combinations of mutations described herein, e.g., M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof. Alternatively, or additionally, in an embodiment, the Fc region comprises (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V; (b) a mutation or a combination of mutations capable of disrupting an Fc effector function, e.g., L234A/L235A (also known as “LALA” mutation), or (c) both (a) and (b).

In another aspect, the disclosure features an antibody molecule that competes with an antibody molecule described herein for binding to Zika virus, e.g., the E protein of Zika virus (e.g., domain III of the E protein).

In an embodiment, the antibody molecule competes with any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12, for binding to Zika virus, e.g., the E protein of Zika virus (e.g., domain III of the E protein).

In another aspect, the disclosure features an antibody molecule that binds to the same (or substantially the same) epitope as, or an epitope that overlaps (or substantially overlaps) with, the epitope of an antibody molecule described herein.

In an embodiment, the antibody molecule binds to the same (or substantially the same) epitope as, or an epitope that overlaps (or substantially overlaps) with, the epitope of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

In another aspect, the disclosure features an antibody molecule that binds to an epitope described herein, e.g., the E protein of Zika virus, e.g., domain III of the E protein (e.g., the lateral ridge of domain III of the E protein).

In another aspect, the disclosure features a method of treating a Zika virus infection. The method includes administering to a subject in need thereof an anti-ZIKV antibody molecule, e.g., an anti-ZIKV antibody molecule described herein, in an amount effective to treat the Zika virus infection, thereby treating the Zika virus infection.

In an embodiment, the antibody molecule is administered at dose of about 1 mg/kg to about 50 mg/kg, e.g., about 5 mg/kg to about 25 mg/kg, about 5 mg/kg to about 20 mg/kg, about 5 mg/kg to about 15 mg/kg, about 5 mg/kg to about 10 mg/kg, about 20 mg/kg to about 25 mg/kg, about 15 mg/kg to about 25 mg/kg, about 10 mg/kg to about 25 mg/kg, about 10 mg/kg to about 40 mg/kg, about 20 mg/kg to about 30 mg/kg, e.g., about 25 mg/kg or less, about 20 mg/kg or less, about 15 mg/kg or less, about 10 mg/kg or less, about 5 mg/kg or less, about 2 mg/kg or less, or about 1 mg/kg or less.

In an embodiment, the antibody molecule is administered to the subject after the subject is exposed to Zika virus, or is known or suspected to be exposed to Zika virus. In an embodiment, the antibody molecule is administered to the subject less than about 96 hours, e.g., less than about 72 hours, less than about 60 hours, less than about 48 hours, less than about 36 hours, less than about 24 hours, less than about 12 hours, or less than about 6 hours, after the subject is exposed to Zika virus, or is known or suspected to be exposed to Zika virus.

In an embodiment, the antibody molecule is administered to the subject prior to the subject being exposed to Zika virus, or known or suspected to be exposed to Zika virus.

In an embodiment, the antibody molecule is administered to the subject after, or in response to, one or more manifestations of a Zika virus infection, e.g., fever, rash, joint pain, headache, vomiting, conjunctivitis, or a combination thereof. In an embodiment, the antibody molecule is administered to the subject less than about 96 hours, e.g., less than about 72 hours, less than about 60 hours, less than about 48 hours, less than about 36 hours, less than about 24 hours, less than about 12 hours, or less than about 6 hours, after the subject exhibits one or more manifestations of Zika virus, e.g., fever, rash, joint pain, headache, vomiting, conjunctivitis, or a combination thereof. In an embodiment, the antibody molecule is administered to the subject prior to onset of one or more manifestations of a Zika virus infection, e.g., fever, rash, joint pain, headache, vomiting, conjunctivitis, or a combination thereof.

In an embodiment, the subject is pregnant human female. In an embodiment, the pregnant human female subject is in the first, second or third trimester of pregnancy. In an embodiment, the antibody molecule is administered as a single dose, e.g., for duration of pregnancy. In another embodiment, the antibody molecule is administered once a trimester or once a month, e.g., for duration of pregnancy.

In an embodiment, the subject is about 24 months of age or younger, e.g., about 18 months of age or younger, about 12 months of age or younger, about 6 months of age or younger, about 3 months of age or younger, about 2 months of age or younger, about 1 months of age or younger, or about 2 weeks of age or younger.

In an embodiment, the subject has, or is at risk of having, Zika fever. In an embodiment, the subject has, or is at risk of having, microcephaly, or is at risk of giving birth to a child having microcephaly. In an embodiment, the subject has, or is at risk of having, Guillain-Barrd syndrome (GBS). In an embodiment, the subject exhibits, or is at risk of exhibiting, one or more neurological abnormalities.

In an embodiment, the subject is at risk of being infected, or has been infected, with dengue virus (DENV), Japanese encephalitis virus (JEV), West Nile virus (WNV), chikungunya virus (CHIKV), or a combination thereof. In an embodiment, the subject has developed, or is detected to have, an anti-DENV antibody, an anti-JEV antibody, an anti-WNV antibody, an anti-CHIKV antibody, or a combination thereof.

In an embodiment, the antibody molecule is administered to the subject by parenteral administration, e.g., by intravenous, intramuscular, or subcutaneous administration.

In an embodiment, the antibody molecule is administered in combination with a second therapeutic agent or different therapeutic modality. In an embodiment, the second therapeutic agent or different therapeutic modality is chosen from a 4-aminoquinoline derivative (e.g., amodiaquine, chloroquine, or hydroxychloroquine), a nucleoside inhibitor (e.g., ribavirin or taribavirin), an interferon (e.g., interferon-alpha, pegylated interferon-alpha-2a, or pegylated interferon-alpha-2b), an RNA-dependent RNA polymerase (RdRp) inhibitor (e.g., BCX4430), an adenosine analogue (e.g., GS-5734 or NITD008), a vaccine (e.g., a Zika vaccine), a second antibody molecule (e.g., a second anti-ZIKV antibody molecule), or a combination thereof.

In an embodiment, the Zika vaccine is chosen from an inactivated purified vaccine, a virus-like particle (VLP) (e.g., with a pRME protein), a YF17DD chimeric vaccine, a DNA vaccine, a live dengue recombinant vaccine, a DNA vaccine (e.g., expressing a VLP), a live recombinant adenovirus, a recombinant Zika viral protein (e.g., plus ALHYDROGEL®), a lentivirus-vectored vaccine, a measles-vectored vaccine, a Zika targeted mutation live attenuated vaccine, a live VSV recombinant vaccine, an E protein (e.g., in a nanoparticle), a synthetic replilink peptide, ChimeriVax (YF17D), or a combination thereof.

In an embodiment, the antibody molecule is administered to reduce a risk of enhancing a flavivirus (e.g., dengue virus) infection in the subject.

In yet another aspect, the disclosure features a method of preventing a Zika virus infection. The method includes administering to a subject in need thereof an anti-ZIKV antibody molecule, e.g., an anti-ZIKV antibody molecule described herein, in an amount effective to prevent the Zika virus infection, thereby preventing the Zika virus infection.

In an embodiment, the antibody molecule is administered at dose of about 1 mg/kg to about 50 mg/kg, e.g., about 5 mg/kg to about 25 mg/kg, about 5 mg/kg to about 20 mg/kg, about 5 mg/kg to about 15 mg/kg, about 5 mg/kg to about 10 mg/kg, about 20 mg/kg to about 25 mg/kg, about 15 mg/kg to about 25 mg/kg, about 10 mg/kg to about 25 mg/kg, about 10 mg/kg to about 40 mg/kg, about 20 mg/kg to about 30 mg/kg, e.g., about 25 mg/kg or less, about 20 mg/kg or less, about 15 mg/kg or less, about 10 mg/kg or less, about 5 mg/kg or less, about 2 mg/kg or less, or about 1 mg/kg or less.

In an embodiment, the antibody molecule is administered to the subject prior to the subject being exposed to Zika virus, e.g., at least about 1 day, at least about 2 days, at least about 3 days, at least about 4 days, at least about 5 days, at least about 6 days, at least about 7 days, at least about 14 days, at least about 21 days, or at least about 28 days, prior to being exposed to Zika virus.

In an embodiment, the antibody molecule is administered to the subject less than about 96 hours, e.g., less than about 72 hours, less than about 60 hours, less than about 48 hours, less than about 36 hours, less than about 24 hours, less than about 12 hours, or less than about 6 hours, after the subject is exposed to Zika virus, or is known or suspected to be exposed to Zika virus.

In an embodiment, the antibody molecule is administered to the subject prior to onset of one or more manifestations of a Zika virus infection, e.g., fever, rash, joint pain, headache, vomiting, conjunctivitis, or a combination thereof.

In an embodiment, the subject is pregnant human female. In an embodiment, the pregnant human female subject is in the first, second or third trimester of pregnancy. In an embodiment, the antibody molecule is administered as a single dose, e.g., for duration of pregnancy. In another embodiment, the antibody molecule is administered once a trimester or once a month, e.g., for duration of pregnancy.

In an embodiment, the subject is about 24 months of age or younger, e.g., about 18 months of age or younger, about 12 months of age or younger, about 6 months of age or younger, about 3 months of age or younger, about 2 months of age or younger, about 1 months of age or younger, or about 2 weeks of age or younger.

In an embodiment, the subject is at risk of having, Zika fever. In an embodiment, the subject is at risk of having microcephaly. In an embodiment, the subject is at risk of having microcephaly, or is at risk of giving birth to a child having microcephaly. In an embodiment, the subject is at risk of having Guillain-Barrd syndrome (GBS). In an embodiment, the subject is at risk of exhibiting one or more neurological abnormalities.

In an embodiment, the subject is at risk of being infected, or has been infected, with dengue virus (DENV), Japanese encephalitis virus (JEV), West Nile virus (WNV), chikungunya virus (CHIKV), or a combination thereof. In an embodiment, the subject has developed, or is detected to have, an anti-DENV antibody, an anti-JEV antibody, an anti-WNV antibody, an anti-CHIKV antibody, or a combination thereof.

In an embodiment, the antibody molecule is administered to the subject by parenteral administration, e.g., by intravenous, intramuscular, or subcutaneous administration.

In an embodiment, the antibody molecule is administered in combination with a second therapeutic agent or different therapeutic modality. In an embodiment, the second therapeutic agent or different therapeutic modality is chosen from a 4-aminoquinoline derivative (e.g., amodiaquine, chloroquine, or hydroxychloroquine), a nucleoside inhibitor (e.g., ribavirin or taribavirin), an interferon (e.g., interferon-alpha, pegylated interferon-alpha-2a, or pegylated interferon-alpha-2b), an RNA-dependent RNA polymerase (RdRp) inhibitor (e.g., BCX4430), an adenosine analogue (e.g., GS-5734 or NITD008), a Zika vaccine, a second anti-ZIKV antibody molecule, or a combination thereof.

In an embodiment, the Zika vaccine is chosen from an inactivated purified vaccine, a virus-like particle (VLP) (e.g., with a pRME protein), a YF17DD chimeric vaccine, a DNA vaccine, a live dengue recombinant vaccine, a DNA vaccine (e.g., expressing a VLP), a live recombinant adenovirus, a recombinant Zika viral protein (e.g., plus ALHYDROGEL®), a lentivirus-vectored vaccine, a measles-vectored vaccine, a Zika targeted mutation live attenuated vaccine, a live VSV recombinant vaccine, an E protein (e.g., in a nanoparticle), a synthetic replilink peptide, ChimeriVax (YF17D), or a combination thereof.

In an embodiment, the antibody molecule is administered to reduce a risk of enhancing a flavivirus (e.g., dengue virus) infection in the subject.

In another aspect, the disclosure features a method of inhibiting Zika virus. The method includes contacting a cell or a subject infected with Zika virus with an anti-ZIKV antibody molecule, e.g., an anti-ZIKV antibody molecule described herein, in an amount effective to inhibit Zika virus, thereby inhibiting Zika virus.

In an embodiment, the antibody molecule is contacted with the cell in vitro, ex vivo, or in vivo. In an embodiment, the method includes administering to the subject the antibody molecule.

In an embodiment, the antibody molecule is administered at dose of about 1 mg/kg to about 50 mg/kg, e.g., about 5 mg/kg to about 25 mg/kg, about 5 mg/kg to about 20 mg/kg, about 5 mg/kg to about 15 mg/kg, about 5 mg/kg to about 10 mg/kg, about 20 mg/kg to about 25 mg/kg, about 15 mg/kg to about 25 mg/kg, about 10 mg/kg to about 25 mg/kg, about 10 mg/kg to about 40 mg/kg, about 20 mg/kg to about 30 mg/kg, e.g., about 25 mg/kg or less, about 20 mg/kg or less, about 15 mg/kg or less, about 10 mg/kg or less, about 5 mg/kg or less, about 2 mg/kg or less, or about 1 mg/kg or less.

In an embodiment, the antibody molecule is administered to the subject prior to the subject being exposed to Zika virus, e.g., at least about 1 day, at least about 2 days, at least about 3 days, at least about 4 days, at least about 5 days, at least about 6 days, at least about 7 days, at least about 14 days, at least about 21 days, or at least about 28 days, prior to being exposed to Zika virus.

In an embodiment, the antibody molecule is administered to the subject after the subject is exposed to Zika virus, or is known or suspected to be exposed to Zika virus, e.g., less than about 96 hours, e.g., less than about 72 hours, less than about 60 hours, less than about 48 hours, less than about 36 hours, less than about 24 hours, less than about 12 hours, or less than about 6 hours, after the subject is exposed to Zika virus, or is known or suspected to be exposed to Zika virus.

In an embodiment, the antibody molecule is administered to the subject prior to onset of one or more manifestations of Zika virus, e.g., fever, rash, joint pain, headache, vomiting, conjunctivitis, or a combination thereof. In an embodiment, the antibody molecule is administered to the subject less than about 96 hours, e.g., less than about 72 hours, less than about 60 hours, less than about 48 hours, less than about 36 hours, less than about 24 hours, less than about 12 hours, or less than about 6 hours, after the subject exhibits one or more manifestations of Zika virus, e.g., fever, rash, joint pain, headache, vomiting, conjunctivitis, or a combination thereof.

In an embodiment, the subject is pregnant human female. In an embodiment, the pregnant human female subject is in the first, second or third trimester of pregnancy. In an embodiment, the antibody molecule is administered as a single dose, e.g., for duration of pregnancy. In another embodiment, the antibody molecule is administered once a trimester or once a month, e.g., for duration of pregnancy.

In an embodiment, the subject is about 24 months of age or younger, e.g., about 18 months of age or younger, about 12 months of age or younger, about 6 months of age or younger, about 3 months of age or younger, about 2 months of age or younger, about 1 months of age or younger, or about 2 weeks of age or younger.

In an embodiment, the subject has, or is at risk of having, Zika fever. In an embodiment, the subject has, or is at risk of having, microcephaly, or is at risk of giving birth to a child having microcephaly. In an embodiment, the subject has, or is at risk of having, Guillain-Barrd syndrome (GBS). In an embodiment, the subject exhibits, or is at risk of exhibiting, one or more neurological abnormalities.

In an embodiment, the subject is at risk of, or has been infected with, dengue virus (DENV), Japanese encephalitis virus (JEV), West Nile virus (WNV), chikungunya virus (CHIKV), or a combination thereof. In an embodiment, the subject has developed, or is detected to have, an anti-DENV antibody, an anti-JEV antibody, an anti-WNV antibody, an anti-CHIKV antibody, or a combination thereof.

In an embodiment, the antibody molecule is administered to the subject by parenteral administration, e.g., by intravenous, intramuscular, or subcutaneous administration.

In an embodiment, the antibody molecule is administered in combination with a second therapeutic agent or different therapeutic modality. In an embodiment, the second therapeutic agent or different therapeutic modality is chosen from a 4-aminoquinoline derivative (e.g., amodiaquine, chloroquine, or hydroxychloroquine), a nucleoside inhibitor (e.g., ribavirin or taribavirin), an interferon (e.g., interferon-alpha, pegylated interferon-alpha-2a, or pegylated interferon-alpha-2b), an RNA-dependent RNA polymerase (RdRp) inhibitor (e.g., BCX4430), a nucleotide analogue (e.g., BCX4430, GS-5734 or NITD008), a Zika vaccine, a second anti-ZIKV antibody molecule, or a combination thereof.

In an embodiment, the Zika vaccine is chosen from an inactivated purified vaccine, a virus-like particle (VLP) (e.g., with a pRME protein), a YF17DD chimeric vaccine, a DNA vaccine, a live dengue recombinant vaccine, a DNA vaccine (e.g., expressing a VLP), a live recombinant adenovirus, a recombinant Zika viral protein (e.g., plus ALHYDROGEL®), a lentivirus-vectored vaccine, a measles-vectored vaccine, a Zika targeted mutation live attenuated vaccine, a live VSV recombinant vaccine, an E protein (e.g., in a nanoparticle), a synthetic replilink peptide, ChimeriVax (YF17D), or a combination thereof.

In an embodiment, the antibody molecule is administered to reduce a risk of enhancing a flavivirus (e.g., dengue virus) infection in the subject.

In yet another aspect, the disclosure features a method of reducing a risk of enhancing a flavivirus (e.g., dengue virus) infection in a subject. The method includes: (a) identify a subject infected, or is at risk of being infected, with Zika virus; and (b) administering to the subject an anti-ZIKV antibody molecule that is not cross-reactive with the flavivirus (e.g., dengue virus), e.g., an anti-ZIKV antibody molecule described herein, thereby reducing the risk of enhancing the flavivirus (e.g., dengue virus) infection.

In an embodiment, the antibody molecule is administered at dose of about 1 mg/kg to about 50 mg/kg, e.g., about 5 mg/kg to about 25 mg/kg, about 5 mg/kg to about 20 mg/kg, about 5 mg/kg to about 15 mg/kg, about 5 mg/kg to about 10 mg/kg, about 20 mg/kg to about 25 mg/kg, about 15 mg/kg to about 25 mg/kg, about 10 mg/kg to about 25 mg/kg, about 10 mg/kg to about 40 mg/kg, about 20 mg/kg to about 30 mg/kg, e.g., about 25 mg/kg or less, about 20 mg/kg or less, about 15 mg/kg or less, about 10 mg/kg or less, about 5 mg/kg or less, about 2 mg/kg or less, or about 1 mg/kg or less.

In an embodiment, the antibody molecule is administered to the subject prior to the subject being exposed to Zika virus, e.g., at least about 1 day, at least about 2 days, at least about 3 days, at least about 4 days, at least about 5 days, at least about 6 days, at least about 7 days, at least about 14 days, at least about 21 days, or at least about 28 days, prior to being exposed to Zika virus.

In an embodiment, the antibody molecule is administered to the subject after the subject is exposed to Zika virus, or is known or suspected to be exposed to Zika virus, e.g., less than about 96 hours, e.g., less than about 72 hours, less than about 60 hours, less than about 48 hours, less than about 36 hours, less than about 24 hours, less than about 12 hours, or less than about 6 hours, after the subject is exposed to Zika virus, or is known or suspected to be exposed to Zika virus.

In an embodiment, the antibody molecule is administered to the subject prior to onset of one or more manifestations of Zika virus, e.g., fever, rash, joint pain, headache, vomiting, conjunctivitis, or a combination thereof. In an embodiment, the antibody molecule is administered to the subject less than about 96 hours, e.g., less than about 72 hours, less than about 60 hours, less than about 48 hours, less than about 36 hours, less than about 24 hours, less than about 12 hours, or less than about 6 hours, after the subject exhibits one or more manifestations of Zika virus, e.g., fever, rash, joint pain, headache, vomiting, conjunctivitis, or a combination thereof.

In an embodiment, the subject is pregnant human female. In an embodiment, the pregnant human female subject is in the first, second or third trimester of pregnancy. In an embodiment, the antibody molecule is administered as a single dose, e.g., for duration of pregnancy. In another embodiment, the antibody molecule is administered once a trimester or once a month, e.g., for duration of pregnancy.

In an embodiment, the subject is about 24 months of age or younger, e.g., about 18 months of age or younger, about 12 months of age or younger, about 6 months of age or younger, about 3 months of age or younger, about 2 months of age or younger, about 1 months of age or younger, or about 2 weeks of age or younger.

In an embodiment, the subject has, or is at risk of having, Zika fever. In an embodiment, the subject has, or is at risk of having, microcephaly, or is at risk of giving birth to a child having microcephaly. In an embodiment, the subject has, or is at risk of having, Guillain-Barré syndrome (GBS). In an embodiment, the subject exhibits one or more neurological abnormalities.

In an embodiment, the subject is at risk of having an infection with dengue virus (DENV), Japanese encephalitis virus (JEV), West Nile virus (WNV), chikungunya virus (CHIKV), or a combination thereof. In an embodiment, the subject is at risk of developing a disorder associated with an infection with dengue virus (DENV), Japanese encephalitis virus (JEV), West Nile virus (WNV), chikungunya virus (CHIKV), or a combination thereof.

In an embodiment, the antibody molecule is administered to the subject by parenteral administration, e.g., by intravenous, intramuscular, or subcutaneous administration.

In an embodiment, the antibody molecule is administered in combination with a second therapeutic agent or different therapeutic modality. In an embodiment, the second therapeutic agent or different therapeutic modality is chosen from a 4-aminoquinoline derivative (e.g., amodiaquine, chloroquine, or hydroxychloroquine), a nucleoside inhibitor (e.g., ribavirin or taribavirin), an interferon (e.g., interferon-alpha, pegylated interferon-alpha-2a, or pegylated interferon-alpha-2b), an RNA-dependent RNA polymerase (RdRp) inhibitor (e.g., BCX4430), a nucleotide analogue (e.g., BCX4430, GS-5734 or NITD008), a Zika vaccine, a second anti-ZIKV antibody molecule, or a combination thereof.

In an embodiment, the Zika vaccine is chosen from an inactivated purified vaccine, a virus-like particle (VLP) (e.g., with a pRME protein), a YF17DD chimeric vaccine, a DNA vaccine, a live dengue recombinant vaccine, a DNA vaccine (e.g., expressing a VLP), a live recombinant adenovirus, a recombinant Zika viral protein (e.g., plus ALHYDROGEL®), a lentivirus-vectored vaccine, a measles-vectored vaccine, a Zika targeted mutation live attenuated vaccine, a live VSV recombinant vaccine, an E protein (e.g., in a nanoparticle), a synthetic replilink peptide, ChimeriVax (YF17D), or a combination thereof.

In an aspect, the disclosure features an anti-ZIKV antibody molecule described herein for treating or preventing a Zika virus infection. In another aspect, the disclosure features an anti-ZIKV antibody molecule described herein for inhibiting Zika virus.

In an aspect, the disclosure features use of an anti-ZIKV antibody molecule described herein for the manufacture of a medicament for treating or preventing a Zika virus infection. In another aspect, the disclosure features use of an anti-ZIKV antibody molecule described herein for the manufacture of a medicament for inhibiting Zika virus.

In an aspect, the disclosure features a method of detecting Zika virus in a sample from a subject. The method includes: (i) contacting the sample (and optionally, a reference sample) with an anti-ZIKV antibody molecule, e.g., an antibody molecule described herein, under conditions that allow interaction of the antibody molecule and Zika virus (or a polypeptide from Zika virus) to occur, and (ii) detecting formation of a complex between the antibody molecule and the sample (and optionally, the reference sample), thereby detecting Zika virus.

In an aspect, the disclosure features an immunogen comprising an epitope described herein, e.g., comprising one or more residues in domain III of a Zika virus E protein. In another aspect, the disclosure features a vaccine comprising an immunogen described herein, or a nucleic acid encoding an immunogen described herein.

In an aspect, the disclosure features a composition, e.g., a pharmaceutical composition, comprising an anti-ZIKV antibody molecule described herein. In another aspect, the disclosure features a composition, e.g., a pharmaceutical composition, comprising a vaccine described herein. In an embodiment, the composition further comprises a pharmaceutical acceptable carrier.

In an aspect, the disclosure features a nucleic acid molecule encoding a heavy chain variable region (VH), a light chain variable region (VL), or both, of an antibody molecule described herein. In another aspect, the disclosure features a nucleic acid molecule encoding an immunogen or vaccine described herein.

In an aspect, the disclosure features a vector comprising a nucleic acid molecule described herein. In another aspect, the disclosure features a cell, e.g., an isolated cell, comprising a nucleic acid molecule described herein or a vector described herein.

In an aspect, the disclosure features a kit comprising an antibody molecule described herein and instructions to use of the antibody molecule. In another aspect, the disclosure features a kit comprising a vaccine described herein and instructions to use the vaccine.

In an aspect, the disclosure features a container comprising an antibody molecule described herein. In another aspect, the disclosure features a container comprising a vaccine described herein.

In an aspect, the disclosure features a method of producing an anti-ZIKV antibody molecule, the method comprising culturing a cell described herein under conditions that allow production of an antibody molecule described herein, thereby producing the antibody molecule. In an embodiment, the method further comprises isolating or purifying the antibody molecule.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIG. 1A depicts a pairwise identity matrix of all E protein residues between selected flavivirus sequences from the 4 serotypes of DENV, and ZIKV, JEV, and WNV.

FIG. 1B depicts a non-metric multidimensional scaling (NMDS) plot for select flavivirus sequences calculated using all residues (top left) or only surface exposed residues (top right). Clustering is also shown for each of the individual domains (bottom panel) calculated using only exposed residues.

FIG. 1C depicts the similarity of surface residues for select flaviviruses mapped to reference structures of DENV1 (left) or DENV4 (right). Residue similarity is defined using a PAM60 substitution matrix, and for clarity of visualization, is capped at a minimum of −5.0 and a maximum of 5.0.

FIG. 1D depicts the result of sequence analysis of E protein domain III from DENV, YFV, ZIKV, WNV, and JEV.

FIG. 2A depicts the epitope on DENV2 dimer highlighted in light grey (indicated as “1”) for EDE antibodies C8 and A11 and WNV FLE antibody E53. The N153 glycan is shown in black (indicated as “2”); for clarity, the N153 glycan on the left-side of the dimer has been removed. Epitope was determined using a 4.5 Å distance cutoff from antibody residues in co-crystal structures.

FIG. 2B depicts the epitope footprint of A11 (circle) on DENV4 structure color coded by similarity to DENV1 (top), and ZIKV (bottom). Greyscale coding uses same scale as shown in FIG. 1C.

FIG. 3 depicts the residues in contact with EDE2 antibody A11. ZIKV residues that are similar to those found in DENV1-4 are positions 71, 72, 73, 97, 98, 99, 101, 102, 103, 104, 113, 152, 153, 154, 155, 157, 245, 246, 247, and 248, with different residues being positions 68, 69, 70, and 249. The N153-glycan is indicated. The residue conservation is shown (middle panel) along with the number of atomic contacts made per epitope position (bottom panel).

FIG. 4 depicts a multiple sequence alignment of E protein for select flaviviruses.

FIG. 5 depicts the exposed residues that are defined based on general surface accessibility in the context of the virion (left). Residues that are considered exposed are shown in spheres (right) in a top-down view (top) or side-view (bottom).

FIG. 6 depicts the similarity of surface residues for select flaviviruses mapped to reference structures of DENV2 (left) or DENV3 (right). Residue similarity is defined using a PAM60 substitution matrix, and for clarity of visualization, is capped at a minimum of −5.0 and a maximum of 5.0.

FIG. 7 depicts the similarity of surface patches for DENV4 and ZIKV plotted for select residues (spheres) tracing along an E protein structure. Surface patches are defined by neighboring residues within 5 Å of the central selected residue. The similarity for a patch is the mean similarity for all residues in the patch.

FIG. 8 depicts pairwise identity matrices for exposed residues in each of the 3 domains of the E protein (bottom). Pairwise identity is also shown for a subset of exposed residues in Domain II which form a region for which ZIKV was shown to have high similarity to DENV4 (residues 58-125 and 214-255 using DENV1 numbering).

FIG. 9A depicts a sequence alignment of prM protein for select flaviviruses.

FIG. 9B depicts a pairwise identity matrix of prM proteins for select viruses for all residues (left) and exposed residues (right).

FIG. 10 depicts a sequence alignment for select flaviviruses, showing region encompassing the Asn153 glycosylation loop.

FIG. 11 depicts a sequence alignment for ZIKV viruses (sorted by date), showing region encompassing the Asn154 glycosylation loop.

FIG. 12A depicts the inhibitory effect of VIS513 on plaque formation in the presence or absence of naïve serum.

FIG. 12B depicts the maintenance of VIS513 activity in the presence of DENV-2 immune serum.

FIG. 12C depicts the inhibitory effect of VIS513 on plaque formation by DENV-2 in the presence heterologous, enhancing immune serum to DENV-1.

FIG. 13A depicts binding of 6 ZIKV scaffold monoclonal antibodies and a dengue EDIII antibody VIS513 to ZIKV EDIII (EDIII-ZV) protein.

FIG. 13B depicts binding of 6 ZIKV scaffold monoclonal antibodies and a dengue EDIII antibody VIS513 to DENV EDIII (EDIII-DV1) protein.

FIG. 14 depicts in vitro neutralization effects of ZIKV scaffold monoclonal antibodies.

FIG. 15A depicts reduced viremia levels in mice inoculated with ZIKV virus and treated with ZIKV monoclonal antibodies ZV-54 and ZV-67, in contrast to animals treated with a control Chikungunya antibody (CHK-166).

FIG. 15B depicts reduced weight loss in animals treated with both ZIKV monoclonal antibodies.

FIG. 15C depicts decreased mortality in animals treated with both ZIKV monoclonal antibodies when compared to control antibody treated animals.

FIG. 16A depicts the identity of each chimeric protein to WNV or ZKV EDIII proteins.

FIG. 16B is a schematic representation of each chimeric protein in relation to WNV and ZIKV ED111 proteins.

FIG. 17 depicts the affinity (K_{D}) of ZIKV monoclonal antibodies calculated using the yeast surface display method.

FIG. 18 depicts binding patterns of 6 ZIKV monoclonal antibodies to WT or chimeric EDIII proteins displayed on the surface of yeast.

FIGS. 19A-19B depict the schematic representation of the results of the epitope mapping experiment from FIG. 18.

FIG. 20A depicts the epitope bound by ZV-54 and ZV-67 as the lateral ridge of EDIII.

FIG. 20B depicts the residues on the chimeric protein EDIII which are critical for binding to ZV-54 and ZV-67. Residues important for binding (indicated as “1”) and residues critical for binding (indicated as “2) are shown.

FIG. 20C depicts a spatially identical epitope on EDIII which is the binding site of a potent neutralizing WNV antibody.

FIG. 21 depicts the three heavy and light chain genes chosen for humanization.

FIG. 22 depicts superior in vitro neutralization from clones 3-2 and 5-1 which were derived from the Series A humanization.

FIG. 23 depicts a model of the humanized A-3/2 antibody docked to the ZIKV EDIII protein.

FIG. 24A depicts the sequence of A-3/2 and the sites for affinity maturation.

FIG. 24B depicts the specific mutations for affinity maturation.

FIG. 25 depicts enhanced neutralization potency of clones obtained from the affinity maturation process.

FIG. 26 depicts migration of lead candidate antibodies in SDS PAGE FIG. 27 depicts migration of lead candidate antibodies in non-reducing SDS PAGE.

FIG. 28 depicts results from a size exclusion chromatography experiment showing appropriate retention time of all lead candidates.

FIG. 29 depicts the thermal stability of all lead candidates.

FIG. 30 depicts lower non-specific binding activity of ZC10 in comparison to higher non-specific binding activity of ZC1 measured with an assay developed by Retrogenix.

FIG. 31A depicts K_{d}, Kₒₙ and K_{off} of Fc engineered antibodies.

FIG. 31B depicts improved binding to FcRn at pH 6.0 and poor binding to FcRn at pH 7.4 of a representative Fc engineered antibody.

FIG. 31C depicts the interaction of the CH2 domain of the antibody Fc with the FcRn molecule.

FIG. 32 depicts biophysical properties of Fc engineered variants.

FIG. 33A depicts improved in vivo half-life of 3 Motavizumab Fc variants in comparison to WT Motavizumab (Mota-WT).

FIG. 33B depicts pharmacokinetic properties of 2 Motavizumab Fc variants and WT Motavizumab.

FIG. 34A depicts improved in vivo half-life of later stage Motavizumab Fc variants in comparison to WT Motavizumab (Mota-WT).

FIG. 34B depicts pharmacokinetic properties of later stage Motavizumab Fc variants and WT Motavizumab.

FIG. 35 depicts longer in vivo half-life of the ZVB Zika antibody.

FIG. 36 depicts a similar half-life of ZKB-LS (ZVB antibody with LS Fc mutation) and ZKB-156 (ZVB antibody with Visterra 156 Fc mutation). Both of these antibodies had a longer half-life than the parental ZVB antibody (ZKB) and WT Motavizumab.

### DETAILED DESCRIPTION

Disclosed herein are antibody molecules that bind to Zika virus, e.g., the envelop (E) protein, e.g., domain III of the E protein, with high affinity and specificity. The antibody molecules described herein can reduce (e.g., inhibit, block, or neutralize) one or more biological activities of Zika virus. While not wishing to be bound by theory, it is believed that in an embodiment, the antibody molecules describe herein reduce the risk of antibody-dependent enhancement of viral infection, by targeting certain region(s) on the E protein, e.g., domain III of the E protein. Further provided herein are vaccines and immunogens that can induce an immune response against, or confers protection against, Zika virus. In an embodiment, the vaccine or immunogen comprises an epitope described herein, e.g., an epitope comprising one or more residues in domain III of the E protein. Nucleic acid molecules encoding the antibody molecules, vaccines or immunogens, expression vectors, host cells, compositions (e.g., pharmaceutical compositions), kits, containers, and methods for making the antibody molecules or vaccines, are also provided herein. The antibody molecules, compositions (e.g., pharmaceutical compositions), and vaccines disclosed herein can be used (alone or in combination with other agents or therapeutic modalities) to treat, prevent and/or diagnose disorders and conditions, e.g., Zika virus infection, and disorders and conditions associated with Zika infection, e.g., Zika fever, microcephaly, Guillain-Barre Syndrome, or neurological abnormalities.

The disclosure herein, is at least in part, based on the analysis of the surface accessible region of the flavivirus E proteins, which indicates that epitopes important for recognition by dominant dengue virus cross-reactive antibodies mostly localized to the fusion loop and domain II of the E protein. These cross-reactive antibodies potentially can cross-react with Zika virus, leading to binding and FcγR-mediated uptake into cells but not neutralization, thereby raising the possibility of antibody-dependent enhancement. Several studies of Zika outbreaks in Yap Island and French Polynesia have indicated that convalescent serum individuals recovering from Zika virus infection contain cross-reactive antibodies to dengue virus (Duffy et al. N Engl J Med. 2009; 360(24): 2536-43). Zika virus is rapidly spreading in regions endemic to dengue virus. Previous infection and/or vaccination with dengue virus could mediate antibody-dependent enhancement (ADE) of disease severity.

The antibody molecules described herein (e.g., Zika virus-specific, neutralizing antibody molecules targeting domain III of the E protein) can have a number of advantages, e.g., compared to antibodies targeting cross-reactive epitopes and traditional vaccine or small molecule approaches. For example, unlike with domain II and the fusion loop, there is little epitope overlap between Zika virus and dengue virus domain III, lowering the potential for cross-reactivity and hence enhancement of viral infection. Most human antibodies bind to fusion loop or domain II, which may result in increased likelihood for generating an antibody response that binds both but does not neutralizaing one or the other, or increased potential to enhance disease and/or increase transplacental transport of ZIKV. Induction of antibody dependent enhancement (ADE) from vaccines is generally irreversible. Autoantibodies, induced by vaccines, can cause Guillain-Barrd syndrome (GBS). Live attenuated vaccine is contraindicated or not desirable for pregnancy. Flavivirus inactivated/subunit vaccine strategies often require multiple boosters and are prone to waning antibody titers. There is also need for high antibody titers to prevent low-level breakthrough to fetus. Small molecule strategies may have high probability of off-target toxicity (e.g., to developing fetus) and/or require repeat dosing. Without wishing to be bound by theory, it is believed that in an embodiment, prophylaxis with a neutralizing antibody early in pregnancy represents a practical and rapid option for protection of mothers throughout pregnancy, as maternal antibody is transferred to fetus, thereby providing protection.

Monoclonal antibodies historically have shown good safety profiles, especially for non-human targets. Monoclonal antibodies can also have longer half-lives, which allow for the potential of single dose administration. The antibody approach described herein can reduce or mitigate ADE.

Antibodies with high neutralizing activity can mediate passive sterilizing immunity. Certain antibodies targeting domain III of the dengue E protein are strongly neutralizing (Beltramello et al. Cell Host & Microbe 2010; 8(3): 271-83; Robinson et al. Cell 2015; 162(3): 493-504). Antibodies directed to domain III are believed to be much less frequently elicited in the human antibody repertoire, as such, exogenous administration of these antibodies does not interfere, and in fact can promote, the neutralizing capacity of human serum. For example, the neutralizing activity of a domain III antibody against dengue virus is greater in the presence of serum than without addition (see Example 7). Targeting a viral epitope that is not normally targeted by the human immune system can have a number of advantages. For example, without wishing to be bound by theory, it is believed that, the antibody molecules described herein do not cause significant immune interference, can attenuate virus, if present, to promote natural immunity, and/or target an epitope that is more refractory to development of resistance. The antibody strategies described herein can have one or more of the following properties: (i) long term protection (e.g., at least 6-9 months); (ii) pre-/post-exposure prophylaxis in high risk populations such as pregnant women; (iii) no enhancement of disease by low/falling titers of the antibody; (iv) well tolerated with no or minimal allergic reactions; or (v) suitable for use in various hosts including immunocompromised patients.

The approach of generating ZIKV neutralizing antibodies can include, e.g., (i) structural and computational assessment of the ZIKV E protein to identify constrained epitopes for antibody targeting (e.g., a region that differs significantly from DENV), (ii) identification of initial antibody scaffolds that target the identified epitope and confirmation of ZIKV E binding and in vitro virus neutralization; (iii) selection of lead antibodies to the predicted epitope, identification of paratope amino acids for affinity enhancement, and humanization of candidate antibodies; (iv) assessment of developability of lead candidate humanized antibodies (e.g., expression, thermal stability, and polyreactivity); and (v) assessment of half-life of lead candidates in animal models and engineer Fc region for extended half-life.

Without wishing to be bound by theory, it is believed that in an embodiment, targeting domain III of the Zika virus E protein can provide an antibody that reacts specifically with Zika virus and that does not cross-react with other members of the Flavivirus family (e.g., a dengue virus). The antibody molecules described herein can be structurally engineered, e.g., in the Fc region to extend half-life. Longer antibody half-life can be engineered by optimizing neonatal Fc receptor (FcRn) binding and/or recycling. In an embodiment, the antibody molecules described herein can result in single-dose protection of at risk populations, including pregnant women. In an embodiment, the antibody molecule is administered as a single dose, e.g., for duration of pregnancy. In another embodiment, the antibody molecule is administered once a trimester or once a month, e.g., for duration of pregnancy.

The disclosure herein can be applied to vaccine approaches to Zika virus and more broadly to the Flavivirus family. A vaccine, either to Zika virus or dengue virus, which elicits cross-reactive antibodies, particularly to domain II, has the potential to enhance disease if such antibodies bind to, but do not neutralize, the heterologous virus. This type of disease enhancement may already be apparent in long-term follow-up of a tetravalent vaccine to dengue, where naïve individuals administered vaccine are at subsequent risk for more severe dengue infection, where protection is not complete (Simmons. N Engl J Med. 2015; 373(13): 1263-4). Urgently needed countermeasures for the rapidly emerging Zika virus epidemic warrants the development of immunoprophylactic measures to treat the most vulnerable populations, including pregnant women, where maternal-fetal transfer of Zika virus leads to substantial risk for the developing fetus.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat”, e.g., a Zika virus infection, means that a subject (e.g., a human) who has been infected with a virus (e.g., Zika virus) and/or experiences a symptom of a disorder associated with the virus (e.g., Zika virus), will, in an embodiment, suffer less severe symptoms and/or will recover faster, when an antibody molecule, e.g., an antibody molecule described herein, is administered than if the antibody molecule were never administered. In an embodiment, when a Zika virus infection is treated, a sample from the subject will show less or no Zika virus after effective treatment for a Zika virus infection. For example, a diagnostic assay using polymerase chain reaction (PCR) or enzyme-linked immunosorbent assay (ELISA) will detect less or no viruses in a biological sample of a subject after administration of an antibody molecule described herein for the effective treatment of a Zika virus infection. Other assays can also be used to monitor treatment in a patient, or to detect the presence, e.g., decreased presence (or absence), of a symptom of a Zika virus infection, after treatment of a Zika virus infection in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., a Zika virus infection. In an embodiment, treatment is of a subject who does not exhibit certain signs of a disorder, e.g., a Zika virus infection, and/or of a subject who exhibits only early signs of a disorder, e.g., a Zika virus infection. In an embodiment, treatment is of a subject who exhibits one or more established signs of a disorder, e.g., a Zika virus infection. In an embodiment, treatment is of a subject diagnosed as suffering from a disorder, e.g., a Zika virus infection.

As used herein, the term “prevent,” a disorder, e.g., a Zika virus infection, means that a subject (e.g., a human) is less likely to have the disorder, e.g., a Zika virus infection, if the subject receives the antibody molecule.

Various aspects of the compositions and methods herein are described in further detail below. Additional definitions are set out throughout the specification.

### Zika Virus

The antibody molecules described herein bind to Zika virus and can be used to inhibit Zika virus, or to treat, prevent, and/or diagnose Zika virus infection. The vaccines and immunogens described herein can induce an immune response against, or confers protection against, Zika virus.

Zika virus (ZIKV) is a member of the virus family Flaviviridae and the genus Flavivirus, transmitted by daytime-active Aedes mosquitoes, such as A. aegypti and A. albopictus. It is one of the two viruses in the Spondweni virus clade (Faye et al. PLoS Negl Trop Dis. 2014; 8(1):e2636). The Zika virion is enveloped, spherical, about 50 nm in diameter. The surface proteins are arranged in an icosahedral-like symmetry. Zika virus is positive-sense ssRNA virus.

Zika virus has a monopartite, linear, ssRNA(+) genome of about 10-11 kilobases. The complete genome sequence of the Zika virus is described, e.g., in Kuno et al. Arch Virol. 2007; 152(4):687-96). The genome is enclosed in a capsid and surrounded by a membrane. The genome 5′ end has a methylated nucleotide cap for canonical cellular translation. The 3′ terminus is not polyadenylated but forms a loop structure. This secondary structure leads to the formation of a subgenomic flavivirus RNA (sfRNA) through genomic RNA degradation by host XRN1. sfRNA is essential for pathogenicity, and may play a role in inhibiting host RIG-I antiviral activity.

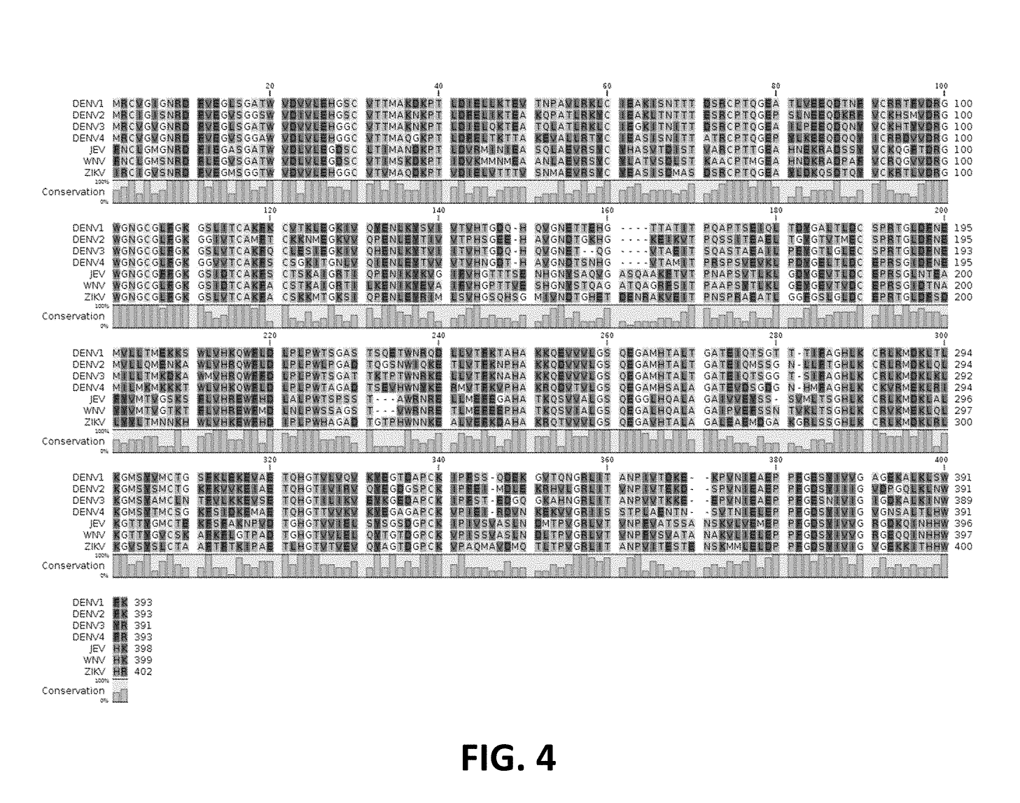

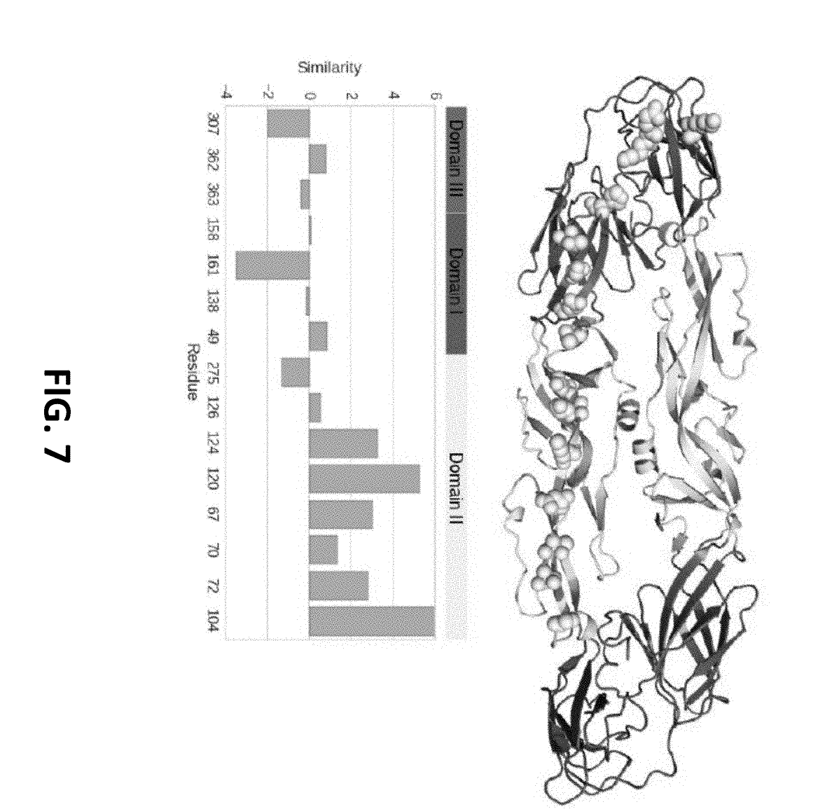

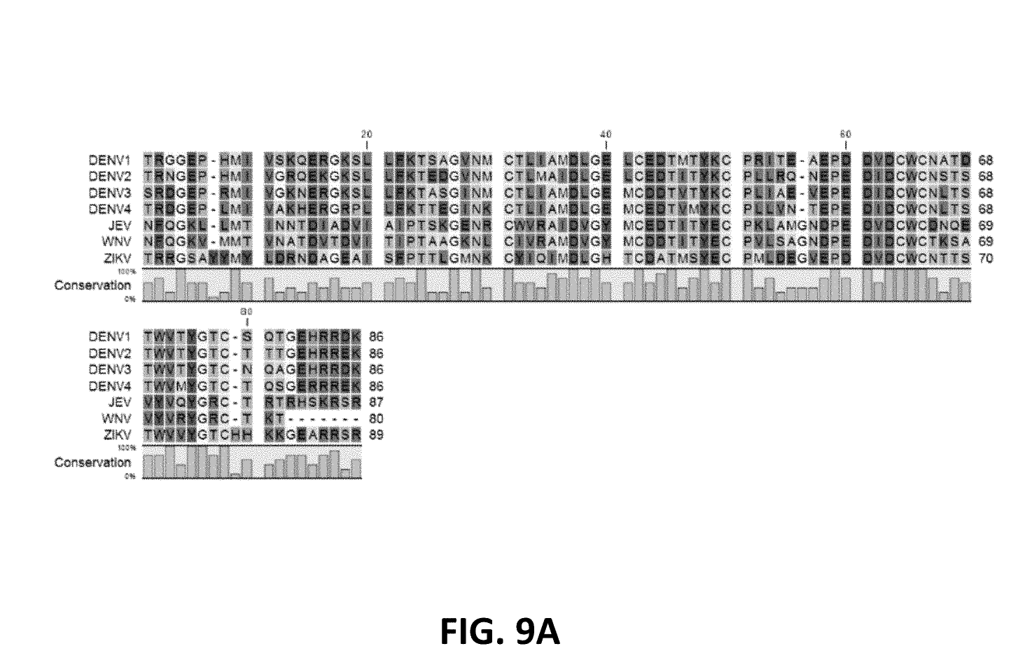

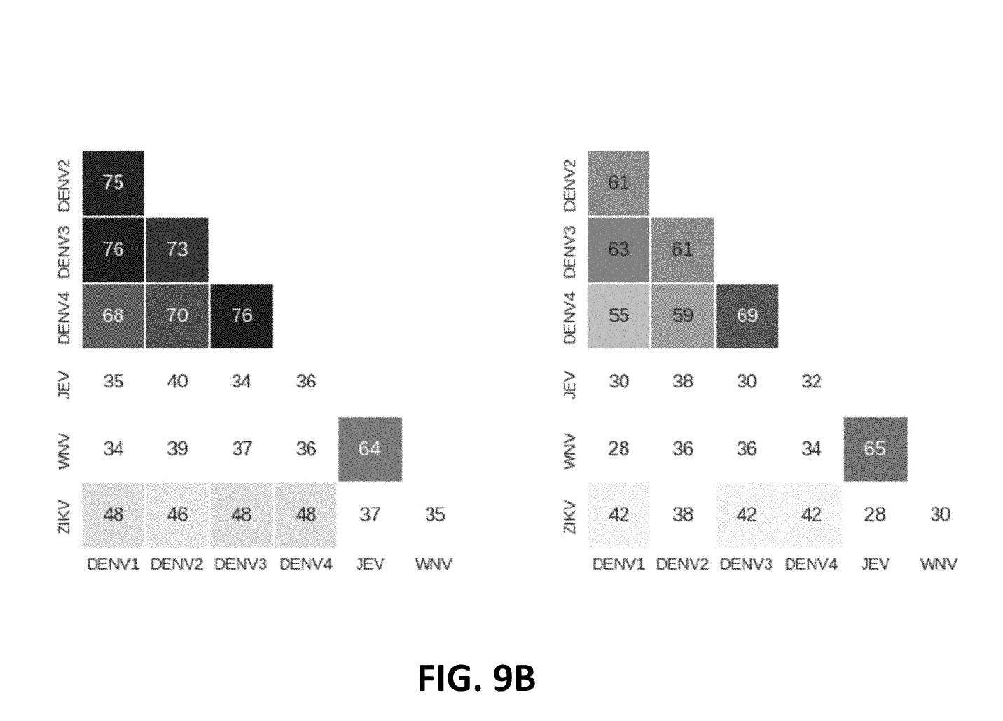

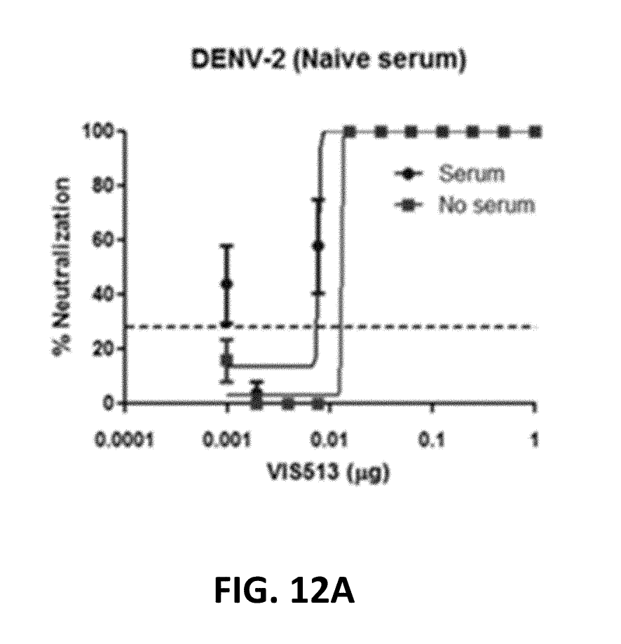

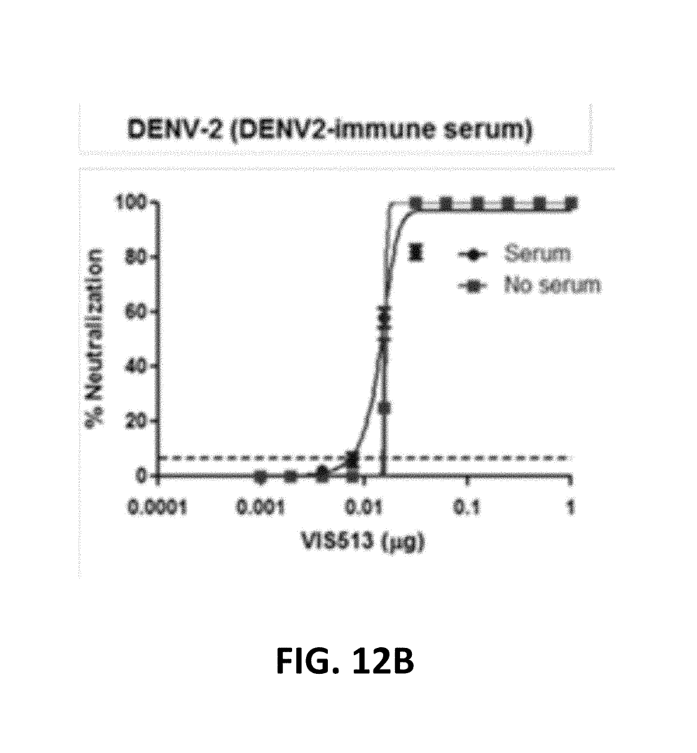

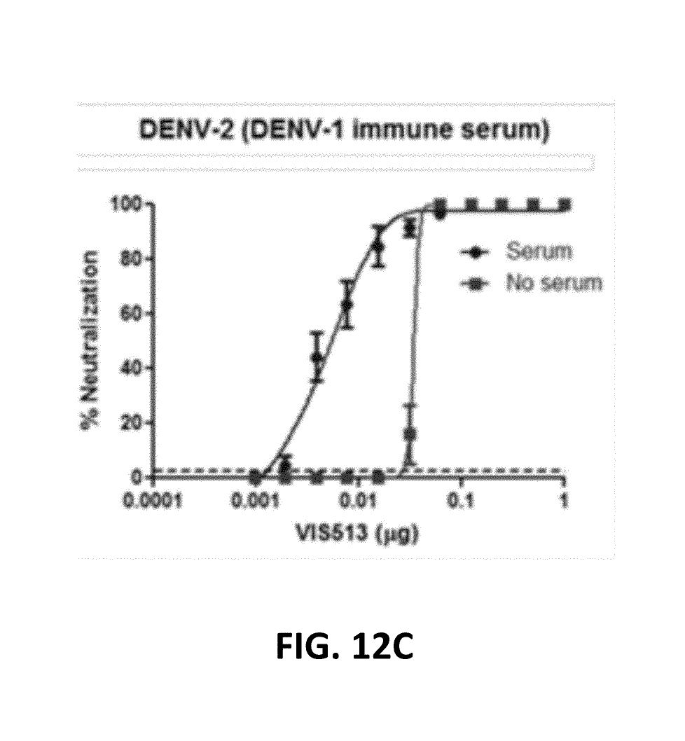

The virion RNA is infectious and serves as both the genome and the viral messenger RNA. The whole genome is translated in a polyprotein of about 3,500 amino acids in length, which is processed co- and post-translationally by host and viral proteases to structural proteins capsid (C), premembrane/membrane (pr/M or prM) and envelop (E), and eight non-structural proteins (NS1, NS2A, NS2B, NS3, NS4A, 2K, NS4B, and NS5). The amino acid sequence for an exemplary Zika virus prM protein is shown in FIG. 9. The amino acid sequence for an exemplary Zika virus E protein is shown in FIG. 4 or SEQ ID NO: 407. The E protein includes domain I, domain II and domain III, and the structure is illustrated in FIG. 7. While not wishing to be bound by theory, it is believed that in an embodiment, the antibody molecules describe herein reduce the risk of antibody-dependent enhancement of viral infection, by targeting certain region(s) on the E protein, e.g., domain III of the E protein. As shown in FIG. 12, a number of Zika virus strains also contain a putative N-linked glycosylation site at position N154 in domain I of the E protein, which may confer neurovirulence. ZIKV E protein sequences are highly homologous. For example, South American sequences cluster with Asian sequences and South American sequences are very similar (Enfissi et al. Lancet. 2016; 387(10015):227-8). Pairwise sequence diversity for all ZIKV sequences examined is greater than 95%.

During replication, attachment of the viral envelope protein E to host receptors mediates internalization into the host cell by apoptotic mimicry. Virus membrane is first fused with host endosomal membrane, and then RNA genome is released into the cytoplasm. The positive-sense genomic ssRNA is translated into a polyprotein, which is cleaved into all structural and non-structural proteins (to yield the replication proteins). Replication takes place at the surface of endoplasmic reticulum in cytoplasmic viral factories. A dsRNA genome is synthesized from the genomic ssRNA(+). The dsRNA genome is transcribed/replicated thereby providing viral mRNAs/new ssRNA(+) genomes. Virus assembly occurs at the endoplasmic reticulum. The virion buds via the host ESCRT complexes at the endoplasmic reticulum, is transported to the Golgi apparatus. The prM protein is cleaved in the Golgi, thereby maturing the virion which is fusion competent. New virions are released by exocytosis.

The antibody molecules described herein can reduce (e.g., inhibit or neutralize) one or more activities of Zika virus. In an embodiment, the antibody molecule reduces binding of the virus to the cell surface. In another embodiment, the antibody molecule reduces fusion between the E protein and the endosomal membrane.

There are two lineages of Zika virus: the African lineage and the Asian lineage (Enfissi et al. Lancet. 2016; 387(10015):227-8). Phylogenetic studies indicate that the virus spreading in the Americas is most closely related to the Asian strain, which circulated in French Polynesia during the 2013-2014 outbreak (Enfissi et al. Lancet. 2016; 387(10015):227-8; Zanluca et al. Mem Inst Oswaldo Cruz. 2015; 110(4):569-72). The complete genome sequence of Zika virus has been published (Kuno et al. Arch Virol. 2007; 152(4):687-96). Western Hemisphere Zika virus is found to be 89% identical to African genotypes, but is most closely related to the strain found in French Polynesia during 2013-2014 (Lanciotti et al. (2016). “Phylogeny of Zika Virus in Western Hemisphere, 2015,” Emerging Infectious Diseases 22 (5)).

The antibody molecules described herein can bind to, and optionally neutralize, Zika virus, e.g., one or more Zika virus strains, e.g., one or more Zika virus strains described herein. Exemplary Zika virus strains (and the GenBank accession numbers for their complete or partial genome sequences) include, but are not limited to, Zika virus isolate Brazil-ZKV2015 (KU497555.1), Zika virus isolate SSABR1 (KU707826.1), Zika virus strain Natal RGN (KU527068.1), Zika virus strain BeH815744 (KU365780.1), Zika virus strain BeH819966 (KU365779.1), Zika virus strain BeH819015 (KU365778.1), Zika virus strain BeH818995 (KU365777.1), Zika virus strain ZikaSPH2015 (KU321639.1), Zika virus isolate Si322 (KU646828.1), Zika virus isolate Si323 (KU646827.1), Zika virus strain 103344 (KU501216.1), Zika virus strain 8375 (KU501217.1), Zika virus strain MRS_OPY_Martinique_PaRi_2015 (KU647676.1), Zika virus isolate Z1106027 (KU312315.1), Zika virus strain PRVABC59 (KU501215.1), Zika virus isolate Z1106032 (KU312313.1), Zika virus isolate Z1106031 (KU312314.1), Zika virus isolate Z1106033 (KU312312), Zika virus strain Haiti/1225/2014 (KU509998.1), Zika virus strain CK-ISL 2014 (KJ634273.1), Zika virus isolate Zika virus/H.sapiens-tc/THA/2014/SV0127-14 (KU681081.3), Zika virus strain H/PF/2013 (KJ776791.1), Zika virus strain PLCal_ZV (KF993678.1), Zika virus isolate Zika virus/H.sapiens-tc/PHL/2012/CPC-0740 (KU681082.3), Zika virus associated with an epidemic, Yap State, Micronesia, 2007 (EU545988.1), Zika virus isolate ARB13565 (KF268948.1), Zika virus isolate ArD_41519 (HQ234501.1), Zika virus isolate IbH_30656 (HQ234500.1), Zika virus isolate P6-740 (HQ234499.1), Zika virus strain (YP002790881.1), Zika virus strain (NC_012532.1), Zika virus isolate MR766-NIID (LC002520.1), Zika virus isolate MR_766 (HQ234498.1), Zika virus strain MR 766 (AY632535.2; DQ859059.1), Zika virus strain ArD158095 (KF383121.1), Zika virus isolate ARB15076 (KF268949.1), Zika virus isolate ARB7701 (KF268950.1), Zika virus strain ArD158084 (KF383119.1), Zika virus strain ArD157995 (KF383118.1), or Zika virus strain ArD7117 (KF383116.1).

Zika virus is transmitted by daytime-active mosquitoes as its vector. It is primarily transmitted by the female Aedes aegypti in order to lay eggs, but has been isolated from a number of arboreal mosquito species in the Aedes genus, such as A. africanus, A. apicoargenteus, A. furcifer, A. hensilli, A. luteocephalus and A. vittatus with an extrinsic incubation period in mosquitoes of about 10 days (Hayes et al. Emerging Infectious Diseases 2009; 15 (9): 1347-50). There are also reported cases indicating that Zika virus could possibly be sexually transmitted (Alexandra et al. “Interim Guidelines for Prevention of Sexual Transmission of Zika Virus—United States, 2016,” Morbidity and Mortality Weekly Report 2016; 65 (Early Release 5 Feb. 2016): 1-2).

### Zika Fever

The antibody molecules described herein can be used to treat, prevent, and/or diagnose a disorder associated with Zika virus infection, e.g., Zika fever. The vaccines and immunogens described herein can induce an immune response against, or confers protection against, a disorder associated with Zika virus infection, e.g., Zika fever.

Zika fever (also known as Zika virus disease) is a disorder caused by the Zika virus (“Zika virus”. WHO. January 2016). Most cases have no symptoms, but when present are usually mild and can resemble dengue fever. Symptoms generally last less than seven days. Symptoms can include, e.g., fever, red eyes, joint pain, headache, a maculopapular rash, or a combination thereof. In some circumstances, Zika virus infection has been linked to microcephaly in newborns of infected mothers and to Guillain-Barré syndrome (GBS). Complications of Zika virus infection can be severe, including, e.g., neurotrophism.

Zika fever is mainly spread via the bite of mosquitoes of the Aedes type. It can also be potentially spread by sex and blood transfusions. Infections in pregnant women may also infect the baby and has been linked to miscarriage and microcephaly. Diagnosis can be performed, e.g., by testing the blood, urine, or saliva for the virus's RNA, e.g., when the person is sick.

The signs and symptoms of Zika fever include, but are not limited to, one or more of: fever, rash, conjunctivitis (red eyes), muscle and joint pain, or headache. The incubation period (the time from exposure to symptoms) for Zika virus disease is not known, but is likely to be a few days to a week. The disease lasts for several days to a week and is usually mild enough that people do not have to go to a hospital.

Zika virus infection can be associated with GBS, which is the rapid onset of muscle weakness that can progress to paralysis. Zika virus infection can also be associated with microcephaly. The disease may be spread from mother-to-child in the womb and cause multiple problems. While not wishing to be bound by theory, it is believed that Zika virus can infect human neural progenitor cells leading to decreased growth and cell death (Tang, et al. Cell Stem Cell. doi:10.1016/j.stem.2016.02.016). Investigators have also found evidence of eye abnormalities such as chorioretinal scarring in newborns with Zika-associated microcephaly. Another described effect has been hydrops fetalis, where there is abnormal accumulation of fluid in the fetus. Causal relationship with serious brain anomalies has been established (Rasmussen et al., N Engl J Med. 2016; 374(20):1981-7; Petersen et al. N Engl J Med. 2016; 374(16):1552-63).

Zika virus can be identified, e.g., by reverse transcriptase PCR (RT-PCR), e.g., in acutely ill patients. Exemplary RT-PCR methods for detection of Zika virus are described, e.g., in Faye, et al. (2008). Journal of Clinical Virology 43 (1): 96-101. Serology for the detection of specific IgM and IgG antibodies to Zika virus can also be used. IgM antibodies can be detectable within 3 days of the onset of illness. Serological cross-reactions with closely related flaviviruses such as dengue and West Nile virus as well as vaccines to flaviviruses are possible.

The World Health Organization (WHO) recommends RT-PCR testing be done on serum collected within 1 to 3 days of symptom onset or on saliva or urine samples collected during the first 3 to 5 days.

The CDC recommends screening some pregnant women even if they do not have symptoms of infection. Pregnant women who have travelled to affected areas should be tested between two and twelve weeks after their return from travel. For women living in affected areas, the CDC has recommended testing at the first prenatal visit with a doctor as well as in the mid-second trimester, though this may be adjusted based on local resources and the local burden of Zika virus. Additional testing should be done if there are any signs of Zika virus disease. Women with positive test results for Zika virus infection should have their fetus monitored by ultrasound every three to four weeks to monitor their anatomy and growth (Oduyebo et al. “Update: Interim Guidelines for Health Care Providers Caring for Pregnant Women and Women of Reproductive Age with Possible Zika Virus Exposure—United States, 2016.” Morbidity and Mortality Weekly Report 65 (05): 1-6).

For infants with suspected congenital Zika virus disease, the CDC recommends testing with both serologic and molecular assays such as RT-PCR, IgM ELISA and plaque reduction neutralization test (PRNT) (Staples et al. “Interim Guidelines for the Evaluation and Testing of Infants with Possible Congenital Zika Virus Infection—United States, 2016”. MMWR. Morbidity and Mortality Weekly Report 65 (3): 63-67). Newborns with a mother who was potentially exposed and who have positive blood tests, microcephaly or intracranial calcifications should have further testing including a thorough physical investigation for neurologic abnormalities, dysmorphic features, splenomegaly, hepatomegaly, and rash or other skin lesions. Other recommended tests are cranial ultrasound, hearing evaluation, and eye examination. Testing should be done for any abnormalities encountered as well as for other congenital infections such as syphilis, toxoplasmosis, rubella, cytomegalovirus infection, lymphocytic choriomeningitis virus infection, and herpes simplex virus.

Exemplary commercially available diagnostic tests for Zika infection include, but are not limited to, PCR-based tests (e.g., Zika virus (single check) PCR kit (Genekam Biotechnology AG, Germany; Cat. # FR325), Zika virus (double check) PCR kit (Genekam Biotechnology AG, Germany; Cat. # FR340), FTD Zika virus (Fast-track Diagnostics, Malta; Cat. # FTD 77), RealStar® Zika Virus RT-PCR kit (altona Diagnostics GmbH, Germany; Cat. #591013), genesig® Advanced Kit (Primerdesign Ltd., UK; Cat. # Path-ZIKV-EASY), Zika PCR kit (MyBiosource, USA; Cat. #MBS598109), ELISA based tests (e.g., Arboviral Fever Mosaic 2 (IgG/IgM) (Euroimmun, Germany; Cat. # FI 2668-1 G/M); Anti-Zika Virus ELISA (IgG/IgM) (Euroimmun, Germany; Cat. # EI 2668-9601 G/M); Zika Virus IgG (ZV-IgG), ELISA Kit (MyBiosource, USA; Cat. # MBS109002); Zika Virus IgG (ZV-IgM), ELISA Kit (MyBiosource, USA; Cat. # MBS109003); or Zika Immunoglobulin M (IgM) Antibody Capture Enzyme-Linked Immunosorbent Assay (Zika MAC-ELISA) (Center for Disease Control and Prevention, USA)), or rapid diagnostics tests (RDTs) (e.g., Zika Virus IgG/IgM Ab Rapid Test (Biocan Diagnostics, Canada; Cat. # B815C).

### Epitope

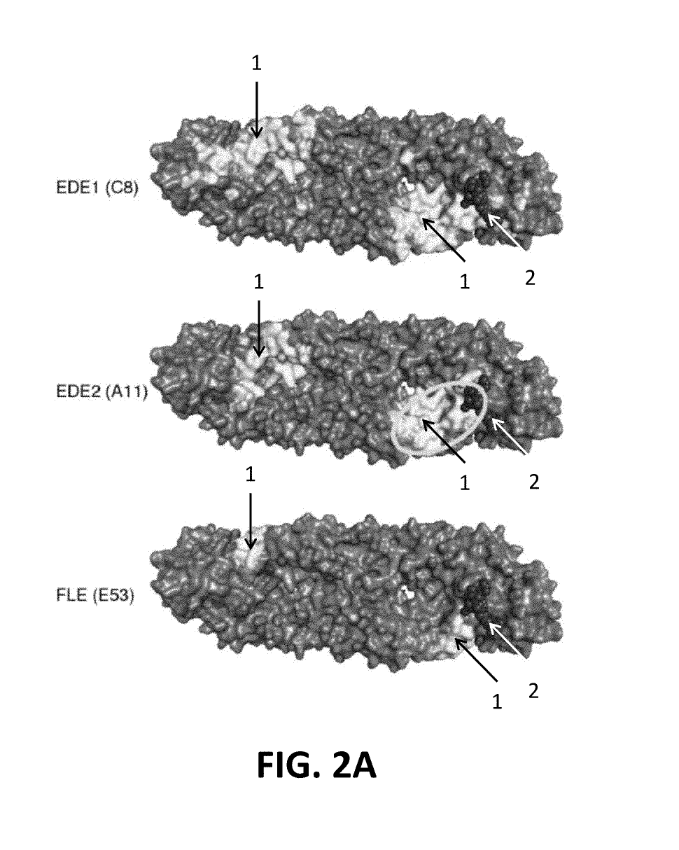

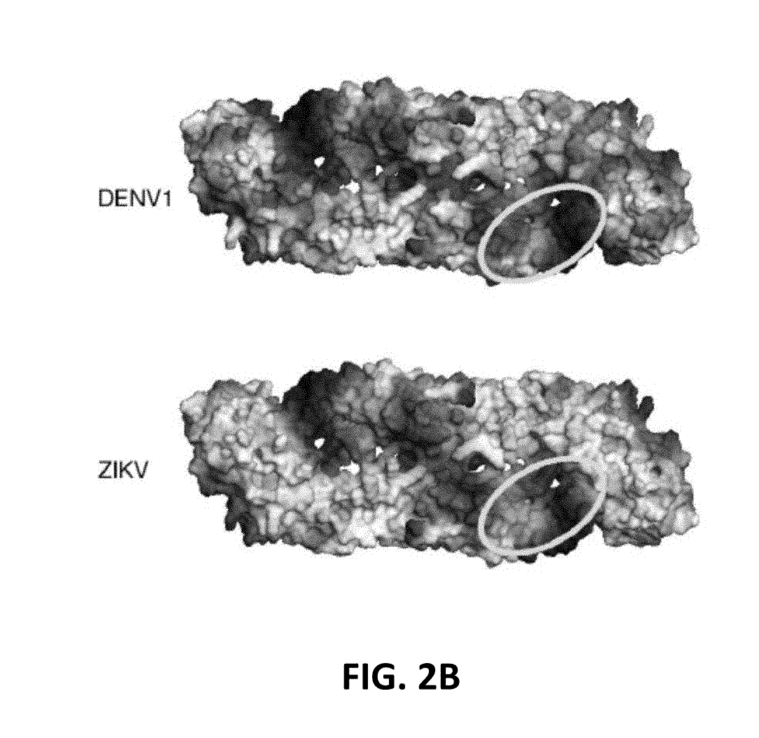

Dominant flavivirus cross-reactive neutralizing epitopes include, e.g., fusion loop epitope (FLE) and envelope dimer epitope (EDE). As shown in FIGS. 2A-2B, the FLE and EDE footprint coincides with the region in Zika virus with disproportionately high similarity. Levels of cross-reactivity and the corresponding epitopes can have important implications for vaccine (e.g., enhancement) and diagnostics (e.g., IgG cross reactivity).

The antibody molecule described herein can bind to an epitope on the E protein of Zika virus, e.g., domain III of the E protein (e.g., the lateral ridge of domain III of the E protein, e.g., including one or more residues in the domain III N-terminal linker (residues 305-311), the BC loop (residues 333-338), the DE loop (residues 365-371), and/or the FG loop (residues 391-395), e.g., as shown in FIG. 4 or SEQ ID NO: 407). Domain III of the E protein is sometimes referred to as EDIII herein.

In an embodiment, the epitope includes one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) amino acid residues in a surface accessible region of the Zika virus E protein. In an embodiment, the epitope is not a cross-reactive epitope, e.g., not reactive with antibodies against other flaviviruses (e.g., dengue virus). In an embodiment, the epitope resides in, or overlaps with, a region that has low similarity (e.g., identity) between Zika virus and one or more other flaviviruses (e.g., dengue virus). For example, the similarity (e.g., identity) can be about 60% or less, e.g., about 50% or less, about 40% or less, about 30% or less, about 20% or less, or about 10% or less.

In an embodiment, the epitope includes one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) residues described herein, e.g., in FIG. 4 or SEQ ID NO: 407. In an embodiment, contacting one or more of the amino acid residues in FIG. 4 or SEQ ID NO: 407 with an antibody molecule described herein inhibits, or substantially inhibits, one or more activities of Zika virus (e.g., inhibits, or substantially inhibits, attachment, membrane fusion, E protein dynamics (e.g., by locking E dimer conformation), or a combination thereof). While not wishing to be bound by theory, it is believed that in an embodiment, at least some of the amino acid residues shown in FIG. 4 or SEQ ID NO: 407 contribute to the binding of the virus to the cell surface, the fusion between the E protein and the endosomal membrane, or both.

In an embodiment, the antibody molecule binds, or substantially binds, to one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) residues of the E protein (e.g., encompassing residues 302-402 as shown in FIG. 4 or SEQ ID NO: 407), e.g., domain III of the E protein (e.g., one or more residues in the lateral ridge of domain III of the E protein, e.g., one or more residues in the domain III N-terminal linker (residues 305-311), the BC loop (residues 333-338), the DE loop (residues 365-371), and/or the FG loop (residues 391-395), e.g., as shown in FIG. 4 or SEQ ID NO: 407).

The vaccines and immunogens described herein can also contain an epitope described herein, e.g., one or more amino acid residues in domain III of the E protein (e.g., the lateral ridge of domain III of the E protein).

Exemplary E protein amino acid sequences are as follows:

>DENV1
(SEQ ID NO: 401)
MRCVGIGNRDFVEGLSGATWVDVVLEHGSCVTTMAKDKPTLDIELLKTEV
TNPAVLRKLCIEAKISNTTTDSRCPTQGEATLVEEQDTNFVCRRTFVDRG
WGNGCGLFGKGSLITCAKFKCVTKLEGKIVQYENLKYSVIVTVHTGDQHQ
VGNETTEHGTTATITPQAPTSEIQLTDYGALTLDCSPRTGLDFNEMVLLT
MEKKSWLVHKQWFLDLPLPWTSGASTSQETWNRQDLLVTFKTAHAKKQEV
VVLGSQEGAMHTALTGATEIQTSGTTTIFAGHLKCRLKMDKLTLKGMSYV
MCTGSFKLEKEVAETQHGTVLVQVKYEGTDAPCKIPFSSQDEKGVTQNGR
LITANPIVTDKEKPVNIEAEPPFGESYIVVGAGEKALKLSWFK
>DENV2
(SEQ ID NO: 402)
MRCIGISNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGNGCGLFGKGGIVTCAMFTCKKNMEGKVVQPENLEYTIVVTPHSGEEHA
VGNDTGKHGKEIKVTPQSSITEAELTGYGTVTMECSPRTGLDFNEMVLLQ
MENKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKVVKEIAETQHGTIVIRVQYEGDGSPCKIPFEIMDLEKRHVLGR
LITVNPIVTEKDSPVNIEAEPPFGDSYIIIGVDPGQLKLNWFK
>DENV3
(SEQ ID NO: 403)
MRCVGVGNRDFVEGLSGATWVDVVLEHGGCVTTMAKNKPTLDIELQKTEA
TQLATLRKLCIEGKITNITTDSRCPTQGEAILPEEQDQNYVCKHTYVDRG
WGNGCGLFGKGSLVTCAKFQCLESIEGKIVQHENLKYTVIITVHTGDQHQ
VGNETQGVTAEITSQASTAEAILPEYGTLGLECSPRTGLDFNEMILLTMK
DKAWMVHRQWFFDLPLPWTSGATTKTPTWNRKELLVTFKNAHAKKQEVVV
LGSQEGAMHTALTGATEIQTSGGTSIFAGHLKCRLKMDKLKLKGMSYAMC
LNTFVLKKEVSETQHGTILIKVEYKGEDAPCKIPFSTEDGQGKAHNGRLI
TANPVVTKKEEPVNIEAEPPFGESNIVIGIGDKALKINWYR
>DENV4
(SEQ ID NO: 404)
MRCVGVGNRDFVEGVSGGAWVDLVLEHGGCVTTMAQGKPTLDFELTKTTA
KEVALLRTYCIEASISNITTATRCPTQGEPYLKEEQDQQYICRRDVVDRG
WGNGCGLFGKGGVVTCAKFSCSGKITGNLVQIENLEYTVVVTVHNGDTHA
VGNDTSNHGVTAMITPRSPSVEVKLPDYGELTLDCEPRSGIDFNEMILMK
MKKKTWLVHKQWFLDLPLPWTAGADTSEVHWNYKERMVTFKVPHAKRQDV
TVLGSQEGAMHSALAGATEVDSGDGNHMFAGHLKCKVRMEKLRIKGMSYT
MCSGKFSIDKEMAETQHGTTVVKVKYEGAGAPCKVPIEIRDVNKEKVVGR
IISSTPLAENTNSVTNIELEPPFGDSYIVIGVGNSALTLHWFR
>JEV
(SEQ ID NO: 405)
FNCLGMGNRDFIEGASGATWVDLVLEGDSCLTIMANDKPTLDVRMINIEA
SQLAEVRSYCYHASVTDISTVARCPTTGEAHNEKRADSSYVCKQGFTDRG
WGNGCGFFGKGSIDTCAKFSCTSKAIGRTIQPENIKYKVGIFVHGTTTSE
NHGNYSAQVGASQAAKFTVTPNAPSVTLKLGDYGEVTLDCEPRSGLNTEA
FYVMTVGSKSFLVHREWFHDLALPWTSPSSTAWRNRELLMEFEGAHATKQ
SVVALGSQEGGLHQALAGAIVVEYSSSVMLTSGHLKCRLKMDKLALKGTT
YGMCTEKFSFAKNPVDTGHGTVVIELSYSGSDGPCKIPIVSVASLNDMTP
VGRLVTVNPFVATSSANSKVLVEMEPPFGDSYIVVGRGDKQINHHWHK
>WNV
(SEQ ID NO: 406)
FNCLGMSNRDFLEGVSGATWVDLVLEGDSCVTIMSKDKPTIDVKMMNMEA
ANLAEVRSYCYLATVSDLSTKAACPTMGEAHNDKRADPAFVCRQGVVDRG
WGNGCGLFGKGSIDTCAKFACSTKAIGRTILKENIKYEVAIFVHGPTTVE
SHGNYSTQAGATQAGRFSITPAAPSYTLKLGEYGEVTVDCEPRSGIDTNA
YYVMTVGTKTFLVHREWFMDLNLPWSSAGSTVWRNRETLMEFEEPHATKQ
SVIALGSQEGALHQALAGAIPVEFSSNTVKLTSGHLKCRVKMEKLQLKGT
TYGVCSKAFKFLGTPADTGHGTVVLELQYTGTDGPCKVPISSVASLNDLT
PVGRLVTVNPFVSVATANAKVLIELEPPFGDSYIVVGRGEQQINHHWHK
>ZIKV
(SEQ ID NO: 407)
IRCIGVSNRDFVEGMSGGTWVDVVLEHGGCVTVMAQDKPTVDIELVTTTV
SNMAEVRSYCYEASISDMASDSRCPTQGEAYLDKQSDTQYVCKRTLVDRG
WGNGCGLFGKGSLVTCAKFACSKKMTGKSIQPENLEYRIMLSVHGSQHSG
MIVNDTGHETDENRAKVEITPNSPRAEATLGGFGSLGLDCEPRTGLDFSD
LYYLTMNNKHWLVHKEWFHDIPLPWHAGADTGTPHWNNKEALVEFKDAHA
KRQTVVVLGSQEGAVHTALAGALEAEMDGAKGRLSSGHLKCRLKMDKLRL
KGVSYSLCTAAFTFTKIPAETLHGTVTVEVQYAGTDGPCKVPAQMAVDMQ
TLTPVGRLITANPVITESTENSKMMLELDPPFGDSYIVIGVGEKKITHHW
HR

Various strategies can be used for epitope mapping. Exemplary methods include low resolution approaches, e.g., using individual chimeric EDIII constructs expressed on yeast surface (epitope “patch” that mAbs recognize), and high resolution approaches, e.g., using library of EDIII mutants expressed on yeast surface, to map the epitope footprint of mAbs.

### Antibody Molecules

Disclosed herein are antibody molecules that bind to Zika virus, e.g., bind to the E protein of a Zika virus, e.g., domain III of the E protein (e.g., the lateral ridge of domain III of the E protein).

As used herein, the term “antibody molecule” refers to a protein, e.g., an immunoglobulin chain or a fragment thereof, comprising at least one immunoglobulin variable domain sequence. The term “antibody molecule” includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody molecule can also be a human, humanized, CDR-grafted, or in vitro generated antibody. The antibody molecule can have a heavy chain constant region chosen from, e.g., IgG1, IgG2, IgG3, or IgG4. The antibody molecule can also have a light chain chosen from, e.g., kappa or lambda. The term “immunoglobulin” (Ig) is used interchangeably with the term “antibody” herein.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

The antibody molecules disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In an embodiment, the following definitions are used: AbM definition of CDR1 of the heavy chain variable domain and Kabat definitions for the other CDRs. In an embodiment, Kabat definitions are used for all CDRs. In addition, embodiments described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, and FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., APRIL, or an epitope thereof. With respect to proteins (or protein mimetics), the antigen-binding region typically includes one or more loops (of at least, e.g., four amino acids or amino acid mimics) that form an interface that binds to the antigen, e.g., the E protein, e.g., domain III of the E protein. Typically, the antigen-binding region of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-ZIKV antibody molecule, e.g., an anti-ZIKV antibody molecule provided herein, to a target, e.g., the E protein, e.g., domain III of the E protein. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In an embodiment, a competition binding assay is a quantitative competition assay. In an embodiment, a first anti-ZIKV antibody molecule is said to compete for binding to the target with a second anti-ZIKV antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

Hybridomas are typically immortal somatic cell hybrids that generate (e.g., secrete) antibodies. Antibody-secreting cell lines can be established routinely and maintained in vitro by fusing two partners, e.g., antibody-secreting cells isolated from immunized animals and myeloma cells. Antibody-secreting cells isolated from immunized animals generally carry the rearranged immunoglobulin genes that encode the desired antibody. Fusions are normally performed with a mixed population of cells isolated from a lymphoid organ of an immunized animal. Myelomas can be induced in a number of strains of mice by injecting mineral oil into the peritoneum. Derivatives of BALB/c myelomas are examples of commonly used partners for fusion. Exemplary myeloma cell lines used for hybridoma construction include, but are not limited to, mouse cell lines (e.g., X63Ag8.653, Sp2/0-Ag14, FO, NSI/1-Ag4-1, NSO/1, or FOX-NY) and rat cell lines (e.g., YB2/0 or IR983F).

The fusion between the myeloma cell and the antibody-producing cell can be effected by any fusogen. For example, the fusion can be performed, e.g., using polyethylene glycol (PEG), Sendai virus, or electric current (electrofusion). During fusion, plates are typically seeded at low density (e.g., one to five cells/well) to allow individual hybridoma cells to be isolated and screened. Exemplary methods to enhance low-density hybridoma growth include, but are not limited to, the use of hybridoma cloning supplements, oxaloacetate pyruvate insulin (OPI solution), recombinant murine IL-6, or feeder layer plates. Unfused myeloma cells can be eliminated by various drug selection methods. In an embodiment, the myeloma partner can have a mutation in one of the enzymes of the salvage pathway of purine nucleotide biosynthesis. For example, selection with 8-azaguanine can yield a cell line harboring a mutated hypoxanthine-guanine phosphoribosyl transferase gene (HPRT). Without wishing to be bound by theory, it is believed that addition of a compound that inhibits the de novo nucleotide synthesis pathway can direct cells to use the salvage pathway. Cells containing a nonfunctional HGPRT protein will die due to selection pressures, whereas hybrids between myelomas with a nonfunctional HPRT and a functional HPRT can continue to grow. Exemplary drugs that can be used for hybridoma selection include, e.g., drugs that inhibit the de novo nucleotide synthesis pathway, e.g., aminopterin, methotrexate, or azaserine.

Screening of desired antibodies can be performed, e.g., using antibody capture assays, antigen capture assays, functional screens, or a combination thereof. An exemplary antibody capture assay can include the following steps: the antigen is bound to a solid substrate; the antibodies in the hybridoma tissue culture supernatant are allowed to bind to the antigen; the unbound antibodies are removed by washing; and then the bound antibodies are detected by a secondary reagent that specifically recognizes the antibody. In an exemplary antigen capture assay, the antibodies in the tissue culture supernatant are bound to a solid phase first, and then the antigen is allowed to react with the antibody. In another exemplary antigen capture assay, the antibody-antigen complex is allowed to form before the binding of the antibody to a solid phase. In either case, the unbound antigen is removed by washing after the antibody-antigen complexes are bound to the solid support, and positives are identified by detecting the antigen. Any property that is unique to the antigen can be used to identify positives. In functional assays, the antibodies in the hybridoma tissue culture supernatants can be used, e.g., to block a reaction, induce a reaction, or as a molecular handle to deplete an essential component of a reaction mix. Blocking or neutralizing antibodies can inhibit the interaction of cells (e.g., receptor/ligand) or block reactions (e.g., chemical or cell signaling events). Antibodies can be blocking either by binding an epitope either directly or indirectly (e.g., sterically hinder the interaction). The screening methods described herein can be performed in a high-throughput format or a non-high-throughput format.

Exemplary methods for generating monoclonal antibodies are also described, e.g., in Greenfield, Antibodies: A Laboratory Manual, Second Edition, Cold Spring Harbor Laboratory Press, pages 201-221, which is incorporated by reference herein.

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In some embodiments, the antibody can be recombinantly produced, e.g., produced by any suitable phage display or combinatorial methods.

Various phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In an embodiment, the antibody molecule is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In an embodiment, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter U.S. Pat. No. 5,225,539), the contents of which is expressly incorporated by reference.

Also provided are humanized antibodies in which specific amino acids have been substituted, deleted or added. Criteria for selecting amino acids from the donor are described in, e.g., U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In an embodiment, the antibody molecule has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In certain embodiments, the antibody molecule has reduced or no ability to bind an Fc receptor. For example, it may be an isotype or subtype, fragment or other mutant, which does not support binding to an Fc receptor, e.g., it has a mutagenized or deleted Fc receptor binding region. In an embodiment, the Fc region of the antibody molecule includes one or more mutations that reduce FcγRI binding and/or antibody-dependent cell-mediated cytotoxicity (ADCC). In an embodiment, the mutation is in the hinge-region motif, e.g., at one or more of L234, L235, G236, or G237. In an embodiment, the mutation is L234A, L235A, or both. In another embodiment, the Fc region of the antibody molecule includes one or more mutations that reduce binding to Clq and/or complement activation. In an embodiment, the mutation is at L322, e.g., an L322A mutation.

In an embodiment, a constant region of the antibody molecule is altered. Methods for altering an antibody constant region are known in the art. Antibody molecules s with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

The antibody molecules described herein can contain an Fc region, e.g., an Fc region described herein, e.g., containing one or more mutations or, or one or more combinations, of mutations described herein.

A reference Fc region amino acid sequence is provided below (e.g., for identification of the mutation positions described herein). Certain positions described herein are underlined and bold.

(SEQ ID NO: 408)

| | |
|---|---|
| 118 | ASTKGPSVFPLAPSSKSTSGGTAALGCLVKDYFPEPVTVSWNSG |
| | ALTSGVHTFPAVL |
| 175 | QSSGLYSLSSVVTVPSSSLGTQTYICNVNHKPSNTKVDKRVEPK |
| | SCDKTHTCPPCPA |
| 230 | PAPELLGGPSVFLFPPKPKDTLMISRTPEVTCVVVDVSHEDPEV |
| | KFNWYVDGVEVHN |
| 287 | AKTKPREEQYNSTYRVVSVLTVLHQDWLNGKEYKCKVSNKALPA |
| | PIEKTISKAKGQP |
| 344 | REPQVYTLPPSREEMTKNQVSLTCLVKGFYPSDIAVEWESNGQP |
| | ENNYKTTPPVLDS |
| 401 | DGSFFLYSKLTVDKSRWQQGNVFSCSVMHEALHNHYTQKSLSLS |
| | PGK |

Exemplary Fc mutations include, but are not limited to, the following.

| | Name | Mutation |
|---|---|---|
| 1 | FcMut045 | T256D/T307R/Q311V |
| 2 | FcMut183 | T256D/Q311V/A378V |
| 3 | FcMut197 | H285N/T307Q/N315D |
| 4 | FcMut213 | H285D/T307Q/A378V |
| 5 | FcMut215 | T307Q/Q311V/A378V |
| 6 | FcMut228 | T256D/N286D/T307R/Q311V/A378V |
| 7 | FcMut156 | FcMut045 + L234A/L235A |

In an embodiment, the only amino acids in the antibody molecule are canonical amino acids. In an embodiment, the antibody molecule comprises naturally-occurring amino acids; analogs, derivatives and congeners thereof; amino acid analogs having variant side chains; and/or all stereoisomers of any of any of the foregoing. The antibody molecule may comprise the D- or L-optical isomers of amino acids and peptidomimetics.

A polypeptide of an antibody molecule described herein may be linear or branched, it may comprise modified amino acids, and it may be interrupted by non-amino acids. The antibody molecule may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody molecule described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-ZIKV antibody can be coupled to a radioactive isotope such as an α-, β-, or γ-emitter, or a β- and γ-emitter.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody molecule are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-dengue antibody molecule may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecules can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., antimicrobial (e.g., antibacterial or bactericidal), immunomodulatory, immunostimularoty, cytotoxic, or cytostatic) agent or moiety. Radioactive isotopes can be used in diagnostic or therapeutic applications. Radioactive isotopes that can be coupled to the antibody molecules include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The present disclosure provides radiolabeled antibody molecules and methods of labeling the same. In an embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

In some aspects, this disclosure provides a method of making an antibody molecule disclosed herein. The method includes: providing an antigen, e.g., the E protein of Zika virus (e.g., domain III of the E protein) or a fragment thereof; obtaining an antibody molecule that specifically binds to the antigen; evaluating efficacy of the antibody molecule in modulating activity of the antigen and/or organism expressing the antigen, e.g., the E protein of Zika virus (e.g., domain III of the E protein). The method can further include administering the antibody molecule, including a derivative thereof (e.g., a humanized antibody molecule) to a subject, e.g., a human.

This disclosure provides an isolated nucleic acid molecule encoding an antibody molecule described herein, or a vector and host cell thereof. The nucleic acid molecule includes, but is not limited to, RNA, genomic DNA and cDNA.

The antibody molecules described herein can have several advantageous properties. For example, the antibody molecules can be used to effectively treat, prevent or diagnose Zika virus infection or a disorder associated with Zika virus infection, e.g., a disorder described herein. In an embodiment, the antibody molecules described herein can achieve effective or optimal inhibition of Zika virus, while reducing the risk of antibody-dependent enhancement of infection, e.g., by targeting certain region(s) on the E protein (e.g., domain III of the E protein).

In an embodiment, the antibody molecule is capable of binding, or substantially binding, to Zika virus, e.g., the E protein of Zika virus (e.g., domain III of the E protein). In an embodiment, the antibody molecule binds to Zika virus, e.g., the E protein of Zika virus (e.g., domain III of the E protein) with high affinity, e.g., with a dissociation constant (K_{D}) of less than about 100 nM, typically about 10 nM, and more typically, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, or 0.01 nM, e.g., as determined by ELISA or SPR. In an embodiment, the antibody molecule binds to Zika virus, e.g., the E protein of Zika virus (e.g., domain III of the E protein) with a K_{off} slower than 1×10⁻⁴, 5×10⁻⁵, or 1×10⁻⁵ s⁻¹. In an embodiment, the antibody molecule binds to Zika virus, e.g., the E protein of Zika virus (e.g., domain III of the E protein) with a Kₒₙ faster than 1×10⁴, 5×10⁴, 1×10⁵, or 5×10⁵ M⁻¹s⁻¹.

In an embodiment, the antibody molecule is capable of inhibiting, or substantially inhibiting, a biological activity of Zika virus, e.g., a function of Zika virus E protein, in vitro, ex vivo, or in vivo. In an embodiment, the antibody molecule inhibits (e.g., neutralizes) Zika virus e.g., in a microneutralization assay. In an embodiment, the antibody molecule inhibits (e.g., neutralizes) Zika virus with an EC50 of about 10 μg/mL or less, e.g., about 9 μg/mL or less, about 8 μg/mL or less, about 7 μg/mL or less, about 6 μg/mL or less, about 5 μg/mL or less, about 4 μg/mL or less, about 3 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a microneutralization assay. In another embodiment, the antibody molecule inhibits (e.g., neutralizes) Zika virus with an EC90 of about 50 μg/mL or less, e.g., about 25 μg/mL or less, about 20 μg/mL or less, about 15 μg/mL or less, about 10 μg/mL or less, about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less, e.g., as determined by a microneutralization assay.

In an embodiment, the antibody molecule does not bind to domain II of the E protein, or binds to domain II of the E protein with low affinity, e.g., with a dissociation constant (K_{D}) of about 500 nM or more, e.g., about 750 nM or more, about 1 μM or more, about 2 μM or more, about 3 μM or more, about 4 μM or more, or about 5 μM or more, e.g., as determined by ELISA or SPR.

In an embodiment, the antibody molecule binds to a linear or conformational epitope on the E protein of Zika virus (e.g., domain III of the E protein). In an embodiment, the antibody molecule binds to an epitope conserved between two or more Zika virus strains. In an embodiment, the antibody molecule binds to an epitope that is not cross-reactive with other flaviviruses (e.g., dengue virus (e.g., DENV1, DENV2, DENV3, and/or DENV4), Japanese encephalitis virus (JEV), and/or West Nile virus (WNV)). In an embodiment, the antibody molecule binds to an epitope described herein.

In an embodiment, the antibody molecule binds, or substantially binds, to the same, similar, or overlapping epitope on the E protein of Zika virus (e.g., domain III of the E protein), as a second antibody molecule (e.g., a second antibody molecule described herein). In an embodiment, the antibody molecule competes with a second antibody molecule (e.g., a second antibody molecule described herein) for binding to Zika virus, e.g., the E protein of Zika virus (e.g., domain III of the E protein).

In an embodiment, the antibody molecule comprising a heavy chain variable region (VH) and a light chain variable region (VL), wherein the VH comprises three heavy chain complementarity determining regions (HCDR1, HCDR2, and HCDR3), and the VL comprises three light chain complementarity determining regions (LCDR1, LCDR2, and LCDR3),

wherein the VH comprises one, two, or all of the following: (i) an HCDR1 comprising the amino acid sequence of SEQ ID NO: 101 or 107, or an amino acid sequence that differs by no more than 1, 2, or 3 amino acid residues therefrom, or has at least 85, 90, 95, 99 or 100% homology therewith; (ii) an HCDR2 comprising the amino acid sequence of any of SEQ ID NOs: 102, 108, or 114, or an amino acid sequence that differs by no more than 1, 2, or 3 amino acid residues therefrom, or has at least 85, 90, 95, 99 or 100% homology therewith; or (iii) an HCDR3 comprising the amino acid sequence of any of SEQ ID NOs: 103, 113, 115, or 116, or an amino acid sequence that differs by no more than 1, 2, or 3 amino acid residues therefrom, or has at least 85, 90, 95, 99 or 100% homology therewith; and

wherein the VL comprises one, two, or all of the following: (i) an LCDR1 comprising the amino acid of any of SEQ ID NOs: 104, 109, 111, or 117, or an amino acid sequence that differs by no more than 1, 2, or 3 amino acid residues therefrom, or has at least 85, 90, 95, 99 or 100% homology therewith; (ii) an LCDR2 comprising the amino acid of any of SEQ ID NOs: 105, 112, or 118, or an amino acid sequence that differs by no more than 1, 2, or 3 amino acid residues therefrom, or has at least 85, 90, 95, 99 or 100% homology therewith; or (iii) an LCDR3 comprising the amino acid sequence of any of SEQ ID NOs: 106, 110, or 119, or an amino acid sequence that differs by no more than 1, 2, or 3 amino acid residues therefrom, or has at least 85, 90, 95, 99 or 100% homology therewith.

In an embodiment, the VH comprises: (i) an HCDR1 comprising the amino acid sequence of SEQ ID NO: 101 or 107; (ii) an HCDR2 comprising the amino acid sequence of any of SEQ ID NOs: 102, 108, or 114; or (iii) an HCDR3 comprising the amino acid sequence of any of SEQ ID NOs: 103, 113, 115, or 116; and the VL comprises: (i) an LCDR1 comprising the amino acid of any of SEQ ID NOs: 104, 109, 111, or 117; (ii) an LCDR2 comprising the amino acid of any of SEQ ID NOs: 105, 112, or 118; or (iii) an LCDR3 comprising the amino acid sequence of any of SEQ ID NOs: 106, 110, or 119.

In an embodiment, the antibody molecule comprising a VH and a VL, wherein the VH comprises three heavy chain complementarity determining regions (HCDR1, HCDR2, and HCDR3), and the VL comprises three light chain complementarity determining regions (LCDR1, LCDR2, and LCDR3),

wherein the VH comprises one, two, or all of the following: (i) an HCDR1 comprising the amino acid sequence of SEQ ID NO: 201 or 203, or an amino acid sequence that differs by no more than 1, 2, or 3 amino acid residues therefrom, or has at least 85, 90, 95, 99 or 100% homology therewith; (ii) an HCDR2 comprising the amino acid sequence of any of SEQ ID NOs: 202, 204, 205, 206, 207, 208, or 209, or an amino acid sequence that differs by no more than 1, 2, or 3 amino acid residues therefrom, or has at least 85, 90, 95, 99 or 100% homology therewith; or (iii) an HCDR3 comprising the amino acid sequence of any of SEQ ID NOs: 103, 113, 115, or 116, or an amino acid sequence that differs by no more than 1, 2, or 3 amino acid residues therefrom, or has at least 85, 90, 95, 99 or 100% homology therewith; and

wherein the VL comprises one, two, or all of the following: (i) an LCDR1 comprising the amino acid of any of SEQ ID NOs: 104, 109, 111, or 117, or an amino acid sequence that differs by no more than 1, 2, or 3 amino acid residues therefrom, or has at least 85, 90, 95, 99 or 100% homology therewith; (ii) an LCDR2 comprising the amino acid of any of SEQ ID NOs: 105, 112, or 118, or an amino acid sequence that differs by no more than 1, 2, or 3 amino acid residues therefrom, or has at least 85, 90, 95, 99 or 100% homology therewith; or (iii) an LCDR3 comprising the amino acid sequence of any of SEQ ID NOs: 106, 110, or 119, or an amino acid sequence that differs by no more than 1, 2, or 3 amino acid residues therefrom, or has at least 85, 90, 95, 99 or 100% homology therewith.

In an embodiment, the VH comprises: (i) an HCDR1 comprising the amino acid sequence of SEQ ID NO: 201 or 203; (ii) an HCDR2 comprising the amino acid sequence of any of SEQ ID NOs: 202, 204, 205, 206, 207, 208, or 209; or (iii) an HCDR3 comprising the amino acid sequence of any of SEQ ID NOs: 103, 113, 115, or 116; and the VL comprises: (i) an LCDR1 comprising the amino acid of any of SEQ ID NOs: 104, 109, 111, or 117; (ii) an LCDR2 comprising the amino acid of any of SEQ ID NOs: 105, 112, or 118; or (iii) an LCDR3 comprising the amino acid sequence of any of SEQ ID NOs: 106, 110, or 119.

In an embodiment, the antibody molecule comprises a heavy chain variable region (VH) comprising the amino acid sequence of any of SEQ ID NOs: 1, 3, 5, 9, 10, 11, 12, 13, 14, 15, 16, 17, or 22, or a an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith.

In an embodiment, the antibody molecule comprises a light chain variable region (VL) comprising the amino acid sequence of any of SEQ ID NOs: 2, 4, 6, 7, 8, 18, 19, 20, or 21, or a an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith.

In an embodiment, the antibody molecule comprises: (i) a VH comprising the amino acid sequence of any of SEQ ID NOs: 1, 3, 5, 9, 10, 11, 12, 13, 14, 15, 16, 17, or 22, or a an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith; and (ii) a VL comprising the amino acid sequence of any of SEQ ID NOs: 2, 4, 6, 7, 8, 18, 19, 20, or 21, or a an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith.

In an embodiment, the antibody molecule comprises: (i) a VH comprising the amino acid sequence of any of SEQ ID NOs: 1, 3, 5, 9, 10, 11, 12, 13, 14, 15, 16, 17, or 22; and (ii) a VL comprising the amino acid sequence of any of SEQ ID NOs: 2, 4, 6, 7, 8, 18, 19, 20, or 21.

In an embodiment, the antibody molecule comprises a VH encoded by a nucleotide sequence comprising any of SEQ ID NOs: 300, 301, 302, 303, 304, 308, 309, 310, 311, 312, or 317, or a nucleotide sequence that differs by no more than 1, 2, 3, 4, 5, 6, 7, 8, 9, or 10 nucleotides therefrom, or has at least 85, 90, 95, 99 or 100% homology therewith.

In an embodiment, the antibody molecule comprises a VL encoded by a nucleotide sequence comprising any of SEQ ID NOs: 305, 306, 307, 313, 314, 315, 316, or 318, or a nucleotide sequence that differs by no more than 1, 2, 3, 4, 5, 6, 7, 8, 9, or 10 nucleotides therefrom, or has at least 85, 90, 95, 99 or 100% homology therewith.

In an embodiment, the antibody molecule comprises (i) a VH encoded by a nucleotide sequence comprising any of SEQ ID NOs: 300, 301, 302, 303, 304, 308, 309, 310, 311, 312, or 317, or a nucleotide sequence that differs by no more than 1, 2, 3, 4, 5, 6, 7, 8, 9, or 10 nucleotides therefrom, or has at least 85, 90, 95, 99 or 100% homology therewith; and (ii) a VL encoded by a nucleotide sequence comprising any of SEQ ID NOs: 305, 306, 307, 313, 314, 315, 316, or 318, or a nucleotide sequence that differs by no more than 1, 2, 3, 4, 5, 6, 7, 8, 9, or 10 nucleotides therefrom, or has at least 85, 90, 95, 99 or 100% homology therewith.

In an embodiment, the antibody molecule comprises (i) a VH encoded by a nucleotide sequence comprising any of SEQ ID NOs: 300, 301, 302, 303, 304, 308, 309, 310, 311, 312, or 317; and (ii) a VL encoded by a nucleotide sequence comprising any of SEQ ID NOs: 305, 306, 307, 313, 314, 315, 316, or 318.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV54 (e.g., SEQ ID NO: 101); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV54 (e.g., SEQ ID NO: 102); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV54 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV54 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV54 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV54 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV54 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV54 (e.g., SEQ ID NO: 102); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV54 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV54 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV54 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV54 (e.g., SEQ ID NO: 106).

In an embodiment, the antibody molecule comprises: (i) a VH comprising: an HCDR1 comprising the amino acid sequence of the HCDR1 of monoclonal antibody ZV54 (e.g., SEQ ID NO: 101); an HCDR2 comprising the amino acid sequence of the HCDR2 of monoclonal antibody ZV54 (e.g., SEQ ID NO: 102); and an HCDR3 comprising the amino acid sequence of the HCDR3 of monoclonal antibody ZV54 (e.g., SEQ ID NO: 103), and (ii) a VL comprising: an LCDR1 comprising the amino acid sequence of the LCDR1 of monoclonal antibody ZV54 (e.g., SEQ ID NO: 104); an LCDR2 comprising the amino acid sequence of the LCDR2 of monoclonal antibody ZV54 (e.g., SEQ ID NO: 105); and an LCDR3 comprising the amino acid sequence of the LCDR3 of monoclonal antibody ZV54 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV54 (e.g., SEQ ID NO: 201); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV54 (e.g., SEQ ID NO: 202); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV54 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV54 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV54 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV54 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV54 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV54 (e.g., SEQ ID NO: 202); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV54 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV54 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV54 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV54 (e.g., SEQ ID NO: 106).

In an embodiment, the antibody molecule comprises: (i) a VH comprising: an HCDR1 comprising the amino acid sequence of the HCDR1 of monoclonal antibody ZV54 (e.g., SEQ ID NO: 201); an HCDR2 comprising the amino acid sequence of the HCDR2 of monoclonal antibody ZV54 (e.g., SEQ ID NO: 202); and an HCDR3 comprising the amino acid sequence of the HCDR3 of monoclonal antibody ZV54 (e.g., SEQ ID NO: 103), and (ii) a VL comprising: an LCDR1 comprising the amino acid sequence of the LCDR1 of monoclonal antibody ZV54 (e.g., SEQ ID NO: 104); an LCDR2 comprising the amino acid sequence of the LCDR2 of monoclonal antibody ZV54 (e.g., SEQ ID NO: 105); and an LCDR3 comprising the amino acid sequence of the LCDR3 of monoclonal antibody ZV54 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZV54 (e.g., SEQ ID NO: 1). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZV54 (e.g., SEQ ID NO: 2).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZV54 (e.g., SEQ ID NO: 1); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZV54 (e.g., SEQ ID NO: 2). In an embodiment, the antibody molecule comprises: (i) a VH comprising the amino acid sequence of the VH of monoclonal antibody ZV54 (e.g., SEQ ID NO: 1); and (ii) a VL comprising the amino acid sequence of the VL of monoclonal antibody ZV54 (e.g., SEQ ID NO: 2).

In an embodiment the antibody molecule comprises monoclonal antibody ZV54. In an embodiment, the antibody molecule comprises a humanized monoclonal antibody ZV54.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV67 (e.g., SEQ ID NO: 107); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV67 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV67 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV67 (e.g., SEQ ID NO: 109); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV67 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV67 (e.g., SEQ ID NO: 110).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV67 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV67 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV67 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV67 (e.g., SEQ ID NO: 109); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV67 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV67 (e.g., SEQ ID NO: 110).

In an embodiment, the antibody molecule comprises: (i) a VH comprising: an HCDR1 comprising the amino acid sequence of the HCDR1 of monoclonal antibody ZV67 (e.g., SEQ ID NO: 107); an HCDR2 comprising the amino acid sequence of the HCDR2 of monoclonal antibody ZV67 (e.g., SEQ ID NO: 108); and an HCDR3 comprising the amino acid sequence of the HCDR3 of monoclonal antibody ZV67 (e.g., SEQ ID NO: 103), and (ii) a VL comprising: an LCDR1 comprising the amino acid sequence of the LCDR1 of monoclonal antibody ZV67 (e.g., SEQ ID NO: 109); an LCDR2 comprising the amino acid sequence of the LCDR2 of monoclonal antibody ZV67 (e.g., SEQ ID NO: 105); and an LCDR3 comprising the amino acid sequence of the LCDR3 of monoclonal antibody ZV67 (e.g., SEQ ID NO: 110).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV67 (e.g., SEQ ID NO: 203); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV67 (e.g., SEQ ID NO: 204); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV67 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV67 (e.g., SEQ ID NO: 109); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV67 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV67 (e.g., SEQ ID NO: 110).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZV67 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZV67 (e.g., SEQ ID NO: 204); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZV67 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZV67 (e.g., SEQ ID NO: 109); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZV67 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZV67 (e.g., SEQ ID NO: 110).

In an embodiment, the antibody molecule comprises: (i) a VH comprising: an HCDR1 comprising the amino acid sequence of the HCDR1 of monoclonal antibody ZV67 (e.g., SEQ ID NO: 203); an HCDR2 comprising the amino acid sequence of the HCDR2 of monoclonal antibody ZV67 (e.g., SEQ ID NO: 204); and an HCDR3 comprising the amino acid sequence of the HCDR3 of monoclonal antibody ZV67 (e.g., SEQ ID NO: 103), and (ii) a VL comprising: an LCDR1 comprising the amino acid sequence of the LCDR1 of monoclonal antibody ZV67 (e.g., SEQ ID NO: 109); an LCDR2 comprising the amino acid sequence of the LCDR2 of monoclonal antibody ZV67 (e.g., SEQ ID NO: 105); and an LCDR3 comprising the amino acid sequence of the LCDR3 of monoclonal antibody ZV67 (e.g., SEQ ID NO: 110).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZV67 (e.g., SEQ ID NO: 3). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZV67 (e.g., SEQ ID NO: 4).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZV67 (e.g., SEQ ID NO: 3); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZV67 (e.g., SEQ ID NO: 4). In an embodiment, the antibody molecule comprises: (i) a VH comprising the amino acid sequence of the VH of monoclonal antibody ZV67 (e.g., SEQ ID NO: 3); and (ii) a VL comprising the amino acid sequence of the VL of monoclonal antibody ZV67 (e.g., SEQ ID NO: 4).

In an embodiment the antibody molecule comprises monoclonal antibody ZV67. In an embodiment, the antibody molecule comprises a humanized monoclonal antibody ZV67.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/1 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/1 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/1 (e.g., SEQ ID NO: 106).

In an embodiment, the antibody molecule comprises: (i) a VH comprising: an HCDR1 comprising the amino acid sequence of the HCDR1 of monoclonal antibody ZA-1/1 (e.g., SEQ ID NO: 101); an HCDR2 comprising the amino acid sequence of the HCDR2 of monoclonal antibody ZA-1/1 (e.g., SEQ ID NO: 108); and an HCDR3 comprising the amino acid sequence of the HCDR3 of monoclonal antibody ZA-1/1 (e.g., SEQ ID NO: 103), and (ii) a VL comprising: an LCDR1 comprising the amino acid sequence of the LCDR1 of monoclonal antibody ZA-1/1 (e.g., SEQ ID NO: 111); an LCDR2 comprising the amino acid sequence of the LCDR2 of monoclonal antibody ZA-1/1 (e.g., SEQ ID NO: 105); and an LCDR3 comprising the amino acid sequence of the LCDR3 of monoclonal antibody ZA-1/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/1 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/1 (e.g., SEQ ID NO: 205); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/1 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/1 (e.g., SEQ ID NO: 205); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/1 (e.g., SEQ ID NO: 106).

In an embodiment, the antibody molecule comprises: (i) a VH comprising: an HCDR1 comprising the amino acid sequence of the HCDR1 of monoclonal antibody ZA-1/1 (e.g., SEQ ID NO: 201); an HCDR2 comprising the amino acid sequence of the HCDR2 of monoclonal antibody ZA-1/1 (e.g., SEQ ID NO: 205); and an HCDR3 comprising the amino acid sequence of the HCDR3 of monoclonal antibody ZA-1/1 (e.g., SEQ ID NO: 103), and (ii) a VL comprising: an LCDR1 comprising the amino acid sequence of the LCDR1 of monoclonal antibody ZA-1/1 (e.g., SEQ ID NO: 111); an LCDR2 comprising the amino acid sequence of the LCDR2 of monoclonal antibody ZA-1/1 (e.g., SEQ ID NO: 105); and an LCDR3 comprising the amino acid sequence of the LCDR3 of monoclonal antibody ZA-1/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-1/1 (e.g., SEQ ID NO: 5). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-1/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-1/1 (e.g., SEQ ID NO: 5); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-1/1 (e.g., SEQ ID NO: 6). In an embodiment, the antibody molecule comprises: (i) a VH comprising the amino acid sequence of the VH of monoclonal antibody ZA-1/1 (e.g., SEQ ID NO: 5); and (ii) a VL comprising the amino acid sequence of the VL of monoclonal antibody ZA-1/1 (e.g., SEQ ID NO: 6).

In an embodiment, the antibody molecule comprises a VH encoded by the nucleotide sequence of SEQ ID NO: 300 (or a nucleotide sequence substantially identical thereto) or a VL encoded by the nucleotide sequence of SEQ ID NO: 305 (or a nucleotide sequence substantially identical thereto), or both.

In an embodiment the antibody molecule comprises monoclonal antibody ZA-1/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/2 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/2 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/2 (e.g., SEQ ID NO: 106).

In an embodiment, the antibody molecule comprises: (i) a VH comprising: an HCDR1 comprising the amino acid sequence of the HCDR1 of monoclonal antibody ZA-1/2 (e.g., SEQ ID NO: 101); an HCDR2 comprising the amino acid sequence of the HCDR2 of monoclonal antibody ZA-1/2 (e.g., SEQ ID NO: 108); and an HCDR3 comprising the amino acid sequence of the HCDR3 of monoclonal antibody ZA-1/2 (e.g., SEQ ID NO: 103), and (ii) a VL comprising: an LCDR1 comprising the amino acid sequence of the LCDR1 of monoclonal antibody ZA-1/2 (e.g., SEQ ID NO: 104); an LCDR2 comprising the amino acid sequence of the LCDR2 of monoclonal antibody ZA-1/2 (e.g., SEQ ID NO: 105); and an LCDR3 comprising the amino acid sequence of the LCDR3 of monoclonal antibody ZA-1/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/2 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/2 (e.g., SEQ ID NO: 205); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/2 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/2 (e.g., SEQ ID NO: 205); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/2 (e.g., SEQ ID NO: 106).

In an embodiment, the antibody molecule comprises: (i) a VH comprising: an HCDR1 comprising the amino acid sequence of the HCDR1 of monoclonal antibody ZA-1/2 (e.g., SEQ ID NO: 201); an HCDR2 comprising the amino acid sequence of the HCDR2 of monoclonal antibody ZA-1/2 (e.g., SEQ ID NO: 205); and an HCDR3 comprising the amino acid sequence of the HCDR3 of monoclonal antibody ZA-1/2 (e.g., SEQ ID NO: 103), and (ii) a VL comprising: an LCDR1 comprising the amino acid sequence of the LCDR1 of monoclonal antibody ZA-1/2 (e.g., SEQ ID NO: 104); an LCDR2 comprising the amino acid sequence of the LCDR2 of monoclonal antibody ZA-1/2 (e.g., SEQ ID NO: 105); and an LCDR3 comprising the amino acid sequence of the LCDR3 of monoclonal antibody ZA-1/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-1/2 (e.g., SEQ ID NO: 5). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-1/2 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-1/2 (e.g., SEQ ID NO: 5); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-1/2 (e.g., SEQ ID NO: 7). In an embodiment, the antibody molecule comprises: (i) a VH comprising the amino acid sequence of the VH of monoclonal antibody ZA-1/2 (e.g., SEQ ID NO: 5); and (ii) a VL comprising the amino acid sequence of the VL of monoclonal antibody ZA-1/2 (e.g., SEQ ID NO: 7).

In an embodiment, the antibody molecule comprises a VH encoded by the nucleotide sequence of SEQ ID NO: 300 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZA-1/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/3 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/3 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/3 (e.g., SEQ ID NO: 106).

In an embodiment, the antibody molecule comprises: (i) a VH comprising: an HCDR1 comprising the amino acid sequence of the HCDR1 of monoclonal antibody ZA-1/3 (e.g., SEQ ID NO: 101); an HCDR2 comprising the amino acid sequence of the HCDR2 of monoclonal antibody ZA-1/3 (e.g., SEQ ID NO: 108); and an HCDR3 comprising the amino acid sequence of the HCDR3 of monoclonal antibody ZA-1/3 (e.g., SEQ ID NO: 103), and (ii) a VL comprising: an LCDR1 comprising the amino acid sequence of the LCDR1 of monoclonal antibody ZA-1/3 (e.g., SEQ ID NO: 111); an LCDR2 comprising the amino acid sequence of the LCDR2 of monoclonal antibody ZA-1/3 (e.g., SEQ ID NO: 112); and an LCDR3 comprising the amino acid sequence of the LCDR3 of monoclonal antibody ZA-1/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/3 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/3 (e.g., SEQ ID NO: 205); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-1/3 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-1/3 (e.g., SEQ ID NO: 205); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-1/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-1/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-1/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-1/3 (e.g., SEQ ID NO: 106).

In an embodiment, the antibody molecule comprises: (i) a VH comprising: an HCDR1 comprising the amino acid sequence of the HCDR1 of monoclonal antibody ZA-1/3 (e.g., SEQ ID NO: 201); an HCDR2 comprising the amino acid sequence of the HCDR2 of monoclonal antibody ZA-1/3 (e.g., SEQ ID NO: 205); and an HCDR3 comprising the amino acid sequence of the HCDR3 of monoclonal antibody ZA-1/3 (e.g., SEQ ID NO: 103), and (ii) a VL comprising: an LCDR1 comprising the amino acid sequence of the LCDR1 of monoclonal antibody ZA-1/3 (e.g., SEQ ID NO: 111); an LCDR2 comprising the amino acid sequence of the LCDR2 of monoclonal antibody ZA-1/3 (e.g., SEQ ID NO: 112); and an LCDR3 comprising the amino acid sequence of the LCDR3 of monoclonal antibody ZA-1/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-1/3 (e.g., SEQ ID NO: 5). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-1/3 (e.g., SEQ ID NO: 8).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-1/3 (e.g., SEQ ID NO: 5); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-1/3 (e.g., SEQ ID NO: 8). In an embodiment, the antibody molecule comprises: (i) a VH comprising the amino acid sequence of the VH of monoclonal antibody ZA-1/3 (e.g., SEQ ID NO: 5); and (ii) a VL comprising the amino acid sequence of the VL of monoclonal antibody ZA-1/3 (e.g., SEQ ID NO: 8).

In an embodiment, the antibody molecule comprises a VH encoded by the nucleotide sequence of SEQ ID NO: 300 (or a nucleotide sequence substantially identical thereto) or a VL encoded by the nucleotide sequence of SEQ ID NO: 307 (or a nucleotide sequence substantially identical thereto), or both.

In an embodiment the antibody molecule comprises monoclonal antibody ZA-1/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/1 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/1 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/1 (e.g., SEQ ID NO: 106).

In an embodiment, the antibody molecule comprises: (i) a VH comprising: an HCDR1 comprising the amino acid sequence of the HCDR1 of monoclonal antibody ZA-2/1 (e.g., SEQ ID NO: 101); an HCDR2 comprising the amino acid sequence of the HCDR2 of monoclonal antibody ZA-2/1 (e.g., SEQ ID NO: 108); and an HCDR3 comprising the amino acid sequence of the HCDR3 of monoclonal antibody ZA-2/1 (e.g., SEQ ID NO: 103), and (ii) a VL comprising: an LCDR1 comprising the amino acid sequence of the LCDR1 of monoclonal antibody ZA-2/1 (e.g., SEQ ID NO: 111); an LCDR2 comprising the amino acid sequence of the LCDR2 of monoclonal antibody ZA-2/1 (e.g., SEQ ID NO: 105); and an LCDR3 comprising the amino acid sequence of the LCDR3 of monoclonal antibody ZA-2/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/1 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/1 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/1 (e.g., SEQ ID NO: 106).

In an embodiment, the antibody molecule comprises: (i) a VH comprising: an HCDR1 comprising the amino acid sequence of the HCDR1 of monoclonal antibody ZA-2/1 (e.g., SEQ ID NO: 201); an HCDR2 comprising the amino acid sequence of the HCDR2 of monoclonal antibody ZA-2/1 (e.g., SEQ ID NO: 206); and an HCDR3 comprising the amino acid sequence of the HCDR3 of monoclonal antibody ZA-2/1 (e.g., SEQ ID NO: 103), and (ii) a VL comprising: an LCDR1 comprising the amino acid sequence of the LCDR1 of monoclonal antibody ZA-2/1 (e.g., SEQ ID NO: 111); an LCDR2 comprising the amino acid sequence of the LCDR2 of monoclonal antibody ZA-2/1 (e.g., SEQ ID NO: 105); and an LCDR3 comprising the amino acid sequence of the LCDR3 of monoclonal antibody ZA-2/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-2/1 (e.g., SEQ ID NO: 9). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-2/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-2/1 (e.g., SEQ ID NO: 9); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-2/1 (e.g., SEQ ID NO: 6). In an embodiment, the antibody molecule comprises: (i) a VH comprising the amino acid sequence of the VH of monoclonal antibody ZA-2/1 (e.g., SEQ ID NO: 9); and (ii) a VL comprising the amino acid sequence of the VL of monoclonal antibody ZA-2/1 (e.g., SEQ ID NO: 6).

In an embodiment, the antibody molecule comprises a VH encoded by the nucleotide sequence of SEQ ID NO: 301 (or a nucleotide sequence substantially identical thereto) or a VL encoded by the nucleotide sequence of SEQ ID NO: 305 (or a nucleotide sequence substantially identical thereto), or both.

In an embodiment the antibody molecule comprises monoclonal antibody ZA-2/1. an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/2 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/2 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/2 (e.g., SEQ ID NO: 106).

In an embodiment, the antibody molecule comprises: (i) a VH comprising: an HCDR1 comprising the amino acid sequence of the HCDR1 of monoclonal antibody ZA-2/2 (e.g., SEQ ID NO: 101); an HCDR2 comprising the amino acid sequence of the HCDR2 of monoclonal antibody ZA-2/2 (e.g., SEQ ID NO: 108); and an HCDR3 comprising the amino acid sequence of the HCDR3 of monoclonal antibody ZA-2/2 (e.g., SEQ ID NO: 103), and (ii) a VL comprising: an LCDR1 comprising the amino acid sequence of the LCDR1 of monoclonal antibody ZA-2/2 (e.g., SEQ ID NO: 104); an LCDR2 comprising the amino acid sequence of the LCDR2 of monoclonal antibody ZA-2/2 (e.g., SEQ ID NO: 105); and an LCDR3 comprising the amino acid sequence of the LCDR3 of monoclonal antibody ZA-2/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/2 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/2 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/2 (e.g., SEQ ID NO: 106).

In an embodiment, the antibody molecule comprises: (i) a VH comprising: an HCDR1 comprising the amino acid sequence of the HCDR1 of monoclonal antibody ZA-2/2 (e.g., SEQ ID NO: 201); an HCDR2 comprising the amino acid sequence of the HCDR2 of monoclonal antibody ZA-2/2 (e.g., SEQ ID NO: 206); and an HCDR3 comprising the amino acid sequence of the HCDR3 of monoclonal antibody ZA-2/2 (e.g., SEQ ID NO: 103), and (ii) a VL comprising: an LCDR1 comprising the amino acid sequence of the LCDR1 of monoclonal antibody ZA-2/2 (e.g., SEQ ID NO: 104); an LCDR2 comprising the amino acid sequence of the LCDR2 of monoclonal antibody ZA-2/2 (e.g., SEQ ID NO: 105); and an LCDR3 comprising the amino acid sequence of the LCDR3 of monoclonal antibody ZA-2/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-2/2 (e.g., SEQ ID NO: 9). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-2/2 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-2/2 (e.g., SEQ ID NO: 9); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-2/2 (e.g., SEQ ID NO: 7). In an embodiment, the antibody molecule comprises: (i) a VH comprising the amino acid sequence of the VH of monoclonal antibody ZA-2/2 (e.g., SEQ ID NO: 9); and (ii) a VL comprising the amino acid sequence of the VL of monoclonal antibody ZA-2/2 (e.g., SEQ ID NO: 7).

In an embodiment, the antibody molecule comprises a VH encoded by the nucleotide sequence of SEQ ID NO: 301 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZA-2/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/3 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/3 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/3 (e.g., SEQ ID NO: 106).

In an embodiment, the antibody molecule comprises: (i) a VH comprising: an HCDR1 comprising the amino acid sequence of the HCDR1 of monoclonal antibody ZA-2/3 (e.g., SEQ ID NO: 101); an HCDR2 comprising the amino acid sequence of the HCDR2 of monoclonal antibody ZA-2/3 (e.g., SEQ ID NO: 108); and an HCDR3 comprising the amino acid sequence of the HCDR3 of monoclonal antibody ZA-2/3 (e.g., SEQ ID NO: 103), and (ii) a VL comprising: an LCDR1 comprising the amino acid sequence of the LCDR1 of monoclonal antibody ZA-2/3 (e.g., SEQ ID NO: 111); an LCDR2 comprising the amino acid sequence of the LCDR2 of monoclonal antibody ZA-2/3 (e.g., SEQ ID NO: 112); and an LCDR3 comprising the amino acid sequence of the LCDR3 of monoclonal antibody ZA-2/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/3 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-2/3 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-2/3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-2/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-2/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-2/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-2/3 (e.g., SEQ ID NO: 106).

In an embodiment, the antibody molecule comprises: (i) a VH comprising: an HCDR1 comprising the amino acid sequence of the HCDR1 of monoclonal antibody ZA-2/3 (e.g., SEQ ID NO: 201); an HCDR2 comprising the amino acid sequence of the HCDR2 of monoclonal antibody ZA-2/3 (e.g., SEQ ID NO: 206); and an HCDR3 comprising the amino acid sequence of the HCDR3 of monoclonal antibody ZA-2/3 (e.g., SEQ ID NO: 103), and (ii) a VL comprising: an LCDR1 comprising the amino acid sequence of the LCDR1 of monoclonal antibody ZA-2/3 (e.g., SEQ ID NO: 111); an LCDR2 comprising the amino acid sequence of the LCDR2 of monoclonal antibody ZA-2/3 (e.g., SEQ ID NO: 112); and an LCDR3 comprising the amino acid sequence of the LCDR3 of monoclonal antibody ZA-2/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-2/3 (e.g., SEQ ID NO: 9). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-2/3 (e.g., SEQ ID NO: 8).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-2/3 (e.g., SEQ ID NO: 9); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-2/3 (e.g., SEQ ID NO: 8). In an embodiment, the antibody molecule comprises: (i) a VH comprising the amino acid sequence of the VH of monoclonal antibody ZA-2/3 (e.g., SEQ ID NO: 9); and (ii) a VL comprising the amino acid sequence of the VL of monoclonal antibody ZA-2/3 (e.g., SEQ ID NO: 8).

In an embodiment, the antibody molecule comprises a VH encoded by the nucleotide sequence of SEQ ID NO: 301 (or a nucleotide sequence substantially identical thereto) or a VL encoded by the nucleotide sequence of SEQ ID NO: 307 (or a nucleotide sequence substantially identical thereto), or both.

In an embodiment the antibody molecule comprises monoclonal antibody ZA-2/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/1 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/1 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/1 (e.g., SEQ ID NO: 106).

In an embodiment, the antibody molecule comprises: (i) a VH comprising: an HCDR1 comprising the amino acid sequence of the HCDR1 of monoclonal antibody ZA-3/1 (e.g., SEQ ID NO: 101); an HCDR2 comprising the amino acid sequence of the HCDR2 of monoclonal antibody ZA-3/1 (e.g., SEQ ID NO: 108); and an HCDR3 comprising the amino acid sequence of the HCDR3 of monoclonal antibody ZA-3/1 (e.g., SEQ ID NO: 103), and (ii) a VL comprising: an LCDR1 comprising the amino acid sequence of the LCDR1 of monoclonal antibody ZA-3/1 (e.g., SEQ ID NO: 111); an LCDR2 comprising the amino acid sequence of the LCDR2 of monoclonal antibody ZA-3/1 (e.g., SEQ ID NO: 105); and an LCDR3 comprising the amino acid sequence of the LCDR3 of monoclonal antibody ZA-3/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/1 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/1 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/1 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/1 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/1 (e.g., SEQ ID NO: 106).

In an embodiment, the antibody molecule comprises: (i) a VH comprising: an HCDR1 comprising the amino acid sequence of the HCDR1 of monoclonal antibody ZA-3/1 (e.g., SEQ ID NO: 201); an HCDR2 comprising the amino acid sequence of the HCDR2 of monoclonal antibody ZA-3/1 (e.g., SEQ ID NO: 207); and an HCDR3 comprising the amino acid sequence of the HCDR3 of monoclonal antibody ZA-3/1 (e.g., SEQ ID NO: 103), and (ii) a VL comprising: an LCDR1 comprising the amino acid sequence of the LCDR1 of monoclonal antibody ZA-3/1 (e.g., SEQ ID NO: 111); an LCDR2 comprising the amino acid sequence of the LCDR2 of monoclonal antibody ZA-3/1 (e.g., SEQ ID NO: 105); and an LCDR3 comprising the amino acid sequence of the LCDR3 of monoclonal antibody ZA-3/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-3/1 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-3/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-3/1 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-3/1 (e.g., SEQ ID NO: 6). In an embodiment, the antibody molecule comprises: (i) a VH comprising the amino acid sequence of the VH of monoclonal antibody ZA-3/1 (e.g., SEQ ID NO: 10); and (ii) a VL comprising the amino acid sequence of the VL of monoclonal antibody ZA-3/1 (e.g., SEQ ID NO: 6).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 305 (or a nucleotide sequence substantially identical thereto), or both.

In an embodiment the antibody molecule comprises monoclonal antibody ZA-3/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/2 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/2 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/2 (e.g., SEQ ID NO: 106).

In an embodiment, the antibody molecule comprises: (i) a VH comprising: an HCDR1 comprising the amino acid sequence of the HCDR1 of monoclonal antibody ZA-3/2 (e.g., SEQ ID NO: 101); an HCDR2 comprising the amino acid sequence of the HCDR2 of monoclonal antibody ZA-3/2 (e.g., SEQ ID NO: 108); and an HCDR3 comprising the amino acid sequence of the HCDR3 of monoclonal antibody ZA-3/2 (e.g., SEQ ID NO: 103), and (ii) a VL comprising: an LCDR1 comprising the amino acid sequence of the LCDR1 of monoclonal antibody ZA-3/2 (e.g., SEQ ID NO: 104); an LCDR2 comprising the amino acid sequence of the LCDR2 of monoclonal antibody ZA-3/2 (e.g., SEQ ID NO: 105); and an LCDR3 comprising the amino acid sequence of the LCDR3 of monoclonal antibody ZA-3/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/2 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/2 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/2 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/2 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/2 (e.g., SEQ ID NO: 106).

In an embodiment, the antibody molecule comprises: (i) a VH comprising: an HCDR1 comprising the amino acid sequence of the HCDR1 of monoclonal antibody ZA-3/2 (e.g., SEQ ID NO: 201); an HCDR2 comprising the amino acid sequence of the HCDR2 of monoclonal antibody ZA-3/2 (e.g., SEQ ID NO: 207); and an HCDR3 comprising the amino acid sequence of the HCDR3 of monoclonal antibody ZA-3/2 (e.g., SEQ ID NO: 103), and (ii) a VL comprising: an LCDR1 comprising the amino acid sequence of the LCDR1 of monoclonal antibody ZA-3/2 (e.g., SEQ ID NO: 104); an LCDR2 comprising the amino acid sequence of the LCDR2 of monoclonal antibody ZA-3/2 (e.g., SEQ ID NO: 105); and an LCDR3 comprising the amino acid sequence of the LCDR3 of monoclonal antibody ZA-3/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-3/2 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-3/2 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-3/2 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-3/2 (e.g., SEQ ID NO: 7). In an embodiment, the antibody molecule comprises: (i) a VH comprising the amino acid sequence of the VH of monoclonal antibody ZA-3/2 (e.g., SEQ ID NO: 10); and (ii) a VL comprising the amino acid sequence of the VL of monoclonal antibody ZA-3/2 (e.g., SEQ ID NO: 7).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZA-3/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/3 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/3 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/3 (e.g., SEQ ID NO: 106).

In an embodiment, the antibody molecule comprises: (i) a VH comprising: an HCDR1 comprising the amino acid sequence of the HCDR1 of monoclonal antibody ZA-3/3 (e.g., SEQ ID NO: 101); an HCDR2 comprising the amino acid sequence of the HCDR2 of monoclonal antibody ZA-3/3 (e.g., SEQ ID NO: 108); and an HCDR3 comprising the amino acid sequence of the HCDR3 of monoclonal antibody ZA-3/3 (e.g., SEQ ID NO: 103), and (ii) a VL comprising: an LCDR1 comprising the amino acid sequence of the LCDR1 of monoclonal antibody ZA-3/3 (e.g., SEQ ID NO: 111); an LCDR2 comprising the amino acid sequence of the LCDR2 of monoclonal antibody ZA-3/3 (e.g., SEQ ID NO: 112); and an LCDR3 comprising the amino acid sequence of the LCDR3 of monoclonal antibody ZA-3/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/3 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/3 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-3/3 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-3/3 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-3/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-3/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-3/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-3/3 (e.g., SEQ ID NO: 106).

In an embodiment, the antibody molecule comprises: (i) a VH comprising: an HCDR1 comprising the amino acid sequence of the HCDR1 of monoclonal antibody ZA-3/3 (e.g., SEQ ID NO: 201); an HCDR2 comprising the amino acid sequence of the HCDR2 of monoclonal antibody ZA-3/3 (e.g., SEQ ID NO: 207); and an HCDR3 comprising the amino acid sequence of the HCDR3 of monoclonal antibody ZA-3/3 (e.g., SEQ ID NO: 103), and (ii) a VL comprising: an LCDR1 comprising the amino acid sequence of the LCDR1 of monoclonal antibody ZA-3/3 (e.g., SEQ ID NO: 111); an LCDR2 comprising the amino acid sequence of the LCDR2 of monoclonal antibody ZA-3/3 (e.g., SEQ ID NO: 112); and an LCDR3 comprising the amino acid sequence of the LCDR3 of monoclonal antibody ZA-3/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-3/3 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-3/3 (e.g., SEQ ID NO: 8).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-3/3 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-3/3 (e.g., SEQ ID NO: 8). In an embodiment, the antibody molecule comprises: (i) a VH comprising the amino acid sequence of the VH of monoclonal antibody ZA-3/3 (e.g., SEQ ID NO: 10); and (ii) a VL comprising the amino acid sequence of the VL of monoclonal antibody ZA-3/3 (e.g., SEQ ID NO: 8).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 307 (or a nucleotide sequence substantially identical thereto), or both.

In an embodiment the antibody molecule comprises monoclonal antibody ZA-3/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/1 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/1 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/1 (e.g., SEQ ID NO: 106).

In an embodiment, the antibody molecule comprises: (i) a VH comprising: an HCDR1 comprising the amino acid sequence of the HCDR1 of monoclonal antibody ZA-4/1 (e.g., SEQ ID NO: 101); an HCDR2 comprising the amino acid sequence of the HCDR2 of monoclonal antibody ZA-4/1 (e.g., SEQ ID NO: 108); and an HCDR3 comprising the amino acid sequence of the HCDR3 of monoclonal antibody ZA-4/1 (e.g., SEQ ID NO: 103), and (ii) a VL comprising: an LCDR1 comprising the amino acid sequence of the LCDR1 of monoclonal antibody ZA-4/1 (e.g., SEQ ID NO: 111); an LCDR2 comprising the amino acid sequence of the LCDR2 of monoclonal antibody ZA-4/1 (e.g., SEQ ID NO: 105); and an LCDR3 comprising the amino acid sequence of the LCDR3 of monoclonal antibody ZA-4/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/1 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/1 (e.g., SEQ ID NO: 208); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/1 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/1 (e.g., SEQ ID NO: 208); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/1 (e.g., SEQ ID NO: 106).

In an embodiment, the antibody molecule comprises: (i) a VH comprising: an HCDR1 comprising the amino acid sequence of the HCDR1 of monoclonal antibody ZA-4/1 (e.g., SEQ ID NO: 201); an HCDR2 comprising the amino acid sequence of the HCDR2 of monoclonal antibody ZA-4/1 (e.g., SEQ ID NO: 208); and an HCDR3 comprising the amino acid sequence of the HCDR3 of monoclonal antibody ZA-4/1 (e.g., SEQ ID NO: 103), and (ii) a VL comprising: an LCDR1 comprising the amino acid sequence of the LCDR1 of monoclonal antibody ZA-4/1 (e.g., SEQ ID NO: 111); an LCDR2 comprising the amino acid sequence of the LCDR2 of monoclonal antibody ZA-4/1 (e.g., SEQ ID NO: 105); and an LCDR3 comprising the amino acid sequence of the LCDR3 of monoclonal antibody ZA-4/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-4/1 (e.g., SEQ ID NO: 11). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-4/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-4/1 (e.g., SEQ ID NO: 11); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-4/1 (e.g., SEQ ID NO: 6). In an embodiment, the antibody molecule comprises: (i) a VH comprising the amino acid sequence of the VH of monoclonal antibody ZA-4/1 (e.g., SEQ ID NO: 11); and (ii) a VL comprising the amino acid sequence of the VL of monoclonal antibody ZA-4/1 (e.g., SEQ ID NO: 6).

In an embodiment, the antibody molecule comprises a VH encoded by the nucleotide sequence of SEQ ID NO: 303 (or a nucleotide sequence substantially identical thereto) or a VL encoded by the nucleotide sequence of SEQ ID NO: 305 (or a nucleotide sequence substantially identical thereto), or both.

In an embodiment the antibody molecule comprises monoclonal antibody ZA-4/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/2 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/2 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/2 (e.g., SEQ ID NO: 106).

In an embodiment, the antibody molecule comprises: (i) a VH comprising: an HCDR1 comprising the amino acid sequence of the HCDR1 of monoclonal antibody ZA-4/2 (e.g., SEQ ID NO: 101); an HCDR2 comprising the amino acid sequence of the HCDR2 of monoclonal antibody ZA-4/2 (e.g., SEQ ID NO: 108); and an HCDR3 comprising the amino acid sequence of the HCDR3 of monoclonal antibody ZA-4/2 (e.g., SEQ ID NO: 103), and (ii) a VL comprising: an LCDR1 comprising the amino acid sequence of the LCDR1 of monoclonal antibody ZA-4/2 (e.g., SEQ ID NO: 104); an LCDR2 comprising the amino acid sequence of the LCDR2 of monoclonal antibody ZA-4/2 (e.g., SEQ ID NO: 105); and an LCDR3 comprising the amino acid sequence of the LCDR3 of monoclonal antibody ZA-4/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/2 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/2 (e.g., SEQ ID NO: 208); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/2 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/2 (e.g., SEQ ID NO: 208); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/2 (e.g., SEQ ID NO: 106).

In an embodiment, the antibody molecule comprises: (i) a VH comprising: an HCDR1 comprising the amino acid sequence of the HCDR1 of monoclonal antibody ZA-4/2 (e.g., SEQ ID NO: 201); an HCDR2 comprising the amino acid sequence of the HCDR2 of monoclonal antibody ZA-4/2 (e.g., SEQ ID NO: 208); and an HCDR3 comprising the amino acid sequence of the HCDR3 of monoclonal antibody ZA-4/2 (e.g., SEQ ID NO: 103), and (ii) a VL comprising: an LCDR1 comprising the amino acid sequence of the LCDR1 of monoclonal antibody ZA-4/2 (e.g., SEQ ID NO: 104); an LCDR2 comprising the amino acid sequence of the LCDR2 of monoclonal antibody ZA-4/2 (e.g., SEQ ID NO: 105); and an LCDR3 comprising the amino acid sequence of the LCDR3 of monoclonal antibody ZA-4/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-4/2 (e.g., SEQ ID NO: 11). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-4/2 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-4/2 (e.g., SEQ ID NO: 11); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-4/2 (e.g., SEQ ID NO: 7). In an embodiment, the antibody molecule comprises: (i) a VH comprising the amino acid sequence of the VH of monoclonal antibody ZA-4/2 (e.g., SEQ ID NO: 11); and (ii) a VL comprising the amino acid sequence of the VL of monoclonal antibody ZA-4/2 (e.g., SEQ ID NO: 7).

In an embodiment, the antibody molecule comprises a VH encoded by the nucleotide sequence of SEQ ID NO: 303 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZA-4/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/3 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/3 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/3 (e.g., SEQ ID NO: 106).

In an embodiment, the antibody molecule comprises: (i) a VH comprising: an HCDR1 comprising the amino acid sequence of the HCDR1 of monoclonal antibody ZA-4/3 (e.g., SEQ ID NO: 101); an HCDR2 comprising the amino acid sequence of the HCDR2 of monoclonal antibody ZA-4/3 (e.g., SEQ ID NO: 108); and an HCDR3 comprising the amino acid sequence of the HCDR3 of monoclonal antibody ZA-4/3 (e.g., SEQ ID NO: 103), and (ii) a VL comprising: an LCDR1 comprising the amino acid sequence of the LCDR1 of monoclonal antibody ZA-4/3 (e.g., SEQ ID NO: 111); an LCDR2 comprising the amino acid sequence of the LCDR2 of monoclonal antibody ZA-4/3 (e.g., SEQ ID NO: 112); and an LCDR3 comprising the amino acid sequence of the LCDR3 of monoclonal antibody ZA-4/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/3 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/3 (e.g., SEQ ID NO: 208); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-4/3 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-4/3 (e.g., SEQ ID NO: 208); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-4/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-4/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-4/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-4/3 (e.g., SEQ ID NO: 106).

In an embodiment, the antibody molecule comprises: (i) a VH comprising: an HCDR1 comprising the amino acid sequence of the HCDR1 of monoclonal antibody ZA-4/3 (e.g., SEQ ID NO: 201); an HCDR2 comprising the amino acid sequence of the HCDR2 of monoclonal antibody ZA-4/3 (e.g., SEQ ID NO: 208); and an HCDR3 comprising the amino acid sequence of the HCDR3 of monoclonal antibody ZA-4/3 (e.g., SEQ ID NO: 103), and (ii) a VL comprising: an LCDR1 comprising the amino acid sequence of the LCDR1 of monoclonal antibody ZA-4/3 (e.g., SEQ ID NO: 111); an LCDR2 comprising the amino acid sequence of the LCDR2 of monoclonal antibody ZA-4/3 (e.g., SEQ ID NO: 112); and an LCDR3 comprising the amino acid sequence of the LCDR3 of monoclonal antibody ZA-4/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-4/3 (e.g., SEQ ID NO: 11). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-4/3 (e.g., SEQ ID NO: 8).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-4/3 (e.g., SEQ ID NO: 11); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-4/3 (e.g., SEQ ID NO: 8). In an embodiment, the antibody molecule comprises: (i) a VH comprising the amino acid sequence of the VH of monoclonal antibody ZA-4/3 (e.g., SEQ ID NO: 11); and (ii) a VL comprising the amino acid sequence of the VL of monoclonal antibody ZA-4/3 (e.g., SEQ ID NO: 8).

In an embodiment, the antibody molecule comprises a VH encoded by the nucleotide sequence of SEQ ID NO: 303 (or a nucleotide sequence substantially identical thereto) or a VL encoded by the nucleotide sequence of SEQ ID NO: 307 (or a nucleotide sequence substantially identical thereto), or both.

In an embodiment the antibody molecule comprises monoclonal antibody ZA-4/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/1 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/1 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/1 (e.g., SEQ ID NO: 106).

In an embodiment, the antibody molecule comprises: (i) a VH comprising: an HCDR1 comprising the amino acid sequence of the HCDR1 of monoclonal antibody ZA-5/1 (e.g., SEQ ID NO: 101); an HCDR2 comprising the amino acid sequence of the HCDR2 of monoclonal antibody ZA-5/1 (e.g., SEQ ID NO: 108); and an HCDR3 comprising the amino acid sequence of the HCDR3 of monoclonal antibody ZA-5/1 (e.g., SEQ ID NO: 103), and (ii) a VL comprising: an LCDR1 comprising the amino acid sequence of the LCDR1 of monoclonal antibody ZA-5/1 (e.g., SEQ ID NO: 111); an LCDR2 comprising the amino acid sequence of the LCDR2 of monoclonal antibody ZA-5/1 (e.g., SEQ ID NO: 105); and an LCDR3 comprising the amino acid sequence of the LCDR3 of monoclonal antibody ZA-5/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/1 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/1 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/1 (e.g., SEQ ID NO: 106).

In an embodiment, the antibody molecule comprises: (i) a VH comprising: an HCDR1 comprising the amino acid sequence of the HCDR1 of monoclonal antibody ZA-5/1 (e.g., SEQ ID NO: 201); an HCDR2 comprising the amino acid sequence of the HCDR2 of monoclonal antibody ZA-5/1 (e.g., SEQ ID NO: 206); and an HCDR3 comprising the amino acid sequence of the HCDR3 of monoclonal antibody ZA-5/1 (e.g., SEQ ID NO: 103), and (ii) a VL comprising: an LCDR1 comprising the amino acid sequence of the LCDR1 of monoclonal antibody ZA-5/1 (e.g., SEQ ID NO: 111); an LCDR2 comprising the amino acid sequence of the LCDR2 of monoclonal antibody ZA-5/1 (e.g., SEQ ID NO: 105); and an LCDR3 comprising the amino acid sequence of the LCDR3 of monoclonal antibody ZA-5/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-5/1 (e.g., SEQ ID NO: 12). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-5/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-5/1 (e.g., SEQ ID NO: 12); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-5/1 (e.g., SEQ ID NO: 6). In an embodiment, the antibody molecule comprises: (i) a VH comprising the amino acid sequence of the VH of monoclonal antibody ZA-5/1 (e.g., SEQ ID NO: 12); and (ii) a VL comprising the amino acid sequence of the VL of monoclonal antibody ZA-5/1 (e.g., SEQ ID NO: 6).

In an embodiment, the antibody molecule comprises a VH encoded by the nucleotide sequence of SEQ ID NO: 304 (or a nucleotide sequence substantially identical thereto) or a VL encoded by the nucleotide sequence of SEQ ID NO: 305 (or a nucleotide sequence substantially identical thereto), or both.

In an embodiment the antibody molecule comprises monoclonal antibody ZA-5/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/2 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/2 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/2 (e.g., SEQ ID NO: 106).

In an embodiment, the antibody molecule comprises: (i) a VH comprising: an HCDR1 comprising the amino acid sequence of the HCDR1 of monoclonal antibody ZA-5/2 (e.g., SEQ ID NO: 101); an HCDR2 comprising the amino acid sequence of the HCDR2 of monoclonal antibody ZA-5/2 (e.g., SEQ ID NO: 108); and an HCDR3 comprising the amino acid sequence of the HCDR3 of monoclonal antibody ZA-5/2 (e.g., SEQ ID NO: 103), and (ii) a VL comprising: an LCDR1 comprising the amino acid sequence of the LCDR1 of monoclonal antibody ZA-5/2 (e.g., SEQ ID NO: 104); an LCDR2 comprising the amino acid sequence of the LCDR2 of monoclonal antibody ZA-5/2 (e.g., SEQ ID NO: 105); and an LCDR3 comprising the amino acid sequence of the LCDR3 of monoclonal antibody ZA-5/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/2 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/2 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/2 (e.g., SEQ ID NO: 106).

In an embodiment, the antibody molecule comprises: (i) a VH comprising: an HCDR1 comprising the amino acid sequence of the HCDR1 of monoclonal antibody ZA-5/2 (e.g., SEQ ID NO: 201); an HCDR2 comprising the amino acid sequence of the HCDR2 of monoclonal antibody ZA-5/2 (e.g., SEQ ID NO: 206); and an HCDR3 comprising the amino acid sequence of the HCDR3 of monoclonal antibody ZA-5/2 (e.g., SEQ ID NO: 103), and (ii) a VL comprising: an LCDR1 comprising the amino acid sequence of the LCDR1 of monoclonal antibody ZA-5/2 (e.g., SEQ ID NO: 104); an LCDR2 comprising the amino acid sequence of the LCDR2 of monoclonal antibody ZA-5/2 (e.g., SEQ ID NO: 105); and an LCDR3 comprising the amino acid sequence of the LCDR3 of monoclonal antibody ZA-5/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-5/2 (e.g., SEQ ID NO: 12). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-5/2 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-5/2 (e.g., SEQ ID NO: 12); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-5/2 (e.g., SEQ ID NO: 7). In an embodiment, the antibody molecule comprises: (i) a VH comprising the amino acid sequence of the VH of monoclonal antibody ZA-5/2 (e.g., SEQ ID NO: 12); and (ii) a VL comprising the amino acid sequence of the VL of monoclonal antibody ZA-5/2 (e.g., SEQ ID NO: 7).

In an embodiment, the antibody molecule comprises a VH encoded by the nucleotide sequence of SEQ ID NO: 304 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZA-5/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/3 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/3 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/3 (e.g., SEQ ID NO: 106).

In an embodiment, the antibody molecule comprises: (i) a VH comprising: an HCDR1 comprising the amino acid sequence of the HCDR1 of monoclonal antibody ZA-5/3 (e.g., SEQ ID NO: 101); an HCDR2 comprising the amino acid sequence of the HCDR2 of monoclonal antibody ZA-5/3 (e.g., SEQ ID NO: 108); and an HCDR3 comprising the amino acid sequence of the HCDR3 of monoclonal antibody ZA-5/3 (e.g., SEQ ID NO: 103), and (ii) a VL comprising: an LCDR1 comprising the amino acid sequence of the LCDR1 of monoclonal antibody ZA-5/3 (e.g., SEQ ID NO: 111); an LCDR2 comprising the amino acid sequence of the LCDR2 of monoclonal antibody ZA-5/3 (e.g., SEQ ID NO: 112); and an LCDR3 comprising the amino acid sequence of the LCDR3 of monoclonal antibody ZA-5/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/3 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/3 (e.g., SEQ ID NO: 112); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZA-5/3 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZA-5/3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZA-5/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZA-5/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZA-5/3 (e.g., SEQ ID NO: 112); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZA-5/3 (e.g., SEQ ID NO: 106).

In an embodiment, the antibody molecule comprises: (i) a VH comprising: an HCDR1 comprising the amino acid sequence of the HCDR1 of monoclonal antibody ZA-5/3 (e.g., SEQ ID NO: 201); an HCDR2 comprising the amino acid sequence of the HCDR2 of monoclonal antibody ZA-5/3 (e.g., SEQ ID NO: 206); and an HCDR3 comprising the amino acid sequence of the HCDR3 of monoclonal antibody ZA-5/3 (e.g., SEQ ID NO: 103), and (ii) a VL comprising: an LCDR1 comprising the amino acid sequence of the LCDR1 of monoclonal antibody ZA-5/3 (e.g., SEQ ID NO: 111); an LCDR2 comprising the amino acid sequence of the LCDR2 of monoclonal antibody ZA-5/3 (e.g., SEQ ID NO: 112); and an LCDR3 comprising the amino acid sequence of the LCDR3 of monoclonal antibody ZA-5/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-5/3 (e.g., SEQ ID NO: 12). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-5/3 (e.g., SEQ ID NO: 8).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZA-5/3 (e.g., SEQ ID NO: 12); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZA-5/3 (e.g., SEQ ID NO: 8). In an embodiment, the antibody molecule comprises: (i) a VH comprising the amino acid sequence of the VH of monoclonal antibody ZA-5/3 (e.g., SEQ ID NO: 12); and (ii) a VL comprising the amino acid sequence of the VL of monoclonal antibody ZA-5/3 (e.g., SEQ ID NO: 8).

In an embodiment, the antibody molecule comprises a VH encoded by the nucleotide sequence of SEQ ID NO: 304 (or a nucleotide sequence substantially identical thereto) or a VL encoded by the nucleotide sequence of SEQ ID NO: 307 (or a nucleotide sequence substantially identical thereto), or both.

In an embodiment the antibody molecule comprises monoclonal antibody ZA-5/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/1 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/1 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/1 (e.g., SEQ ID NO: 106).

In an embodiment, the antibody molecule comprises: (i) a VH comprising: an HCDR1 comprising the amino acid sequence of the HCDR1 of monoclonal antibody ZB-1/1 (e.g., SEQ ID NO: 107); an HCDR2 comprising the amino acid sequence of the HCDR2 of monoclonal antibody ZB-1/1 (e.g., SEQ ID NO: 108); and an HCDR3 comprising the amino acid sequence of the HCDR3 of monoclonal antibody ZB-1/1 (e.g., SEQ ID NO: 103), and (ii) a VL comprising: an LCDR1 comprising the amino acid sequence of the LCDR1 of monoclonal antibody ZB-1/1 (e.g., SEQ ID NO: 111); an LCDR2 comprising the amino acid sequence of the LCDR2 of monoclonal antibody ZB-1/1 (e.g., SEQ ID NO: 105); and an LCDR3 comprising the amino acid sequence of the LCDR3 of monoclonal antibody ZB-1/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/1 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/1 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/1 (e.g., SEQ ID NO: 106).

In an embodiment, the antibody molecule comprises: (i) a VH comprising: an HCDR1 comprising the amino acid sequence of the HCDR1 of monoclonal antibody ZB-1/1 (e.g., SEQ ID NO: 203); an HCDR2 comprising the amino acid sequence of the HCDR2 of monoclonal antibody ZB-1/1 (e.g., SEQ ID NO: 206); and an HCDR3 comprising the amino acid sequence of the HCDR3 of monoclonal antibody ZB-1/1 (e.g., SEQ ID NO: 103), and (ii) a VL comprising: an LCDR1 comprising the amino acid sequence of the LCDR1 of monoclonal antibody ZB-1/1 (e.g., SEQ ID NO: 111); an LCDR2 comprising the amino acid sequence of the LCDR2 of monoclonal antibody ZB-1/1 (e.g., SEQ ID NO: 105); and an LCDR3 comprising the amino acid sequence of the LCDR3 of monoclonal antibody ZB-1/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/1 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/1 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/1 (e.g., SEQ ID NO: 6). In an embodiment, the antibody molecule comprises: (i) a VH comprising the amino acid sequence of the VH of monoclonal antibody ZB-1/1 (e.g., SEQ ID NO: 13); and (ii) a VL comprising the amino acid sequence of the VL of monoclonal antibody ZB-1/1 (e.g., SEQ ID NO: 6).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZB-1/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/2 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/2 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/2 (e.g., SEQ ID NO: 106).

In an embodiment, the antibody molecule comprises: (i) a VH comprising: an HCDR1 comprising the amino acid sequence of the HCDR1 of monoclonal antibody ZB-1/2 (e.g., SEQ ID NO: 107); an HCDR2 comprising the amino acid sequence of the HCDR2 of monoclonal antibody ZB-1/2 (e.g., SEQ ID NO: 108); and an HCDR3 comprising the amino acid sequence of the HCDR3 of monoclonal antibody ZB-1/2 (e.g., SEQ ID NO: 103), and (ii) a VL comprising: an LCDR1 comprising the amino acid sequence of the LCDR1 of monoclonal antibody ZB-1/2 (e.g., SEQ ID NO: 117); an LCDR2 comprising the amino acid sequence of the LCDR2 of monoclonal antibody ZB-1/2 (e.g., SEQ ID NO: 105); and an LCDR3 comprising the amino acid sequence of the LCDR3 of monoclonal antibody ZB-1/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/2 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/2 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/2 (e.g., SEQ ID NO: 106).

In an embodiment, the antibody molecule comprises: (i) a VH comprising: an HCDR1 comprising the amino acid sequence of the HCDR1 of monoclonal antibody ZB-1/2 (e.g., SEQ ID NO: 203); an HCDR2 comprising the amino acid sequence of the HCDR2 of monoclonal antibody ZB-1/2 (e.g., SEQ ID NO: 206); and an HCDR3 comprising the amino acid sequence of the HCDR3 of monoclonal antibody ZB-1/2 (e.g., SEQ ID NO: 103), and (ii) a VL comprising: an LCDR1 comprising the amino acid sequence of the LCDR1 of monoclonal antibody ZB-1/2 (e.g., SEQ ID NO: 117); an LCDR2 comprising the amino acid sequence of the LCDR2 of monoclonal antibody ZB-1/2 (e.g., SEQ ID NO: 105); and an LCDR3 comprising the amino acid sequence of the LCDR3 of monoclonal antibody ZB-1/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/2 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/2 (e.g., SEQ ID NO: 18).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/2 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/2 (e.g., SEQ ID NO: 18). In an embodiment, the antibody molecule comprises: (i) a VH comprising the amino acid sequence of the VH of monoclonal antibody ZB-1/2 (e.g., SEQ ID NO: 13); and (ii) a VL comprising the amino acid sequence of the VL of monoclonal antibody ZB-1/2 (e.g., SEQ ID NO: 18).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 314 (or a nucleotide sequence substantially identical thereto), or both.

In an embodiment the antibody molecule comprises monoclonal antibody ZB-1/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/3 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/3 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/3 (e.g., SEQ ID NO: 106).

In an embodiment, the antibody molecule comprises: (i) a VH comprising: an HCDR1 comprising the amino acid sequence of the HCDR1 of monoclonal antibody ZB-1/3 (e.g., SEQ ID NO: 107); an HCDR2 comprising the amino acid sequence of the HCDR2 of monoclonal antibody ZB-1/3 (e.g., SEQ ID NO: 108); and an HCDR3 comprising the amino acid sequence of the HCDR3 of monoclonal antibody ZB-1/3 (e.g., SEQ ID NO: 103), and (ii) a VL comprising: an LCDR1 comprising the amino acid sequence of the LCDR1 of monoclonal antibody ZB-1/3 (e.g., SEQ ID NO: 111); an LCDR2 comprising the amino acid sequence of the LCDR2 of monoclonal antibody ZB-1/3 (e.g., SEQ ID NO: 118); and an LCDR3 comprising the amino acid sequence of the LCDR3 of monoclonal antibody ZB-1/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/3 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/3 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/3 (e.g., SEQ ID NO: 106).

In an embodiment, the antibody molecule comprises: (i) a VH comprising: an HCDR1 comprising the amino acid sequence of the HCDR1 of monoclonal antibody ZB-1/3 (e.g., SEQ ID NO: 203); an HCDR2 comprising the amino acid sequence of the HCDR2 of monoclonal antibody ZB-1/3 (e.g., SEQ ID NO: 206); and an HCDR3 comprising the amino acid sequence of the HCDR3 of monoclonal antibody ZB-1/3 (e.g., SEQ ID NO: 103), and (ii) a VL comprising: an LCDR1 comprising the amino acid sequence of the LCDR1 of monoclonal antibody ZB-1/3 (e.g., SEQ ID NO: 111); an LCDR2 comprising the amino acid sequence of the LCDR2 of monoclonal antibody ZB-1/3 (e.g., SEQ ID NO: 118); and an LCDR3 comprising the amino acid sequence of the LCDR3 of monoclonal antibody ZB-1/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/3 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/3 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/3 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/3 (e.g., SEQ ID NO: 19). In an embodiment, the antibody molecule comprises: (i) a VH comprising the amino acid sequence of the VH of monoclonal antibody ZB-1/3 (e.g., SEQ ID NO: 13); and (ii) a VL comprising the amino acid sequence of the VL of monoclonal antibody ZB-1/3 (e.g., SEQ ID NO: 19).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 315 (or a nucleotide sequence substantially identical thereto), or both.

In an embodiment the antibody molecule comprises monoclonal antibody ZB-1/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/4 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/4 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/4 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/4 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/4 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/4 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/4 (e.g., SEQ ID NO: 119).

In an embodiment, the antibody molecule comprises: (i) a VH comprising: an HCDR1 comprising the amino acid sequence of the HCDR1 of monoclonal antibody ZB-1/4 (e.g., SEQ ID NO: 107); an HCDR2 comprising the amino acid sequence of the HCDR2 of monoclonal antibody ZB-1/4 (e.g., SEQ ID NO: 108); and an HCDR3 comprising the amino acid sequence of the HCDR3 of monoclonal antibody ZB-1/4 (e.g., SEQ ID NO: 103), and (ii) a VL comprising: an LCDR1 comprising the amino acid sequence of the LCDR1 of monoclonal antibody ZB-1/4 (e.g., SEQ ID NO: 111); an LCDR2 comprising the amino acid sequence of the LCDR2 of monoclonal antibody ZB-1/4 (e.g., SEQ ID NO: 105); and an LCDR3 comprising the amino acid sequence of the LCDR3 of monoclonal antibody ZB-1/4 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/4 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/4 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/4 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-1/4 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-1/4 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-1/4 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-1/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-1/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-1/4 (e.g., SEQ ID NO: 119).

In an embodiment, the antibody molecule comprises: (i) a VH comprising: an HCDR1 comprising the amino acid sequence of the HCDR1 of monoclonal antibody ZB-1/4 (e.g., SEQ ID NO: 203); an HCDR2 comprising the amino acid sequence of the HCDR2 of monoclonal antibody ZB-1/4 (e.g., SEQ ID NO: 206); and an HCDR3 comprising the amino acid sequence of the HCDR3 of monoclonal antibody ZB-1/4 (e.g., SEQ ID NO: 103), and (ii) a VL comprising: an LCDR1 comprising the amino acid sequence of the LCDR1 of monoclonal antibody ZB-1/4 (e.g., SEQ ID NO: 111); an LCDR2 comprising the amino acid sequence of the LCDR2 of monoclonal antibody ZB-1/4 (e.g., SEQ ID NO: 105); and an LCDR3 comprising the amino acid sequence of the LCDR3 of monoclonal antibody ZB-1/4 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/4 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/4 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-1/4 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-1/4 (e.g., SEQ ID NO: 20). In an embodiment, the antibody molecule comprises: (i) a VH comprising the amino acid sequence of the VH of monoclonal antibody ZB-1/4 (e.g., SEQ ID NO: 13); and (ii) a VL comprising the amino acid sequence of the VL of monoclonal antibody ZB-1/4 (e.g., SEQ ID NO: 20).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZB-1/4.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/1 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/1 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/1 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/1 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/1 (e.g., SEQ ID NO: 106).

In an embodiment, the antibody molecule comprises: (i) a VH comprising: an HCDR1 comprising the amino acid sequence of the HCDR1 of monoclonal antibody ZB-2/1 (e.g., SEQ ID NO: 107); an HCDR2 comprising the amino acid sequence of the HCDR2 of monoclonal antibody ZB-2/1 (e.g., SEQ ID NO: 108); and an HCDR3 comprising the amino acid sequence of the HCDR3 of monoclonal antibody ZB-2/1 (e.g., SEQ ID NO: 113), and (ii) a VL comprising: an LCDR1 comprising the amino acid sequence of the LCDR1 of monoclonal antibody ZB-2/1 (e.g., SEQ ID NO: 111); an LCDR2 comprising the amino acid sequence of the LCDR2 of monoclonal antibody ZB-2/1 (e.g., SEQ ID NO: 105); and an LCDR3 comprising the amino acid sequence of the LCDR3 of monoclonal antibody ZB-2/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/1 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/1 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/1 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/1 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/1 (e.g., SEQ ID NO: 106).

In an embodiment, the antibody molecule comprises: (i) a VH comprising: an HCDR1 comprising the amino acid sequence of the HCDR1 of monoclonal antibody ZB-2/1 (e.g., SEQ ID NO: 203); an HCDR2 comprising the amino acid sequence of the HCDR2 of monoclonal antibody ZB-2/1 (e.g., SEQ ID NO: 206); and an HCDR3 comprising the amino acid sequence of the HCDR3 of monoclonal antibody ZB-2/1 (e.g., SEQ ID NO: 113), and (ii) a VL comprising: an LCDR1 comprising the amino acid sequence of the LCDR1 of monoclonal antibody ZB-2/1 (e.g., SEQ ID NO: 111); an LCDR2 comprising the amino acid sequence of the LCDR2 of monoclonal antibody ZB-2/1 (e.g., SEQ ID NO: 105); and an LCDR3 comprising the amino acid sequence of the LCDR3 of monoclonal antibody ZB-2/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/1 (e.g., SEQ ID NO: 14). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/1 (e.g., SEQ ID NO: 14); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/1 (e.g., SEQ ID NO: 6). In an embodiment, the antibody molecule comprises: (i) a VH comprising the amino acid sequence of the VH of monoclonal antibody ZB-2/1 (e.g., SEQ ID NO: 14); and (ii) a VL comprising the amino acid sequence of the VL of monoclonal antibody ZB-2/1 (e.g., SEQ ID NO: 6).

In an embodiment, the antibody molecule comprises a VH encoded by the nucleotide sequence of SEQ ID NO: 309 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZB-2/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/2 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/2 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/2 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/2 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/2 (e.g., SEQ ID NO: 106).

In an embodiment, the antibody molecule comprises: (i) a VH comprising: an HCDR1 comprising the amino acid sequence of the HCDR1 of monoclonal antibody ZB-2/2 (e.g., SEQ ID NO: 107); an HCDR2 comprising the amino acid sequence of the HCDR2 of monoclonal antibody ZB-2/2 (e.g., SEQ ID NO: 108); and an HCDR3 comprising the amino acid sequence of the HCDR3 of monoclonal antibody ZB-2/2 (e.g., SEQ ID NO: 113), and (ii) a VL comprising: an LCDR1 comprising the amino acid sequence of the LCDR1 of monoclonal antibody ZB-2/2 (e.g., SEQ ID NO: 117); an LCDR2 comprising the amino acid sequence of the LCDR2 of monoclonal antibody ZB-2/2 (e.g., SEQ ID NO: 105); and an LCDR3 comprising the amino acid sequence of the LCDR3 of monoclonal antibody ZB-2/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/2 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/2 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/2 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/2 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/2 (e.g., SEQ ID NO: 106).

In an embodiment, the antibody molecule comprises: (i) a VH comprising: an HCDR1 comprising the amino acid sequence of the HCDR1 of monoclonal antibody ZB-2/2 (e.g., SEQ ID NO: 203); an HCDR2 comprising the amino acid sequence of the HCDR2 of monoclonal antibody ZB-2/2 (e.g., SEQ ID NO: 206); and an HCDR3 comprising the amino acid sequence of the HCDR3 of monoclonal antibody ZB-2/2 (e.g., SEQ ID NO: 113), and (ii) a VL comprising: an LCDR1 comprising the amino acid sequence of the LCDR1 of monoclonal antibody ZB-2/2 (e.g., SEQ ID NO: 117); an LCDR2 comprising the amino acid sequence of the LCDR2 of monoclonal antibody ZB-2/2 (e.g., SEQ ID NO: 105); and an LCDR3 comprising the amino acid sequence of the LCDR3 of monoclonal antibody ZB-2/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/2 (e.g., SEQ ID NO: 14). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/2 (e.g., SEQ ID NO: 18).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/2 (e.g., SEQ ID NO: 14); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/2 (e.g., SEQ ID NO: 18). In an embodiment, the antibody molecule comprises: (i) a VH comprising the amino acid sequence of the VH of monoclonal antibody ZB-2/2 (e.g., SEQ ID NO: 14); and (ii) a VL comprising the amino acid sequence of the VL of monoclonal antibody ZB-2/2 (e.g., SEQ ID NO: 18).

In an embodiment, the antibody molecule comprises a VH encoded by the nucleotide sequence of SEQ ID NO: 309 (or a nucleotide sequence substantially identical thereto) or a VL encoded by the nucleotide sequence of SEQ ID NO: 314 (or a nucleotide sequence substantially identical thereto), or both.

In an embodiment the antibody molecule comprises monoclonal antibody ZB-2/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/3 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/3 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/3 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/3 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/3 (e.g., SEQ ID NO: 106).

In an embodiment, the antibody molecule comprises: (i) a VH comprising: an HCDR1 comprising the amino acid sequence of the HCDR1 of monoclonal antibody ZB-2/3 (e.g., SEQ ID NO: 107); an HCDR2 comprising the amino acid sequence of the HCDR2 of monoclonal antibody ZB-2/3 (e.g., SEQ ID NO: 108); and an HCDR3 comprising the amino acid sequence of the HCDR3 of monoclonal antibody ZB-2/3 (e.g., SEQ ID NO: 113), and (ii) a VL comprising: an LCDR1 comprising the amino acid sequence of the LCDR1 of monoclonal antibody ZB-2/3 (e.g., SEQ ID NO: 111); an LCDR2 comprising the amino acid sequence of the LCDR2 of monoclonal antibody ZB-2/3 (e.g., SEQ ID NO: 118); and an LCDR3 comprising the amino acid sequence of the LCDR3 of monoclonal antibody ZB-2/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/3 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/3 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/3 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/3 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/3 (e.g., SEQ ID NO: 106).

In an embodiment, the antibody molecule comprises: (i) a VH comprising: an HCDR1 comprising the amino acid sequence of the HCDR1 of monoclonal antibody ZB-2/3 (e.g., SEQ ID NO: 203); an HCDR2 comprising the amino acid sequence of the HCDR2 of monoclonal antibody ZB-2/3 (e.g., SEQ ID NO: 206); and an HCDR3 comprising the amino acid sequence of the HCDR3 of monoclonal antibody ZB-2/3 (e.g., SEQ ID NO: 113), and (ii) a VL comprising: an LCDR1 comprising the amino acid sequence of the LCDR1 of monoclonal antibody ZB-2/3 (e.g., SEQ ID NO: 111); an LCDR2 comprising the amino acid sequence of the LCDR2 of monoclonal antibody ZB-2/3 (e.g., SEQ ID NO: 118); and an LCDR3 comprising the amino acid sequence of the LCDR3 of monoclonal antibody ZB-2/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/3 (e.g., SEQ ID NO: 14). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/3 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/3 (e.g., SEQ ID NO: 14); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/3 (e.g., SEQ ID NO: 19). In an embodiment, the antibody molecule comprises: (i) a VH comprising the amino acid sequence of the VH of monoclonal antibody ZB-2/3 (e.g., SEQ ID NO: 14); and (ii) a VL comprising the amino acid sequence of the VL of monoclonal antibody ZB-2/3 (e.g., SEQ ID NO: 19).

In an embodiment, the antibody molecule comprises a VH encoded by the nucleotide sequence of SEQ ID NO: 309 (or a nucleotide sequence substantially identical thereto) or a VL encoded by the nucleotide sequence of SEQ ID NO: 315 (or a nucleotide sequence substantially identical thereto), or both.

In an embodiment the antibody molecule comprises monoclonal antibody ZB-2/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/4 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/4 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/4 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/4 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/4 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/4 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/4 (e.g., SEQ ID NO: 119).

In an embodiment, the antibody molecule comprises: (i) a VH comprising: an HCDR1 comprising the amino acid sequence of the HCDR1 of monoclonal antibody ZB-2/4 (e.g., SEQ ID NO: 107); an HCDR2 comprising the amino acid sequence of the HCDR2 of monoclonal antibody ZB-2/4 (e.g., SEQ ID NO: 108); and an HCDR3 comprising the amino acid sequence of the HCDR3 of monoclonal antibody ZB-2/4 (e.g., SEQ ID NO: 113), and (ii) a VL comprising: an LCDR1 comprising the amino acid sequence of the LCDR1 of monoclonal antibody ZB-2/4 (e.g., SEQ ID NO: 111); an LCDR2 comprising the amino acid sequence of the LCDR2 of monoclonal antibody ZB-2/4 (e.g., SEQ ID NO: 105); and an LCDR3 comprising the amino acid sequence of the LCDR3 of monoclonal antibody ZB-2/4 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/4 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/4 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/4 (e.g., SEQ ID NO: 113).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-2/4 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-2/4 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-2/4 (e.g., SEQ ID NO: 113), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-2/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-2/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-2/4 (e.g., SEQ ID NO: 119).

In an embodiment, the antibody molecule comprises: (i) a VH comprising: an HCDR1 comprising the amino acid sequence of the HCDR1 of monoclonal antibody ZB-2/4 (e.g., SEQ ID NO: 203); an HCDR2 comprising the amino acid sequence of the HCDR2 of monoclonal antibody ZB-2/4 (e.g., SEQ ID NO: 206); and an HCDR3 comprising the amino acid sequence of the HCDR3 of monoclonal antibody ZB-2/4 (e.g., SEQ ID NO: 113), and (ii) a VL comprising: an LCDR1 comprising the amino acid sequence of the LCDR1 of monoclonal antibody ZB-2/4 (e.g., SEQ ID NO: 111); an LCDR2 comprising the amino acid sequence of the LCDR2 of monoclonal antibody ZB-2/4 (e.g., SEQ ID NO: 105); and an LCDR3 comprising the amino acid sequence of the LCDR3 of monoclonal antibody ZB-2/4 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/4 (e.g., SEQ ID NO: 14). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/4 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-2/4 (e.g., SEQ ID NO: 14); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-2/4 (e.g., SEQ ID NO: 20). In an embodiment, the antibody molecule comprises: (i) a VH comprising the amino acid sequence of the VH of monoclonal antibody ZB-2/4 (e.g., SEQ ID NO: 14); and (ii) a VL comprising the amino acid sequence of the VL of monoclonal antibody ZB-2/4 (e.g., SEQ ID NO: 20).

In an embodiment, the antibody molecule comprises a VH encoded by the nucleotide sequence of SEQ ID NO: 309 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZB-2/4.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/1 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/1 (e.g., SEQ ID NO: 114); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/1 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/1 (e.g., SEQ ID NO: 114); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/1 (e.g., SEQ ID NO: 106).

In an embodiment, the antibody molecule comprises: (i) a VH comprising: an HCDR1 comprising the amino acid sequence of the HCDR1 of monoclonal antibody ZB-3/1 (e.g., SEQ ID NO: 107); an HCDR2 comprising the amino acid sequence of the HCDR2 of monoclonal antibody ZB-3/1 (e.g., SEQ ID NO: 114); and an HCDR3 comprising the amino acid sequence of the HCDR3 of monoclonal antibody ZB-3/1 (e.g., SEQ ID NO: 103), and (ii) a VL comprising: an LCDR1 comprising the amino acid sequence of the LCDR1 of monoclonal antibody ZB-3/1 (e.g., SEQ ID NO: 111); an LCDR2 comprising the amino acid sequence of the LCDR2 of monoclonal antibody ZB-3/1 (e.g., SEQ ID NO: 105); and an LCDR3 comprising the amino acid sequence of the LCDR3 of monoclonal antibody ZB-3/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/1 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/1 (e.g., SEQ ID NO: 209); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/1 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/1 (e.g., SEQ ID NO: 209); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/1 (e.g., SEQ ID NO: 106).

In an embodiment, the antibody molecule comprises: (i) a VH comprising: an HCDR1 comprising the amino acid sequence of the HCDR1 of monoclonal antibody ZB-3/1 (e.g., SEQ ID NO: 203); an HCDR2 comprising the amino acid sequence of the HCDR2 of monoclonal antibody ZB-3/1 (e.g., SEQ ID NO: 209); and an HCDR3 comprising the amino acid sequence of the HCDR3 of monoclonal antibody ZB-3/1 (e.g., SEQ ID NO: 103), and (ii) a VL comprising: an LCDR1 comprising the amino acid sequence of the LCDR1 of monoclonal antibody ZB-3/1 (e.g., SEQ ID NO: 111); an LCDR2 comprising the amino acid sequence of the LCDR2 of monoclonal antibody ZB-3/1 (e.g., SEQ ID NO: 105); and an LCDR3 comprising the amino acid sequence of the LCDR3 of monoclonal antibody ZB-3/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/1 (e.g., SEQ ID NO: 15). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/1 (e.g., SEQ ID NO: 15); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/1 (e.g., SEQ ID NO: 6). In an embodiment, the antibody molecule comprises: (i) a VH comprising the amino acid sequence of the VH of monoclonal antibody ZB-3/1 (e.g., SEQ ID NO: 15); and (ii) a VL comprising the amino acid sequence of the VL of monoclonal antibody ZB-3/1 (e.g., SEQ ID NO: 6).

In an embodiment, the antibody molecule comprises a VH encoded by the nucleotide sequence of SEQ ID NO: 310 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZB-3/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/2 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/2 (e.g., SEQ ID NO: 114); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/2 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/2 (e.g., SEQ ID NO: 114); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/2 (e.g., SEQ ID NO: 106).

In an embodiment, the antibody molecule comprises: (i) a VH comprising: an HCDR1 comprising the amino acid sequence of the HCDR1 of monoclonal antibody ZB-3/2 (e.g., SEQ ID NO: 107); an HCDR2 comprising the amino acid sequence of the HCDR2 of monoclonal antibody ZB-3/2 (e.g., SEQ ID NO: 114); and an HCDR3 comprising the amino acid sequence of the HCDR3 of monoclonal antibody ZB-3/2 (e.g., SEQ ID NO: 103), and (ii) a VL comprising: an LCDR1 comprising the amino acid sequence of the LCDR1 of monoclonal antibody ZB-3/2 (e.g., SEQ ID NO: 117); an LCDR2 comprising the amino acid sequence of the LCDR2 of monoclonal antibody ZB-3/2 (e.g., SEQ ID NO: 105); and an LCDR3 comprising the amino acid sequence of the LCDR3 of monoclonal antibody ZB-3/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/2 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/2 (e.g., SEQ ID NO: 209); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/2 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/2 (e.g., SEQ ID NO: 209); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/2 (e.g., SEQ ID NO: 106).

In an embodiment, the antibody molecule comprises: (i) a VH comprising: an HCDR1 comprising the amino acid sequence of the HCDR1 of monoclonal antibody ZB-3/2 (e.g., SEQ ID NO: 203); an HCDR2 comprising the amino acid sequence of the HCDR2 of monoclonal antibody ZB-3/2 (e.g., SEQ ID NO: 209); and an HCDR3 comprising the amino acid sequence of the HCDR3 of monoclonal antibody ZB-3/2 (e.g., SEQ ID NO: 103), and (ii) a VL comprising: an LCDR1 comprising the amino acid sequence of the LCDR1 of monoclonal antibody ZB-3/2 (e.g., SEQ ID NO: 117); an LCDR2 comprising the amino acid sequence of the LCDR2 of monoclonal antibody ZB-3/2 (e.g., SEQ ID NO: 105); and an LCDR3 comprising the amino acid sequence of the LCDR3 of monoclonal antibody ZB-3/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/2 (e.g., SEQ ID NO: 15). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/2 (e.g., SEQ ID NO: 18).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/2 (e.g., SEQ ID NO: 15); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/2 (e.g., SEQ ID NO: 18). In an embodiment, the antibody molecule comprises: (i) a VH comprising the amino acid sequence of the VH of monoclonal antibody ZB-3/2 (e.g., SEQ ID NO: 15); and (ii) a VL comprising the amino acid sequence of the VL of monoclonal antibody ZB-3/2 (e.g., SEQ ID NO: 18).

In an embodiment, the antibody molecule comprises a VH encoded by the nucleotide sequence of SEQ ID NO: 310 (or a nucleotide sequence substantially identical thereto) or a VL encoded by the nucleotide sequence of SEQ ID NO: 314 (or a nucleotide sequence substantially identical thereto), or both.

In an embodiment the antibody molecule comprises monoclonal antibody ZB-3/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/3 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/3 (e.g., SEQ ID NO: 114); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/3 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/3 (e.g., SEQ ID NO: 114); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/3 (e.g., SEQ ID NO: 106).

In an embodiment, the antibody molecule comprises: (i) a VH comprising: an HCDR1 comprising the amino acid sequence of the HCDR1 of monoclonal antibody ZB-3/3 (e.g., SEQ ID NO: 107); an HCDR2 comprising the amino acid sequence of the HCDR2 of monoclonal antibody ZB-3/3 (e.g., SEQ ID NO: 114); and an HCDR3 comprising the amino acid sequence of the HCDR3 of monoclonal antibody ZB-3/3 (e.g., SEQ ID NO: 103), and (ii) a VL comprising: an LCDR1 comprising the amino acid sequence of the LCDR1 of monoclonal antibody ZB-3/3 (e.g., SEQ ID NO: 111); an LCDR2 comprising the amino acid sequence of the LCDR2 of monoclonal antibody ZB-3/3 (e.g., SEQ ID NO: 118); and an LCDR3 comprising the amino acid sequence of the LCDR3 of monoclonal antibody ZB-3/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/3 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/3 (e.g., SEQ ID NO: 209); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/3 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/3 (e.g., SEQ ID NO: 209); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/3 (e.g., SEQ ID NO: 106).

In an embodiment, the antibody molecule comprises: (i) a VH comprising: an HCDR1 comprising the amino acid sequence of the HCDR1 of monoclonal antibody ZB-3/3 (e.g., SEQ ID NO: 203); an HCDR2 comprising the amino acid sequence of the HCDR2 of monoclonal antibody ZB-3/3 (e.g., SEQ ID NO: 209); and an HCDR3 comprising the amino acid sequence of the HCDR3 of monoclonal antibody ZB-3/3 (e.g., SEQ ID NO: 103), and (ii) a VL comprising: an LCDR1 comprising the amino acid sequence of the LCDR1 of monoclonal antibody ZB-3/3 (e.g., SEQ ID NO: 111); an LCDR2 comprising the amino acid sequence of the LCDR2 of monoclonal antibody ZB-3/3 (e.g., SEQ ID NO: 118); and an LCDR3 comprising the amino acid sequence of the LCDR3 of monoclonal antibody ZB-3/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/3 (e.g., SEQ ID NO: 15). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/3 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/3 (e.g., SEQ ID NO: 15); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/3 (e.g., SEQ ID NO: 19). In an embodiment, the antibody molecule comprises: (i) a VH comprising the amino acid sequence of the VH of monoclonal antibody ZB-3/3 (e.g., SEQ ID NO: 15); and (ii) a VL comprising the amino acid sequence of the VL of monoclonal antibody ZB-3/3 (e.g., SEQ ID NO: 19).

In an embodiment, the antibody molecule comprises a VH encoded by the nucleotide sequence of SEQ ID NO: 310 (or a nucleotide sequence substantially identical thereto) or a VL encoded by the nucleotide sequence of SEQ ID NO: 315 (or a nucleotide sequence substantially identical thereto), or both.

In an embodiment the antibody molecule comprises monoclonal antibody ZB-3/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/4 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/4 (e.g., SEQ ID NO: 114); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/4 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/4 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/4 (e.g., SEQ ID NO: 114); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/4 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/4 (e.g., SEQ ID NO: 119).

In an embodiment, the antibody molecule comprises: (i) a VH comprising: an HCDR1 comprising the amino acid sequence of the HCDR1 of monoclonal antibody ZB-3/4 (e.g., SEQ ID NO: 107); an HCDR2 comprising the amino acid sequence of the HCDR2 of monoclonal antibody ZB-3/4 (e.g., SEQ ID NO: 114); and an HCDR3 comprising the amino acid sequence of the HCDR3 of monoclonal antibody ZB-3/4 (e.g., SEQ ID NO: 103), and (ii) a VL comprising: an LCDR1 comprising the amino acid sequence of the LCDR1 of monoclonal antibody ZB-3/4 (e.g., SEQ ID NO: 111); an LCDR2 comprising the amino acid sequence of the LCDR2 of monoclonal antibody ZB-3/4 (e.g., SEQ ID NO: 105); and an LCDR3 comprising the amino acid sequence of the LCDR3 of monoclonal antibody ZB-3/4 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/4 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/4 (e.g., SEQ ID NO: 209); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/4 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-3/4 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-3/4 (e.g., SEQ ID NO: 209); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-3/4 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-3/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-3/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-3/4 (e.g., SEQ ID NO: 119).

In an embodiment, the antibody molecule comprises: (i) a VH comprising: an HCDR1 comprising the amino acid sequence of the HCDR1 of monoclonal antibody ZB-3/4 (e.g., SEQ ID NO: 203); an HCDR2 comprising the amino acid sequence of the HCDR2 of monoclonal antibody ZB-3/4 (e.g., SEQ ID NO: 209); and an HCDR3 comprising the amino acid sequence of the HCDR3 of monoclonal antibody ZB-3/4 (e.g., SEQ ID NO: 103), and (ii) a VL comprising: an LCDR1 comprising the amino acid sequence of the LCDR1 of monoclonal antibody ZB-3/4 (e.g., SEQ ID NO: 111); an LCDR2 comprising the amino acid sequence of the LCDR2 of monoclonal antibody ZB-3/4 (e.g., SEQ ID NO: 105); and an LCDR3 comprising the amino acid sequence of the LCDR3 of monoclonal antibody ZB-3/4 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/4 (e.g., SEQ ID NO: 15). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/4 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-3/4 (e.g., SEQ ID NO: 15); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-3/4 (e.g., SEQ ID NO: 20). In an embodiment, the antibody molecule comprises: (i) a VH comprising the amino acid sequence of the VH of monoclonal antibody ZB-3/4 (e.g., SEQ ID NO: 15); and (ii) a VL comprising the amino acid sequence of the VL of monoclonal antibody ZB-3/4 (e.g., SEQ ID NO: 20).

In an embodiment, the antibody molecule comprises a VH encoded by the nucleotide sequence of SEQ ID NO: 310 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZB-3/4.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/1 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/1 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/1 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/1 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/1 (e.g., SEQ ID NO: 106).

In an embodiment, the antibody molecule comprises: (i) a VH comprising: an HCDR1 comprising the amino acid sequence of the HCDR1 of monoclonal antibody ZB-4/1 (e.g., SEQ ID NO: 107); an HCDR2 comprising the amino acid sequence of the HCDR2 of monoclonal antibody ZB-4/1 (e.g., SEQ ID NO: 108); and an HCDR3 comprising the amino acid sequence of the HCDR3 of monoclonal antibody ZB-4/1 (e.g., SEQ ID NO: 115), and (ii) a VL comprising: an LCDR1 comprising the amino acid sequence of the LCDR1 of monoclonal antibody ZB-4/1 (e.g., SEQ ID NO: 111); an LCDR2 comprising the amino acid sequence of the LCDR2 of monoclonal antibody ZB-4/1 (e.g., SEQ ID NO: 105); and an LCDR3 comprising the amino acid sequence of the LCDR3 of monoclonal antibody ZB-4/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/1 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/1 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/1 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/1 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/1 (e.g., SEQ ID NO: 106).

In an embodiment, the antibody molecule comprises: (i) a VH comprising: an HCDR1 comprising the amino acid sequence of the HCDR1 of monoclonal antibody ZB-4/1 (e.g., SEQ ID NO: 203); an HCDR2 comprising the amino acid sequence of the HCDR2 of monoclonal antibody ZB-4/1 (e.g., SEQ ID NO: 206); and an HCDR3 comprising the amino acid sequence of the HCDR3 of monoclonal antibody ZB-4/1 (e.g., SEQ ID NO: 115), and (ii) a VL comprising: an LCDR1 comprising the amino acid sequence of the LCDR1 of monoclonal antibody ZB-4/1 (e.g., SEQ ID NO: 111); an LCDR2 comprising the amino acid sequence of the LCDR2 of monoclonal antibody ZB-4/1 (e.g., SEQ ID NO: 105); and an LCDR3 comprising the amino acid sequence of the LCDR3 of monoclonal antibody ZB-4/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/1 (e.g., SEQ ID NO: 16). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/1 (e.g., SEQ ID NO: 16); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/1 (e.g., SEQ ID NO: 6). In an embodiment, the antibody molecule comprises: (i) a VH comprising the amino acid sequence of the VH of monoclonal antibody ZB-4/1 (e.g., SEQ ID NO: 16); and (ii) a VL comprising the amino acid sequence of the VL of monoclonal antibody ZB-4/1 (e.g., SEQ ID NO: 6).

In an embodiment, the antibody molecule comprises a VH encoded by the nucleotide sequence of SEQ ID NO: 311 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZB-4/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/2 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/2 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/2 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/2 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/2 (e.g., SEQ ID NO: 106).

In an embodiment, the antibody molecule comprises: (i) a VH comprising: an HCDR1 comprising the amino acid sequence of the HCDR1 of monoclonal antibody ZB-4/2 (e.g., SEQ ID NO: 107); an HCDR2 comprising the amino acid sequence of the HCDR2 of monoclonal antibody ZB-4/2 (e.g., SEQ ID NO: 108); and an HCDR3 comprising the amino acid sequence of the HCDR3 of monoclonal antibody ZB-4/2 (e.g., SEQ ID NO: 115), and (ii) a VL comprising: an LCDR1 comprising the amino acid sequence of the LCDR1 of monoclonal antibody ZB-4/2 (e.g., SEQ ID NO: 117); an LCDR2 comprising the amino acid sequence of the LCDR2 of monoclonal antibody ZB-4/2 (e.g., SEQ ID NO: 105); and an LCDR3 comprising the amino acid sequence of the LCDR3 of monoclonal antibody ZB-4/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/2 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/2 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/2 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/2 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/2 (e.g., SEQ ID NO: 106).

In an embodiment, the antibody molecule comprises: (i) a VH comprising: an HCDR1 comprising the amino acid sequence of the HCDR1 of monoclonal antibody ZB-4/2 (e.g., SEQ ID NO: 203); an HCDR2 comprising the amino acid sequence of the HCDR2 of monoclonal antibody ZB-4/2 (e.g., SEQ ID NO: 206); and an HCDR3 comprising the amino acid sequence of the HCDR3 of monoclonal antibody ZB-4/2 (e.g., SEQ ID NO: 115), and (ii) a VL comprising: an LCDR1 comprising the amino acid sequence of the LCDR1 of monoclonal antibody ZB-4/2 (e.g., SEQ ID NO: 117); an LCDR2 comprising the amino acid sequence of the LCDR2 of monoclonal antibody ZB-4/2 (e.g., SEQ ID NO: 105); and an LCDR3 comprising the amino acid sequence of the LCDR3 of monoclonal antibody ZB-4/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/2 (e.g., SEQ ID NO: 16). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/2 (e.g., SEQ ID NO: 18).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/2 (e.g., SEQ ID NO: 16); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/2 (e.g., SEQ ID NO: 18). In an embodiment, the antibody molecule comprises: (i) a VH comprising the amino acid sequence of the VH of monoclonal antibody ZB-4/2 (e.g., SEQ ID NO: 16); and (ii) a VL comprising the amino acid sequence of the VL of monoclonal antibody ZB-4/2 (e.g., SEQ ID NO: 18).

In an embodiment, the antibody molecule comprises a VH encoded by the nucleotide sequence of SEQ ID NO: 311 (or a nucleotide sequence substantially identical thereto) or a VL encoded by the nucleotide sequence of SEQ ID NO: 314 (or a nucleotide sequence substantially identical thereto), or both.

In an embodiment the antibody molecule comprises monoclonal antibody ZB-4/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/3 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/3 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/3 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/3 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/3 (e.g., SEQ ID NO: 106).

In an embodiment, the antibody molecule comprises: (i) a VH comprising: an HCDR1 comprising the amino acid sequence of the HCDR1 of monoclonal antibody ZB-4/3 (e.g., SEQ ID NO: 107); an HCDR2 comprising the amino acid sequence of the HCDR2 of monoclonal antibody ZB-4/3 (e.g., SEQ ID NO: 108); and an HCDR3 comprising the amino acid sequence of the HCDR3 of monoclonal antibody ZB-4/3 (e.g., SEQ ID NO: 115), and (ii) a VL comprising: an LCDR1 comprising the amino acid sequence of the LCDR1 of monoclonal antibody ZB-4/3 (e.g., SEQ ID NO: 111); an LCDR2 comprising the amino acid sequence of the LCDR2 of monoclonal antibody ZB-4/3 (e.g., SEQ ID NO: 118); and an LCDR3 comprising the amino acid sequence of the LCDR3 of monoclonal antibody ZB-4/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/3 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/3 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/3 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/3 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/3 (e.g., SEQ ID NO: 106).

In an embodiment, the antibody molecule comprises: (i) a VH comprising: an HCDR1 comprising the amino acid sequence of the HCDR1 of monoclonal antibody ZB-4/3 (e.g., SEQ ID NO: 203); an HCDR2 comprising the amino acid sequence of the HCDR2 of monoclonal antibody ZB-4/3 (e.g., SEQ ID NO: 206); and an HCDR3 comprising the amino acid sequence of the HCDR3 of monoclonal antibody ZB-4/3 (e.g., SEQ ID NO: 115), and (ii) a VL comprising: an LCDR1 comprising the amino acid sequence of the LCDR1 of monoclonal antibody ZB-4/3 (e.g., SEQ ID NO: 111); an LCDR2 comprising the amino acid sequence of the LCDR2 of monoclonal antibody ZB-4/3 (e.g., SEQ ID NO: 118); and an LCDR3 comprising the amino acid sequence of the LCDR3 of monoclonal antibody ZB-4/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/3 (e.g., SEQ ID NO: 16). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/3 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/3 (e.g., SEQ ID NO: 16); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/3 (e.g., SEQ ID NO: 19). In an embodiment, the antibody molecule comprises: (i) a VH comprising the amino acid sequence of the VH of monoclonal antibody ZB-4/3 (e.g., SEQ ID NO: 16); and (ii) a VL comprising the amino acid sequence of the VL of monoclonal antibody ZB-4/3 (e.g., SEQ ID NO: 19).

In an embodiment, the antibody molecule comprises a VH encoded by the nucleotide sequence of SEQ ID NO: 311 (or a nucleotide sequence substantially identical thereto) or a VL encoded by the nucleotide sequence of SEQ ID NO: 315 (or a nucleotide sequence substantially identical thereto), or both.

In an embodiment the antibody molecule comprises monoclonal antibody ZB-4/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/4 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/4 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/4 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/4 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/4 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/4 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/4 (e.g., SEQ ID NO: 119).

In an embodiment, the antibody molecule comprises: (i) a VH comprising: an HCDR1 comprising the amino acid sequence of the HCDR1 of monoclonal antibody ZB-4/4 (e.g., SEQ ID NO: 107); an HCDR2 comprising the amino acid sequence of the HCDR2 of monoclonal antibody ZB-4/4 (e.g., SEQ ID NO: 108); and an HCDR3 comprising the amino acid sequence of the HCDR3 of monoclonal antibody ZB-4/4 (e.g., SEQ ID NO: 115), and (ii) a VL comprising: an LCDR1 comprising the amino acid sequence of the LCDR1 of monoclonal antibody ZB-4/4 (e.g., SEQ ID NO: 111); an LCDR2 comprising the amino acid sequence of the LCDR2 of monoclonal antibody ZB-4/4 (e.g., SEQ ID NO: 105); and an LCDR3 comprising the amino acid sequence of the LCDR3 of monoclonal antibody ZB-4/4 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/4 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/4 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/4 (e.g., SEQ ID NO: 115).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-4/4 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-4/4 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-4/4 (e.g., SEQ ID NO: 115), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-4/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-4/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-4/4 (e.g., SEQ ID NO: 119).

In an embodiment, the antibody molecule comprises: (i) a VH comprising: an HCDR1 comprising the amino acid sequence of the HCDR1 of monoclonal antibody ZB-4/4 (e.g., SEQ ID NO: 203); an HCDR2 comprising the amino acid sequence of the HCDR2 of monoclonal antibody ZB-4/4 (e.g., SEQ ID NO: 206); and an HCDR3 comprising the amino acid sequence of the HCDR3 of monoclonal antibody ZB-4/4 (e.g., SEQ ID NO: 115), and (ii) a VL comprising: an LCDR1 comprising the amino acid sequence of the LCDR1 of monoclonal antibody ZB-4/4 (e.g., SEQ ID NO: 111); an LCDR2 comprising the amino acid sequence of the LCDR2 of monoclonal antibody ZB-4/4 (e.g., SEQ ID NO: 105); and an LCDR3 comprising the amino acid sequence of the LCDR3 of monoclonal antibody ZB-4/4 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/4 (e.g., SEQ ID NO: 16). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/4 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-4/4 (e.g., SEQ ID NO: 16); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-4/4 (e.g., SEQ ID NO: 20). In an embodiment, the antibody molecule comprises: (i) a VH comprising the amino acid sequence of the VH of monoclonal antibody ZB-4/4 (e.g., SEQ ID NO: 16); and (ii) a VL comprising the amino acid sequence of the VL of monoclonal antibody ZB-4/4 (e.g., SEQ ID NO: 20).

In an embodiment, the antibody molecule comprises a VH encoded by the nucleotide sequence of SEQ ID NO: 311 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZB-4/4.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/1 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/1 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/1 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/1 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/1 (e.g., SEQ ID NO: 106).

In an embodiment, the antibody molecule comprises: (i) a VH comprising: an HCDR1 comprising the amino acid sequence of the HCDR1 of monoclonal antibody ZB-5/1 (e.g., SEQ ID NO: 107); an HCDR2 comprising the amino acid sequence of the HCDR2 of monoclonal antibody ZB-5/1 (e.g., SEQ ID NO: 108); and an HCDR3 comprising the amino acid sequence of the HCDR3 of monoclonal antibody ZB-5/1 (e.g., SEQ ID NO: 116), and (ii) a VL comprising: an LCDR1 comprising the amino acid sequence of the LCDR1 of monoclonal antibody ZB-5/1 (e.g., SEQ ID NO: 111); an LCDR2 comprising the amino acid sequence of the LCDR2 of monoclonal antibody ZB-5/1 (e.g., SEQ ID NO: 105); and an LCDR3 comprising the amino acid sequence of the LCDR3 of monoclonal antibody ZB-5/1 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/1 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/1 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/1 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/1 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/1 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/1 (e.g., SEQ ID NO: 106).

In an embodiment, the antibody molecule comprises: (i) a VH comprising: an HCDR1 comprising the amino acid sequence of the HCDR1 of monoclonal antibody ZB-5/1 (e.g., SEQ ID NO: 203); an HCDR2 comprising the amino acid sequence of the HCDR2 of monoclonal antibody ZB-5/1 (e.g., SEQ ID NO: 206); and an HCDR3 comprising the amino acid sequence of the HCDR3 of monoclonal antibody ZB-5/1 (e.g., SEQ ID NO: 116), and (ii) a VL comprising: an LCDR1 comprising the amino acid sequence of the LCDR1 of monoclonal antibody ZB-5/1 (e.g., SEQ ID NO: 111); an LCDR2 comprising the amino acid sequence of the LCDR2 of monoclonal antibody ZB-5/1 (e.g., SEQ ID NO: 105); and an LCDR3 comprising the amino acid sequence of the LCDR3 of monoclonal antibody ZB-5/1 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/1 (e.g., SEQ ID NO: 17). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/1 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/1 (e.g., SEQ ID NO: 17); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/1 (e.g., SEQ ID NO: 6). In an embodiment, the antibody molecule comprises: (i) a VH comprising the amino acid sequence of the VH of monoclonal antibody ZB-5/1 (e.g., SEQ ID NO: 17); and (ii) a VL comprising the amino acid sequence of the VL of monoclonal antibody ZB-5/1 (e.g., SEQ ID NO: 6).

In an embodiment, the antibody molecule comprises a VH encoded by the nucleotide sequence of SEQ ID NO: 312 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZB-5/1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/2 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/2 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/2 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/2 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/2 (e.g., SEQ ID NO: 106).

In an embodiment, the antibody molecule comprises: (i) a VH comprising: an HCDR1 comprising the amino acid sequence of the HCDR1 of monoclonal antibody ZB-5/2 (e.g., SEQ ID NO: 107); an HCDR2 comprising the amino acid sequence of the HCDR2 of monoclonal antibody ZB-5/2 (e.g., SEQ ID NO: 108); and an HCDR3 comprising the amino acid sequence of the HCDR3 of monoclonal antibody ZB-5/2 (e.g., SEQ ID NO: 116), and (ii) a VL comprising: an LCDR1 comprising the amino acid sequence of the LCDR1 of monoclonal antibody ZB-5/2 (e.g., SEQ ID NO: 117); an LCDR2 comprising the amino acid sequence of the LCDR2 of monoclonal antibody ZB-5/2 (e.g., SEQ ID NO: 105); and an LCDR3 comprising the amino acid sequence of the LCDR3 of monoclonal antibody ZB-5/2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/2 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/2 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/2 (e.g., SEQ ID NO: 117); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/2 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/2 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/2 (e.g., SEQ ID NO: 117); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/2 (e.g., SEQ ID NO: 106).

In an embodiment, the antibody molecule comprises: (i) a VH comprising: an HCDR1 comprising the amino acid sequence of the HCDR1 of monoclonal antibody ZB-5/2 (e.g., SEQ ID NO: 203); an HCDR2 comprising the amino acid sequence of the HCDR2 of monoclonal antibody ZB-5/2 (e.g., SEQ ID NO: 206); and an HCDR3 comprising the amino acid sequence of the HCDR3 of monoclonal antibody ZB-5/2 (e.g., SEQ ID NO: 116), and (ii) a VL comprising: an LCDR1 comprising the amino acid sequence of the LCDR1 of monoclonal antibody ZB-5/2 (e.g., SEQ ID NO: 117); an LCDR2 comprising the amino acid sequence of the LCDR2 of monoclonal antibody ZB-5/2 (e.g., SEQ ID NO: 105); and an LCDR3 comprising the amino acid sequence of the LCDR3 of monoclonal antibody ZB-5/2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/2 (e.g., SEQ ID NO: 17). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/2 (e.g., SEQ ID NO: 18).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/2 (e.g., SEQ ID NO: 17); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/2 (e.g., SEQ ID NO: 18). In an embodiment, the antibody molecule comprises: (i) a VH comprising the amino acid sequence of the VH of monoclonal antibody ZB-5/2 (e.g., SEQ ID NO: 17); and (ii) a VL comprising the amino acid sequence of the VL of monoclonal antibody ZB-5/2 (e.g., SEQ ID NO: 18).

In an embodiment, the antibody molecule comprises a VH encoded by the nucleotide sequence of SEQ ID NO: 312 (or a nucleotide sequence substantially identical thereto) or a VL encoded by the nucleotide sequence of SEQ ID NO: 314 (or a nucleotide sequence substantially identical thereto), or both.

In an embodiment the antibody molecule comprises monoclonal antibody ZB-5/2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/3 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/3 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/3 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/3 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/3 (e.g., SEQ ID NO: 106).

In an embodiment, the antibody molecule comprises: (i) a VH comprising: an HCDR1 comprising the amino acid sequence of the HCDR1 of monoclonal antibody ZB-5/3 (e.g., SEQ ID NO: 107); an HCDR2 comprising the amino acid sequence of the HCDR2 of monoclonal antibody ZB-5/3 (e.g., SEQ ID NO: 108); and an HCDR3 comprising the amino acid sequence of the HCDR3 of monoclonal antibody ZB-5/3 (e.g., SEQ ID NO: 116), and (ii) a VL comprising: an LCDR1 comprising the amino acid sequence of the LCDR1 of monoclonal antibody ZB-5/3 (e.g., SEQ ID NO: 111); an LCDR2 comprising the amino acid sequence of the LCDR2 of monoclonal antibody ZB-5/3 (e.g., SEQ ID NO: 118); and an LCDR3 comprising the amino acid sequence of the LCDR3 of monoclonal antibody ZB-5/3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/3 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/3 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/3 (e.g., SEQ ID NO: 118); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/3 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/3 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/3 (e.g., SEQ ID NO: 118); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/3 (e.g., SEQ ID NO: 106).

In an embodiment, the antibody molecule comprises: (i) a VH comprising: an HCDR1 comprising the amino acid sequence of the HCDR1 of monoclonal antibody ZB-5/3 (e.g., SEQ ID NO: 203); an HCDR2 comprising the amino acid sequence of the HCDR2 of monoclonal antibody ZB-5/3 (e.g., SEQ ID NO: 206); and an HCDR3 comprising the amino acid sequence of the HCDR3 of monoclonal antibody ZB-5/3 (e.g., SEQ ID NO: 116), and (ii) a VL comprising: an LCDR1 comprising the amino acid sequence of the LCDR1 of monoclonal antibody ZB-5/3 (e.g., SEQ ID NO: 111); an LCDR2 comprising the amino acid sequence of the LCDR2 of monoclonal antibody ZB-5/3 (e.g., SEQ ID NO: 118); and an LCDR3 comprising the amino acid sequence of the LCDR3 of monoclonal antibody ZB-5/3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/3 (e.g., SEQ ID NO: 17). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/3 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/3 (e.g., SEQ ID NO: 17); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/3 (e.g., SEQ ID NO: 19). In an embodiment, the antibody molecule comprises: (i) a VH comprising the amino acid sequence of the VH of monoclonal antibody ZB-5/3 (e.g., SEQ ID NO: 17); and (ii) a VL comprising the amino acid sequence of the VL of monoclonal antibody ZB-5/3 (e.g., SEQ ID NO: 19).

In an embodiment, the antibody molecule comprises a VH encoded by the nucleotide sequence of SEQ ID NO: 312 (or a nucleotide sequence substantially identical thereto) or a VL encoded by the nucleotide sequence of SEQ ID NO: 315 (or a nucleotide sequence substantially identical thereto), or both.

In an embodiment the antibody molecule comprises monoclonal antibody ZB-5/3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/4 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/4 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/4 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/4 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/4 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/4 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/4 (e.g., SEQ ID NO: 119).

In an embodiment, the antibody molecule comprises: (i) a VH comprising: an HCDR1 comprising the amino acid sequence of the HCDR1 of monoclonal antibody ZB-5/4 (e.g., SEQ ID NO: 107); an HCDR2 comprising the amino acid sequence of the HCDR2 of monoclonal antibody ZB-5/4 (e.g., SEQ ID NO: 108); and an HCDR3 comprising the amino acid sequence of the HCDR3 of monoclonal antibody ZB-5/4 (e.g., SEQ ID NO: 116), and (ii) a VL comprising: an LCDR1 comprising the amino acid sequence of the LCDR1 of monoclonal antibody ZB-5/4 (e.g., SEQ ID NO: 111); an LCDR2 comprising the amino acid sequence of the LCDR2 of monoclonal antibody ZB-5/4 (e.g., SEQ ID NO: 105); and an LCDR3 comprising the amino acid sequence of the LCDR3 of monoclonal antibody ZB-5/4 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/4 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/4 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/4 (e.g., SEQ ID NO: 116).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/4 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZB-5/4 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZB-5/4 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZB-5/4 (e.g., SEQ ID NO: 116), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZB-5/4 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZB-5/4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZB-5/4 (e.g., SEQ ID NO: 119).

In an embodiment, the antibody molecule comprises: (i) a VH comprising: an HCDR1 comprising the amino acid sequence of the HCDR1 of monoclonal antibody ZB-5/4 (e.g., SEQ ID NO: 203); an HCDR2 comprising the amino acid sequence of the HCDR2 of monoclonal antibody ZB-5/4 (e.g., SEQ ID NO: 206); and an HCDR3 comprising the amino acid sequence of the HCDR3 of monoclonal antibody ZB-5/4 (e.g., SEQ ID NO: 116), and (ii) a VL comprising: an LCDR1 comprising the amino acid sequence of the LCDR1 of monoclonal antibody ZB-5/4 (e.g., SEQ ID NO: 111); an LCDR2 comprising the amino acid sequence of the LCDR2 of monoclonal antibody ZB-5/4 (e.g., SEQ ID NO: 105); and an LCDR3 comprising the amino acid sequence of the LCDR3 of monoclonal antibody ZB-5/4 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/4 (e.g., SEQ ID NO: 17). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/4 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZB-5/4 (e.g., SEQ ID NO: 17); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZB-5/4 (e.g., SEQ ID NO: 20). In an embodiment, the antibody molecule comprises: (i) a VH comprising the amino acid sequence of the VH of monoclonal antibody ZB-5/4 (e.g., SEQ ID NO: 17); and (ii) a VL comprising the amino acid sequence of the VL of monoclonal antibody ZB-5/4 (e.g., SEQ ID NO: 20).

In an embodiment, the antibody molecule comprises a VH encoded by the nucleotide sequence of SEQ ID NO: 312 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZB-5/4.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC1 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC1 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC1 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC1 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC1 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC1 (e.g., SEQ ID NO: 119).

In an embodiment, the antibody molecule comprises: (i) a VH comprising: an HCDR1 comprising the amino acid sequence of the HCDR1 of monoclonal antibody ZC1 (e.g., SEQ ID NO: 107); an HCDR2 comprising the amino acid sequence of the HCDR2 of monoclonal antibody ZC1 (e.g., SEQ ID NO: 108); and an HCDR3 comprising the amino acid sequence of the HCDR3 of monoclonal antibody ZC1 (e.g., SEQ ID NO: 103), and (ii) a VL comprising: an LCDR1 comprising the amino acid sequence of the LCDR1 of monoclonal antibody ZC1 (e.g., SEQ ID NO: 111); an LCDR2 comprising the amino acid sequence of the LCDR2 of monoclonal antibody ZC1 (e.g., SEQ ID NO: 105); and an LCDR3 comprising the amino acid sequence of the LCDR3 of monoclonal antibody ZC1 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC1 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC1 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC1 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC1 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC1 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC1 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC1 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC1 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC1 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC1 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC1 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC1 (e.g., SEQ ID NO: 119).

In an embodiment, the antibody molecule comprises: (i) a VH comprising: an HCDR1 comprising the amino acid sequence of the HCDR1 of monoclonal antibody ZC1 (e.g., SEQ ID NO: 203); an HCDR2 comprising the amino acid sequence of the HCDR2 of monoclonal antibody ZC1 (e.g., SEQ ID NO: 206); and an HCDR3 comprising the amino acid sequence of the HCDR3 of monoclonal antibody ZC1 (e.g., SEQ ID NO: 103), and (ii) a VL comprising: an LCDR1 comprising the amino acid sequence of the LCDR1 of monoclonal antibody ZC1 (e.g., SEQ ID NO: 111); an LCDR2 comprising the amino acid sequence of the LCDR2 of monoclonal antibody ZC1 (e.g., SEQ ID NO: 105); and an LCDR3 comprising the amino acid sequence of the LCDR3 of monoclonal antibody ZC1 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC1 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC1 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC1 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC1 (e.g., SEQ ID NO: 20). In an embodiment, the antibody molecule comprises: (i) a VH comprising the amino acid sequence of the VH of monoclonal antibody ZC1 (e.g., SEQ ID NO: 13); and (ii) a VL comprising the amino acid sequence of the VL of monoclonal antibody ZC1 (e.g., SEQ ID NO: 20).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZC1.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC2 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC2 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC2 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC2 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC2 (e.g., SEQ ID NO: 106).

In an embodiment, the antibody molecule comprises: (i) a VH comprising: an HCDR1 comprising the amino acid sequence of the HCDR1 of monoclonal antibody ZC2 (e.g., SEQ ID NO: 107); an HCDR2 comprising the amino acid sequence of the HCDR2 of monoclonal antibody ZC2 (e.g., SEQ ID NO: 108); and an HCDR3 comprising the amino acid sequence of the HCDR3 of monoclonal antibody ZC2 (e.g., SEQ ID NO: 103), and (ii) a VL comprising: an LCDR1 comprising the amino acid sequence of the LCDR1 of monoclonal antibody ZC2 (e.g., SEQ ID NO: 104); an LCDR2 comprising the amino acid sequence of the LCDR2 of monoclonal antibody ZC2 (e.g., SEQ ID NO: 105); and an LCDR3 comprising the amino acid sequence of the LCDR3 of monoclonal antibody ZC2 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC2 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC2 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC2 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC2 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC2 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC2 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC2 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC2 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC2 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC2 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC2 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC2 (e.g., SEQ ID NO: 106).

In an embodiment, the antibody molecule comprises: (i) a VH comprising: an HCDR1 comprising the amino acid sequence of the HCDR1 of monoclonal antibody ZC2 (e.g., SEQ ID NO: 203); an HCDR2 comprising the amino acid sequence of the HCDR2 of monoclonal antibody ZC2 (e.g., SEQ ID NO: 206); and an HCDR3 comprising the amino acid sequence of the HCDR3 of monoclonal antibody ZC2 (e.g., SEQ ID NO: 103), and (ii) a VL comprising: an LCDR1 comprising the amino acid sequence of the LCDR1 of monoclonal antibody ZC2 (e.g., SEQ ID NO: 104); an LCDR2 comprising the amino acid sequence of the LCDR2 of monoclonal antibody ZC2 (e.g., SEQ ID NO: 105); and an LCDR3 comprising the amino acid sequence of the LCDR3 of monoclonal antibody ZC2 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC2 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC2 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC2 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC2 (e.g., SEQ ID NO: 7). In an embodiment, the antibody molecule comprises: (i) a VH comprising the amino acid sequence of the VH of monoclonal antibody ZC2 (e.g., SEQ ID NO: 13); and (ii) a VL comprising the amino acid sequence of the VL of monoclonal antibody ZC2 (e.g., SEQ ID NO: 7).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZC2.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC3 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC3 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC3 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC3 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC3 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC3 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC3 (e.g., SEQ ID NO: 106).

In an embodiment, the antibody molecule comprises: (i) a VH comprising: an HCDR1 comprising the amino acid sequence of the HCDR1 of monoclonal antibody ZC3 (e.g., SEQ ID NO: 107); an HCDR2 comprising the amino acid sequence of the HCDR2 of monoclonal antibody ZC3 (e.g., SEQ ID NO: 108); and an HCDR3 comprising the amino acid sequence of the HCDR3 of monoclonal antibody ZC3 (e.g., SEQ ID NO: 103), and (ii) a VL comprising: an LCDR1 comprising the amino acid sequence of the LCDR1 of monoclonal antibody ZC3 (e.g., SEQ ID NO: 111); an LCDR2 comprising the amino acid sequence of the LCDR2 of monoclonal antibody ZC3 (e.g., SEQ ID NO: 105); and an LCDR3 comprising the amino acid sequence of the LCDR3 of monoclonal antibody ZC3 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC3 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC3 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC3 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC3 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC3 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC3 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC3 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC3 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC3 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC3 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC3 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC3 (e.g., SEQ ID NO: 106).

In an embodiment, the antibody molecule comprises: (i) a VH comprising: an HCDR1 comprising the amino acid sequence of the HCDR1 of monoclonal antibody ZC3 (e.g., SEQ ID NO: 203); an HCDR2 comprising the amino acid sequence of the HCDR2 of monoclonal antibody ZC3 (e.g., SEQ ID NO: 206); and an HCDR3 comprising the amino acid sequence of the HCDR3 of monoclonal antibody ZC3 (e.g., SEQ ID NO: 103), and (ii) a VL comprising: an LCDR1 comprising the amino acid sequence of the LCDR1 of monoclonal antibody ZC3 (e.g., SEQ ID NO: 111); an LCDR2 comprising the amino acid sequence of the LCDR2 of monoclonal antibody ZC3 (e.g., SEQ ID NO: 105); and an LCDR3 comprising the amino acid sequence of the LCDR3 of monoclonal antibody ZC3 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC3 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC3 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC3 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC3 (e.g., SEQ ID NO: 6). In an embodiment, the antibody molecule comprises: (i) a VH comprising the amino acid sequence of the VH of monoclonal antibody ZC3 (e.g., SEQ ID NO: 13); and (ii) a VL comprising the amino acid sequence of the VL of monoclonal antibody ZC3 (e.g., SEQ ID NO: 6).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZC3.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC4 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC4 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC4 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC4 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC4 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC4 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC4 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC4 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC4 (e.g., SEQ ID NO: 119).

In an embodiment, the antibody molecule comprises: (i) a VH comprising: an HCDR1 comprising the amino acid sequence of the HCDR1 of monoclonal antibody ZC4 (e.g., SEQ ID NO: 107); an HCDR2 comprising the amino acid sequence of the HCDR2 of monoclonal antibody ZC4 (e.g., SEQ ID NO: 108); and an HCDR3 comprising the amino acid sequence of the HCDR3 of monoclonal antibody ZC4 (e.g., SEQ ID NO: 103), and (ii) a VL comprising: an LCDR1 comprising the amino acid sequence of the LCDR1 of monoclonal antibody ZC4 (e.g., SEQ ID NO: 104); an LCDR2 comprising the amino acid sequence of the LCDR2 of monoclonal antibody ZC4 (e.g., SEQ ID NO: 105); and an LCDR3 comprising the amino acid sequence of the LCDR3 of monoclonal antibody ZC4 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC4 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC4 (e.g., SEQ ID NO: 206); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC4 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC4 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC4 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC4 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC4 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC4 (e.g., SEQ ID NO: 206); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC4 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC4 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC4 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC4 (e.g., SEQ ID NO: 119).

In an embodiment, the antibody molecule comprises: (i) a VH comprising: an HCDR1 comprising the amino acid sequence of the HCDR1 of monoclonal antibody ZC4 (e.g., SEQ ID NO: 203); an HCDR2 comprising the amino acid sequence of the HCDR2 of monoclonal antibody ZC4 (e.g., SEQ ID NO: 206); and an HCDR3 comprising the amino acid sequence of the HCDR3 of monoclonal antibody ZC4 (e.g., SEQ ID NO: 103), and (ii) a VL comprising: an LCDR1 comprising the amino acid sequence of the LCDR1 of monoclonal antibody ZC4 (e.g., SEQ ID NO: 104); an LCDR2 comprising the amino acid sequence of the LCDR2 of monoclonal antibody ZC4 (e.g., SEQ ID NO: 105); and an LCDR3 comprising the amino acid sequence of the LCDR3 of monoclonal antibody ZC4 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC4 (e.g., SEQ ID NO: 13). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC4 (e.g., SEQ ID NO: 21).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC4 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC4 (e.g., SEQ ID NO: 21). In an embodiment, the antibody molecule comprises: (i) a VH comprising the amino acid sequence of the VH of monoclonal antibody ZC4 (e.g., SEQ ID NO: 13); and (ii) a VL comprising the amino acid sequence of the VL of monoclonal antibody ZC4 (e.g., SEQ ID NO: 21).

In an embodiment, the antibody molecule comprises a VH encoded by the nucleotide sequence of SEQ ID NO: 308 (or a nucleotide sequence substantially identical thereto) or a VL encoded by the nucleotide sequence of SEQ ID NO: 318 (or a nucleotide sequence substantially identical thereto), or both.

In an embodiment the antibody molecule comprises monoclonal antibody ZC4.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC5 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC5 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC5 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC5 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC5 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC5 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC5 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC5 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC5 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC5 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC5 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC5 (e.g., SEQ ID NO: 119).

In an embodiment, the antibody molecule comprises: (i) a VH comprising: an HCDR1 comprising the amino acid sequence of the HCDR1 of monoclonal antibody ZC5 (e.g., SEQ ID NO: 101); an HCDR2 comprising the amino acid sequence of the HCDR2 of monoclonal antibody ZC5 (e.g., SEQ ID NO: 108); and an HCDR3 comprising the amino acid sequence of the HCDR3 of monoclonal antibody ZC5 (e.g., SEQ ID NO: 103), and (ii) a VL comprising: an LCDR1 comprising the amino acid sequence of the LCDR1 of monoclonal antibody ZC5 (e.g., SEQ ID NO: 111); an LCDR2 comprising the amino acid sequence of the LCDR2 of monoclonal antibody ZC5 (e.g., SEQ ID NO: 105); and an LCDR3 comprising the amino acid sequence of the LCDR3 of monoclonal antibody ZC5 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC5 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC5 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC5 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC5 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC5 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC5 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC5 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC5 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC5 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC5 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC5 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC5 (e.g., SEQ ID NO: 119).

In an embodiment, the antibody molecule comprises: (i) a VH comprising: an HCDR1 comprising the amino acid sequence of the HCDR1 of monoclonal antibody ZC5 (e.g., SEQ ID NO: 201); an HCDR2 comprising the amino acid sequence of the HCDR2 of monoclonal antibody ZC5 (e.g., SEQ ID NO: 207); and an HCDR3 comprising the amino acid sequence of the HCDR3 of monoclonal antibody ZC5 (e.g., SEQ ID NO: 103), and (ii) a VL comprising: an LCDR1 comprising the amino acid sequence of the LCDR1 of monoclonal antibody ZC5 (e.g., SEQ ID NO: 111); an LCDR2 comprising the amino acid sequence of the LCDR2 of monoclonal antibody ZC5 (e.g., SEQ ID NO: 105); and an LCDR3 comprising the amino acid sequence of the LCDR3 of monoclonal antibody ZC5 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC5 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC5 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC5 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC5 (e.g., SEQ ID NO: 20). In an embodiment, the antibody molecule comprises: (i) a VH comprising the amino acid sequence of the VH of monoclonal antibody ZC5 (e.g., SEQ ID NO: 10); and (ii) a VL comprising the amino acid sequence of the VL of monoclonal antibody ZC5 (e.g., SEQ ID NO: 20).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZC5.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC6 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC6 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC6 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC6 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC6 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC6 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC6 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC6 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC6 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC6 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC6 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC6 (e.g., SEQ ID NO: 106).

In an embodiment, the antibody molecule comprises: (i) a VH comprising: an HCDR1 comprising the amino acid sequence of the HCDR1 of monoclonal antibody ZC6 (e.g., SEQ ID NO: 101); an HCDR2 comprising the amino acid sequence of the HCDR2 of monoclonal antibody ZC6 (e.g., SEQ ID NO: 108); and an HCDR3 comprising the amino acid sequence of the HCDR3 of monoclonal antibody ZC6 (e.g., SEQ ID NO: 103), and (ii) a VL comprising: an LCDR1 comprising the amino acid sequence of the LCDR1 of monoclonal antibody ZC6 (e.g., SEQ ID NO: 104); an LCDR2 comprising the amino acid sequence of the LCDR2 of monoclonal antibody ZC6 (e.g., SEQ ID NO: 105); and an LCDR3 comprising the amino acid sequence of the LCDR3 of monoclonal antibody ZC6 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC6 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC6 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC6 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC6 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC6 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC6 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC6 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC6 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC6 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC6 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC6 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC6 (e.g., SEQ ID NO: 106).

In an embodiment, the antibody molecule comprises: (i) a VH comprising: an HCDR1 comprising the amino acid sequence of the HCDR1 of monoclonal antibody ZC6 (e.g., SEQ ID NO: 201); an HCDR2 comprising the amino acid sequence of the HCDR2 of monoclonal antibody ZC6 (e.g., SEQ ID NO: 207); and an HCDR3 comprising the amino acid sequence of the HCDR3 of monoclonal antibody ZC6 (e.g., SEQ ID NO: 103), and (ii) a VL comprising: an LCDR1 comprising the amino acid sequence of the LCDR1 of monoclonal antibody ZC6 (e.g., SEQ ID NO: 104); an LCDR2 comprising the amino acid sequence of the LCDR2 of monoclonal antibody ZC6 (e.g., SEQ ID NO: 105); and an LCDR3 comprising the amino acid sequence of the LCDR3 of monoclonal antibody ZC6 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC6 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC6 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC6 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC6 (e.g., SEQ ID NO: 7). In an embodiment, the antibody molecule comprises: (i) a VH comprising the amino acid sequence of the VH of monoclonal antibody ZC6 (e.g., SEQ ID NO: 10); and (ii) a VL comprising the amino acid sequence of the VL of monoclonal antibody ZC6 (e.g., SEQ ID NO: 7).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZC6.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC7 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC7 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC7 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC7 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC7 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC7 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC7 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC7 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC7 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC7 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC7 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC7 (e.g., SEQ ID NO: 106).

In an embodiment, the antibody molecule comprises: (i) a VH comprising: an HCDR1 comprising the amino acid sequence of the HCDR1 of monoclonal antibody ZC7 (e.g., SEQ ID NO: 101); an HCDR2 comprising the amino acid sequence of the HCDR2 of monoclonal antibody ZC7 (e.g., SEQ ID NO: 108); and an HCDR3 comprising the amino acid sequence of the HCDR3 of monoclonal antibody ZC7 (e.g., SEQ ID NO: 103), and (ii) a VL comprising: an LCDR1 comprising the amino acid sequence of the LCDR1 of monoclonal antibody ZC7 (e.g., SEQ ID NO: 111); an LCDR2 comprising the amino acid sequence of the LCDR2 of monoclonal antibody ZC7 (e.g., SEQ ID NO: 105); and an LCDR3 comprising the amino acid sequence of the LCDR3 of monoclonal antibody ZC7 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC7 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC7 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC7 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC7 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC7 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC7 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC7 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC7 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC7 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC7 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC7 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC7 (e.g., SEQ ID NO: 106).

In an embodiment, the antibody molecule comprises: (i) a VH comprising: an HCDR1 comprising the amino acid sequence of the HCDR1 of monoclonal antibody ZC7 (e.g., SEQ ID NO: 201); an HCDR2 comprising the amino acid sequence of the HCDR2 of monoclonal antibody ZC7 (e.g., SEQ ID NO: 207); and an HCDR3 comprising the amino acid sequence of the HCDR3 of monoclonal antibody ZC7 (e.g., SEQ ID NO: 103), and (ii) a VL comprising: an LCDR1 comprising the amino acid sequence of the LCDR1 of monoclonal antibody ZC7 (e.g., SEQ ID NO: 111); an LCDR2 comprising the amino acid sequence of the LCDR2 of monoclonal antibody ZC7 (e.g., SEQ ID NO: 105); and an LCDR3 comprising the amino acid sequence of the LCDR3 of monoclonal antibody ZC7 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC7 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC7 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC7 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC7 (e.g., SEQ ID NO: 6). In an embodiment, the antibody molecule comprises: (i) a VH comprising the amino acid sequence of the VH of monoclonal antibody ZC7 (e.g., SEQ ID NO: 10); and (ii) a VL comprising the amino acid sequence of the VL of monoclonal antibody ZC7 (e.g., SEQ ID NO: 6).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZC7.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC8 (e.g., SEQ ID NO: 101; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC8 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC8 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC8 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC8 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC8 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC8 (e.g., SEQ ID NO: 101); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC8 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC8 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC8 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC8 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC8 (e.g., SEQ ID NO: 119).

In an embodiment, the antibody molecule comprises: (i) a VH comprising: an HCDR1 comprising the amino acid sequence of the HCDR1 of monoclonal antibody ZC8 (e.g., SEQ ID NO: 101); an HCDR2 comprising the amino acid sequence of the HCDR2 of monoclonal antibody ZC8 (e.g., SEQ ID NO: 108); and an HCDR3 comprising the amino acid sequence of the HCDR3 of monoclonal antibody ZC8 (e.g., SEQ ID NO: 103), and (ii) a VL comprising: an LCDR1 comprising the amino acid sequence of the LCDR1 of monoclonal antibody ZC8 (e.g., SEQ ID NO: 104); an LCDR2 comprising the amino acid sequence of the LCDR2 of monoclonal antibody ZC8 (e.g., SEQ ID NO: 105); and an LCDR3 comprising the amino acid sequence of the LCDR3 of monoclonal antibody ZC8 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC8 (e.g., SEQ ID NO: 201; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC8 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC8 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC8 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC8 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC8 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC8 (e.g., SEQ ID NO: 201); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC8 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC8 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC8 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC8 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC8 (e.g., SEQ ID NO: 119).

In an embodiment, the antibody molecule comprises: (i) a VH comprising: an HCDR1 comprising the amino acid sequence of the HCDR1 of monoclonal antibody ZC8 (e.g., SEQ ID NO: 201); an HCDR2 comprising the amino acid sequence of the HCDR2 of monoclonal antibody ZC8 (e.g., SEQ ID NO: 207); and an HCDR3 comprising the amino acid sequence of the HCDR3 of monoclonal antibody ZC8 (e.g., SEQ ID NO: 103), and (ii) a VL comprising: an LCDR1 comprising the amino acid sequence of the LCDR1 of monoclonal antibody ZC8 (e.g., SEQ ID NO: 104); an LCDR2 comprising the amino acid sequence of the LCDR2 of monoclonal antibody ZC8 (e.g., SEQ ID NO: 105); and an LCDR3 comprising the amino acid sequence of the LCDR3 of monoclonal antibody ZC8 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC8 (e.g., SEQ ID NO: 10). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC8 (e.g., SEQ ID NO: 21).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC8 (e.g., SEQ ID NO: 10); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC8 (e.g., SEQ ID NO: 21). In an embodiment, the antibody molecule comprises: (i) a VH comprising the amino acid sequence of the VH of monoclonal antibody ZC8 (e.g., SEQ ID NO: 10); and (ii) a VL comprising the amino acid sequence of the VL of monoclonal antibody ZC8 (e.g., SEQ ID NO: 21).

In an embodiment, the antibody molecule comprises a VH encoded by the nucleotide sequence of SEQ ID NO: 302 (or a nucleotide sequence substantially identical thereto) or a VL encoded by the nucleotide sequence of SEQ ID NO: 318 (or a nucleotide sequence substantially identical thereto), or both.

In an embodiment the antibody molecule comprises monoclonal antibody ZC8.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC9 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC9 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC9 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC9 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC9 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC9 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC9 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC9 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC9 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC9 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC9 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC9 (e.g., SEQ ID NO: 119).

In an embodiment, the antibody molecule comprises: (i) a VH comprising: an HCDR1 comprising the amino acid sequence of the HCDR1 of monoclonal antibody ZC9 (e.g., SEQ ID NO: 107); an HCDR2 comprising the amino acid sequence of the HCDR2 of monoclonal antibody ZC9 (e.g., SEQ ID NO: 108); and an HCDR3 comprising the amino acid sequence of the HCDR3 of monoclonal antibody ZC9 (e.g., SEQ ID NO: 103), and (ii) a VL comprising: an LCDR1 comprising the amino acid sequence of the LCDR1 of monoclonal antibody ZC9 (e.g., SEQ ID NO: 111); an LCDR2 comprising the amino acid sequence of the LCDR2 of monoclonal antibody ZC9 (e.g., SEQ ID NO: 105); and an LCDR3 comprising the amino acid sequence of the LCDR3 of monoclonal antibody ZC9 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC9 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC9 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC9 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC9 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC9 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC9 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC9 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC9 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC9 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC9 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC9 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC9 (e.g., SEQ ID NO: 119).

In an embodiment, the antibody molecule comprises: (i) a VH comprising: an HCDR1 comprising the amino acid sequence of the HCDR1 of monoclonal antibody ZC9 (e.g., SEQ ID NO: 203); an HCDR2 comprising the amino acid sequence of the HCDR2 of monoclonal antibody ZC9 (e.g., SEQ ID NO: 207); and an HCDR3 comprising the amino acid sequence of the HCDR3 of monoclonal antibody ZC9 (e.g., SEQ ID NO: 103), and (ii) a VL comprising: an LCDR1 comprising the amino acid sequence of the LCDR1 of monoclonal antibody ZC9 (e.g., SEQ ID NO: 111); an LCDR2 comprising the amino acid sequence of the LCDR2 of monoclonal antibody ZC9 (e.g., SEQ ID NO: 105); and an LCDR3 comprising the amino acid sequence of the LCDR3 of monoclonal antibody ZC9 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC9 (e.g., SEQ ID NO: 22). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC9 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC9 (e.g., SEQ ID NO: 22); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC9 (e.g., SEQ ID NO: 20). In an embodiment, the antibody molecule comprises: (i) a VH comprising the amino acid sequence of the VH of monoclonal antibody ZC9 (e.g., SEQ ID NO: 22); and (ii) a VL comprising the amino acid sequence of the VL of monoclonal antibody ZC9 (e.g., SEQ ID NO: 20).

In an embodiment, the antibody molecule comprises a VH encoded by the nucleotide sequence of SEQ ID NO: 317 (or a nucleotide sequence substantially identical thereto) or a VL encoded by the nucleotide sequence of SEQ ID NO: 316 (or a nucleotide sequence substantially identical thereto), or both.

In an embodiment the antibody molecule comprises monoclonal antibody ZC9.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC10 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC10 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC10 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC10 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC10 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC10 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC10 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC10 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC10 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC10 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC10 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC10 (e.g., SEQ ID NO: 106).

In an embodiment, the antibody molecule comprises: (i) a VH comprising: an HCDR1 comprising the amino acid sequence of the HCDR1 of monoclonal antibody ZC10 (e.g., SEQ ID NO: 107); an HCDR2 comprising the amino acid sequence of the HCDR2 of monoclonal antibody ZC10 (e.g., SEQ ID NO: 108); and an HCDR3 comprising the amino acid sequence of the HCDR3 of monoclonal antibody ZC10 (e.g., SEQ ID NO: 103), and (ii) a VL comprising: an LCDR1 comprising the amino acid sequence of the LCDR1 of monoclonal antibody ZC10 (e.g., SEQ ID NO: 104); an LCDR2 comprising the amino acid sequence of the LCDR2 of monoclonal antibody ZC10 (e.g., SEQ ID NO: 105); and an LCDR3 comprising the amino acid sequence of the LCDR3 of monoclonal antibody ZC10 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC10 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC10 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC10 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC10 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC10 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC10 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC10 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC10 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC10 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC10 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC10 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC10 (e.g., SEQ ID NO: 106).

In an embodiment, the antibody molecule comprises: (i) a VH comprising: an HCDR1 comprising the amino acid sequence of the HCDR1 of monoclonal antibody ZC10 (e.g., SEQ ID NO: 203); an HCDR2 comprising the amino acid sequence of the HCDR2 of monoclonal antibody ZC10 (e.g., SEQ ID NO: 207); and an HCDR3 comprising the amino acid sequence of the HCDR3 of monoclonal antibody ZC10 (e.g., SEQ ID NO: 103), and (ii) a VL comprising: an LCDR1 comprising the amino acid sequence of the LCDR1 of monoclonal antibody ZC10 (e.g., SEQ ID NO: 104); an LCDR2 comprising the amino acid sequence of the LCDR2 of monoclonal antibody ZC10 (e.g., SEQ ID NO: 105); and an LCDR3 comprising the amino acid sequence of the LCDR3 of monoclonal antibody ZC10 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC10 (e.g., SEQ ID NO: 22). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC10 (e.g., SEQ ID NO: 7).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC10 (e.g., SEQ ID NO: 22); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC10 (e.g., SEQ ID NO: 7). In an embodiment, the antibody molecule comprises: (i) a VH comprising the amino acid sequence of the VH of monoclonal antibody ZC10 (e.g., SEQ ID NO: 22); and (ii) a VL comprising the amino acid sequence of the VL of monoclonal antibody ZC10 (e.g., SEQ ID NO: 7).

In an embodiment, the antibody molecule comprises a VH encoded by the nucleotide sequence of SEQ ID NO: 317 (or a nucleotide sequence substantially identical thereto) or a VL encoded by the nucleotide sequence of SEQ ID NO: 306 (or a nucleotide sequence substantially identical thereto), or both.

In an embodiment the antibody molecule comprises monoclonal antibody ZC10.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC11 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC11 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC11 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC11 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC11 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC11 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC11 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC11 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC11 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC11 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC11 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC11 (e.g., SEQ ID NO: 106).

In an embodiment, the antibody molecule comprises: (i) a VH comprising: an HCDR1 comprising the amino acid sequence of the HCDR1 of monoclonal antibody ZC11 (e.g., SEQ ID NO: 107); an HCDR2 comprising the amino acid sequence of the HCDR2 of monoclonal antibody ZC11 (e.g., SEQ ID NO: 108); and an HCDR3 comprising the amino acid sequence of the HCDR3 of monoclonal antibody ZC11 (e.g., SEQ ID NO: 103), and (ii) a VL comprising: an LCDR1 comprising the amino acid sequence of the LCDR1 of monoclonal antibody ZC11 (e.g., SEQ ID NO: 111); an LCDR2 comprising the amino acid sequence of the LCDR2 of monoclonal antibody ZC11 (e.g., SEQ ID NO: 105); and an LCDR3 comprising the amino acid sequence of the LCDR3 of monoclonal antibody ZC11 (e.g., SEQ ID NO: 106).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC11 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC11 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC11 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC11 (e.g., SEQ ID NO: 111); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC11 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC11 (e.g., SEQ ID NO: 106).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC11 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC11 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC11 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC11 (e.g., SEQ ID NO: 111); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC11 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC11 (e.g., SEQ ID NO: 106).

In an embodiment, the antibody molecule comprises: (i) a VH comprising: an HCDR1 comprising the amino acid sequence of the HCDR1 of monoclonal antibody ZC11 (e.g., SEQ ID NO: 203); an HCDR2 comprising the amino acid sequence of the HCDR2 of monoclonal antibody ZC11 (e.g., SEQ ID NO: 207); and an HCDR3 comprising the amino acid sequence of the HCDR3 of monoclonal antibody ZC11 (e.g., SEQ ID NO: 103), and (ii) a VL comprising: an LCDR1 comprising the amino acid sequence of the LCDR1 of monoclonal antibody ZC11 (e.g., SEQ ID NO: 111); an LCDR2 comprising the amino acid sequence of the LCDR2 of monoclonal antibody ZC11 (e.g., SEQ ID NO: 105); and an LCDR3 comprising the amino acid sequence of the LCDR3 of monoclonal antibody ZC11 (e.g., SEQ ID NO: 106).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC11 (e.g., SEQ ID NO: 22). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC11 (e.g., SEQ ID NO: 6).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC11 (e.g., SEQ ID NO: 22); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC11 (e.g., SEQ ID NO: 6). In an embodiment, the antibody molecule comprises: (i) a VH comprising the amino acid sequence of the VH of monoclonal antibody ZC11 (e.g., SEQ ID NO: 22); and (ii) a VL comprising the amino acid sequence of the VL of monoclonal antibody ZC11 (e.g., SEQ ID NO: 6).

In an embodiment, the antibody molecule comprises a VH encoded by the nucleotide sequence of SEQ ID NO: 317 (or a nucleotide sequence substantially identical thereto) or a VL encoded by the nucleotide sequence of SEQ ID NO: 313 (or a nucleotide sequence substantially identical thereto), or both.

In an embodiment the antibody molecule comprises monoclonal antibody ZC11.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC12 (e.g., SEQ ID NO: 107; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC12 (e.g., SEQ ID NO: 108); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC12 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC12 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC12 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC12 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC12 (e.g., SEQ ID NO: 107); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC12 (e.g., SEQ ID NO: 108); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC12 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC12 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC12 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC12 (e.g., SEQ ID NO: 119).

In an embodiment, the antibody molecule comprises: (i) a VH comprising: an HCDR1 comprising the amino acid sequence of the HCDR1 of monoclonal antibody ZC12 (e.g., SEQ ID NO: 107); an HCDR2 comprising the amino acid sequence of the HCDR2 of monoclonal antibody ZC12 (e.g., SEQ ID NO: 108); and an HCDR3 comprising the amino acid sequence of the HCDR3 of monoclonal antibody ZC12 (e.g., SEQ ID NO: 103), and (ii) a VL comprising: an LCDR1 comprising the amino acid sequence of the LCDR1 of monoclonal antibody ZC12 (e.g., SEQ ID NO: 104); an LCDR2 comprising the amino acid sequence of the LCDR2 of monoclonal antibody ZC12 (e.g., SEQ ID NO: 105); and an LCDR3 comprising the amino acid sequence of the LCDR3 of monoclonal antibody ZC12 (e.g., SEQ ID NO: 119).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC12 (e.g., SEQ ID NO: 203; (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC12 (e.g., SEQ ID NO: 207); or (iii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC12 (e.g., SEQ ID NO: 103).

In an embodiment, the antibody molecule comprises a VL, wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC12 (e.g., SEQ ID NO: 104); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC12 (e.g., SEQ ID NO: 105); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC12 (e.g., SEQ ID NO: 119).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ZC12 (e.g., SEQ ID NO: 203); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ZC12 (e.g., SEQ ID NO: 207); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ZC12 (e.g., SEQ ID NO: 103), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ZC12 (e.g., SEQ ID NO: 104); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ZC12 (e.g., SEQ ID NO: 105); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ZC12 (e.g., SEQ ID NO: 119).

In an embodiment, the antibody molecule comprises: (i) a VH comprising: an HCDR1 comprising the amino acid sequence of the HCDR1 of monoclonal antibody ZC12 (e.g., SEQ ID NO: 203); an HCDR2 comprising the amino acid sequence of the HCDR2 of monoclonal antibody ZC12 (e.g., SEQ ID NO: 207); and an HCDR3 comprising the amino acid sequence of the HCDR3 of monoclonal antibody ZC12 (e.g., SEQ ID NO: 103), and (ii) a VL comprising: an LCDR1 comprising the amino acid sequence of the LCDR1 of monoclonal antibody ZC12 (e.g., SEQ ID NO: 104); an LCDR2 comprising the amino acid sequence of the LCDR2 of monoclonal antibody ZC12 (e.g., SEQ ID NO: 105); and an LCDR3 comprising the amino acid sequence of the LCDR3 of monoclonal antibody ZC12 (e.g., SEQ ID NO: 119).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC12 (e.g., SEQ ID NO: 22). In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC12 (e.g., SEQ ID NO: 21).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ZC12 (e.g., SEQ ID NO: 22); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ZC12 (e.g., SEQ ID NO: 21). In an embodiment, the antibody molecule comprises: (i) a VH comprising the amino acid sequence of the VH of monoclonal antibody ZC12 (e.g., SEQ ID NO: 22); and (ii) a VL comprising the amino acid sequence of the VL of monoclonal antibody ZC12 (e.g., SEQ ID NO: 21).

In an embodiment, the antibody molecule comprises a VH encoded by the nucleotide sequence of SEQ ID NO: 317 (or a nucleotide sequence substantially identical thereto) or a VL encoded by the nucleotide sequence of SEQ ID NO: 318 (or a nucleotide sequence substantially identical thereto), or both.

In an embodiment the antibody molecule comprises monoclonal antibody ZC12.

Amino acid and nucleotide sequences of exemplary antibody molecules are described in Tables 1 and 2, respectively.

TABLE 1
The amino acid sequences of the heavy chain variable region (VH) and light chain variable region
VL) of the exemplary anti-ZIKV antibodies are provided as follows. CDRs, defined according to the
Kabat system or Chothia system, are listed. CDRs, defined according to the Kabat system, are also
underlined and bold.

| | Chain | | | SEQ ID | | | SEQ ID |
|---|---|---|---|---|---|---|---|
| Antibody | ID | Chain | Amino Acid Sequence | NO | Chothia CDR | | NO |
| ZV54 | | VH | QVQLQQSGVGLARPGTSVKLSCKASGYSFTTYGISWV | 1 | HCDR1 | GYSFTTY | 101 |
| Mouse | | | TQRPGQGLEWIGVIYPRSNNTYYNERFRGKATLTADK | | HCDR2 | YPRSNN | 102 |
| | | | SSSSAYLELRGLTAEDSAVYFCARENYGSVYWGQGTT | | HCDR3 | ENYGSVY | 103 |
| | | | LTVSS | | | | |
| | | VL | DIVMTQSQKFMSTSVGDRVTITCKASQSVGTAVAWYH | 2 | LCDR1 | KASQSVGTAVA | 104 |
| | | | QKPGQSPKLLIYSASNRYTGVPDRFTGSGSGTDFTLT | | LCDR2 | SASNRYT | 105 |
| | | | ITYMQSEDLADYFCQQFSNYPFTFGGGTKLEIK | | LCDR3 | QQFSNYPFT | 106 |
| ZV67 | | VH | QVQLQQSGTGLARPGASVKLSCKASGYTFTSYGISWV | 3 | HCDR1 | GYTFTSY | 107 |
| Mouse | | | TQRAGQGLEWIGVIYPRSGNTYYNEKFRGKATLTADK | | HCDR2 | YPRSGN | 108 |
| | | | SSSSAYMELRGLTAEDSAVYFCARENYGSVYWGQGTT | | HCDR3 | ENYGSVY | 103 |
| | | | LTVSS | | | | |
| | | VL | DIVMTQSQKFMSTSVGDRVSITCKASQNVGTAVAWYQ | 4 | LCDR1 | KASQHVGTAVA | 109 |
| | | | QKPGQSPKLLIYSASNRYTGVPDRFTGSGSGTDFTLT | | LCDR2 | SASNRYT | 105 |
| | | | ISHMQSEDLADYFCQQFSSYPYTFGGGTKLEIK | | LCDR3 | QQFSSYPYT | 110 |
| ZA-1/1 | A | VH | QVQLVQSGAEVKKPGSSVKVSCKASGYSFTTYGISWV | 5 | HCDR1 | GYSFTTY | 101 |
| | series | | RQAPGQGLEWMGVIYPRSGNTYYAQKFQGRVTITADK | | HCDR2 | YPRSGN | 108 |
| | VH1 | | STSTAYMELSSLRSEDTAVYFCARENYGSVYWGQGTL | | HCDR3 | ENYGSVY | 103 |
| | | | VTVSS | | | | |
| | A | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTEGGGTKVEIK | | LCDR3 | QQFSNYPFT | 106 |
| ZA-1/2 | A | VH | QVQLVQSGAEVKKPGSSVKVSCKASGYSFTTYGISWV | 5 | HCDR1 | GYSFTTY | 101 |
| | series | | RQAPGQGLEWMGVIYPRSGNTYYAQKFQGRVTITADK | | HCDR2 | YPRSGN | 108 |
| | VH1 | | STSTAYMELSSLRSEDTAVYFCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTEGGGTKVEIK | | LCDR3 | QQFSNYPFT | 106 |
| ZA-1/3 | A | VH | QVQLVQSGAEVKKPGSSVKVSCKASGYSFTTYGISWV | 5 | HCDR1 | GYSFTTY | 101 |
| | series | | RQAPGQGLEWMGVIYPRSGNTYYAQKFQGRVTITADK | | HCDR2 | YPRSGN | 108 |
| | VH1 | | STSTAYMELSSLRSEDTAVYFCARENYGSVYWGQGTL | | HCDR3 | ENYGSVY | 103 |
| | | | VTVSS | | | | |
| | A | VL | DIRMTQSPSSFSASTGDRVTITCRASQSVGTAVAWYQ | 8 | LCDR1 | RASQSVGTAVA | 111 |
| | series | | QKPGKAPKLLIYSASNLYTGVPSRFSGSGSGTDFTLT | | LCDR2 | SASNLYT | 112 |
| | VL3 | | ISCLQSEDFATYFCQQFSNYPFTFGGGTKVEIK | | LCDR3 | QQFSNYPFT | 106 |
| ZA-2/1 | A | VH | QVQLVQSGAEVKKPGSSVKVSCKASGYSFTTYGISWV | 9 | HCDR1 | GYSFTTY | 101 |
| | series | | RQAPGQGLEWMGVIYPRSGNTYYAEKFRGRVTITADK | | HCDR2 | YPRSGN | 108 |
| | VH2 | | STSTAYMELSSLRSEDTAVYYCARENYGSVYWGQGTL | | HCDR3 | ENYGSVY | 103 |
| | | | VTVSS | | | | |
| | A | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZA-2/2 | A | VH | QVQLVQSGAEVKKPGSSVKVSCKASGYSFTTYGISWV | 9 | HCDR1 | GYSFTTY | 101 |
| | series | | RQAPGQGLEWMGVIYPRSGNTYYAEKFRGRVTITADK | | HCDR2 | YPRSGN | 108 |
| | VH2 | | STSTAYMELSSLRSEDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZA-2/3 | A | VH | QVQLVQSGAEVKKPGSSVKVSCKASGYSFTTYGISWV | 9 | HCDR1 | GYSFTTY | 101 |
| | series | | RQAPGQGLEWMGVIYPRSGNTYYAEKFRGRVTITADK | | HCDR2 | YPRSGN | 108 |
| | VH2 | | STSTAYMELSSLRSEDTAVYYCARENYGSVYWGQGTL | | HCDR3 | ENYGSVY | 103 |
| | | | VTVSS | | | | |
| | A | VL | DIRMTQSPSSFSASTGDRVTITCRASQSVGTAVAWYQ | 8 | LCDR1 | RASQSVGTAVA | 111 |
| | series | | QKPGKAPKLLIYSASNLYTGVPSRFSGSGSGTDFTLT | | LCDR2 | SASNLYT | 112 |
| | VL3 | | ISCLQSEDFATYFCQQFSNYPFTFGGGTKVEIK | | LCDR3 | QQFSNYPFT | 106 |
| ZA-3/1 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | A | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZA-3/2 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZA-3/3 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | A | VL | DIRMTQSPSSFSASTGDRVTITCRASQSVGTAVAWYQ | 8 | LCDR1 | RASQSVGTAVA | 111 |
| | series | | QKPGKAPKLLIYSASNLYTGVPSRFSGSGSGTDFTLT | | LCDR2 | SASNLYT | 112 |
| | VL3 | | ISCLQSEDFATYFCQQFSNYPFTFGGGTKVEIK | | LCDR3 | QQFSNYPFT | 106 |
| ZA-4/1 | A | VH | QVQLVQSGAEVKKPGASVKVSCKASGYSFTTYGISWV | 11 | HCDR1 | GYSFTTY | 101 |
| | series | | RQAPGQGLEWMGVIYPRSGNTYYAEKLRGRVTMTADK | | HCDR2 | YPRSGN | 108 |
| | VH4 | | STSTAYMELRSLRSDDTAVYYCARENYGSVYWGQGTL | | HCDR3 | ENYGSVY | 103 |
| | | | VTVSS | | | | |
| | A | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZA-4/2 | A | VH | QVQLVQSGAEVKKPGASVKVSCKASGYSFTTYGISWV | 11 | HCDR1 | GYSFTTY | 101 |
| | series | | RQAPGQGLEWMGVIYPRSGNTYYAEKLRGRVTMTADK | | HCDR2 | YPRSGN | 108 |
| | VH4 | | STSTAYMELRSLRSDDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZA-4/3 | A | VH | QVQLVQSGAEVKKPGASVKVSCKASGYSFTTYGISWV | 11 | HCDR1 | GYSFTTY | 101 |
| | series | | RQAPGQGLEWMGVIYPRSGNTYYAEKLRGRVTMTADK | | HCDR2 | YPRSGN | 108 |
| | VH4 | | STSTAYMELRSLRSDDTAVYYCARENYGSVYWGQGTL | | HCDR3 | ENYGSVY | 103 |
| | | | VTVSS | | | | |
| | A | VL | DIRMTQSPSSFSASTGDRVTITCRASQSVGTAVAWYQ | 8 | LCDR1 | RASQSVGTAVA | 111 |
| | series | | QKPGKAPKLLIYSASNLYTGVPSRFSGSGSGTDFTLT | | LCDR2 | SASNLYT | 112 |
| | VL3 | | ISCLQSEDFATYFCQQFSNYPFTFGGGTKVEIK | | LCDR3 | QQFSNYPFT | 106 |
| ZA-5/1 | A | VH | QVQLVQSGAEVKKPGASVKVSCKASGYSFTTYGISWV | 12 | HCDR1 | GYSFTTY | 101 |
| | series | | RQATGQGLEWMGVIYPRSGNTYYAEKFRGRVTMTANK | | HCDR2 | YPRSGN | 108 |
| | VH5 | | SISTAYMELSSLRSEDTAVYYCARENYGSVYWGQGTL | | HCDR3 | ENYGSVY | 103 |
| | | | VTVSS | | | | |
| | A | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZA-5/2 | A | VH | QVQLVQSGAEVKKPGASVKVSCKASGYSFTTYGISWV | 12 | HCDR1 | GYSFTTY | 101 |
| | series | | RQATGQGLEWMGVIYPRSGNTYYAEKFRGRVTMTANK | | HCDR2 | YPRSGN | 108 |
| | VH5 | | SISTAYMELSSLRSEDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZA-5/3 | A | VH | QVQLVQSGAEVKKPGASVKVSCKASGYSFTTYGISWV | 12 | HCDR1 | GYSFTTY | 101 |
| | series | | RQATGQGLEWMGVIYPRSGNTYYAEKFRGRVTMTANK | | HCDR2 | YPRSGN | 108 |
| | VH5 | | SISTAYMELSSLRSEDTAVYYCARENYGSVYWGQGTL | | HCDR3 | ENYGSVY | 103 |
| | | | VTVSS | | | | |
| | A | VL | DIRMTQSPSSFSASTGDRVTITCRASQSVGTAVAWYQ | 8 | LCDR1 | RASQSVGTAVA | 111 |
| | series | | QKPGKAPKLLIYSASNLYTGVPSRFSGSGSGTDFTLT | | LCDR2 | SASNLYT | 112 |
| | VL3 | | ISCLQSEDFATYFCQQFSNYPFTFGGGTKVEIK | | LCDR3 | QQFSNYPFT | 106 |
| ZB-1/1 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZB-1/2 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGEAVAWYQ | 18 | LCDR1 | RASQSVGEAVA | 117 |
| | series | | QKPGQAPRLLIYSASNRYTGIPARFSGSGSGTEFTLT | | LCDR2 | SASNRYT | 105 |
| | VL2 | | ISSLQSEDFADYFCQQFSNYPFTFGGGTKVEIK | | LCDR3 | QQFSNYPFT | 106 |
| ZB-1/3 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 19 | LCDR1 | RASQSVGTAVA | 111 |
| | series | | QKPGQAPRLLIYSASDRYTGIPARFSGSGSGTEFTLT | | LCDR2 | SASDRYT | 118 |
| | VL3 | | ISSLQSEDFADYFCQQFSNYPFTFGGGTKVEIK | | LCDR3 | QQFSNYPFT | 106 |
| ZB-1/4 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZB-2/1 | B | VH | QVQLVQSGAEVKKPGASVKVSCKASGYTFTSYGISWV | 14 | HCDR1 | GYTFTSY | 107 |
| | series | | RQATGQGLEWMGVIYPRSGNTYYAEKFRGRVTMTADK | | HCDR2 | YPRSGN | 108 |
| | VH2 | | SISTAYMELSSLRSEDTAVYYCAREDYGSVYWGQGTL | | HCDR3 | EDYGSVY | 113 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZB-2/2 | B | VH | QVQLVQSGAEVKKPGASVKVSCKASGYTFTSYGISWV | 14 | HCDR1 | GYTFTSY | 107 |
| | series | | RQATGQGLEWMGVIYPRSGNTYYAEKFRGRVTMTADK | | HCDR2 | YPRSGN | 108 |
| | VH2 | | SISTAYMELSSLRSEDTAVYYCAREDYGSVYWGQGTL | | HCDR3 | EDYGSVY | 113 |
| | | | VTVSS | | | | |
| | B | VL | EIVMTQSPATLSVSPGERATLSCRASQSVGEAVAWYQ | 18 | LCDR1 | RASQSVGEAVA | 117 |
| | series | | QKPGQAPRLLIYSASNRYTGIPARFSGSGSGTEFTLT | | LCDR2 | SASNRYT | 105 |
| | VL2 | | ISSLQSEDFADYFCQQFSNYPFTFGGGTKVEIK | | LCDR3 | QQFSNYPFT | 106 |
| ZB-2/3 | B | VH | QVQLVQSGAEVKKPGASVKVSCKASGYTFTSYGISWV | 14 | HCDR1 | GYTFTSY | 107 |
| | series | | RQATGQGLEWMGVIYPRSGNTYYAEKFRGRVTMTADK | | HCDR2 | YPRSGN | 108 |
| | VH2 | | SISTAYMELSSLRSEDTAVYYCAREDYGSVYWGQGTL | | HCDR3 | EDYGSVY | 113 |
| | | | VTVSS | | | | |
| | B | VL | EIVMTQSPATLSVSPGERATLSCRASQSVGTAVAWYQ | 19 | LCDR1 | RASQSVGTAVA | 111 |
| | series | | QKPGQAPRLLIYSASDRYTGIPARFSGSGSGTEFTLT | | LCDR2 | SASDRYT | 118 |
| | VL3 | | ISSLQSEDFADYFCQQFSNYPFTFGGGTKVEIK | | LCDR3 | QQFSNYPFT | 106 |
| ZB-2/4 | B | VH | QVQLVQSGAEVKKPGASVKVSCKASGYTFTSYGISWV | 14 | HCDR1 | GYTFTSY | 107 |
| | series | | RQATGQGLEWMGVIYPRSGNTYYAEKFRGRVTMTADK | | HCDR2 | YPRSGN | 108 |
| | VH2 | | SISTAYMELSSLRSEDTAVYYCAREDYGSVYWGQGTL | | HCDR3 | EDYGSVY | 113 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZB-3/1 | B | VH | QVQLVQSGAEVKKPGASVKVSCKASGYTFTSYGISWV | 15 | HCDR1 | GYTFTSY | 107 |
| | series | | RQATGQGLEWMGVIYPRSGHTYYAEKFRGRVTMTADK | | HCDR2 | YPRSGH | 114 |
| | VH3 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZB-3/2 | B | VH | QVQLVQSGAEVKKPGASVKVSCKASGYTFTSYGISWV | 15 | HCDR1 | GYTFTSY | 107 |
| | series | | RQATGQGLEWMGVIYPRSGHTYYAEKFRGRVTMTADK | | HCDR2 | YPRSGH | 114 |
| | VH3 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGEAVAWYQ | 18 | LCDR1 | RASQSVGEAVA | 117 |
| | series | | QKPGQAPRLLIYSASNRYTGIPARFSGSGSGTEFTLT | | LCDR2 | SASNRYT | 105 |
| | VL2 | | ISSLQSEDFADYFCQQFSNYPFTFGGGTKVEIK | | LCDR3 | QQFSNYPFT | 106 |
| ZB-3/3 | B | VH | QVQLVQSGAEVKKPGASVKVSCKASGYTFTSYGISWV | 15 | HCDR1 | GYTFTSY | 107 |
| | series | | RQATGQGLEWMGVIYPRSGHTYYAEKFRGRVTMTADK | | HCDR2 | YPRSGH | 114 |
| | VH3 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 19 | LCDR1 | RASQSVGTAVA | 111 |
| | series | | QKPGQAPRLLIYSASDRYTGIPARFSGSGSGTEFTLT | | LCDR2 | SASDRYT | 118 |
| | VL3 | | ISSLQSEDFADYFCQQFSNYPFTFGGGTKVEIK | | LCDR3 | QQFSNYPFT | 106 |
| ZB-3/4 | B | VH | QVQLVQSGAEVKKPGASVKVSCKASGYTFTSYGISWV | 15 | HCDR1 | GYTFTSY | 107 |
| | series | | RQATGQGLEWMGVIYPRSGHTYYAEKFRGRVTMTADK | | HCDR2 | YPRSGH | 114 |
| | VH3 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZB-4/1 | B | VH | QVQLVQSGAEVKKPGASVKVSCKASGYTFTSYGISWV | 16 | HCDR1 | GYTFTSY | 107 |
| | series | | RQATGQGLEWMGVIYPRSGNTYYAEKFRGRVTMTADK | | HCDR2 | YPRSGN | 108 |
| | VH4 | | SISTAYMELSSLRSEDTAVYYCARENYGSTYWGQGTL | | HCDR3 | ENYGSTY | 115 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZB-4/2 | B | VH | QVQLVQSGAEVKKPGASVKVSCKASGYTFTSYGISWV | 16 | HCDR1 | GYTFTSY | 107 |
| | series | | RQATGQGLEWMGVIYPRSGNTYYAEKFRGRVTMTADK | | HCDR2 | YPRSGN | 108 |
| | VH4 | | SISTAYMELSSLRSEDTAVYYCARENYGSTYWGQGTL | | HCDR3 | ENYGSTY | 115 |
| | | | VTVSS | | | | |
| | B | VL | EIVMTQSPATLSVSPGERATLSCRASQSVGEAVAWYQ | 18 | LCDR1 | RASQSVGEAVA | 117 |
| | series | | QKPGQAPRLLIYSASNRYTGIPARFSGSGSGTEFTLT | | LCDR2 | SASNRYT | 105 |
| | VL2 | | ISSLQSEDFADYFCQQFSNYPFTFGGGTKVEIK | | LCDR3 | QQFSNYPFT | 106 |
| ZB-4/3 | B | VH | QVQLVQSGAEVKKPGASVKVSCKASGYTFTSYGISWV | 16 | HCDR1 | GYTFTSY | 107 |
| | series | | RQATGQGLEWMGVIYPRSGNTYYAEKFRGRVTMTADK | | HCDR2 | YPRSGN | 108 |
| | VH4 | | SISTAYMELSSLRSEDTAVYYCARENYGSTYWGQGTL | | HCDR3 | ENYGSTY | 115 |
| | | | VTVSS | | | | |
| | B | VL | EIVMTQSPATLSVSPGERATLSCRASQSVGTAVAWYQ | 19 | LCDR1 | RASQSVGTAVA | 111 |
| | series | | QKPGQAPRLLIYSASDRYTGIPARFSGSGSGTEFTLT | | LCDR2 | SASDRYT | 118 |
| | VL3 | | ISSLQSEDFADYFCQQFSNYPFTFGGGTKVEIK | | LCDR3 | QQFSNYPFT | 106 |
| ZB-4/4 | B | VH | QVQLVQSGAEVKKPGASVKVSCKASGYTFTSYGISWV | 16 | HCDR1 | GYTFTSY | 107 |
| | series | | RQATGQGLEWMGVIYPRSGNTYYAEKFRGRVTMTADK | | HCDR2 | YPRSGN | 108 |
| | VH4 | | SISTAYMELSSLRSEDTAVYYCARENYGSTYWGQGTL | | HCDR3 | ENYGSTY | 115 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZB-5/1 | B | VH | QVQLVQSGAEVKKPGASVKVSCKASGYTFTSYGISWV | 17 | HCDR1 | GYTFTSY | 107 |
| | series | | RQATGQGLEWMGVIYPRSGNTYYAEKFRGRVTMTADK | | HCDR2 | YPRSGN | 108 |
| | VH5 | | SISTAYMELSSLRSEDTAVYYCARENYGSDYWGQGTL | | HCDR3 | ENYGSDY | 116 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZB-5/2 | B | VH | QVQLVQSGAEVKKPGASVKVSCKASGYTFTSYGISWV | 17 | HCDR1 | GYTFTSY | 107 |
| | series | | RQATGQGLEWMGVIYPRSGNTYYAEKFRGRVTMTADK | | HCDR2 | YPRSGN | 108 |
| | VH5 | | SISTAYMELSSLRSEDTAVYYCARENYGSDYWGQGTL | | HCDR3 | ENYGSDY | 116 |
| | | | VTVSS | | | | |
| | B | VL | EIVMTQSPATLSVSPGERATLSCRASQSVGEAVAWYQ | 18 | LCDR1 | RASQSVGEAVA | 117 |
| | series | | QKPGQAPRLLIYSASNRYTGIPARFSGSGSGTEFTLT | | LCDR2 | SASNRYT | 105 |
| | VL2 | | ISSLQSEDFADYFCQQFSNYPFTFGGGTKVEIK | | LCDR3 | QQFSNYPFT | 106 |
| ZB-5/3 | B | VH | QVQLVQSGAEVKKPGASVKVSCKASGYTFTSYGISWV | 17 | HCDR1 | GYTFTSY | 107 |
| | series | | RQATGQGLEWMGVIYPRSGNTYYAEKFRGRVTMTADK | | HCDR2 | YPRSGN | 108 |
| | VH5 | | SISTAYMELSSLRSEDTAVYYCARENYGSDYWGQGTL | | HCDR3 | ENYGSDY | 116 |
| | | | VTVSS | | | | |
| | B | VL | EIVMTQSPATLSVSPGERATLSCRASQSVGTAVAWYQ | 19 | LCDR1 | RASQSVGTAVA | 111 |
| | series | | QKPGQAPRLLIYSASDRYTGIPARFSGSGSGTEFTLT | | LCDR2 | SASDRYT | 118 |
| | VL3 | | ISSLQSEDFADYFCQQFSNYPFTFGGGTKVEIK | | LCDR3 | QQFSNYPFT | 106 |
| ZB-5/4 | B | VH | QVQLVQSGAEVKKPGASVKVSCKASGYTFTSYGISWV | 17 | HCDR1 | GYTFTSY | 107 |
| | series | | RQATGQGLEWMGVIYPRSGNTYYAEKFRGRVTMTADK | | HCDR2 | YPRSGN | 108 |
| | VH5 | | SISTAYMELSSLRSEDTAVYYCARENYGSDYWGQGTL | | HCDR3 | ENYGSDY | 116 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZC1 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZC2 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZC3 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZC4 | B | VH | QVQLVQSGAEVKKPGASVKVSCKASGYTFTSYGISWV | 13 | HCDR1 | GYTFTSY | 107 |
| | series | | RQATGQGLEWMGVIYPRSGNTYYAEKFRGRVTMTADK | | HCDR2 | YPRSGN | 108 |
| | VH1 | | SISTAYMELSSLRSEDTAVYYCARENYGSVYWGQGTL | | HCDR3 | ENYGSVY | 103 |
| | | | VTVSS | | | | |
| | C | VL | DIQMTQSPSSLSASVGDRVTITCKASQSVGTAVAWYQ | 21 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL1 | | ISSLQPEDFATYFCQQFYNYPFTFGGGTKVEIK | | HCDR3 | QQFYNYPFT | 119 |
| ZC5 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZC6 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZC7 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZC8 | A | VH | QVQLVQSGAEVKKPGASVKVSCKASGYSFTTYGISWV | 10 | HCDR1 | GYSFTTY | 101 |
| | series | | RQAPGQGLEWMGVIYPRSGNTYYAQKLQGRVTMTTDT | | HCDR2 | YPRSGN | 108 |
| | VH3 | | STSTAYMELRSLRSDDTAVYYCARENYGSVYWGQGTL | | HCDR3 | ENYGSVY | 103 |
| | | | VTVSS | | | | |
| | C | VL | DIQMTQSPSSLSASVGDRVTITCKASQSVGTAVAWYQ | 21 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL1 | | ISSLQPEDFATYFCQQFYNYPFTFGGGTKVEIK | | HCDR3 | QQFYNYPFT | 119 |
| ZC9 | C | VH | QVQLVQSGAEVKKPGASVKVSCKASGYTFTSYGISWV | 22 | HCDR1 | GYTFTSY | 107 |
| | series | | RQAPGQGLEWMGVIYPRSGNTYYAQKLQGRVTMTTDT | | HCDR2 | YPRSGN | 108 |
| | VH1 | | STSTAYMELRSLRSDDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 20 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL4 | | ISSLQSEDFADYFCQQFYNYPFTFGGGTKVEIK | | LCDR3 | QQFYNYPFT | 119 |
| ZC10 | C | VH | QVQLVQSGAEVKKPGASVKVSCKASGYTFTSYGISWV | 22 | HCDR1 | GYTFTSY | 107 |
| | series | | RQAPGQGLEWMGVIYPRSGNTYYAQKLQGRVTMTTDT | | HCDR2 | YPRSGN | 108 |
| | VH1 | | STSTAYMELRSLRSDDTAVYYCARENYGSVYWGQGTL | | HCDR3 | ENYGSVY | 103 |
| | | | VTVSS | | | | |
| | A | VL | DIQMTQSPSSLSASVGDRVTITCKASQSVGTAVAWYQ | 7 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL2 | | ISSLQPEDFATYFCQQFSNYPFTFGGGTKVEIK | | LCDR3 | QQFSNYPFT | 106 |
| ZC11 | C | VH | QVQLVQSGAEVKKPGASVKVSCKASGYTFTSYGISWV | 22 | HCDR1 | GYTFTSY | 107 |
| | series | | RQAPGQGLEWMGVIYPRSGNTYYAQKLQGRVTMTTDT | | HCDR2 | YPRSGN | 108 |
| | VH1 | | STSTAYMELRSLRSDDTAVYYCARENYGSVYWGQGTL | | HCDR3 | ENYGSVY | 103 |
| | | | VTVSS | | | | |
| | B | VL | EIVMTQSPATLSVSPGERATLSCRASQSVGTAVAWYQ | 6 | LCDR1 | RASQSVGTAVA | 111 |
| | series | | QKPGQAPRLLIYSASNRYTGIPARFSGSGSGTEFTLT | | LCDR2 | SASNRYT | 105 |
| | VL1 | | ISSLQSEDFADYFCQQFSNYPFTFGGGTKVEIK | | LCDR3 | QQFSNYPFT | 106 |
| ZC12 | C | VH | QVQLVQSGAEVKKPGASVKVSCKASGYTFTSYGISWV | 22 | HCDR1 | GYTFTSY | 107 |
| | series | | RQAPGQGLEWMGVIYPRSGNTYYAQKLQGRVTMTTDT | | HCDR2 | YPRSGN | 108 |
| | VH1 | | STSTAYMELRSLRSDDTAVYYCARENYGSVYWGQGTL | | HCDR3 | ENYGSVY | 103 |
| | | | VTVSS | | | | |
| | C | VL | DIQMTQSPSSLSASVGDRVTITCKASQSVGTAVAWYQ | 21 | LCDR1 | KASQSVGTAVA | 104 |
| | series | | QKPGKAPKLLIYSASNRYTGVPSRFSGSGSGTDFTLT | | LCDR2 | SASNRYT | 105 |
| | VL1 | | ISSLQPEDFATYFCQQFYNYPFTFGGGTKVEIK | | LCDR3 | QQFYNYPFT | 119 |
| | | | | | | | SEQ ID |
| | | | | Antibody | Kabat CDR | | NO |
| | | | | ZV54 | HCDR1 | TYGIS | 201 |
| | | | | Mouse | HCDR2 | VIYPRSHNTYYNERFRG | 202 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZV67 | HCDR1 | SYGIS | 203 |
| | | | | Mouse | HCDR2 | VIYPRSGNTYYNEKFRG | 204 |
| | | | | | HCDR3 | ENYGSVY | 103 |
| | | | | | LCDR1 | KASQNVGTAVA | 109 |
| | | | | | LCDR2 | SASNRYT | 105 |
| | | | | | LCDR3 | QQFSSYPYT | 110 |
| | | | | ZA-1/1 | HCDR1 | TYGIS | 201 |
| | | | | | HCDR2 | VIYPRSGNTYYAQKFQG | 205 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZA-1/2 | HCDR1 | TYGIS | 201 |
| | | | | | HCDR2 | VIYPRSGNTYYAQKFQG | 205 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZA-1/3 | HCDR1 | TYGIS | 201 |
| | | | | | HCDR2 | VIYPRSGNTYYAQKFQG | 205 |
| | | | | | HCDR3 | ENYGSVY | 103 |
| | | | | | LCDR1 | RASQSVGTAVA | 111 |
| | | | | | LCDR2 | SASNLYT | 112 |
| | | | | | LCDR3 | QQFSNYPFT | 106 |
| | | | | ZA-2/1 | HCDR1 | TYGIS | 201 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZA-2/2 | HCDR1 | TYGIS | 201 |
| | | | | | HCDR2 | VIYPRSGNTYYAEKFRG | 206 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZA-2/3 | HCDR1 | TYGIS | 201 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNLYT | 112 |
| | | | | | LCDR3 | QQFSNYPFT | 106 |
| | | | | ZA-3/1 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZA-3/2 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZA-3/3 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | RASQSVGTAVA | 111 |
| | | | | | LCDR2 | SASNLYT | 112 |
| | | | | | LCDR3 | QQFSNYPFT | 106 |
| | | | | ZA-4/1 | HCDR1 | TYGIS | 201 |
| | | | | | HCDR2 | VIYPRSGNTYYAEKLRG | 208 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZA-4/2 | HCDR1 | TYGIS | 201 |
| | | | | | HCDR2 | VIYPRSGNTYYAEKLRG | 208 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZA-4/3 | HCDR1 | TYGIS | 201 |
| | | | | | HCDR2 | VIYPRSGNTYYAEKLRG | 208 |
| | | | | | HCDR3 | ENYGSVY | 103 |
| | | | | | LCDR1 | RASQSVGTAVA | 111 |
| | | | | | LCDR2 | SASNLYT | 112 |
| | | | | | LCDR3 | QQFSNYPFT | 106 |
| | | | | ZA-5/1 | HCDR1 | TYGIS | 201 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZA-5/2 | HCDR1 | TYGIS | 201 |
| | | | | | HCDR2 | VIYPRSGNTYYAEKFRG | 206 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZA-5/3 | HCDR1 | TYGIS | 201 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNLYT | 112 |
| | | | | | LCDR3 | QQFSNYPFT | 106 |
| | | | | ZB-1/1 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZB-1/2 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | RASQSVGEAVA | 117 |
| | | | | | LCDR2 | SASNRYT | 105 |
| | | | | | LCDR3 | QQFSNYPFT | 106 |
| | | | | ZB-1/3 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASDRYT | 118 |
| | | | | | LCDR3 | QQFSNYPFT | 106 |
| | | | | ZB-1/4 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZB-2/1 | HCDR1 | SYGIS | 203 |
| | | | | | HCDR2 | VIYPRSGNTYYAEKFRG | 206 |
| | | | | | HCDR3 | EDYGSVY | 113 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZB-2/2 | HCDR1 | SYGIS | 203 |
| | | | | | HCDR2 | VIYPRSGNTYYAEKFRG | 206 |
| | | | | | HCDR3 | EDYGSVY | 113 |
| | | | | | LCDR1 | RASQSVGEAVA | 117 |
| | | | | | LCDR2 | SASNRYT | 105 |
| | | | | | LCDR3 | QQFSNYPFT | 106 |
| | | | | ZB-2/3 | HCDR1 | SYGIS | 203 |
| | | | | | HCDR2 | VIYPRSGNTYYAEKFRG | 206 |
| | | | | | HCDR3 | EDYGSVY | 113 |
| | | | | | LCDR1 | RASQSVGTAVA | 111 |
| | | | | | LCDR2 | SASDRYT | 118 |
| | | | | | LCDR3 | QQFSNYPFT | 106 |
| | | | | ZB-2/4 | HCDR1 | SYGIS | 203 |
| | | | | | HCDR2 | VIYPRSGNTYYAEKFRG | 206 |
| | | | | | HCDR3 | EDYGSVY | 113 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZB-3/1 | HCDR1 | SYGIS | 203 |
| | | | | | HCDR2 | VIYPRSGHTYYAEKFRG | 209 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZB-3/2 | HCDR1 | SYGIS | 203 |
| | | | | | HCDR2 | VIYPRSGHTYYAEKFRG | 209 |
| | | | | | HCDR3 | ENYGSVY | 103 |
| | | | | | LCDR1 | RASQSVGEAVA | 117 |
| | | | | | LCDR2 | SASNRYT | 105 |
| | | | | | LCDR3 | QQFSNYPFT | 106 |
| | | | | ZB-3/3 | HCDR1 | SYGIS | 203 |
| | | | | | HCDR2 | VIYPRSGHTYYAEKFRG | 209 |
| | | | | | HCDR3 | ENYGSVY | 103 |
| | | | | | LCDR1 | RASQSVGTAVA | 111 |
| | | | | | LCDR2 | SASDRYT | 118 |
| | | | | | LCDR3 | QQFSNYPFT | 106 |
| | | | | ZB-3/4 | HCDR1 | SYGIS | 203 |
| | | | | | HCDR2 | VIYPRSGHTYYAEKFRG | 209 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZB-4/1 | HCDR1 | SYGIS | 203 |
| | | | | | HCDR2 | VIYPRSGNTYYAEKFRG | 206 |
| | | | | | HCDR3 | ENYGSTY | 115 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZB-4/2 | HCDR1 | SYGIS | 203 |
| | | | | | HCDR2 | VIYPRSGNTYYAEKFRG | 206 |
| | | | | | HCDR3 | ENYGSTY | 115 |
| | | | | | LCDR1 | RASQSVGEAVA | 117 |
| | | | | | LCDR2 | SASNRYT | 105 |
| | | | | | LCDR3 | QQFSNYPFT | 106 |
| | | | | ZB-4/3 | HCDR1 | SYGIS | 203 |
| | | | | | HCDR2 | VIYPRSGNTYYAEKFRG | 206 |
| | | | | | HCDR3 | ENYGSTY | 115 |
| | | | | | LCDR1 | RASQSVGTAVA | 111 |
| | | | | | LCDR2 | SASDRYT | 118 |
| | | | | | LCDR3 | QQFSNYPFT | 106 |
| | | | | ZB-4/4 | HCDR1 | SYGIS | 203 |
| | | | | | HCDR2 | VIYPRSGNTYYAEKFRG | 206 |
| | | | | | HCDR3 | ENYGSTY | 115 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZB-5/1 | HCDR1 | SYGIS | 203 |
| | | | | | HCDR2 | VIYPRSGNTYYAEKFRG | 206 |
| | | | | | HCDR3 | ENYGSDY | 116 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZB-5/2 | HCDR1 | SYGIS | 203 |
| | | | | | HCDR2 | VIYPRSGNTYYAEKFRG | 206 |
| | | | | | HCDR3 | ENYGSDY | 116 |
| | | | | | LCDR1 | RASQSVGEAVA | 117 |
| | | | | | LCDR2 | SASNRYT | 105 |
| | | | | | LCDR3 | QQFSNYPFT | 106 |
| | | | | ZB-5/3 | HCDR1 | SYGIS | 203 |
| | | | | | HCDR2 | VIYPRSGNTYYAEKFRG | 206 |
| | | | | | HCDR3 | ENYGSDY | 116 |
| | | | | | LCDR1 | RASQSVGTAVA | 111 |
| | | | | | LCDR2 | SASDRYT | 118 |
| | | | | | LCDR3 | QQFSNYPFT | 106 |
| | | | | ZB-5/4 | HCDR1 | SYGIS | 203 |
| | | | | | HCDR2 | VIYPRSGNTYYAEKFRG | 206 |
| | | | | | HCDR3 | ENYGSDY | 116 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZC1 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZC2 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZC3 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZC4 | HCDR1 | SYGIS | 203 |
| | | | | | HCDR2 | VIYPRSGNTYYAEKFRG | 206 |
| | | | | | HCDR3 | ENYGSVY | 103 |
| | | | | | LCDR1 | KASQSVGTAVA | 104 |
| | | | | | LCDR2 | SASNRYT | 105 |
| | | | | | HCDR3 | QQFYNYPFT | 119 |
| | | | | ZC5 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZC6 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZC7 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZC8 | HCDR1 | TYGIS | 201 |
| | | | | | HCDR2 | VIYPRSGNTYYAQKLQG | 207 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFYNYPFT | 119 |
| | | | | ZC9 | HCDR1 | SYGIS | 203 |
| | | | | | HCDR2 | VIYPRSGNTYYAQKLQG | 207 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFYNYPFT | 119 |
| | | | | ZC10 | HCDR1 | SYGIS | 203 |
| | | | | | HCDR2 | VIYPRSGNTYYAQKLQG | 207 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFSNYPFT | 106 |
| | | | | ZC11 | HCDR1 | SYGIS | 203 |
| | | | | | HCDR2 | VIYPRSGNTYYAQKLQG | 207 |
| | | | | | HCDR3 | ENYGSVY | 103 |
| | | | | | LCDR1 | RASQSVGTAVA | 111 |
| | | | | | LCDR2 | SASNRYT | 105 |
| | | | | | LCDR3 | QQFSNYPFT | 106 |
| | | | | ZC12 | HCDR1 | SYGIS | 203 |
| | | | | | HCDR2 | VIYPRSGNTYYAQKLQG | 207 |
| | | | | | HCDR3 | ENYGSVY | 103 |
| | | | | | LCDR1 | KASQSVGTAVA | 104 |
| | | | | | LCDR2 | SASNRYT | 105 |
| | | | | | LCDR3 | QQFYNYPFT | 119 |

TABLE 2
The nucleotide sequences of the heavy chain variable region (VH) and light chain variable region
(VL) of the exemplary anti-ZIKV antibodies are provided as follows.

| | | | | SEQ |
|---|---|---|---|---|
| | | | | ID |
| Design | Chain | Name | Nucleotide Sequence | NO |
| A | Heavy | VH1 | CAAGTCCAACTCGTGCAATCCGGGGCAGAAGTGAAGAAACCAGGTAGCTCAGTGAAGGTATCATGTAAAGCAAGCGGGTAC | 300 |
| | | | AGTTTCACGACCTACGGTATCAGTTGGGTACGGCAGGCTCCGGGACAGGGTTTGGAATGGATGGGTGTAATTTACCCTAGA | |
| | | | AGCGGTAATACATACTATGCTCAGAAGTTTCAAGGGAGGGTCACGATTACAGCCGACAAAAGTACAAGCACCGCCTACATG | |
| | | | GAGCTGTCATCCCTGCGATCAGAAGATACGGCTGTCTACTTTTGCGCCCGAGAGAATTATGGCTCCGTCTACTGGGGTCAG | |
| | | | GGCACGCTTGTCACAGTAAGTTCC | |
| A | Heavy | VH2 | CAGGTTCAGTTGGTGCAGTCTGGAGCGGAAGTAAAGAAGCCTGGTTCCAGTGTGAAGGTAAGTTGTAAGGCTAGCGGTTAC | 301 |
| | | | AGCTTCACAACTTACGGCATAAGTTGGGTCAGACAAGCGCCGGGGCAAGGGCTCGAATGGATGGGAGTCATATATCCCCGG | |
| | | | TCTGGCAACACCTATTACGCTGAGAAGTTTCGAGGTCGAGTCACAATAACGGCGGATAAAAGTACCTCCACTGCCTACATG | |
| | | | GAGTTGTCATCTCTTCGATCTGAGGATACGGCTGTCTACTACTGCGCACGAGAAAATTATGGCTCCGTCTACTGGGGACAG | |
| | | | GGGACATTGGTCACGGTCAGTAGT | |
| A | Heavy | VH3 | CAAGTACAACTCGTACAAAGTGGCGCAGAGGTGAAAAAACCCGGAGCTTCAGTGAAGGTCAGTTGTAAGGCATCTGGTTAC | 302 |
| | | | TCCTTCACCACTTACGGAATAAGTTGGGTACGGCAGGCACCGGGCCAGGGGCTTGAGTGGATGGGAGTGATTTACCCCCGC | |
| | | | AGCGGTAATACCTATTATGCACAAAAACTCCAGGGGCGGGTAACCATGACTACCGACACTTCTACTTCTACTGCGTACATG | |
| | | | GAGTTGAGATCATTGAGGTCTGATGATACAGCGGTTTACTACTGCGCTCGAGAGAACTATGGTTCCGTGTATTGGGGCCAA | |
| | | | GGCACATTGGTAACAGTTTCTTCC | |
| A | Heavy | VH4 | CAGGTTCAACTGGTTCAATCTGGGGCTGAAGTCAAGAAACCAGGCGCGTCAGTGAAAGTCTCTTGCAAAGCGTCTGGTTAT | 303 |
| | | | TCTTTCACGACATACGGTATATCATGGGTTCGACAAGCCCCTGGACAGGGTTTGGAATGGATGGGGGTCATTTATCCCAGA | |
| | | | AGTGGCAATACCTATTATGCGGAGAAGCTCAGGGGCCGGGTAACGATGACCGCGGACAAAAGTACCAGCACCGCTTATATG | |
| | | | GAACTTCGATCTCTCAGATCAGATGACACGGCAGTCTACTACTGCGCCCGCGAAAACTACGGGTCAGTGTACTGGGGGCAG | |
| | | | GGCACTCTGGTCACTGTATCTAGT | |
| A | Heavy | VH5 | CAGGTACAACTCGTGCAGAGCGGGGCCGAAGTCAAGAAGCCCGGCGCATCCGTTAAAGTATCATGCAAGGCGTCAGGATAT | 304 |
| | | | TCTTTCACAACTTATGGCATTTCTTGGGTTCGGCAGGCTACCGGCCAAGGTTTGGAATGGATGGGCGTTATCTACCCTAGG | |
| | | | TCCGGAAATACCTATTATGCTGAAAAGTTTAGAGGTCGCGTTACTATGACCGCAAACAAGTCAATTTCTACCGCCTACATG | |
| | | | GAACTCTCAAGCCTGAGGAGTGAGGACACTGCTGTATATTACTGTGCGCGGGAAAATTATGGGAGTGTGTACTGGGGCCAG | |
| | | | GGGACTTTGGTTACGGTTTCATCC | |
| A | Light | VL1 | GAGATAGTTATGACCCAGAGCCCAGCTACATTGTCCGTGTCCCCCGGTGAAAGAGCCACGCTGAGTTGTAGAGCGTCACAG | 305 |
| | | | AGTGTAGGGACGGCGGTTGCGTGGTATCAACAGAAGCCAGGGCAGGCACCAAGGCTCTTGATTTATTCAGCATCCAATCGA | |
| | | | TACACTGGCATTCCAGCTAGATTCTCAGGTTCTGGTTCAGGCACGGAATTTACATTGACAATCTCATCCCTGCAATCAGAA | |
| | | | GATTTCGCCGATTATTTCTGTCAACAATTCTCCAATTACCCATTCACGTTTGGAGGAGGGACAAAAGTGGAAATCAAA | |
| A | Light | VL2 | GACATTCAAATGACACAGAGTCCTTCTAGCCTTAGCGCCTCAGTAGGTGACAGGGTTACGATCACCTGCAAAGCGAGCCAG | 306 |
| | | | AGCGTTGGAACTGCAGTTGCGTGGTATCAGCAAAAGCCTGGGAAGGCTCCCAAGCTGCTCATATATTCCGCCTCAAACCGC | |
| | | | TATACAGGAGTTCCCAGCAGATTTTCAGGATCTGGATCTGGCACGGATTTCACGCTGACCATCAGTTCACTGCAACCGGAA | |
| | | | GACTTCGCTACCTATTTTTGTCAGCAGTTTTCAAACTACCCTTTTACTTTCGGGGGTGGAACCAAAGTGGAAATTAAA | |
| A | Light | VL3 | GACATCAGAATGACTCAAAGTCCCTCTAGTTTTTCAGCCTCTACAGGCGATCGAGTTACTATAACGTGTCGGGCTAGCCAG | 307 |
| | | | TCCGTTGGAACCGCCGTCGCCTGGTATCAGCAAAAACCTGGAAAGGCCCCGAAATTGCTGATATACTCTGCTTCTAATCTT | |
| | | | TACACTGGCGTTCCCTCCAGGTTTTCTGGCTCAGGGAGCGGTACTGATTTTACGCTGACAATTTCTTGTCTTCAATCAGAG | |
| | | | GACTTCGCCACCTATTTTTGTCAACAGTTTTCAAATTACCCGTTTACGTTCGGCGGCGGGACCAAGGTCGAAATTAAG | |
| B | Heavy | VH1 | CAAGTGCAACTCGTTCAGTCAGGCGCAGAGGTCAAGAAGCCTGGTGCATCCGTCAAAGTTTCATGCAAGGCCTCCGGATAT | 308 |
| | | | ACATTCACCTCCTATGGAATAAGCTGGGTCCGGCAGGCAACCGGCCAGGGTCTTGAATGGATGGGGGTGATATATCCAAGA | |
| | | | TCCGGAAACACATACTACGCCGAGAAATTTCGTGGGCGCGTCACCATGACAGCCGACAAAAGCATATCTACCGCCTATATG | |
| | | | GAACTTTCTAGCCTTCGTTCTGAGGATACAGCAGTGTATTATTGTGCCAGGGAGAACTACGGATCTGTCTATTGGGGCCAG | |
| | | | GGTACACTCGTAACAGTTAGCAGC | |
| B | Heavy | VH2 | CAAGTGCAACTCGTTCAGTCAGGCGCAGAGGTCAAGAAGCCTGGTGCATCCGTCAAAGTTTCATGCAAGGCCTCCGGATAT | 309 |
| | | | ACATTCACCTCCTATGGAATAAGCTGGGTCCGGCAGGCAACCGGCCAGGGTCTTGAATGGATGGGGGTGATATATCCAAGA | |
| | | | TCCGGAAACACATACTACGCCGAGAAATTTCGTGGGCGCGTCACCATGACAGCCGACAAAAGCATATCTACCGCCTATATG | |
| | | | GAACTTTCTAGCCTTCGTTCTGAGGATACAGCAGTGTATTATTGTGCCAGGGAGGACTACGGATCTGTCTATTGGGGCCAG | |
| | | | GGTACACTCGTAACAGTTAGCAGC | |
| B | Heavy | VH3 | CAAGTGCAACTCGTTCAGTCAGGCGCAGAGGTCAAGAAGCCTGGTGCATCCGTCAAAGTTTCATGCAAGGCCTCCGGATAT | 310 |
| | | | ACATTCACCTCCTATGGAATAAGCTGGGTCCGGCAGGCAACCGGCCAGGGTCTTGAATGGATGGGGGTGATATATCCAAGA | |
| | | | TCCGGACACACATACTACGCCGAGAAATTTCGTGGGCGCGTCACCATGACAGCCGACAAAAGCATATCTACCGCCTATATG | |
| | | | GAACTTTCTAGCCTTCGTTCTGAGGATACAGCAGTGTATTATTGTGCCAGGGAGAACTACGGATCTGTCTATTGGGGCCAG | |
| | | | GGTACACTCGTAACAGTTAGCAGC | |
| B | Heavy | VH4 | CAAGTGCAACTCGTTCAGTCAGGCGCAGAGGTCAAGAAGCCTGGTGCATCCGTCAAAGTTTCATGCAAGGCCTCCGGATAT | 311 |
| | | | ACATTCACCTCCTATGGAATAAGCTGGGTCCGGCAGGCAACCGGCCAGGGTCTTGAATGGATGGGGGTGATATATCCAAGA | |
| | | | TCCGGAAACACATACTACGCCGAGAAATTTCGTGGGCGCGTCACCATGACAGCCGACAAAAGCATATCTACCGCCTATATG | |
| | | | GAACTTTCTAGCCTTCGTTCTGAGGATACAGCAGTGTATTATTGTGCCAGGGAGAACTACGGATCTACCTATTGGGGCCAG | |
| | | | GGTACACTCGTAACAGTTAGCAGC | |
| B | Heavy | VH5 | CAAGTGCAACTCGTTCAGTCAGGCGCAGAGGTCAAGAAGCCTGGTGCATCCGTCAAAGTTTCATGCAAGGCCTCCGGATAT | 312 |
| | | | ACATTCACCTCCTATGGAATAAGCTGGGTCCGGCAGGCAACCGGCCAGGGTCTTGAATGGATGGGGGTGATATATCCAAGA | |
| | | | TCCGGAAACACATACTACGCCGAGAAATTTCGTGGGCGCGTCACCATGACAGCCGACAAAAGCATATCTACCGCCTATATG | |
| | | | GAACTTTCTAGCCTTCGTTCTGAGGATACAGCAGTGTATTATTGTGCCAGGGAGAACTACGGATCTGACTATTGGGGCCAG | |
| | | | GGTACACTCGTAACAGTTAGCAGC | |
| B | Light | VL1 | GAAATCGTAATGACCCAGAGTCCTGCCACTCTGTCAGTGTCTCCAGGTGAGCGTGCAACTCTCTCTTGCCGCGCCTCTCAA | 313 |
| | | | TCTGTGGGGACCGCTGTGGCATGGTACCAGCAAAAACCCGGACAGGCCCCTCGTCTTCTCATCTATTCTGCATCTAACCGA | |
| | | | TACACTGGGATCCCTGCCAGGTTTTCTGGGAGTGGGTCAGGAACCGAATTTACCCTCACAATTTCTTCTTTGCAAAGCGAA | |
| | | | GATTTTGCCGATTACTTCTGCCAACAATTCTCCAACTACCCATTTACCTTTGGTGGAGGTACTAAAGTGGAGATAAAA | |
| B | Light | VL2 | GAAATCGTAATGACCCAGAGTCCTGCCACTCTGTCAGTGTCTCCAGGTGAGCGTGCAACTCTCTCTTGCCGCGCCTCTCAA | 314 |
| | | | TCTGTGGGGGAGGCTGTGGCATGGTACCAGCAAAAACCCGGACAGGCCCCTCGTCTTCTCATCTATTCTGCATCTAACCGA | |
| | | | TACACTGGGATCCCTGCCAGGTTTTCTGGGAGTGGGTCAGGAACCGAATTTACCCTCACAATTTCTTCTTTGCAAAGCGAA | |
| | | | GATTTTGCCGATTACTTCTGCCAACAATTCTCCAACTACCCATTTACCTTTGGTGGAGGTACTAAAGTGGAGATAAAA | |
| B | Light | VL3 | GAAATCGTAATGACCCAGAGTCCTGCCACTCTGTCAGTGTCTCCAGGTGAGCGTGCAACTCTCTCTTGCCGCGCCTCTCAA | 315 |
| | | | TCTGTGGGGACCGCTGTGGCATGGTACCAGCAAAAACCCGGACAGGCCCCTCGTCTTCTCATCTATTCTGCATCTGACCGA | |
| | | | TACACTGGGATCCCTGCCAGGTTTTCTGGGAGTGGGTCAGGAACCGAATTTACCCTCACAATTTCTTCTTTGCAAAGCGAA | |
| | | | GATTTTGCCGATTACTTCTGCCAACAATTCTCCAACTACCCATTTACCTTTGGTGGAGGTACTAAAGTGGAGATAAAA | |
| B | Light | VL4 | GAAATCGTAATGACCCAGAGTCCTGCCACTCTGTCAGTGTCTCCAGGTGAGCGTGCAACTCTCTCTTGCCGCGCCTCTCAA | 316 |
| | | | TCTGTGGGGACCGCTGTGGCATGGTACCAGCAAAAACCCGGACAGGCCCCTCGTCTTCTCATCTATTCTGCATCTAACCGA | |
| | | | TACACTGGGATCCCTGCCAGGTTTTCTGGGAGTGGGTCAGGAACCGAATTTACCCTCACAATTTCTTCTTTGCAAAGCGAA | |
| | | | GATTTTGCCGATTACTTCTGCCAACAATTCTACAACTACCCATTTACCTTTGGTGGAGGTACTAAAGTGGAGATAAAA | |
| C | Heavy | VH1 | CAAGTACAACTCGTACAAAGTGGCGCAGAGGTGAAAAAACCCGGAGCTTCAGTGAAGGTCAGTTGTAAGGCATCTGGTTAC | 317 |
| | | | ACCTTCACCTCTTACGGAATAAGTTGGGTACGGCAGGCACCGGGCCAGGGGCTTGAGTGGATGGGAGTGATTTACCCCCGC | |
| | | | AGCGGTAATACCTATTATGCACAAAAACTCCAGGGGCGGGTAACCATGACTACCGACACTTCTACTTCTACTGCGTACATG | |
| | | | GAGTTGAGATCATTGAGGTCTGATGATACAGCGGTTTACTACTGCGCTCGAGAGAACTATGGTTCCGTGTATTGGGGCCAA | |
| | | | GGCACATTGGTAACAGTTTCTTCC | |
| C | Light | VL1 | GACATTCAAATGACACAGAGTCCTTCTAGCCTTAGCGCCTCAGTAGGTGACAGGGTTACGATCACCTGCAAAGCGAGCCAG | 318 |
| | | | AGCGTTGGAACTGCAGTTGCGTGGTATCAGCAAAAGCCTGGGAAGGCTCCCAAGCTGCTCATATATTCCGCCTCAAACCGC | |
| | | | TATACAGGAGTTCCCAGCAGATTTTCAGGATCTGGATCTGGCACGGATTTCACGCTGACCATCAGTTCACTGCAACCGGAA | |
| | | | GACTTCGCTACCTATTTTTGTCAGCAGTTTTACAACTACCCTTTTACTTTCGGGGGTGGAACCAAAGTGGAAATTAAA | |

### Vaccines and Immunogens

Antibodies of the invention have elucidated epitopes that are useful for inducing immunity to, and in an embodiment, provide protection from, Zika virus. These epitopes are, in an embodiment, referred to herein as “immunogens.” In an embodiment, the immunogen induces immunity, and in another embodiment, confers protection against one or more Zika virus strains. An immunogen, as the term is used herein, can comprise a polypeptide having sufficient sequence and three dimensional features of an E protein of Zika virus, e.g., domain III of the E protein, to allow binding of an antibody molecule described herein, to the immunogen. In an embodiment, the immunogen comprises the epitope of an antibody molecule described herein. In an embodiment, the immunogen does not bind to an antibody that is cross-reactive with two or more flaviviruses (e.g., Zika virus and dengue virus). In an embodiment, an antibody molecule described herein binds the immunogen with at least 50, 60, 70, 80, 90, 95, or 99% of the affinity with which it binds a native Zika virus E protein. In an embodiment, the epitope of an antibody molecule described herein is the immunodominant epitope on the immunogen.

As used herein, the term “vaccine,” in an embodiment, refers to a preparation comprising an immunogen, or a nucleic acid encoding an immunogen, that can induce formation of antibodies or immunity against the immunogen or an organism, e.g., Zika virus. The vaccine can include dead or weakened virus or antigenic determinants from the organism, e.g., Zika virus. Typically, the vaccine will include one or more additional components, e.g., carriers, adjuvants and the like. In an embodiment, a vaccine comprises a plurality of immunogens, or a nucleic acid encoding a plurality of immunogens.

Immunogens disclosed herein, and vaccines including an immunogen, or nucleic acids encoding immunogens, can be used to elicit an immune response, in a subject, e.g., a human subject, against Zika virus described herein. In an embodiment, the vaccine confers protection against one or more of the Zika virus strains described herein, e.g., it decreases the chance of developing an infection or the symptom of an infection, or moderates the severity of an infection. Vaccines described herein can comprise a polypeptide comprising an immunogen, a nucleic acid encoding a polypeptide comprising an immunogen, a particle (e.g., a VLP, liposome, or nanoparticle) comprising an immunogen or a nucleic acid encoding an immunogen. Vaccines can comprise live or inactivated, e.g., replication deficient, viruses.

As used herein, the term “immunogen” or “antigenic formulation” or “antigenic composition” can refer to a preparation which, when administered to a vertebrate, e.g., a mammal, e.g., a human, can induce an immune response.

An immunogen, e.g., a polypeptide, or VLP, liposome, or nanoparticle comprising an immunogen, can be formulated into compositions that further comprise a pharmaceutically acceptable carrier or excipient. A nucleic acid that encodes an immunogen can be formulated into a composition that further comprises a pharmaceutically acceptable carrier or excipient. A carrier or excipient is a pharmaceutical agent that does not itself induce the production of an immune response harmful to the animal receiving the composition and which may be administered as a vaccine component without causing undue toxicity. As used herein, the term “pharmaceutically acceptable vaccine component” includes components, e.g., a carrier, that have been approved by a regulatory agency of the Federal or a state government or listed in the U.S. Pharmacopeia, European Pharmacopeia, or other generally recognized pharmacopeia for use in mammals, e.g., in humans. Non-limiting examples of pharmaceutically acceptable carriers are saline, buffered saline, dextrose, water, glycerol, sterile isotonic aqueous buffer, mannitol, lactose, starch, magnesium stearate, sodium saccharine, cellulose, magnesium carbonate, and combinations thereof. In an embodiment, the formulation can be used for administration of the vaccine to humans. In an embodiment, the formulation is sterile, free from particulate matter, and/or non-pyrogenic. The vaccine may also include one or more of: a wetting agent, an emulsifying agent, and a buffering agent. The vaccine can be in solid form, e.g., a lyophilized powder, liquid solution, suspension, emulsion, tablet, pill, capsule, sustained release formulation, or powder.

In an embodiment, vaccines may include one or more adjuvants. Adjuvants are agents that enhance immune responses, and their use is known in the art (see, e.g., “Vaccine Design: The Subunit and Adjuvant Approach,” Pharmaceutical Biotechnology, Volume 6, Eds. Powell and Newman, Plenum Press, New York and London, 1995). Non-limiting examples of adjuvants are complete Freund's adjuvant (CFA), incomplete Freund's adjuvant (IFA), squalene, squalane, aluminum hydroxide, aluminum salts, calcium salts, and saponin fractions derived from the bark of the South American tree Quillaja Saponaria Molina (e.g., QS21). In an embodiment, the adjuvant may be an emulsion comprising oil and water. The oil phase may comprise squalene, squalane, and/or a surfactant. The surfactant may be a non-ionic surfactant, e.g., a mono- or di-Ci2-C24-fatty acid ester of sorbitan or mannide.

Synthetic variants of molecules recognized by Toll-Like Receptors (TLRs) may also be used as adjuvants. TLRs help the body to distinguish between self and non-self molecules by recognizing molecular patterns associated with pathogens. Molecules recognized by TLRs include double-stranded RNA, lipopolysaccharides, single-stranded RNA with viral-specific or bacterial-specific modifications, and DNA with viral-specific or bacterial-specific modifications. Synthetic molecules that mimic the properties of these naturally-occurring molecules recognized by TLRs help to trigger an immune response and therefore can be used as adjuvants. Non-limiting examples of such synthetic molecules include polyriboinosinic:polyribocytidylic acid (poly (I:C)), double-stranded nucleic acids with at least one locked nucleic acid nucleoside, attenuated lipid A derivatives (ALDs) (e.g., monophosphoryl lipid A and 3-deacyl monophosphoryl lipid A), and imiquimod.

Vaccines may be formulated with or administered in combination with the administration of immune stimulators. Immune stimulators are molecules that increase the response of the immune system. Non-limiting examples of immune stimulators are cytokines, lymphokines, and chemokines that have immunostimulatory, immunopotentiating, and pro-inflammatory activities, such as interleukins (e.g., IL-I, IL-2, IL-3, IL-4, IL-12, IL-13), growth factors (e.g., granulocyte-macrophage (GM)-colony stimulating factor (CSF)); and other immunostimulatory molecules, such as macrophage inflammatory factor, Flt3 ligand, B7.1; B7.2, etc. Immune stimulators may be administered in the same formulation as the VLPs or may be administered separately. Immune stimulators may be administered as proteins or as nucleic acids from which the immunostimulatory protein can be expressed.

Generally, vaccines will be administered in an effective amount or quantity sufficient to stimulate an immune response against Zika virus. Vaccine dosage can be adjusted within this range based on clinical factors, e.g., age, physical condition, body weight, sex, diet, and time of administration.

Methods of administering a vaccine include enteral and parenteral administration. They can also be provided by epidural or mucosal administration (e.g., intranasal and oral or pulmonary routes or by suppositories). They can be provided by inhalation or direct contact with the buccal or nasal cavities. In an embodiment, a vaccine is administered intramuscularly, intravenously, subcutaneously, transdermally, or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and by any convenient means, for example, by injection using a needle and syringe or a needle-less injection device, by drops, via an aerosol comprising large particles, or by spray into the upper respiratory tract. A vaccine may be administered together with other biologically active agents, for example, immunogenic agents, for example, antivirals and/or antibiotics. In an embodiment, a vaccine is administered so as to target certain tissues in order to elicit an immune response at the site of immunization.

A vaccine may be administered on a dosage schedule, for example, by sequential administrations to subject. In an embodiment, a first dose of the composition is followed after a period of time by a second dose. The period of time between the first and second doses may be anywhere from two weeks to one year, for example, about 1, about 2, about 3, about 4, about 5 to about 6 months. In an embodiment, the second dose of the composition is followed by a third dose administered a period of time after the first dose. The period of time between the first and third doses may be anywhere from about three months to about two years or more, for example, about 4, about 5, or about 6 months, or about 7 months to about one year. In an embodiment, the second, third, or higher dose is administered when the levels of specific immunoglobulins in the serum, urine, and/or mucosal secretions of the subject drop below a threshold. In an embodiment, the period of time between the first and second doses is about one month and the period of time between the first and third doses is about six months. In another embodiment, the period of time between the first and second doses is about six months. In an embodiment, for example, when the subject is a neonate or infant, doses can be administered throughout childhood. Other factors that put the subject at increased risk of infection, for example, subjects who are health care workers, day care workers, family members of young children, the elderly, and/or individuals with compromised cardiopulmonary function, may influence the dosage schedule, for example, may require the subject to have more doses or more frequent doses. When multiple doses are required, the doses may be administered by the same or different routes.

One skilled in the art can readily determine the dosage of the vaccine. For example, the dosage may be determined by identifying doses that elicit a protective or therapeutic immune response, for example, by measuring the level of specific immunoglobulins in the serum or measuring the inhibitory ratio of antibodies in samples of serum, urine, or mucosal secretions from a subject. Dosages can be determined from studies in animals, for example, in guinea pigs, hamsters, ferrets, chinchillas, mice, or rats. An animal need not be a natural host to a particular infectious agent to serve as a subject in studies of the disease caused by said infection agent. Dosages can also be determined from clinical studies in humans, which are routine in the art. The skilled artisan will understand that the route of administration will affect the dosage. Dosages can also be calculated from dose-response curves obtained from in vitro studies or studies using animal models.

A vaccine can be administered a subject that does not have a disease caused by Zika virus infection or has not been and is not currently infected with an Zika virus infection, e.g., a vaccine can be administered to a subject at risk for infection. A vaccine can be administered to a subject infected with a first Zika virus strain, e.g., to protect against infection with a second Zika virus strain. In an embodiment, the vaccine is protective against the first strain. In an embodiment, the vaccine is not protective against the first strain. In an embodiment, the subject is a female human, e.g., a pregnant female, or a female between the age of 16 and 50, e.g., between the age of 18 and 35. In an embodiment, the subject is immune-compromised. In an embodiment, the subject is a health care provider, e.g., a physician, nurse, or aid.

Vaccines can be administered either alone or in combination with one or more other therapy or agent, e.g., the administration of a second or additional agent, e.g., to prevent or delay or minimize one or more symptoms or effects of Zika virus infection. In an embodiment, the combination can result in a lower dose of the vaccine or of the other therapy being needed, which, in an embodiment, can reduce side effects. In an embodiment, the combination can result in enhanced delivery or efficacy of one or both agents. The agents or therapies can be administered at the same time (e.g., as a single formulation that is administered to a patient or as two separate formulations administered concurrently) or sequentially in any order. Such second or additional agents include other vaccines, anti-viral agents, and/or antibodies. In a typical embodiment, the second or additional agent is not co-formulated with the vaccine, though in others it is.

The second or additional agent can be, for example, for treatment or prevention of Zika virus infection. For example, a vaccine provided herein can be administered in combination with another vaccine, e.g., a mixture (e.g., a cocktail) of Zika viral peptides to stimulate the patient's immune system to prevent infection with particular strains of Zika virus. In an embodiment, the vaccine provided herein is administered in combination with a vaccine for dengue virus, Japanese encephalitis virus, West Nile virus, or a combination thereof. In another embodiment, the vaccine provided herein is administered in combination with a therapy (e.g., an antibody molecule or small molecule) for dengue virus, Japanese encephalitis virus, West Nile virus, or a combination thereof. In other examples, the second or additional agent is an anti-viral agent, a pain reliever, an anti-inflammatory, an antibiotic, a steroidal agent, a second therapeutic antibody molecule, an adjuvant, a protease or glycosidase. In an embodiment,

A vaccine can be provided in a virus-like particle (VLP). A VLP is a structure that shares some component and structural similarity to a virus but generally is not infectious. VLPs typically lack a viral genome and therefore cannot reproduce. VLPs can be produced by cloning and co-expressing one or more viral proteins, typically including an antigenic protein of interest, in a cell, and recovering from the cells VLPs that include the antigenic protein of interest.

VLPs can be produced by culturing host cells into which one or more constructs that enable the expression of exogenous polypeptides have been introduced. The exogenous proteins may be polypeptides identical to or derived from the polypeptides of the influenza virus, e.g., the E protein, fragments of the E protein, or variants of the E protein. The expression construct may contain one or more additional elements, e.g., a marker, e.g., a selectable marker, or an origin of replication. Methods to grow cells for production of VLPs include, but are not limited to, batch, batch-fed, continuous and perfusion cell culture techniques. Methods and reagents may be used to increase efficiency of VLP production. For example, a leader sequence, e.g., a signal sequence, may be added to one or more exogenous polypeptides, e.g., the E protein, to facilitate transport of the exogenous polypeptide(s) within the host cell.

VLPs can be isolated and purified using methods known in the art, such as density gradient centrifugation, filtration, ion exchange chromatography, and gel filtration chromatography. Using the methods described above, VLPs are produced by host cells and secreted into the culture medium. A typical stepwise procedure for isolating and purifying VLPs from the culture medium involves (1) ultrafiltration of the culture medium to concentrate VLPs, (2) diafiltration of VLPs to remove components of the culture medium, (3) centrifugation of VLPs on a sucrose density gradient to remove cellular debris and particulate matter, and (4) anion exchange chromatography of VLPs to remove nucleic acids.

A vaccine may be incorporated into, or packaged in, a vesicle. Typically, vesicles have an aqueous compartment enclosed by one or more bilayers comprising amphipathic molecules (e.g., fatty acids, lipids, steroids, etc.). A vaccine may be contained within the aqueous core of the vesicle or may be localized to the amphipathic bilayer.

In an embodiment, the amphipathic molecules of the vesicle are nonionic, e.g., a nonionic surfactant. For example, the nonionic amphipathic molecule may be a glyercol-based, ester-linked surfactant. Such glycerol esters may comprise one of two higher aliphatic acyl groups, e.g., an acyl group containing at least ten carbon atoms in each acyl moiety. Surfactants based on such glycerol esters may comprise more than one glycerol unit, e.g., 2, 3, 4, or 5 glycerol units. Glycerol monoesters may be used, e.g., those containing a C12-C20 alkanoyl or alkenoyl moiety, for example caproyl, lauroyl, myristoyl, palmitoyl, oleyl or stearoyl. An exemplary ester linked surfactant based on glycerol is 1-monopalmitoyl glycerol.

The nonionic amphipathic molecule of the vesicle bilayer may also be an ether-linked surfactant. For example, ether-linked surfactants based on glycerol or a glycol having a lower aliphatic glycol of up to 4 carbon atoms, such as ethylene glycol, may be used. Surfactants based on such glycols may comprise more than one glycol unit, e.g., 2, 3, 4, or 5 glycol units (e.g., diglycolcetyl ether and/or polyoxyethylene-3-lauryl ether). Glycol or glycerol monoethers may be used, including those containing a C12-C20 alkanyl or alkenyl moiety, for example capryl, lauryl, myristyl, cetyl, oleyl or stearyl. For examples of ethylene oxide condensation products that can be used as amphipathic molecules, see PCT Publication No. WO88/06882 (e.g., polyoxyethylene higher aliphatic ether and amine surfactants). Non-limiting examples of ether-linked surfactants are 1-monocetyl glycerol ether and diglycolcetyl ether.

In an embodiment, the vesicle comprising a nonionic surfactant may also comprise an ionic amphipathic molecule. For example, an ionic amphiphile may cause the vesicles become negatively charged, which may help stabilize the vesicles and promote dispersion. Ionic amphipathic molecules that can be incorporated into vesicles include, but are not limited to, higher alkanoic and alkenoic acids (e.g., palmitic acid, oleic acid) and other compounds containing acidic groups, for example, phosphates, (e.g., dialkyl phosphates, e.g., dicetylphosphate, or phosphatidic acid or phosphatidyl serine) and sulphate monoesters (e.g., higher alkyl sulphates, e.g., cetylsulphate). The ionic amphiphile may be present at between 1% and 30%, between 2% and 20%, or between 5% and 15% the amount, by weight, of nonionic surfactant. In an embodiment, the vesicle may further comprise a high-molecular weight hydrophobic molecule capable of forming a bilayer, for example, a steroid, for example, cholesterol. The presence of the steroid may facilitate formation of the bilayer, for example, by conferring physical properties on the bilayer. The steroid may be present at between 20% and 120%, between 25% and 90%, or between 35% and 75% amount, by weight, of nonionic surfactant. In an embodiment, the vesicle may be a bilosome (see, e.g., U.S. Pat. No. 5,876,721). As used herein, “bilosomes” are vesicles that comprise non-ionic surfactants and transport enhancing molecules which facilitate the transport of lipid-like molecules across mucosal membranes.

Methods for preparing vesicles comprising nonionic surfactants are known in the art. The skilled artisan will understand that such methods may be used to prepare vesicles comprising an immunogen.

An immunogen can be provided in a Zika virus. In an embodiment, the immunogen is incorporated in an E protein, e.g., an E protein that differs from wildtype. In an embodiment, the E protein that comprises the immunogen is other than a wild type sequence, e.g., an engineered sequence. It can be incorporated into a virion by supplying the polypeptide in trans during production of the virion or the genome of the virus can be engineered to produce it. In either case, viral particles comprising the immunogen are produced. In an embodiment, the virus is engineered to have an attenuated phenotype, e.g., the virus can have no, or only very low levels of, replication in human cells. In an embodiment, the virus is inactivated. Inactivation methods include contact with denaturants, e.g., formalin, heat, or detergent. An immunogen can be provided in a non-Zika virus, e.g., the non-Zika virus vector can be a Newcastle disease virus, a vaccinia virus, an adenovirus, adeno-associated virus (AAV), retrovirus, or lentivirus.

### Animal Models

The antibody molecules, vaccines or immunogens, as described herein, can be evaluated in vivo, e.g., using various animal models. For example, an animal model can be used to test the efficacy of an antibody molecule in inhibiting Zika virus and/or in treating or preventing a disorder described herein, e.g., a Zika virus infection, or the efficacy of a vaccine or immunogen in inducing an immune response against Zika virus. Animal models can also be used, e.g., to investigate for side effects, measure concentrations of antibody molecules in situ, or demonstrate correlations between an Zika virus activity and a disorder described herein (e.g., a Zika virus infection).

Exemplary animal models for Zika virus that can be used for evaluating an antibody molecule, vaccine or immunogen described herein include, but are not limited to, mouse models (e.g., as described in Dick, Trans. R. Soc. Trop. Med. Hyg. 46, 521-534 (1952); Bell et al. Arch. Gesamte Virusforsch 35, 183-193 (1971). These models can include transgenic AG129 mice, which have deficient anti-viral cytokine responses and thus are susceptible to viral infection (Johnson & Roehrig, J Virol. 73, 783-786 (1999)).

Exemplary types of animals that can be used to evaluate the antibody molecules, vaccines, or immunogens described herein include, but are not limited to, mice, rats, rabbits, guinea pigs, and non-human primates (e.g., monkeys).

### Pharmaceutical Compositions and Kits

In some aspects, this disclosure provides compositions, e.g., pharmaceutically acceptable compositions, which include an antibody molecule or vaccine, described herein, formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In certain embodiments, less than about 5%, e.g., less than about 4%, 3%, 2%, or 1% of the antibody molecules in the pharmaceutical composition are present as aggregates. In other embodiments, at least about 95%, e.g., at least about 96%, 97%, 98%, 98.5%, 99%, 99.5%, 99.8%, or more of the antibody molecules in the pharmaceutical composition are present as monomers. In some embodiments, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some embodiments, the antibody molecule is administered by intravenous infusion or injection. In certain embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule or antimicrobial peptide can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject's diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody molecule and the particular therapeutic, prophylactic, or diagnostic effect to be achieved, and (b) the limitations inherent in the art of compounding such an antibody molecule for the treatment of sensitivity in individuals.

An exemplary, non-limiting range for a therapeutically, prophylactically, or diagnostically effective amount of an antibody molecule is about 0.1-50 mg/kg, e.g., about 0.1-30 mg/kg, e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. The antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², e.g., about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount,” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual. A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing Zika virus infection. Alternatively, this property of a composition can be evaluated by examining the ability of the antibody molecule to inhibit Zika virus, e.g., by an in vitro assay.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., a Zika virus infection, can be diagnosed in vitro, ex vivo, or in vivo.

Also within this disclosure is a kit that comprises an antibody molecule or vaccine, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

In one embodiment, the antibody molecule or vaccine is packaged in a kit. In an embodiment, the kit comprises two containers, one of which contains an antibody molecule or vaccine as a lyophilized powder and the other of which contains a liquid for resuspending the antibody molecule or vaccine. In an embodiment, the kit comprises two containers, one of which contains the vaccine, and the other of which contains an adjuvant. The kit may contain a notice as required by governmental agency regulating the manufacture, use, and sale of pharmaceuticals or biological products, the notice indicating that the composition has been approved for manufacture, use, and/or sale for administration to humans.

The vaccine may be supplied in a hermetically-sealed container. The vaccine may be provided as a liquid or as a lyophilized powder that can be reconstituted by the addition, e.g., of water or saline, to a concentration suitable for administration to a subject.

### Nucleic Acids

The present disclosure also features nucleic acids comprising nucleotide sequences that encode the antibody molecules (e.g., heavy and light chain variable regions and CDRs of the antibody molecules), as described herein.

For example, the present disclosure features a first and second nucleic acid encoding heavy and light chain variable regions, respectively, of an antibody molecule chosen from one or more of the antibody molecules disclosed herein, or a portion of an antibody molecule, e.g., the variable regions. The nucleic acid comprises a nucleotide sequence encoding any one of the amino acid sequences herein, or a sequence substantially identical thereto (e.g., a sequence at least about 85%, 90%, 95%, 99% or more identical thereto, or which differs by no more than 3, 6, 15, 30, or 45 nucleotides from the sequences herein).

In certain embodiments, the nucleic acid comprises a nucleotide sequence encoding at least one, two, or three CDRs from a heavy chain variable region of an antibody molecule described herein, or encoding an amino acid sequence substantially homologous thereto (e.g., a sequence at least about 85%, 90%, 95%, 99% or more identical thereto, and/or having one or more substitutions, e.g., conserved substitutions). In some embodiments, the nucleic acid comprises a nucleotide sequence encoding at least one, two, or three CDRs from a light chain variable region of an antibody molecule described herein, or encoding an amino acid sequence substantially homologous thereto (e.g., a sequence at least about 85%, 90%, 95%, 99% or more identical thereto, and/or having one or more substitutions, e.g., conserved substitutions). In some embodiments, the nucleic acid comprises a nucleotide sequence encoding at least one, two, three, four, five, or six CDRs from heavy and light chain variable regions of an antibody molecule described herein, or encoding an amino acid sequence substantially homologous thereto (e.g., a sequence at least about 85%, 90%, 95%, 99% or more identical thereto, and/or having one or more substitutions, e.g., conserved substitutions).

In certain embodiments, the nucleic acid comprises a nucleotide sequence encoding at least one, two, or three CDRs from a heavy chain variable region of an antibody molecule described herein, or comprises a nucleotide sequence substantially homologous thereto (e.g., a sequence at least about 85%, 90%, 95%, 99% or more identical thereto, and/or capable of hybridizing under the stringency conditions described herein). In some embodiments, the nucleic acid comprises a nucleotide sequence encoding at least one, two, or three CDRs from a light chain variable region of an antibody molecule described herein, or comprises a nucleotide sequence substantially homologous thereto (e.g., a sequence at least about 85%, 90%, 95%, 99% or more identical thereto, and/or capable of hybridizing under the stringency conditions described herein). In certain embodiments, the nucleic acid comprises a nucleotide sequence encoding at least one, two, three, four, five, or six CDRs from heavy and light chain variable regions of an antibody molecule described herein, or comprises a nucleotide sequence substantially homologous thereto (e.g., a sequence at least about 85%, 90%, 95%, 99% or more identical thereto, and/or capable of hybridizing under the stringency conditions described herein).

In certain embodiments, the nucleic acid comprises a nucleotide sequence encoding a heavy chain variable region of an antibody molecule described herein, or encoding an amino acid sequence substantially homologous thereto (e.g., a sequence at least about 85%, 90%, 95%, 99% or more identical thereto, and/or having one or more substitutions, e.g., conserved substitutions). In some embodiments, the nucleic acid comprises a nucleotide sequence encoding a light chain variable region of an antibody molecule described herein, or encoding an amino acid sequence substantially homologous thereto (e.g., a sequence at least about 85%, 90%, 95%, 99% or more identical thereto, and/or having one or more substitutions, e.g., conserved substitutions). In some embodiments, the nucleic acid comprises a nucleotide sequence encoding a heavy chain variable region and a light chain variable region of an antibody molecule described herein, or encoding an amino acid sequence substantially homologous thereto (e.g., a sequence at least about 85%, 90%, 95%, 99% or more identical thereto, and/or having one or more substitutions, e.g., conserved substitutions).

In certain embodiments, the nucleic acid comprises a nucleotide sequence encoding a heavy chain variable region of an antibody molecule described herein, or comprises a nucleotide sequence substantially homologous thereto (e.g., a sequence at least about 85%, 90%, 95%, 99% or more identical thereto, and/or capable of hybridizing under the stringency conditions described herein). In some embodiments, the nucleic acid comprises a nucleotide sequence encoding a light chain variable region of an antibody molecule described herein, or comprises a nucleotide sequence substantially homologous thereto (e.g., a sequence at least about 85%, 90%, 95%, 99% or more identical thereto, and/or capable of hybridizing under the stringency conditions described herein). In certain embodiments, the nucleic acid comprises a nucleotide sequence encoding a heavy variable region and a light chain variable region of an antibody molecule described herein, or comprises a nucleotide sequence substantially homologous thereto (e.g., a sequence at least about 85%, 90%, 95%, 99% or more identical thereto, and/or capable of hybridizing under the stringency conditions described herein).

In some embodiments, the nucleic acid comprises a portion of a nucleotide sequence encoding, for example, a variable region (e.g., VH or VL); an antigen binding fragment; one, two, or three or more CDRs; or one, two, three, or four or more framework regions, of an antibody molecule described herein.

The present disclosure also features nucleic acids comprising nucleotide sequences that encode the immunogens described herein. The vaccines described herein can also be in the form of nucleic acids, or encoded by nucleic acids.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In some aspects, the disclosure features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an antibody molecule described herein or encoding an immunogen described herein.

In an embodiment, the vector comprises a nucleic acid encoding an antibody molecule described herein. In an embodiment, the vector comprises a nucleic acid encoding a heavy chain variable region of an antibody molecule described herein. In an embodiment, the vector comprises a nucleic acid encoding a light chain variable region of an antibody molecule described herein. In an embodiment, the vector comprises a nucleic acid encoding a heavy chain variable region and a light chain variable region of an antibody molecule described herein. In another embodiment, the vector comprises a nucleotide sequence described herein.

In an embodiment, the vector comprises a nucleic acid encoding an immunogen described herein. In an embodiment, the vaccine described herein comprises a vector or is a vector-based vaccine.

Numerous vector systems can be employed. The vectors include, but are not limited to, a virus, plasmid, cosmid, lambda phage or a yeast artificial chromosome (YAC). For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule or vaccines produced are known to those skilled in the art, and may be varied or optimized depending upon the specific expression vector and mammalian host cell employed, based upon the present description.

### Cells

The present disclosure also provides host cells comprising a nucleic acid encoding an antibody molecule as described herein or a nucleic acid encoding an immunogen described herein. For example, the host cells may comprise a nucleic acid having a nucleotide sequence described herein, or a nucleotide sequence substantially homologous thereto (e.g., a sequence at least about 85%, 90%, 95%, 99% or more identical thereto, and/or capable of hybridizing under the stringency conditions described herein), or a portion of one of said nucleic acids. Additionally, the host cells may comprise a nucleic acid encoding an amino acid sequence of an antibody molecule described herein, or encoding an amino acid sequence substantially homologous thereto (e.g., a sequence at least about 80%, 85%, 90%, 95%, 99% or more identical thereto, and/or having one or more substitutions, e.g., conserved substitutions), or a portion of one of said sequences.

In some embodiments, the host cells are genetically engineered to comprise nucleic acids encoding the antibody molecule described herein.

In certain embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses of Antibody Molecules

The antibody molecules disclosed herein, as well as the pharmaceutical compositions disclosed herein, have in vitro, ex vivo, and/or in vivo therapeutic, prophylactic, and/or diagnostic utilities. In certain embodiments, the antibody molecule described herein is used for treating, preventing, or diagnosing a disorder, e.g., a disorder described herein (e.g., a Zika virus infection or a disorder associated with Zika virus infection), in a subject that is at risk of being infected, or has been infected, with another flavivirus (e.g., a flavivirus described herein). In certain embodiments, the antibody molecule described herein is used for treating, preventing, or diagnosing a disorder, e.g., a disorder described herein (e.g., a Zika virus infection or a disorder associated with Zika virus infection), in a subject that is at risk of being infected, or has been infected, with dengue virus (DENV), chikungunya virus (CHIKV), or both, and/or has received a dengue vaccine, a chikungunya vaccine, or both. For example, the subject may have developed an anti-DENV antibody, an anti-CHIKV antibody, or both.

In an embodiment, the antibody molecule reduces (e.g., inhibits, blocks, or neutralizes) one or more biological activities of Zika virus. For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of Zika virus. In an embodiment, the antibody molecule binds to the E protein of Zika virus, e.g., domain III of the E protein. Accordingly, in an aspect, the disclosure provides a method of treating, preventing, or diagnosing a disorder, e.g., a disorder described herein (e.g., a Zika virus infection or a disorder associated with Zika virus infection), in a subject, comprising administering to the subject an antibody molecule described herein, such that the disorder is treated, prevented, or diagnosed. For example, the disclosure provides a method comprising contacting the antibody molecule described herein with cells in culture, e.g. in vitro or ex vivo, or administering the antibody molecule described herein to a subject, e.g., in vivo, to treat, prevent, or diagnose a disorder, e.g., a disorder associated with Zika virus (e.g., a Zika virus infection or a disorder associated with Zika virus infection).

The vaccines and immunogens disclosed herein also have therapeutic and/or prophylactic utilities. Accordingly, in an aspect, the disclosure features a method of inducing immunity to Zika virus (e.g., one or more Zika virus strains), or preventing, delaying or reducing infection with Zika virus (e.g., one or more virus Zika strains), or symptom thereof, in a subject. The method comprises administering to the subject, a vaccine described herein. In certain embodiments, the vaccine described herein is used for treating and/or preventing a disorder, e.g., a disorder described herein (e.g., a Zika virus infection or a disorder associated with Zika virus infection), in a subject that is at risk of being infected, or has been infected, with another flavivirus (e.g., a flavivirus described herein). In certain embodiments, the vaccine described herein is used for treating and/or preventing a disorder, e.g., a disorder described herein (e.g., a Zika virus infection or a disorder associated with Zika virus infection), in a subject that is at risk of being infected, or has been infected, with dengue virus (DENV), chikungunya virus (CHIKV), or both, and/or has received a dengue vaccine, a chikungunya vaccine, or both. For example, the subject may have developed an anti-DENV antibody, an anti-CHIKV antibody, or both.

As used herein, the term “subject” is intended to include human and non-human animals. In some embodiments, the subject is a human subject, e.g., a human patient having a disorder described herein (e.g., a Zika virus infection or a disorder associated with Zika virus infection), or at risk of having a disorder described herein (e.g., Zika virus infection or a disorder associated with Zika virus infection). The term “non-human animals” includes mammals and non-mammals, such as non-human primates. In some embodiments, the subject is a human. In certain embodiments, the subject is a pregnant female. In some embodiments, the subject is not pregnant at the time the antibody molecule is administered. In some embodiments, the subject is not pregnant at the time the antibody molecule is administered, and is pregnant when infected with Zika virus. The methods and compositions described herein are suitable for treating human patients a disorder described herein (e.g., Zika virus infection or a disorder associated with Zika virus infection). Patients having a disorder described herein include those who have developed a disorder described herein (e.g., Zika virus infection) but are (at least temporarily) asymptomatic, patients who have exhibited a symptom of a disorder described herein (e.g., Zika virus infection), or patients having a disorder related to or associated with a disorder described herein (e.g., a Zika virus infection). Exemplary disorders related or associated with Zika virus include, but are not limited to, Zika fever, microcephaly, Guillain-Barrd syndrome (GBS), or a neurological abnormality.

### Methods of Treating or Preventing Disorders

The antibody molecules or vaccines described herein can be used to treat or prevent Zika virus infection, disorders or conditions associated with Zika virus infection, or symptoms thereof.

Exemplary disorders or conditions that can be associated with Zika virus include, but are not limited to Zika fever, microcephaly, Guillain-Barrd syndrome (GBS), a neurological abnormality, or a combination thereof.

The antibody molecules described herein are typically administered at a frequency that keeps a therapeutically effective level of antibody molecules in the patient's system until the patient recovers. For example, the antibody molecules may be administered at a frequency that achieves a serum concentration sufficient for at least about 1, 2, 5, 10, 20, 30, or 40 antibody molecules to bind each Zika virus. In an embodiment, the antibody molecules are administered every 1, 2, 3, 4, 5, 6, or 7 days, every 1, 2, 3, 4, 5, or 6 weeks, or every 1, 2, 3, 4, 5, or 6 months.

In an embodiment, the antibody molecule is administered at a dose or dose range of about 1 mg/kg to about 50 mg/kg, e.g., about 5 mg/kg to about 25 mg/kg, about 5 mg/kg to about 20 mg/kg, about 5 mg/kg to about 15 mg/kg, about 5 mg/kg to about 10 mg/kg, about 20 mg/kg to about 25 mg/kg, about 15 mg/kg to about 25 mg/kg, about 10 mg/kg to about 25 mg/kg, about 10 mg/kg to about 40 mg/kg, about 20 mg/kg to about 30 mg/kg, e.g., about 25 mg/kg or less, about 20 mg/kg or less, about 15 mg/kg or less, about 10 mg/kg or less, about 5 mg/kg or less, about 2 mg/kg or less, or about 1 mg/kg or less.

In an embodiment, the antibody molecule is administered as a flat or fixed dose, e.g., from about 1000 mg to about 5000 mg, e.g., from about 1500 mg to about 4500 mg, from about 2000 mg to about 4000 mg, about 2500 mg to about 3500 mg, about 2000 mg to about 3000 mg, about 3000 mg to about 4000 mg, about 2000 mg to about 2500 mg, or about 2500 mg to about 3000 mg, e.g., about 1500 mg, about 2000 mg, about 2500 mg, about 3000 mg, about 3500 mg, about 4000 mg, or about 4500 mg.

In an embodiment, the antibody molecule is administered as a single dose. In an embodiment, the antibody molecule is administered as a single dose for duration of pregnancy. In another embodiment, the antibody molecule is administered once a trimester for duration of pregnancy. In yet another embodiment, the antibody molecule is administered once a week, once two weeks, once a month, or once two months, e.g., for duration of pregnancy. In an embodiment, the antibody molecule is administered once a month for during of pregnancy.

In an embodiment, the antibody molecule is administered as a therapeutic dose. In another embodiment, the antibody molecule is administered as a prophylactic dose.

Methods of administering various antibody molecules or vaccines are known in the art and are described herein. Suitable dosages of the antibody molecules or vaccines used may depend on the age and weight of the subject and the particular drug used.

The antibody molecules can be used by themselves or conjugated to a second agent, e.g., an anti-viral agent, toxin, or protein, e.g., a second anti-ZIKV antibody molecule. This method includes: administering the antibody molecule, alone or conjugated to a second agent, to a subject requiring such treatment. The antibody molecules can be used to deliver a variety of therapeutic agents, e.g., anti-viral agent, toxin, or protein, or mixtures thereof.

### Combination Therapies

The antibody molecules or vaccines described herein can be used in combination with other therapies. For example, the combination therapy can include an antibody molecule or a vaccine, co-formulated with, and/or co-administered with, one or more additional therapeutic agents, e.g., one or more additional therapeutic agents described herein. In other embodiments, the antibody molecule or vaccine is administered in combination with another therapeutic treatment modality, e.g., another therapeutic treatment modality described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In an embodiment, the antibody molecule or vaccine is administered in combination with a second or additional therapeutic agent to treat or prevent Zika virus infection or to inhibit Zika virus in a subject. In an embodiment, the antibody molecule or vaccine, and the second or additional agent are administered such that one or more of the following is achieved: therapeutic levels, or therapeutic effects, of one overlap the other; detectable levels of both are present at the same time; or the therapeutic effect is greater than what would be seen in the absence of either the antibody molecule or vaccine, or the second or additional agent. In an embodiment, each agent will be administered at a dose and on a time schedule determined for that agent.

In certain embodiments, an antibody molecule described herein is administered, and the second or additional therapeutic agent is a second antibody molecule, e.g., an antibody molecule different from a first antibody molecule. Exemplary antibody molecules that can be used in combination include, but are not limited to, an antibody molecule to a Zika viral protein (e.g., an antibody molecule to capsid (C), premembrane/membrane (pr/M), envelop (E), NS1, NS2A, NS2B, NS3, NS4A, 2K, NS4B, NS5, or a combination thereof), or an antibody molecule to a host cell protein associated with Zika virus infection.

In an embodiment, the antibody molecule or vaccine is administered in combination with a 4-aminoquinoline derivative (e.g., amodiaquine, chloroquine, or hydroxychloroquine). In an embodiment, the 4-aminoquinoline derivative is administered at a dose from about 5 mg/kg to about 50 mg/kg, e.g., about 10 mg/kg to about 45 mg/kg, about 15 mg/kg to about 40 mg/kg, about 20 mg/kg to about 35 mg/kg, about 25 mg/kg to about 30 mg/kg, about 25 mg/kg to about 35 mg/kg, or about 15 to about 25 mg/kg, e.g., every two days, daily, twice a day, or three times a day. In an embodiment, the antibody molecule is administered as a flat or fixed dose, e.g., from about 1000 mg to about 5000 mg, e.g., from about 1500 mg to about 4500 mg, from about 2000 mg to about 4000 mg, about 2500 mg to about 3500 mg, about 2000 mg to about 3000 mg, about 3000 mg to about 4000 mg, about 2000 mg to about 2500 mg, or about 2500 mg to about 3000 mg, e.g., about 1500 mg, about 2000 mg, about 2500 mg, about 3000 mg, about 3500 mg, about 4000 mg, or about 4500 mg. In an embodiment, the antibody molecule is administered as a single dose.

In an embodiment, the antibody molecule or vaccine is administered in combination with a nucleoside inhibitor (e.g., ribavirin or taribavirin). In an embodiment, the nucleoside inhibitor is administered at a dose from about 200 mg to about 800 mg, e.g., about 300 mg to about 700 mg, about 400 mg to about 600 mg, about 200 mg to about 400 mg, or about 300 mg and about 500 mg, e.g., every two days, daily, twice a day, or three times a day.

In an embodiment, the antibody molecule or vaccine is administered in combination with an interferon (e.g., interferon-alpha, pegylated interferon-alpha-2a, or pegylated interferon-alpha-2b). In an embodiment, the interferon is administered at a dose from about 0.05 to about 0.25 mg, e.g., about 0.08 mg to about 0.2 mg, about 0.1 mg to about 0.18 mg, about 0.12 mg to about 0.16 mg, about 0.08 mg to about 0.1 mg, about 0.12 mg to about 0.15 mg, or about 0.16 mg to about 0.2 mg, e.g., twice a week, weekly, every two weeks, or every three weeks.

In an embodiment, the antibody molecule or vaccine is administered in combination with an RNA-dependent RNA polymerase (RdRp) inhibitor (e.g., BCX4430).

In an embodiment, the antibody molecule or vaccine is administered in combination with a nucleotide analogue (e.g., an adenosine analogue), e.g., BCX4430, NITD008, or GS-5734.

In an embodiment, an antibody molecule (e.g., an antibody molecule described herein) is administered in combination with a vaccine (e.g., a vaccine described herein).

Other exemplary Zika vaccines include, but are not limited to an inactivated purified vaccine, a virus-like particle (VLP) (e.g., with a pRME protein), a YF17DD chimeric vaccine, a DNA vaccine, a live dengue recombinant vaccine, a DNA vaccine (e.g., expressing a VLP), a live recombinant adenovirus, a recombinant Zika viral protein (e.g., plus ALHYDROGEL®), a lentivirus-vectored vaccine, a measles-vectored vaccine, a Zika targeted mutation live attenuated vaccine, a live VSV recombinant vaccine, an E protein (e.g., in a nanoparticle), a synthetic replilink peptide, ChimeriVax (YF17D), or a combination thereof.

In an embodiment, the antibody molecule described herein is administered in combination with a therapy (e.g., an antibody molecule or small molecule) for dengue virus, Japanese encephalitis virus, West Nile virus, chikungunya virus, or a combination thereof. In another embodiment, the antibody molecule described herein is administered in combination with a vaccine for dengue virus, Japanese encephalitis virus, West Nile virus, chikungunya virus, or a combination thereof.

In an embodiment, the antibody molecule or vaccine, and the second or additional agent, are provided as separate formulations, and the step of administering includes sequentially administering the antibody molecule or vaccine, and the second or additional agent. The sequential administrations can be provided on the same day (e.g., within one hour of one another or at least 3, 6, or 12 hours apart) or on different days.

In an embodiment, the antibody molecule or vaccine, and the second or additional agent, are each administered as a plurality of doses separated in time. The antibody molecule or vaccine, and the second or additional agent, are generally each administered according to a regimen. The regimen for one or both may have a regular periodicity. The regimen for the antibody molecule or vaccine, can have a different periodicity from the regimen for the second or additional agent, e.g., one can be administered more frequently than the other. In one implementation, one of the antibody molecule or vaccine, and the second or additional agent, is administered once weekly and the other once monthly. In another implementation, one of the antibody molecule or vaccine, and the second or additional agent, is administered continuously, e.g., over a period of more than 30 minutes but less than 1, 2, 4, or 12 hours, and the other is administered as a bolus. In an embodiment, sequential administrations are administered. The time between administration of the one agent and another agent can be minutes, hours, days, or weeks. The use of an antibody molecule or vaccine described herein can also be used to reduce the dosage of another therapy, e.g., to reduce the side-effects associated with another agent that is being administered. Accordingly, a combination can include administering a second or additional agent at a dosage at least 10, 20, 30, or 50% lower than would be used in the absence of the antibody molecule or vaccine. The antibody molecule or vaccine, and the second or additional agent, can be administered by any appropriate method, e.g., subcutaneously, intramuscularly, or intravenously.

In an embodiment, each of the antibody molecule or vaccine, and the second or additional agent, is administered at the same dose as each is prescribed for monotherapy. In another embodiment, the antibody molecule or vaccine, is administered at a dosage that is equal to or less than an amount required for efficacy if administered alone. Likewise, the second or additional agent can be administered at a dosage that is equal to or less than an amount required for efficacy if administered alone.

Exemplary therapies that can be used in combination with an antibody molecule, composition, or vaccine described herein to treat or prevent disorders are also described in the section of “Methods of Treating or Preventing Disorders” herein.

### Methods of Diagnosis

In some aspects, the present disclosure provides a diagnostic method for detecting the presence of Zika virus in vitro (e.g., in a biological sample, such as a biopsy or blood sample) or in vivo (e.g., in vivo imaging in a subject). The method includes: (i) contacting the sample with an antibody molecule described herein, or administering to the subject, the antibody molecule; (optionally) (ii) contacting a reference sample, e.g., a control sample (e.g., a control biological sample, such as a biopsy or blood sample) or a control subject with an antibody molecule described herein; and (iii) detecting formation of a complex between the antibody molecule and Zika virus or a Zika viral protein (e.g., the E protein) in the sample or subject, or the control sample or subject, wherein a change, e.g., a statistically significant change, in the formation of the complex in the sample or subject relative to the control sample or subject is indicative of the presence of Zika virus in the sample. The antibody molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting bacteria includes, but is not limited to, cells, cell lysates, proteins or membrane extracts of cells, body fluids such as blood, or tissue samples such as biopsies.

Complex formation between the antibody molecule, and Zika virus or a Zika viral protein (e.g., the E protein), can be detected by measuring or visualizing either the antibody molecule bound to Zika virus or the Zika viral protein (e.g., the E protein) or unbound antibody molecule. Any suitable detection assays can be used, and conventional detection assays include an enzyme-linked immunosorbent assays (ELISA), a radioimmunoassay (RIA) or tissue immunohistochemistry. Alternative to labeling the antibody molecule, the presence of Zika virus or a Zika viral protein (e.g., the E protein) can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of Zika virus or a Zika viral protein (e.g., the E protein) in the sample is inversely proportional to the amount of labeled standard bound to the antibody molecule.

The diagnostic methods described herein can be used to replace other methods of detecting Zika virus, e.g., a PCR-based detection method. The diagnostic methods described herein can also be used in combination with other methods detecting Zika virus, e.g., a PCR-based method or ELISA-based method, as described herein.

The antibody molecules described herein can be used to diagnose disorders that can be treated or prevented by the antibody molecules described herein. The detection or diagnostic methods described herein can be used in combination with other methods described herein to treat or prevent a disorder described herein.

### EXAMPLES

### Example 1: Development of Anti-Zika Antibody Molecules

To provide structural context to recent clinical and histopathologic findings of Zika virus, a detailed analysis was completed for the surface accessible region of the Flavivirus E protein, the major virus surface protein responsible for virus binding and fusion as well as the primary target for currently known neutralizing antibodies. The structural analysis reveals at least two findings that are importance for devising strategies to control the virus. First, it was shown that surface regions of the E protein that are important for recognition by dominant cross-reactive antibodies against dengue viruses (DENV) cluster disproportionately with ZIKV, but not the related West Nile and Japanese encephalitis viruses. This epitope clustering may allow for greater recognition of ZIKV by antibodies from DENV immune or, potentially, DENV-vaccinated individuals which likely exhibit dampened neutralization potency and/or avidity to ZIKV, and which in turn could enhance viral infection. Second, recent strains of ZIKV demonstrate a structural hallmark linked to acquisition of a neuroinvasive phenotype that was first documented for West Nile virus. These findings suggest DENV infection or vaccination may alter susceptibility to ZIKV replication and potentially disease, and indicate a vaccine or antibody strategy focused to the more dissimilar E protein domain III can reduce risks arising from cross-reactive antibodies.

### Methods

Obtaining Sequences.

Sequences were obtained for Zika Virus (ZIKV), Japanese Encephalitis Virus (JEV), and West Nile Virus (WNV). Protein sequences were obtained from GenBank searching based on the virus name and limiting the size of the sequence to greater than 350 amino acids and discarding patent sequences.

A profile HMM was generated for select flavivirus E proteins using an alignment of DENV1-4, JEV, WNV, and ZIKV E-proteins. This HMM was used to identity E proteins from the list of sequences downloaded from GenBank using the HMMalign functionality in HMMER3 (http://hmmer.org v3.1b1).

Given the high degree of similarity within each serotype, the following sequences were chosen as representative members for each flavivirus (with GenBank accession): ZIKV (AMH87239: Brazil/2015); JEV (PDB:3P54); WNV (AET05737), DENV3 (PDB:1UZG); DENV1 (P17763); DENV2 (ABA61185); DENV4 (ABD17415.1).

Defining Surface Residues.

Surface residues were defined using a sidechain surface exposure >25% based on the structure of the E protein dimer and calculated using Discovery Studio. Residues were then user-curated to remove exposed residues that were on the side of the E protein facing the lipid bilayer (inaccessible to antibody binding). Visual inspection was used to curate the list and to add additional residues that fell below the 25% threshold but which were visually determined to be sufficiently exposed. Surface exposed positions were defined using the DENV2 structure and this consistent set of exposed residues was applied for all other flaviviruses based on a multiple sequence alignment of each flavivirus that was generated using Discovery Studio (FIG. 4).

Non-Metric Multidimensional Scaling.

A non-metric multidimensional scaling method was employed to facilitate analysis of the pairwise similarity matrix for select flaviviruses, in a manner similar to antigenic mapping of DENV and other viruses (Katzelnick et al. Science 349, 1338-1343 (2015)). The pairwise similarity matrix for the flaviviruses was calculated using a PAM60 substitution matrix to score the sequence alignment of each pair of sequences. The non-metric multidimensional scaling was applied as implemented in sckit-learn (Scikit-learn: Machine Learning in Python, Pedregosa et al., JMLR 12, pp. 2825-2830, 2011.)

Epitope Analysis of Known Cross-Neutralizing Antibodies.

Epitope residues for cross-neutralizing antibodies were determined using a 4.5 Å distance cutoff from antibody residues in co-crystal structures (4UTA.pdb for C8, 4UTB for A11, and 3150.pdb for E53).

### Results

The Flavivirus genus comprises a number of viruses that can cause human disease, including dengue virus (DENV), West Nile (WNV), Zika virus (ZIKV), yellow fever (YFV), and Japanese encephalitis (JEV) viruses. While antibodies elicited from one flavivirus infection often show some cross-reactivity with other flaviviruses, such group-reactive antibodies do not appear to provide extensive protection across the family. Noteworthy, in the case of dengue, infection from one of the four serotypes confers long-term protection to the infecting serotype but not to the other three serotypes (Sabin et al. The American Journal of Tropical Medicine and Hygiene 1, 30-50 (1952)). Indeed, the risk of severe disease is significantly heightened for a serotype-heterologous secondary infection (Burke et al. The American Journal of Tropical Medicine and Hygiene 38, 172-180 (1988);

Mizumoto et al. Journal of Vector Borne Diseases 51, 153-164 (2014); Guzman, M. G. et al. American Journal of Epidemiology 152, 793-799; discussion 804 (2000); Vaughn et al. The Journal of Infectious Diseases 181, 2-9 (2000)). One explanation for this observation derives from the concept of original antigenic sin, first described for influenza (Kim et al. Journal of immunology 183, 3294-3301 (2009)). In this case, memory B cells (and possibly T cells) from a first infection are able to recognize, albeit poorly, similar epitopes on a heterologous virus, initiating a misdirected humoral memory recall process. Through a mechanism termed antibody-dependent enhancement (ADE) with either pre-existing cross-reactive antibodies in plasma or nascent antibodies generated during the recall response, such cross-reactive, non-neutralizing antibodies cause enhanced virus replication and increased virus titers through FcγR-mediated uptake of virus-immune complexes (Halstead, The Yale Journal of Biology and Medicine 42, 350-362 (1970); Halstead, Advances in Virus Research 60, 421-467 (2003)). This mechanism is supported by in vivo studies in animals (Zellweger et al. Cell Host & Microbe 7, 128-139 (2010); Ng et al. PLoS Pathogens 10, e1004031 (2014); Goncalvez et al. Proceedings of the National Academy of Sciences of the United States of America 104, 9422-9427 (2007); Balsitis et al. PLoS Pathogens 6, e1000790 (2010)). ADE is often associated with prM antibodies, or other cross-reactive or poorly neutralizing antibodies. With the understanding of the potential enhancing capacity of cross-reactive antibodies as well as differences in neuroinvasive disease phenotype among the flaviviruses, a structure-based analysis of accessible residues of ZIKV was performed and their similarity to other flaviviruses was assessed.

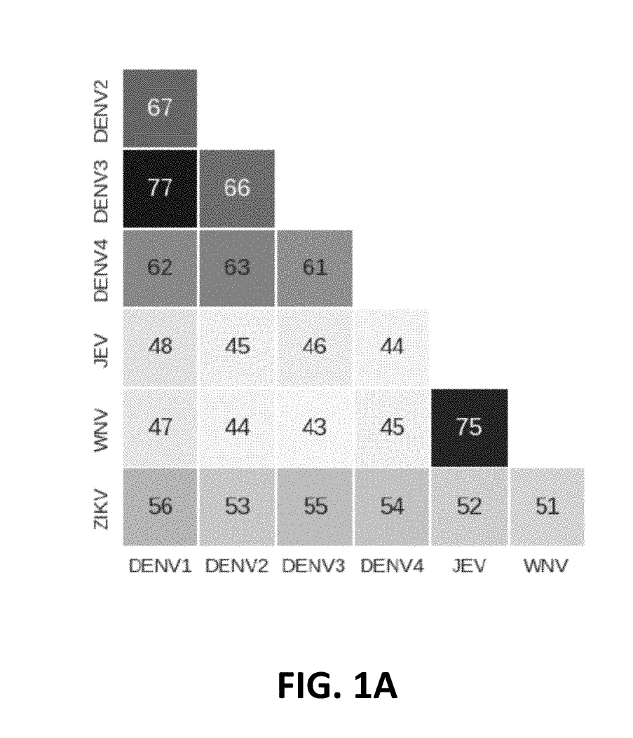

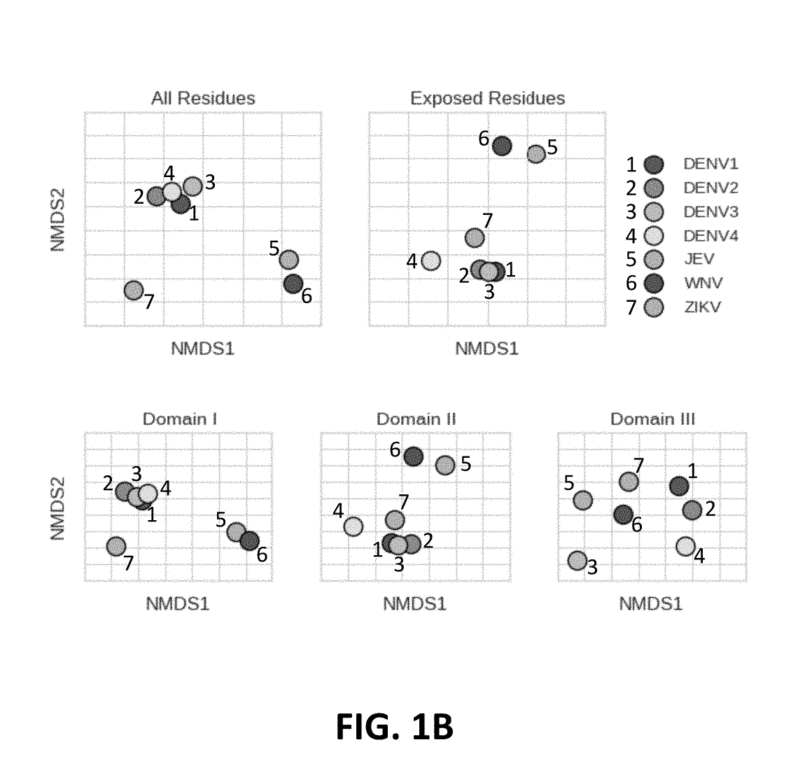

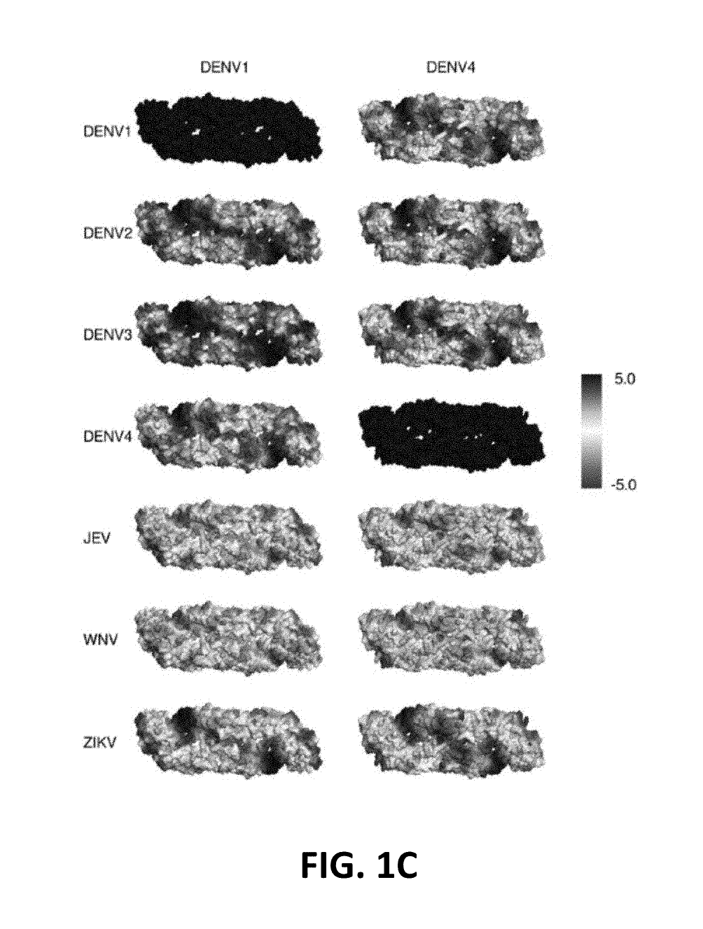

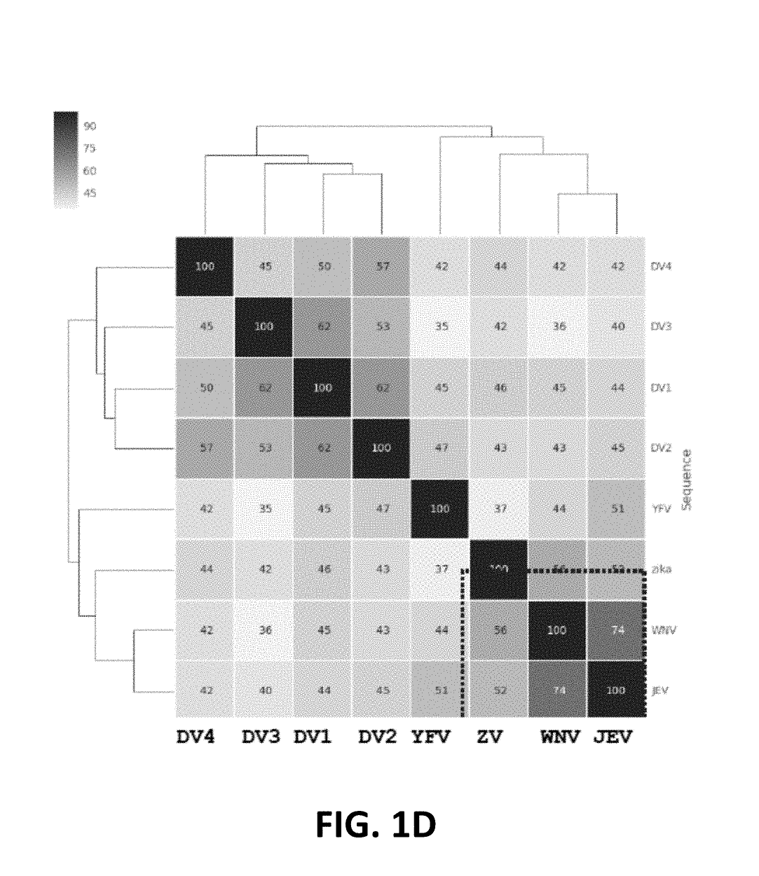

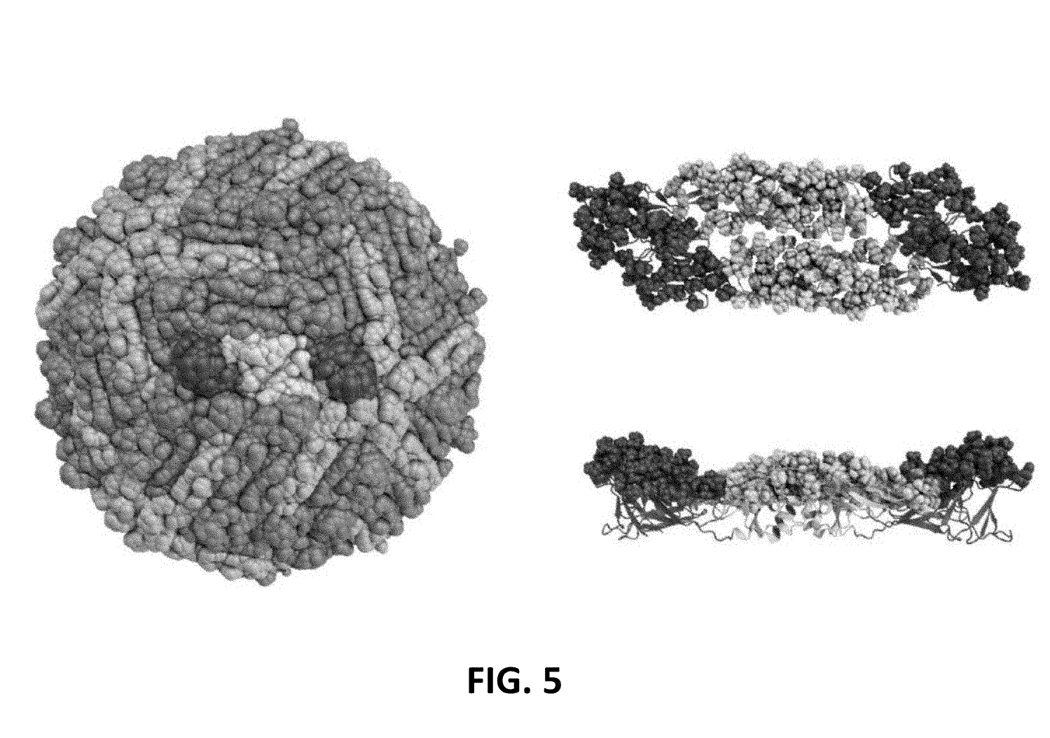

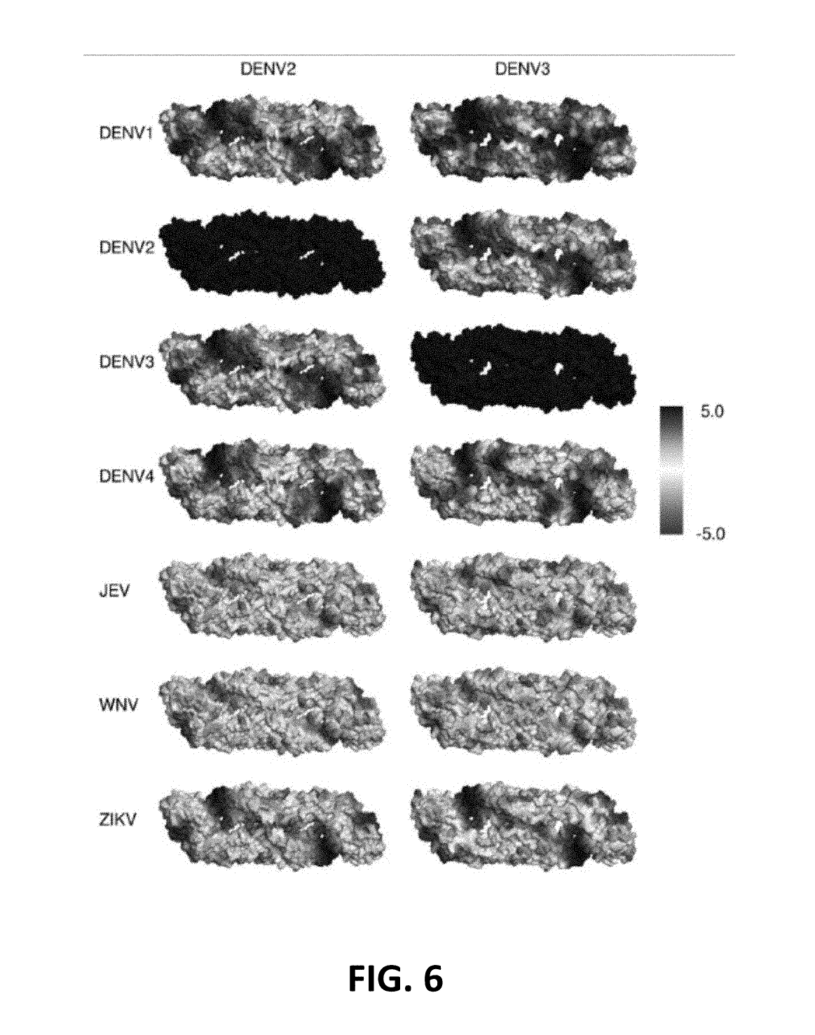

A sequence alignment of the E protein between flaviviruses shows DENV to be slightly more related to ZIKV than to other medically significant flaviviruses, such as JEV or WNV (FIGS. 1A and 4). Notably, people are protected from disease in cases of tertiary and quaternary DENV infections, despite serotypes sharing only 60-80% sequence identity in the E protein, potentially due in part to increased avidity of highly cross-reactive DENV-neutralizing antibodies observed after secondary infection (Zompi et al. PLoS Neglected Tropical Diseases 6, e1568 (2012); Tsai et al. Journal of Virology 87, 12562-12575 (2013); Smith et al. Journal of Virology 86, 2665-2675 (2012)). To compare ZIKV to other flaviviruses in the context of epitopes recognized by cross-reactive antibodies, the similarities of flaviviruses were first analyzed both for the entire E protein sequence and also for surface exposed residues in the context of the virion (FIG. 5). A clustering analysis was performed by applying multidimensional scaling of the pairwise sequence similarities, in a manner established for antigenic mapping of DENV and other viruses (Katzelnick et al. Science 349, 1338-1343 (2015)). Given the ability of cross-reactive antibodies to recognize highly similar but not identical epitopes, a PAM60 substitution matrix was utilized to score substitutions by amino acid relatedness and thus account for tolerance of biophysically similar amino acids in epitopes. As shown in FIG. 1B, the full E protein of ZIKV clusters separately from the DENV serotypes and WNV/JEV. However, when only surface exposed residues of E protein are considered, ZIKV clusters with the DENV complex. Surprisingly, when inspected by individual E domains, the clustering analysis reveals the greatest similarity between ZIKV and DENV is localized to domain II, with high dissimilarity in domain III (FIG. 1B). As shown in FIG. 1D, analysis of domain III indicates that the closest match is to WNV (56% identity with no insertions or deletions), with lower identity/homology to DENV1-4 observed.

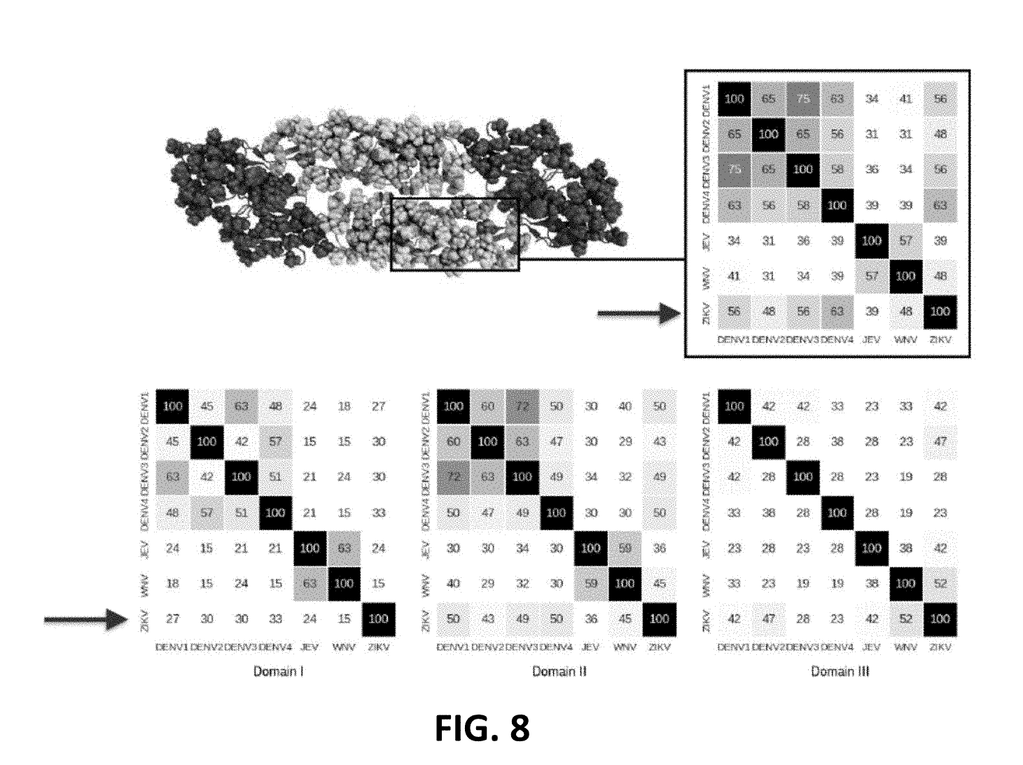

To confirm and extend these initial observations, surface residue sequence similarity was mapped between ZIKV and other flaviviruses to corresponding structures of E protein. Consistent with the clustering analysis, sequence relatedness between ZIKV and DENV maps heterogeneously across domains, with low similarity in domain III and high similarity in domain II, particularly in a region extending beyond the fusion loop (FIGS. 1C and 8-9). While the fusion loop motif in domain II is highly conserved across flaviviruses, ZIKV exhibits a surface patch beyond the fusion loop with significant similarity to DENV, in contrast to other encephalitic flaviviruses. When comparing the surfaces of domain II, the similarity of ZIKV with DENV4 is comparable to the similarity of other DENV serotypes to DENV4 (FIG. 8). A similar analysis of prM, the other exposed protein on the flavivirus virion, which elicits predominantly non-neutralizing antibodies (Dejnirattisai et al. Science 328, 745-748 (2010)), did not show a similar clustering or surface similarity to DENV (FIGS. 9A-9B). These analyses demonstrate that ZIKV surface residues, and particularly those in the domain II region, cluster disproportionately with DENV compared to other flaviviruses, an observation not evident from entire E protein sequence comparison.

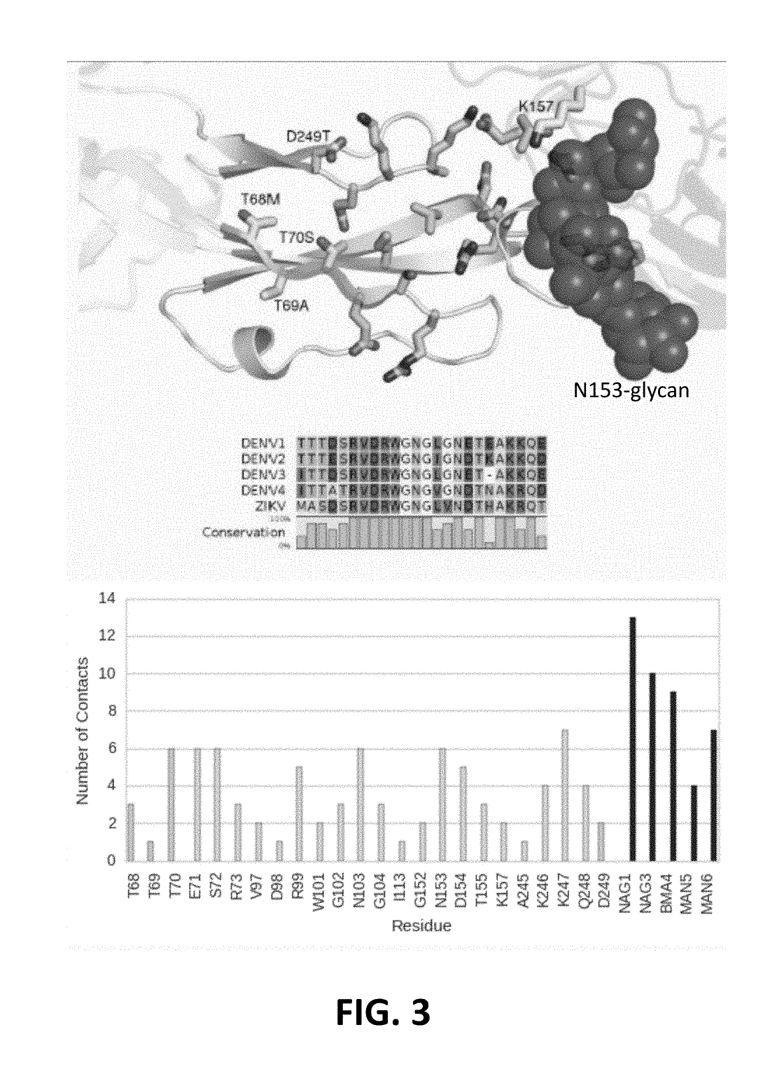

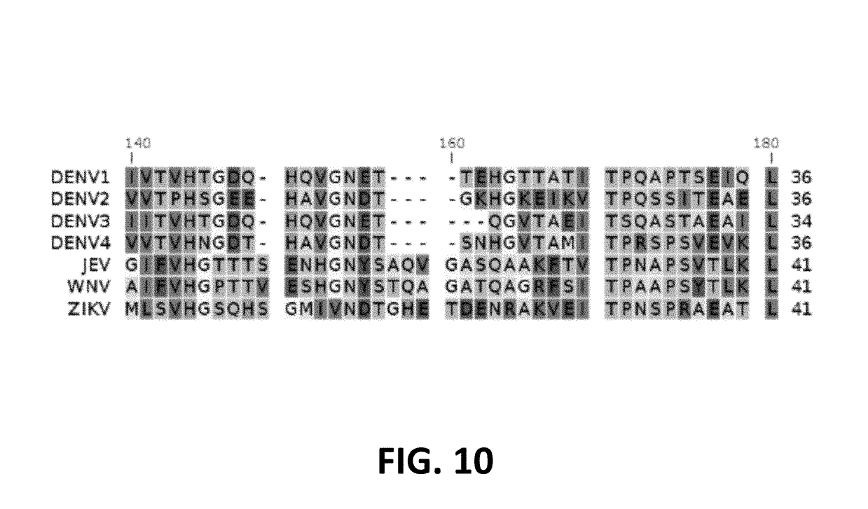

Studies characterizing antibody responses from human DENV infection have identified two principal types of cross-reactive neutralizing antibodies to E protein. These groups of dominant cross-reactive DENV antibodies map to the (1) fusion loop epitope (FLE) and (2) “envelope dimer epitope” (EDE) (Dejnirattisai et al. Nature Immunology 16, 170-177 (2015)). Noteworthy, these epitopes have considerable overlap (FIG. 2A) (Rouvinski et al. Nature 520, 109-113 (2015)). Remarkably, the FLE and EDE regions coincide with the regions identified as having a disproportionately high similarity between ZIKV and DENV (FIG. 2B). These cross-reactive antibodies are tolerant to amino acid differences between DENV serotypes; inspection of the epitope for one example EDE antibody, A1126, shows that ZIKV also shares an overall high degree of similarity for contact residues (FIG. 3). Residues that are different between ZIKV and DENV are localized to the periphery of the epitope and make few contacts to the antibody, suggesting that this surface region in ZIKV may engage, albeit more weakly, with this class of antibodies. Since the N153 glycan makes multiple contacts with EDE antibodies, the position and role of the comparable glycan in ZIKV were also investigated. Surprisingly, despite significant structural differences in this glycosylation loop across flaviviruses (FIG. 10), the glycan is always positioned in a similar location and orientation (Kanai et al. Journal of Virology 80, 11000-11008 (2006)). Given the similarity of ZIKV to WNV in the glycosylation loop, the glycan in ZIKV is expected to adopt a similar presentation, and therefore contacts made by the antibody to the glycan are also likely conserved. Overall, this high level of similarity between ZIKV and DENV in epitopes recognized by DENV cross-reactive antibodies indicates a risk for greater cross-reactivity of DENV antibodies to ZIKV compared to other flaviviruses, which provides an explanation for the problems in distinguishing DENV and ZIKV immunoglobulin responses by current serological tests (Lanciotti et al. Emerging Infectious Diseases 14, 1232-1239 (2008)).

Collectively, these analyses suggest that DENV cross-reactive neutralizing antibodies can engage ZIKV with affinities disproportionate to the overall sequence similarity of ZIKV and DENV. However, small differences between ZIKV and DENV in antibody contact residues can cause reduced affinity relative to DENV of cross-reactive DENV antibodies. As sub-neutralizing concentrations of antibodies can cause ADE in vitro and in vivo (Balsitis et al. PLoS Pathogens 6, e1000790 (2010)), the apparent affinity reduction in cross-reactive antibodies to ZIKV relative to DENV not only can impact neutralization potency detrimentally but also can promote antibody-enhanced infection. Noteworthy, in the context of human cytomegalovirus (CMV) infection, which can be transmitted from maternal to fetal compartment through infection of the uterine-placental interface, low avidity/neutralizing anti-CMV antibodies correlate with higher risk of fetal infection (Maidji et al. The American Journal of Pathology 168, 1210-1226 (2006); Furione et al. Journal of Medical Virology 85, 1960-1967 (2013); Visentin et al. Clinical Infectious Diseases: An Official Publication of the Infectious Diseases Society of America 55, 497-503 (2012)). In the case of ZIKV and as explained herein, low avidity/neutralizing activity cross-reactive antibodies developed from previous DENV infection may function analogously to similar antibodies to CMV and promote enhanced maternal-fetal transmission possibly through enhanced neonatal FcR (FcRn) uptake.

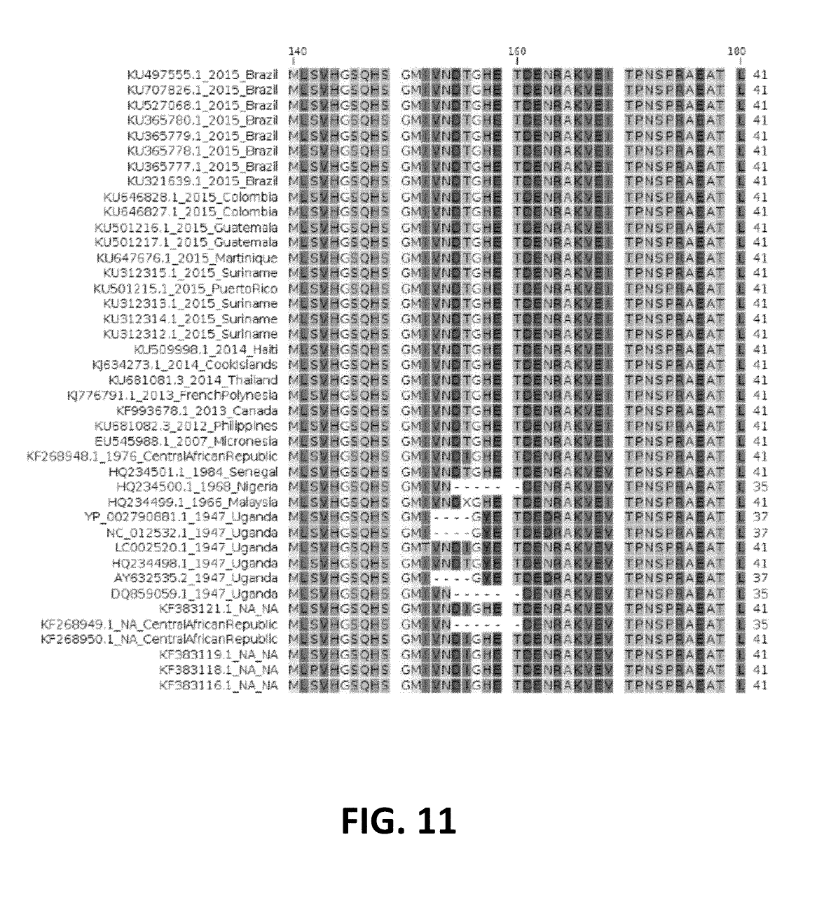

Next, potential structural changes in ZIKV across a temporal axis were investigated, given the recent nature of the Zika outbreak. All ZIKV sequences deposited in GenBank as of February 2016 were first aligned, with a focus on E protein. Few changes representing substantial structural differences exist between the sequences. First, a Ser/Phe difference at position 285 is observed, however, this position is not temporally correlated across strains and is located on the side of E protein proximal to the lipid membrane, rendering it inaccessible to engagement with host factors. Second, a substantial E protein sequence length change (insertion/deletion) of 4-6 amino acids in the 150-160 region of E protein was identified: older ZIKV strains exhibit a mixture of insertion/deletion in this region, whereas more recent ZIKV strains exclusively exhibit a longer envelope sequence (FIG. 11). Previous analysis of changes in this loop has suggested that these changes are likely real and may have occurred recurrently in ZIKV evolution, although the loss of glycosylation may also be an artifact of serial passaging of ZIKV (Faye et al. PLoS Neglected Tropical Diseases 8, e2636 (2014)). Of note, the longer sequence introduces a putative N-linked glycosylation site at position N154 in domain I, as described above. The longer sequence motif shows greater similarity to the corresponding sequence in encephalitic flaviviruses, including WNV and JEV, but not to the DENV serocomplex.

Acquisition of this glycosylation site may impact multiple critical properties of virus including neuroinvasive potential, epitope accessibility of dominant flavivirus cross-reactive neutralizing antibodies (described above), and recognition by host attachment factors. Investigations of virulence differences between WNV strains have demonstrated that increased neurovirulence is associated with the presence of the N154 glycosylation site (Shirato et al. The Journal of General Virology 85, 3637-3645 (2004); Beasley et al. Journal of Virology 79, 8339-8347 (2005)). Evolutionary analysis indicates that the appearance of this glycosylation site has been under selective pressure (Estrada-Franco et al. Emerging Infectious Diseases 9, 1604-1607 (2003)), and control studies directly comparing strains with and without this glycosylation site demonstrate that the presence of this glycosylation is important for virulence and neuroinvasiveness in animals (Beasley et al. Journal of Virology 79, 8339-8347 (2005)). A similar phenotype in mice is observed when the homologous glycan is lost in dengue virus (Kawano et al. Journal of Virology 67, 6567-6575 (1993)). In addition to its potential role in neuroinvasion, the emergence of the Asn154 glycosylation site is likely to serve other important roles in the propagation of the virus, based on its function in encephalitic flaviviruses such as WNV and JEV. The presence of this glycosylation site has been documented to influence recognition of flaviviruses by mammalian cell surface receptors, including DC-SIGN and DC-SIGNR (Martina et al. Virus Research 135, 64-71 (2008)). Additionally, Asn154 has been shown to influence assembly and infectivity of virions and virion stability of WNV37. Finally, other hallmark mutations associated with the gain of a neurovirulent phenotype, such as D390H and E71D (Sanchez & Ruiz, The Journal of General Virology 77 (Pt 10), 2541-2545 (1996); Bray et al. Journal of Virology 72, 1647-1651 (1998)), are present in ZIKV.

The analysis highlighting regions of structural and epitope relatedness of ZIKV and DENV suggests that the presence of cross-reactive antibodies, elicited by either prior DENV infection or by vaccination to DENV, may enhance primary ZIKV infection through promotion of FcγR-mediated uptake leading to antibody-dependent enhancement. Correspondingly, the presence of non- or weakly neutralizing antibodies could enhance transplacental transport to the developing fetus through FcRn binding. It is noted that South and Central America are endemic with dengue, with many individuals having been exposed to infection from multiple DENV serotypes, resulting in complex, heterologous serum reactivity across the Flaviviridae virus family. Through mechanisms of original antigenic sin and shared epitopes as described herein, DENV immune individuals may be at greater risk of disease and higher virus titers from ZIKV infection. Additionally, more recent strains have acquired an N-linked glycosylation site, which in the context of WNV, has been specifically linked to neuroinvasiveness.

This analysis has implications for vaccine approaches to ZIKV and more broadly to the Flaviviridae. A vaccine, either to ZIKV or DENV, that elicits cross-reactive antibodies, particularly to domain II, has the potential to enhance disease if such antibodies bind to, but do not neutralize, the heterologous virus. This type of disease enhancement may already be apparent in long-term follow-up of a tetravalent vaccine to dengue, where naïve individuals administered vaccine appear at subsequent risk for more severe dengue infection, where protection is not complete (Simmons, The New England Journal of Medicine 373, 1263-1264 (2015)). The emergence of ZIKV may warrant vaccine approaches that concentrate the immune response to regions of the E protein such as domain III, which was shown to have high dissimilarity across flaviviruses, which decreases risk of cross-reactivity. Alternatively, development of countermeasures such as Fc-modified monoclonal antibodies (Pedraz et al. The Pediatric Infectious Disease Journal 22, 823-827 (2003)) or small molecules (Baeten et al. The New England Journal of Medicine 367, 399-410 (2012)) that demonstrate the ability to prophylactically protect against disease may represent an effective approach to prevention and therapy.

### Example 2: Generation of Yeast Display Immunolibrary of scFv Antibody Fragments Capable of Binding to Specific Epitopes on Domain III of the Zika E Protein

This example describes a protocol for generation of yeast display immunolibrary of scFv antibody fragments capable of binding to specific epitopes on domain III of the Zika E protein.

Briefly, two distinct immunization strategies are used to generate a broad immune response for selection of antibodies targeting domain III. First, MAVS (mitochondrial antiviral signaling) knockout mice are infected with a sublethal ZIKV infection (˜10⁵ infectious particles) and spleens are harvested from convalescent animals. As an alternative strategy, BALB/c mice are immunized with recombinantly produced domain III of the E protein, using a prime-boost strategy. The immunization strategy involves 10 μg of domain III injected i.p. once on days 0, 7, 14, and 21 with tail vein immunization on days 32 and 33 prior to isolation of spleen on day 35. To assess the quality of the immune response bleed on day 28 and ELISA analysis of the polyclonal response for binding to both recombinantly produced domain III and full length E protein are determined.

### Example 3: Optimization of the Binding Capability of Selected Monoclonal scFvs by Antibody Engineering

This example describes a protocol for optimization of the binding capability of selected monoclonal scFvs by antibody engineering.

After identification of the lead antibody candidates from mouse immunization, antibody engineering is deployed to optimize the affinity and specificity of leads. First, the structure of the antibody is modeled using homology modeling and employing canonical structures for the CDR loops, including use of the Prediction of Immunoglobulin Structure (PIGS) server. The modelled structure of the antibody binding to the domain III is used to calculate the various inter-residue, inter-atomic contacts across the antigen-antibody interface, including putative hydrogen bonds (including water-bridged ones), disulfide bonds, pi-bonds, polar interactions, salt bridges, and Van der Waals interactions (non-hydrogen), as described previously (Soundararajan et al. Sci Rep 2011; 1: 200). The interactions between a CDR residue and its neighboring epitope residues are represented by a 2D network graph as a visual aid. Structural inspection identifies paratope residues that can potentially be altered to enhance affinity and specificity. Then, through a series of iterations between network-guided design and experimental data, starting with an antibody scaffold from mouse immunization, ˜20-50 constructs are systemically produced and tested for affinity testing using a binding ELISA. This approach has been used towards designing broadly neutralizing antibodies against influenza A hemagglutinin and dengue E protein (Tharakaraman et al. Proc Natl Acad Sci USA 2015; Robinson et al. Cell 2015; 162(3): 493-504). For example, use of this approach in dengue resulted in five amino acid substitutions and one amino acid deletion to enhance binding affinity 13,000-fold to DENV4 while retaining or improving affinity to DENV1-3 (Robinson et al. Cell 2015; 162(3): 493-504). An antibody with 10-1000 nM binding affinity can be optimized to have measured Kd values of <1 nM.

Further, the structures can be diversified by a semi-rational method of introducing diversity (e.g., NNK or trinucleotides) at selected positions using gene synthesis. This procedure allows for scFvs with modified binding characteristics that develop affinity to epitopes structurally related to but slightly different from its original binding partner.

### Example 4: Fe Engineering to Optimize the Half-Life of Lead Antibody Candidates and to Eliminate ADE Potential

This example describes a protocol for Fc engineering to optimize the half-life of lead antibody candidates and to eliminate ADE potential.

To allow for the development and use of any antibody construct against ZIKV, enhancement of antibody half-life is of benefit. While human or humanized monoclonal antibodies typically have an extended half-life of 8-20 days, half-life can be enhanced by at least 2-5 fold. Analysis of an Fc-FcRn crystal structure has led to discovery of a number of Fc mutations that confer FcRn affinity enhancement (Oganesyan et al. The Journal of biological chemistry 2014; 289(11): 7812-24). However, many of these mutants increase affinity to FcRn not just at pH 6.0 but also pH 7.4; as such these mutations are largely not beneficial in extending serum half-life. Similarly other FcRn affinity-enhancing mutations have been found to compromise stability or other effector functions. Using the high-resolution crystal structure of the human FcRn receptor in complex with the human IgG1-Fc domain, a set of mutations that are predicted to enhance this interaction at pH 6.0 with minimal effect at pH 7.4 were identified, by targeting the periphery of Fc-FcRn interaction and increasing the protein-protein interface without introducing additional polar contacts (hydrogen bonds and salt bridges). These mutations or a combination of mutations include, e.g., M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof. Alternatively, or additionally, in an embodiment, the Fc region comprises (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V; (b) a mutation or a combination of mutations capable of disrupting an Fc effector function, e.g., L234A/L235A (also known as “LALA” mutation), or (c) both (a) and (b). The subtle nature of these changes is less likely to impact the stability of the antibody. These mutations can be introduced and combined with the beneficial mutations to identify the optimized Fc candidate. The IgGs with engineered Fc are assayed for improved half-life in humanized mice (containing human FcRn). In parallel, the mutants are evaluated for their thermal stability. Finally, to eliminate the potential for ADE, mutations that will abrogate the binding of antibody to Fcγ receptors (the so-called LALA mutation) can be introduced or the antibody can be produced in the context of an IgG2 framework (Williams et al. PLoS Pathogens 2013; 9(2): e1003157).

Alternatively, the known YTE mutation can be used (Oganesyan et al. The Journal of Biological Chemistry 2014; 289(11): 7812-24). Further, while half-life extension can allow for a single dose prophylactic, even without this, dosing can be completed once monthly with an unmodified antibody.

### Example 5: Demonstration of In Vitro Activity and In Vivo Protective Capacity of Monoclonal Antibody Leads

This example provides a protocol for demonstration of in vitro activity and in vivo protective capacity of monoclonal antibody leads.

For in vitro assessment, strains that are available from the World Reference Center for Emerging Viruses and Arboviruses (WRCEVA) that capture geographic and potential sequence diversity in ZIKV can be utilized. The E protein of all procured ZIKV strains can be sequenced to understand sequence diversity in both the entire E protein and, specifically, domain III. Based on previous experience with DENV, a focus-forming unit (FFU)-based test (i.e., immunostaining of viral replication foci) can provide the most quantitative information across virus strains. ZIKV strains are initially be titered by FFU assay. Briefly, to titer the virus, serial dilutions of virus are incubated for 2 hours with Vero cell monolayers in 24-well plates at 37° C., after which one ml of viscous overlay (DMEM/F12 containing 1% Aquacide II [EMD Millipore]) is added to the monolayer. After a 4-6 day incubation period at 37° C., the overlay is removed, cells are fixed with formalin, and foci are visualized by immunostaining with TrueBlue Peroxidase Substrate (KPL). In vitro neutralization is then be performed by microneutralization assay, similar to that previously described (Guy et al. The American journal of tropical medicine and hygiene 2009; 80(2): 302-11; Vorndam and Beltran. The American journal of tropical medicine and hygiene 2002; 66(2): 208-12). Serially diluted antibody is mixed with an equal volume of diluted virus (approximately 200 FFU/well) and incubated for 1 hour at 37° C. Readout is immunostaining followed by TMB peroxidase substrate (KPL) and an absorbance (600 nm) measurement in a plate reader (Molecular Devices). 100% infection corresponds to the average OD600 in wells not exposed to antibody. Four-parameter logistic model is used to fit the data. EC50 values represent the concentration of antibody at 50% virus neutralization.

For initial assessment of antibody efficacy in vivo, IFNAR knockout mice are used. Two experiments are performed. First, the prophylactic efficacy of 1-3 antibody leads is tested by i.p. infection of purified antibody preparations at 5 mg/kg 24 hours prior to introduction of 10⁶ infectious particles of ZIKV. There are 10 animals per cohort and two control groups are included—uninfected and infected animals administered an isotype control antibody. Animals are monitored for 10 days for weight loss and signs of infection. A subset of animals (n=5) are sacrificed on day 4 to measure viral titer. As an additional efficacy assessment, the therapeutic activity of the antibody leads is also be assessed. In this case, antibody is administered +24 h.p.i. with group sizes and endpoints being identical to those outlined above. Based on this assessment, a lead candidate is selected for humanization, biophysical characterization, and cell line development.

### Example 6: Neutralizing Activity of an E Protein Domain III Antibody

Antibodies directed to E protein domain III are believed to be much less frequently elicited in the human antibody repertoire, as such, exogenous administration of these antibodies does not interfere, and in fact can promote, the neutralizing capacity of human serum. This was demonstrated with dengue virus (DENV) where the neutralizing activity of an exemplary domain III antibody (VIS513) is greater in the presence of serum than without addition.

VIS513 neutralization is enhanced in the presence of heterologous immune sera. Peak enhancing titer for DENV-1 immune sera was determined using THP-1 cells. At this dilution, enhancing immune serum was pre-complexed to DENV-2. Different concentrations of VIS513 were then incubated with complexed DENV and allowed to infect THP-1 cells. Supernatants were harvested and infectious particles were determined by plaque assay. As shown in FIG. 12A, VIS513 inhibited plaque formation in the presence or absence of naïve serum. FIG. 12B shows that the activity of VIS513 was maintained in the presence of DENV-2 immune serum. As shown in FIG. 12C, VIS513 was able to inhibit plaque formation by DENV-2 in the presence heterologous, enhancing immune serum to DENV-1, with the EC50 shifted to a lower concentration than in the absence of serum.

### Example 7: Binding, Neutralization and In Vivo Protection by ZIKV Antibodies

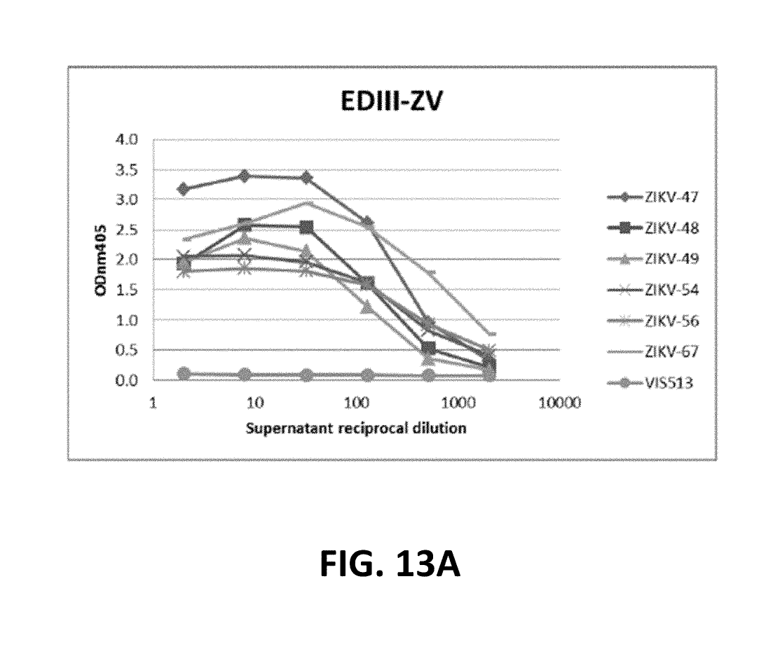

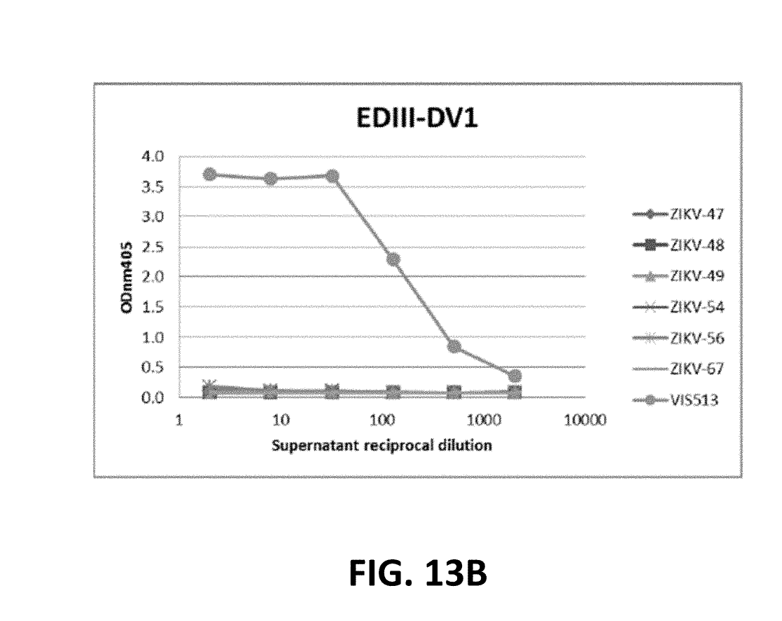

To determine binding specificities of the scaffold antibodies, one microgram per milliliter of ZIKV or DENV EDIII protein was coated to 96 well plates (100 μl/well) at 4° C. overnight. Plates were washed and varying dilutions of hybridoma supernatant (containing antibody) from each of the six ZIKV antibodies was added to the wells of the plate. As a control, varying dilutions of VIS513, a dengue EDIII specific antibody was also applied. Antibody supernatant was incubated for 1 hour at room temperature and plates were subsequently washed to remove unbound antibody. Goat anti-human antibody conjugated to alkaline phosphatase was added to the plate and incubated for 1 hour at room temperature. Plates were again washed and developed using PNPP substrate. Plates were assessed at 405 nm using a Spectramax spectrophotometer and data were analyzed using the SoftMax software. As shown in FIGS. 13A-13B, all six of the ZIKV antibodies specifically bind to the ZIKV EDIII protein with minimal cross-reactivity with the DENV EDIII protein.

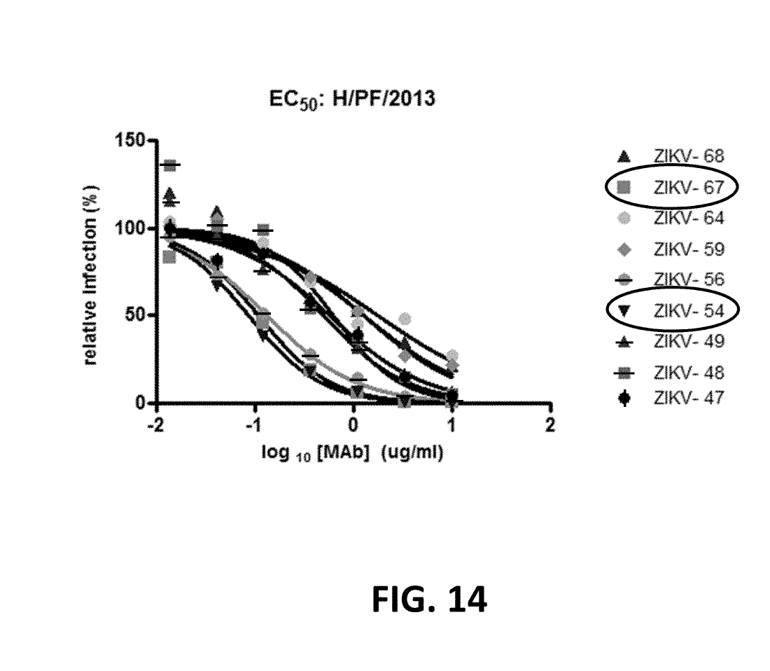

The ability of scaffold antibodies to neutralize Zika virus in vitro was assessed with a microneutralization assay. Serial dilutions of mAbs were incubated with 100 FFU of ZIKV strain A/PF/2013 for 1 hour at 37° C. MAb virus complexes were added to Vero cell monolayers in 96-well plates. After 90 minutes, cells were overlaid with 1% (w/v) methylcellulose in MEM supplemented with 4% FBS. Plates were harvested 40 hours later and fixed with 1% paraformaldehyde in PBS. The plates were incubated sequentially with 500 ng/mL of ZV-16 (Zika reactive antibody) and HRP-conjugated goat anti-mouse IgG in PBS supplemented with 0.1% saponin and 0.1% BSA. ZIKV-infected foci were visualized using TrueBlue peroxidase substrate (KPL) and quantitated on an ImmunoSpot 5.0.37 macroanalyzer (Cellular Technologies). FIG. 14 shows the neutralization effect of all antibodies tested.

Next, experiments to determine the in vivo efficacy of the scaffold antibodies were performed. Briefly, C57BL/6 mice (4- to 5-week-old, Jackson Laboratories) were inoculated with ZIKV by subcutaneous (footpad) route with 105 FFU of mouse-adapted ZIKV strain Dakar in a volume of 50 mL. One day prior, mice were treated with 2 mg of an Ifnar (type 1 interferon receptor) blocking mAb (MAR1-5A3) by intraperitoneal injection (Lazear et al., Cell Host Microbe. 2016; 19(5):720-30). ZIKV mAbs were administered as a single 250 μg dose 1 day before infection via an intraperitoneal route. Serum samples were obtained at day 3 after ZIKV infection and extracted with the Viral RNA Mini Kit (QIAGEN). ZIKV RNA levels were determined by TaqMan one-step qRT-PCR on an ABI 7500 Fast Instrument using published primers and conditions (Lanciotti et al., Emerg Infect Dis. 2008; 14(8): 1232-9). Animals were also weighed daily and monitored for signs or morbidity and mortality.

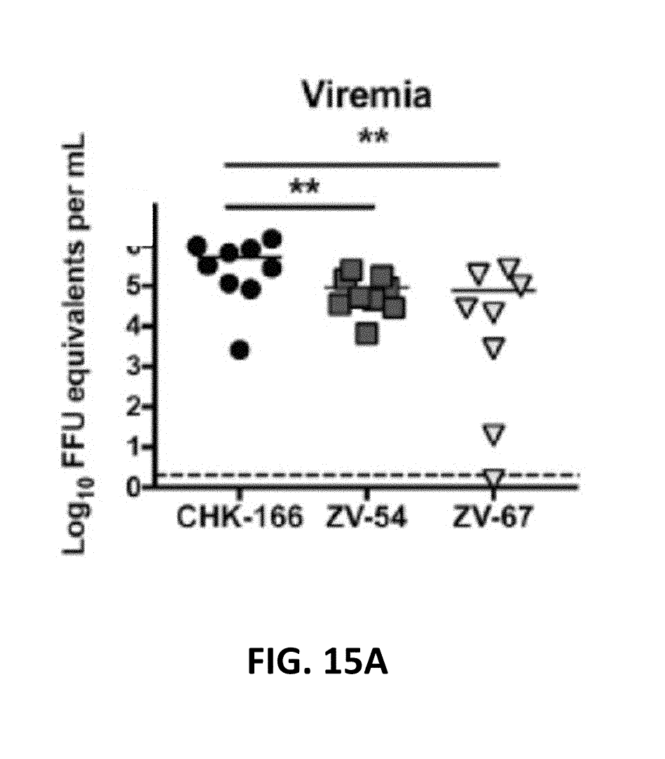

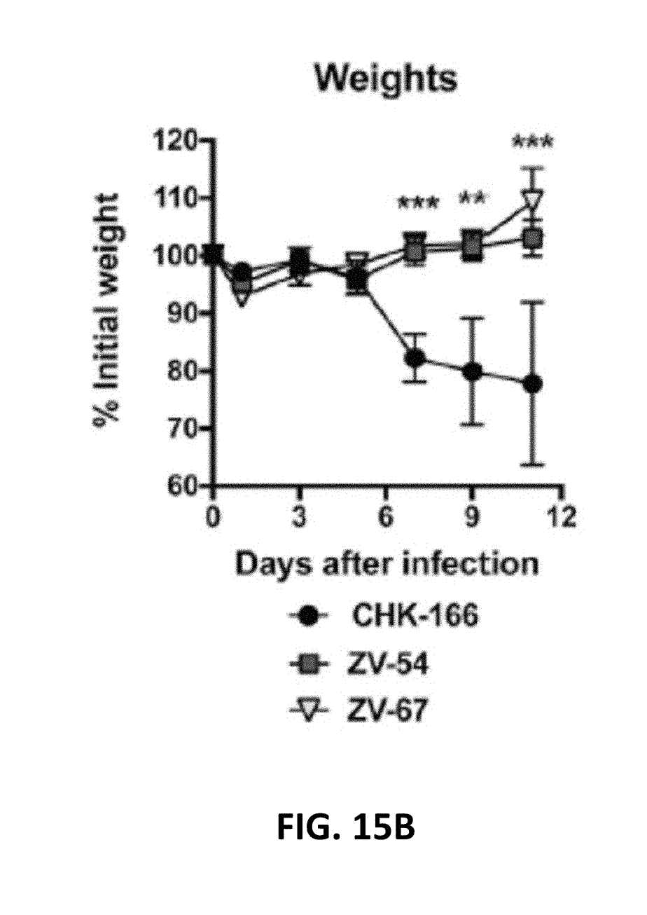

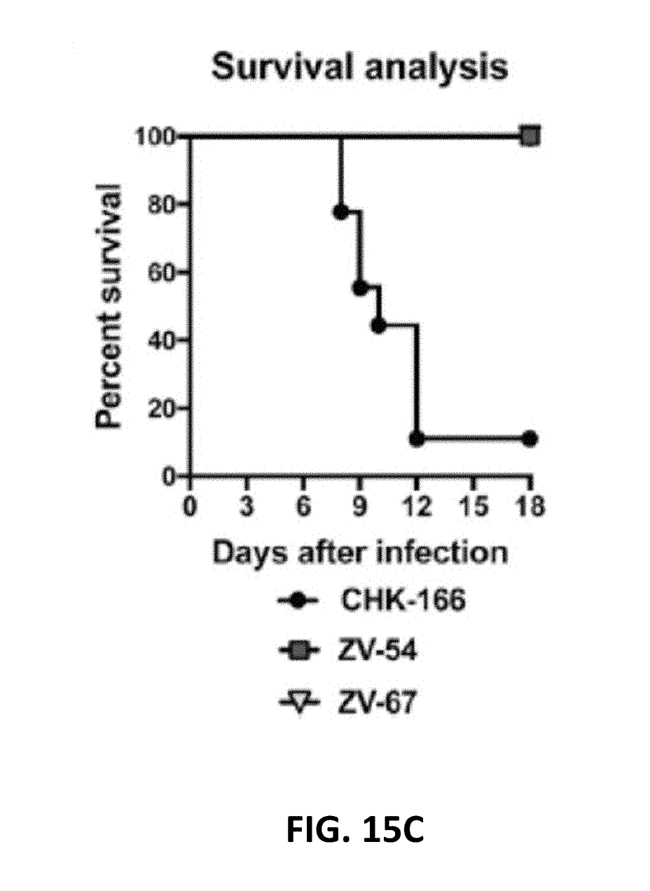

FIG. 15A shows reduced levels of viremia in animals treated with two ZIKV mAbs, ZV-54 and ZV-67, in comparison with animals treated with a control Chikungunya antibody (CHK-166). Both the ZIKV mAbs tested demonstrated protection from infection as assessed by the absence of weight loss 6-12 days post-infection (FIG. 15B), and prolonged survival compared to the control antibody (FIG. 15C).

### Example 8: Yeast Display, Affinity Measurement, and Epitope Mapping Studies

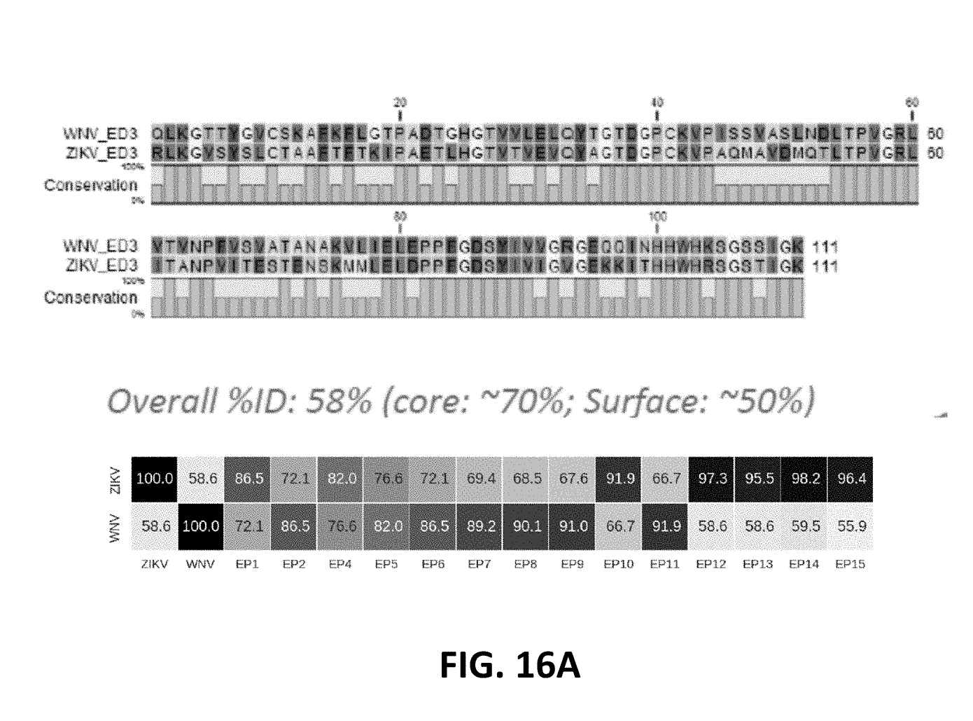

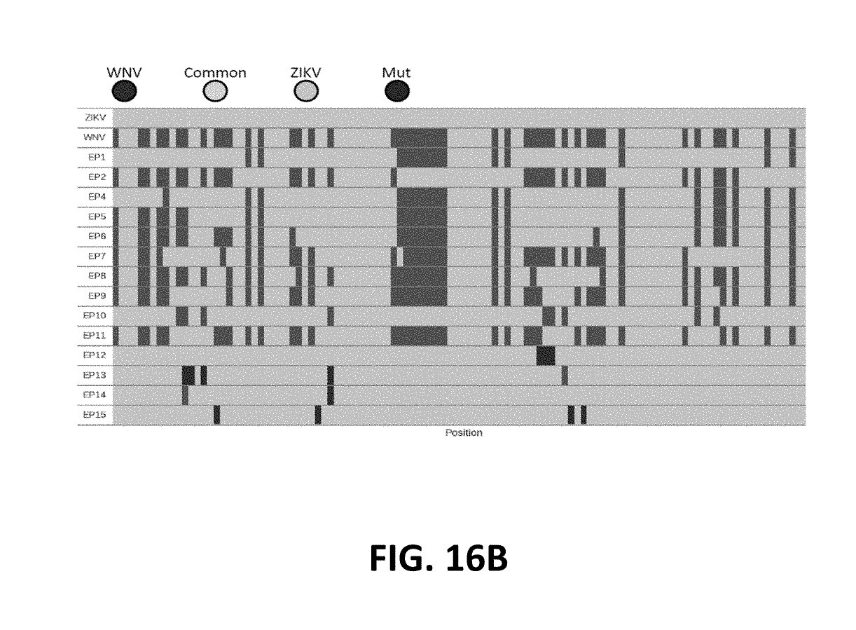

An EDIII yeast display platform was used to grossly map the epitope of lead ZIKV mAbs. Briefly, genes encoding West Nile virus (WNV) and ZIKV EDIIIs were purchased and cloned into yeast display expression vector. In this platform, the EDIII gene is fused to the gene encoding the yeast Aga2 protein which allows for EDIII to be captured on the yeast cell surface. In addition to wildtype EDIII from WNV and ZIKV, genes encoding ZIKV/WNV and ZIKV/DENV chimeric EDIII proteins containing patches of amino acids transplanted between the varying EDIII proteins were generated. Given that none of the ZIKV mAbs recognize either WNV or DENV EDIII, these chimera-containing transplanted patches were used to determine which region of the ZIKV EDIII is bound by each of the ZIKV EDIII specific antibodies. FIG. 16A shows the percent identity of each chimeric protein to either WNV or ZIKV EDIII. FIG. 16B is a schematic representation of each of the recombinant chimeric EDIII proteins. Sequences of chimeric proteins (EP1 to EP15, SEQ ID NOS: 251-265, respectively) are presented in Table 3.

TABLE 3
Recombinant EDIII proteins used in yeast display

| | |
|---|---|
| EP1 | RLKGVSYSLCTAAFTFTKIPADTGHGTVTVEVQYAGTDG |
| | PCKVPASSVASLNDLTPVGRLVTVNPVITESTENSKMML |
| | ELEPPFGDSYIVIGVGEKKITHHWHKSGSSIGK |
| EP2 | QLKGTTYGVCSKAFKFLGTPAETLHGTVVLELQYTGTDG |
| | PCKVPIQMAVDMQTLTPVGRLITANPFVSVATANAKVLI |
| | ELDPPFGDSYIVVGRGEQQINHHWHRSGSTIGK |
| EP3 | RLKGVSYSLCTAAFTFTKIPAETQHGTVTVEVQYAGTDG |
| | PCKVPASTQDEKGATQNGRLITANPVITESTENSKMMLE |
| | AEPPFGESYIVIGVGEKKITHSWFKKGSSIGK |
| EP4 | RLKGVSYSVCTAAFTFTKIPADTGHGTVTVEVQYAGTDG |
| | PCKVPASSVASLNDLTPVGRLVTVNPVITESTENSKMML |
| | ELEPPFGDSYIVIGRGEQQINHHWHKSGSSIGK |
| EP5 | QLKGTTYGVCSKAFTFTKIPADTGHGTVTVEVQYAGTDG |
| | PCKVPASSVASLNDLTPVGRLVTVNPVITESTENSKMML |
| | ELEPPFGDSYIVIGRGEQQINHHWHKSGSSIGK |
| EP6 | QLKGTTYGVCSKAFTFLGTPADTGHGTVVVEVQYAGTDG |
| | PCKVPASSVASLNDLTPVGRLVTVNPVITESTENSKMLL |
| | ELEPPFGDSYIVIGRGEQQINHHWHKSGSSIGK |
| EP7 | QLKGTTYGLCTAAFTFTGIPADTGHGTVVLELQYAGTDG |
| | PCKVPIQSVASLNDLTPVGRLVTVNPFVSVATANAKVLI |
| | ELEPPFGDSYIVVGVGEKKITHHWHKSGSSIGK |
| EP8 | QLKGTTYGVCSKAFKFTKTPADTGHGTVTLELQYTGTDG |
| | PCKVPISSVASLNDLTPVGRLVTVNPVVTESTENSKMMI |
| | ELEPPFGDSYIVVGRGEQQINHHWHKSGSSIGK |
| EP9 | QLKGTTYGVCTAAFTFTKTPADTGHGTVVLELQYAGTDG |
| | PCKVPISSVASLNDLTPVGRLVTVNPFVSESTENAKVLI |
| | ELEPPFGDSYIVVGRGEKQINHHWHKSGSSIGK |
| EP10 | RLKGVSYSLCSKAFKFTKIPAETLHGTVTVEVQYTGTDG |
| | PCKVPAQMAVDMQTLTPVGRLITANPVITVATANSKMML |
| | ELDPPFGDSYIVIGRGEQKITHHWHRSGSTIGK |
| EP11 | QLKGTTYGVCTAAFTFLGTPADTGHGTVVLELQYAGTDG |
| | PCKVPISSVASLNDLTPVGRLVTVNPFVSESTENAKVLI |
| | ELEPPFGDSYIVVGVGEKQINHHWHKSGSSIGK |
| EP12 | RLKGVSYSLCTAAFTFTKIPAETLHGTVTVEVQYAGTDG |
| | PCKVPAQMAVDMQTLTPVGRLITANPVIENKTENSKMML |
| | ELDPPFGDSYIVIGVGEKKITHHWHRSGSTIGK |
| EP13 | RLKGVSYSLCTEKFSFTKIPAETLHGTVTVEVQYEGTDG |
| | PCKVPAQMAVDMQTLTPVGRLITANPVITESTANSKMML |
| | ELDPPFGDSYIVIGVGEKKITHHWHRSGSTIGK |
| EP14 | RLKGVSYSLCTKAFTFTKIPAETLHGTVTVEVQYYGTDG |
| | PCKVPAQMAVDMQTLTPVGRLITANPVITESTENSKMML |
| | ELDPPFGDSYIVIGVGEKKITHHWHRSGSTIGK |
| EP15 | RLKGVSYSLCTAAFTFDKIPAETLHGTVTVEVKYAGTDG |
| | PCKVPAQMAVDMQTLTPVGRLITANPVITESTEESSMML |
| | ELDPPFGDSYIVIGVGEKKITHHWHRSGSTIGK |

Note: Regular font = ZIKV sequence; Underlined = WNV sequence; Italicized and underlined = DENV sequence; Bold and underlined = mutation not found in any of the virus EDIII

The genes encoding each chimeric protein, as well as the wildtype proteins, were transfected into yeast cells and yeast were plated and incubated for 3 days at 30° C. to produce individual colonies. Four colonies from each transfection event were grown in liquid culture and induced to express the EDIII on the cell surface. Each of the four induced cultures of yeast clones for each of the 15 Ep mutants as well as wildtype EDIII from WNV and ZIKV were used for subsequent flow cytometry experiments.

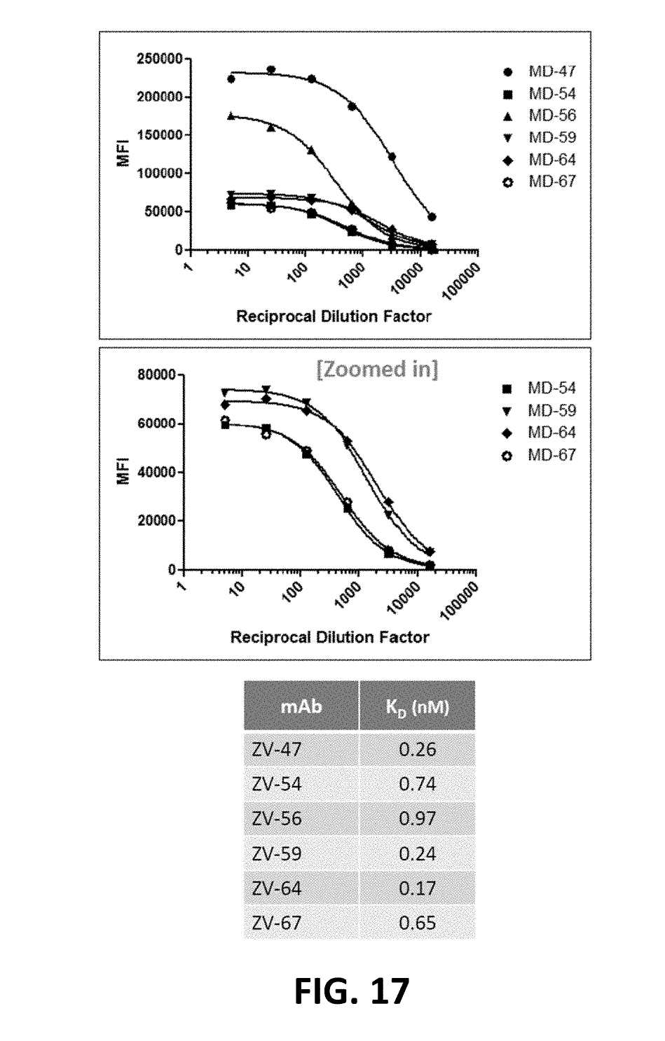

As an initial experiment, the affinity (Kd) of each of the four ZIKV mAbs was determined. Varying dilutions of hybridoma supernatants were incubated with each of the four yeast clones expressing wild type EDIII from ZIKV followed by goat anti-mouse IgG conjugated to Alexa-647 to detect ZIKV antibody binding. Additionally, each EDIII protein contains a c-myc epitope tag. To assess yeast expression of ZIKV EDIII constructs, yeast were also incubated with chicken anti-myc antibody followed by goat anti-chicken Alexa-488. Stained yeast was analyzed by flow cytometry. Clones that demonstrated excellent EDIII expression (some clones don't express, that's why four clones are assessed) as determined by c-myc staining were selected for subsequent analysis. For each antibody, the fluorescence intensity of Alexa-647 for each dilution was plotted and the results are shown in FIG. 17. A four parameter fit of each curve was employed to determine the 50% maximal binding dilution. Supernatant from the hybridomas for each of the six antibodies was quantified for antibody concentration using biolayer interferometry (Octet) employing anti-mouse IgG biosensors. By applying the concentration for each supernatant to the 50% maximal binding dilution of the supernatant, a concentration for the Kd was determined (FIG. 17).

Once the Kd of each antibody was determined, mAb(s) binding to the various chimeric proteins could be assessed. First, supernatant from all 6 antibodies was mixed as a cocktail and applied to each of the four clones from each of the 15 epitope mutants (Ep1 to Ep15). Antibody binding was detected by goat anti-mouse IgG conjugated to Alexa-647. Expression of EDIII was assessed using chicken anti-myc antibody followed by goat anti-chicken Alexa-488. Stained cells were assessed by flow cytometry for antibody binding as well as EDIII expression. At least one clone for all 15 constructs demonstrated robust EDIII expression. Numerous chimeric constructs displayed on yeast showed binding of anti-ZIKV mAbs. At this stage, all the cocktails showed is that one of the six antibodies interacted with the chimeric EDIII protein displayed on yeast and this chimeric protein would be of use in the epitope mapping exercise. For the 5 constructs that did not show staining with the antibody cocktail (EP3-EP6, EP8), it was unclear if none of the antibodies bound these proteins or the proteins had become misfolded. As such, these 5 constructs were not included in the subsequent analysis.

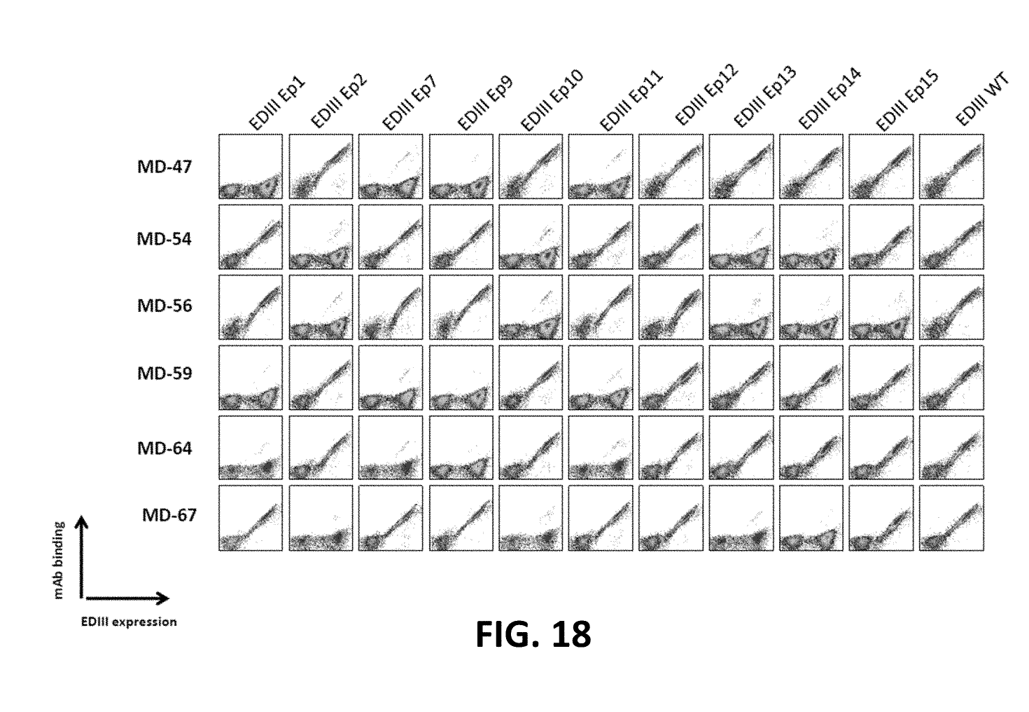

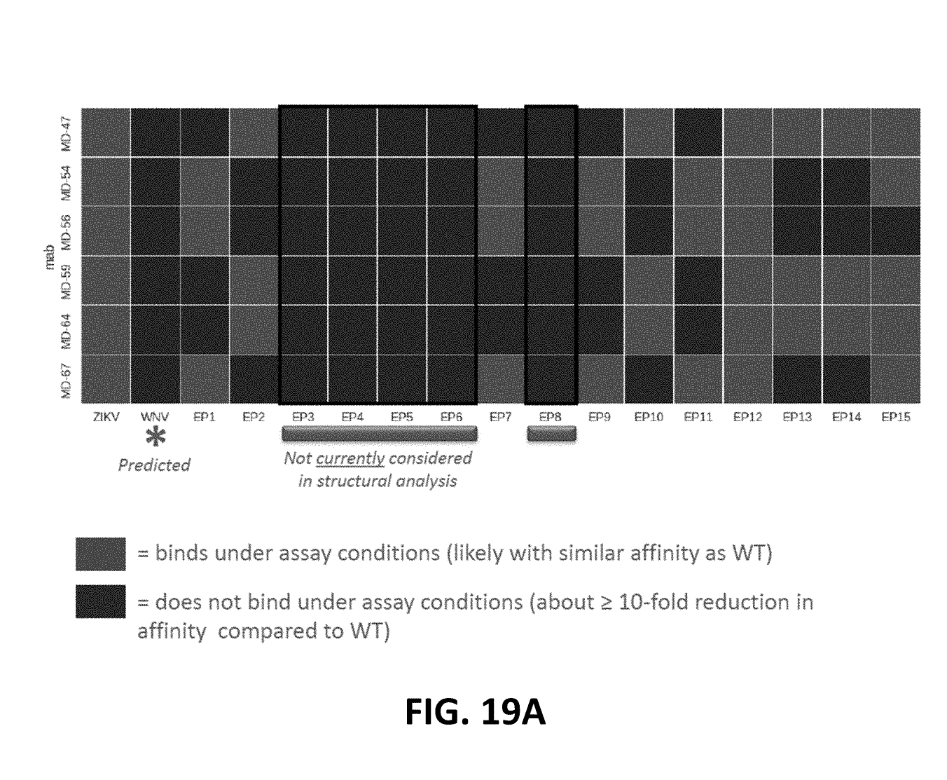

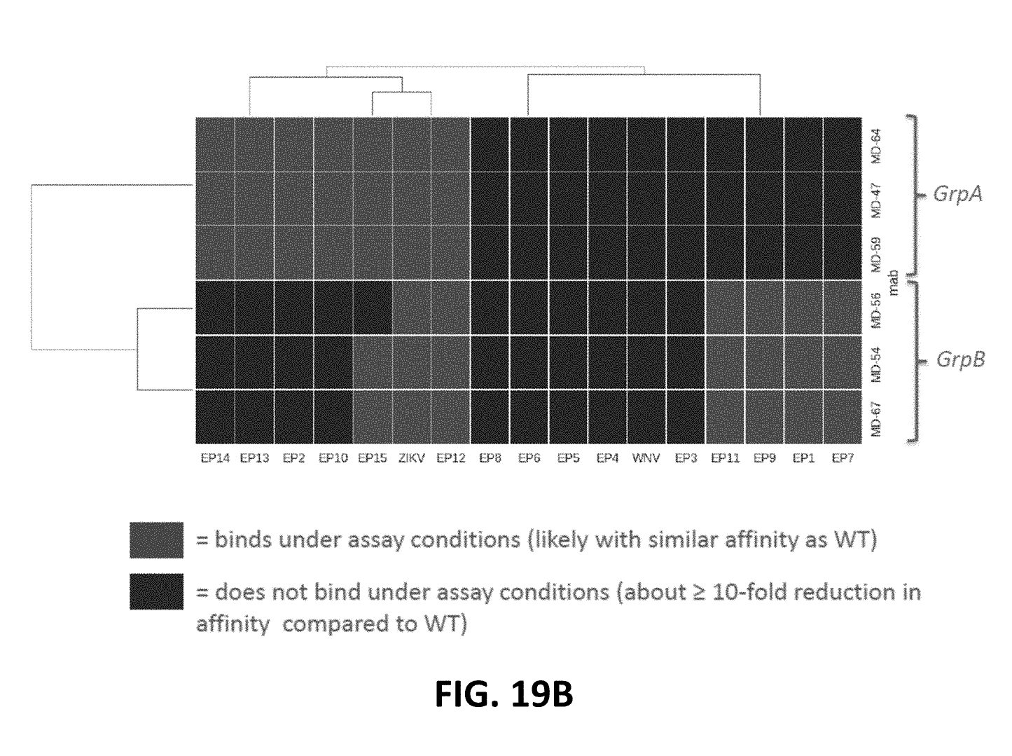

A single concentration of each of the six ZIKV mAbs, representing the concentration that gives 80% binding to wild-type ZIKV EDIII was applied to all yeast clones expressing the eleven EP mutants followed by incubation with goat anti-mouse IgG conjugated to Alexa-647. Cells were also stained with chicken anti-myc antibody followed by goat anti-chicken Alexa-488. Cells were analyzed for flow cytometry and binding of each ZIKV mAb to each chimeric protein assessed (FIG. 18). Varying patterns of staining were observed and the results are shown graphically in FIGS. 19A-19B. ZV-47, ZV-59, and ZV-64 demonstrated an identical pattern in the mapping exercise (FIGS. 19A-19B). A distinct binding pattern was seen for ZV-54 and ZV-67. ZV-56 mapped very similarly to ZV-54 and ZV-67 with the exception that ZV-56 did not bind to chimeric protein Ep15.

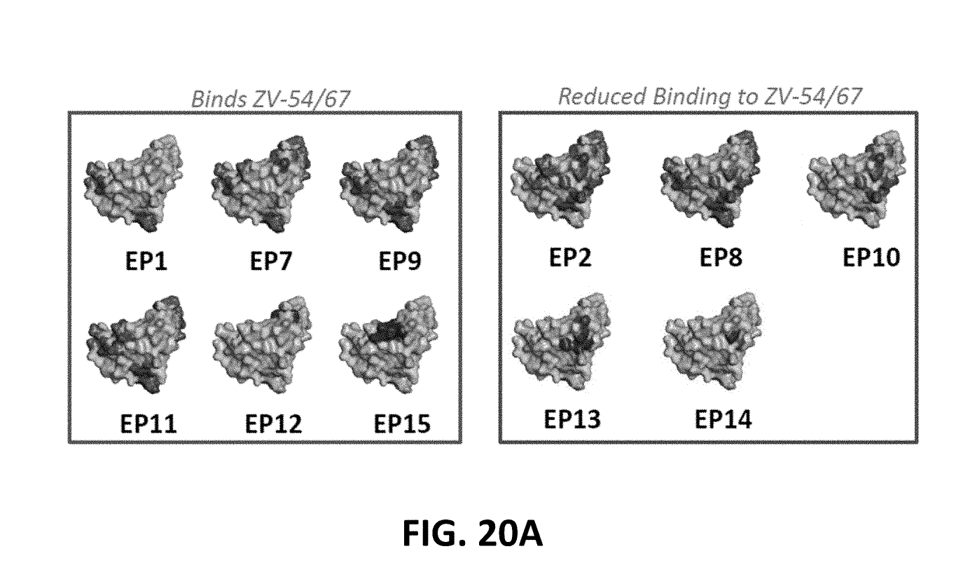

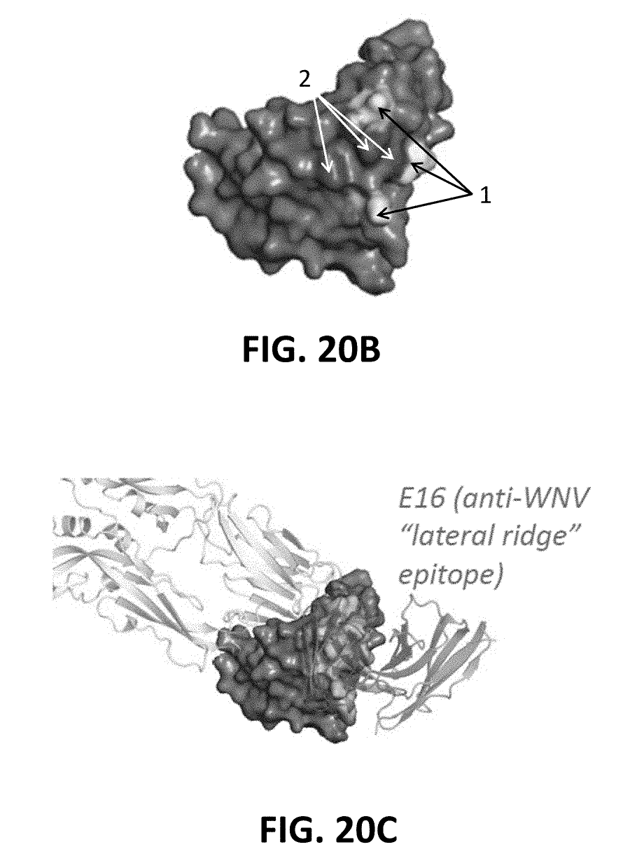

Based on the structural analysis, combined with the empirical mapping data, it was determined that ZV-54 and ZV-67 bound to the lateral ridge of EDIII, which is a preferred neutralizing epitope. FIG. 20A is a representation of how the epitope of these two mAbs was determined. In the space filling models, pink residues were derived from ZIKV, dark blue residues were derived from WNV and red residues represent mutations not found in either virus but selected based on the structural analysis. Light blue amino acids represent positions that were identical in the WNV and ZIKV EDIII. Based on this structural mapping it was clear which residues of ZIKV EDIII were required for ZV-54 and ZV-67 binding. These residues are shown (important for binding as indicated as “1”) and critical for binding as indicated as “2”) in the space filling model at the bottom left of FIG. 20B. The mapped epitope is spatially identical to the epitope identified for a potent neutralizing antibody of WNV that recognizes the lateral ridge of WMV EDIII (FIG. 20C).

### Example 9: Humanization of ZIKV Antibodies

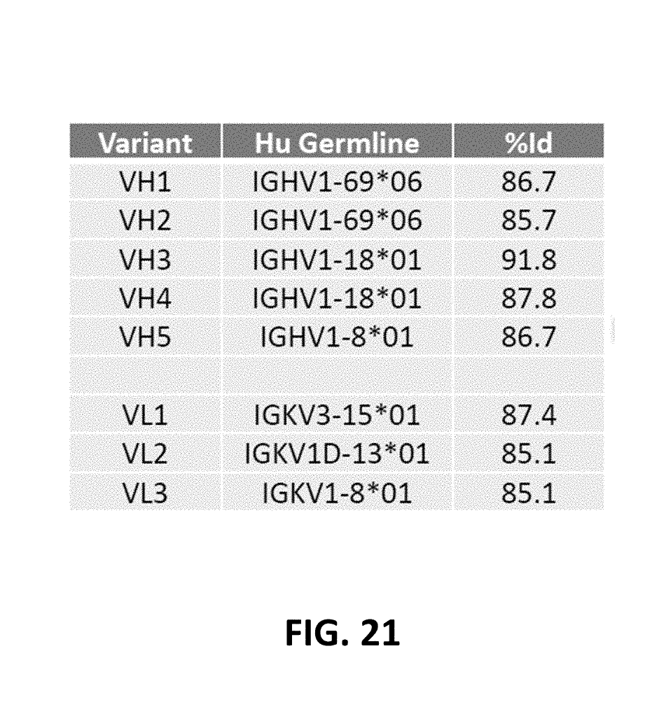

Using humanization technology, along with antibody modeling, human germline heavy and light chain genes were selected for humanization of ZV-54/67. Three heavy and three light chain genes were chosen as scaffolds for humanization (FIG. 21). Briefly, CDRs from the mouse ZIKV antibodies were grafted onto the human heavy and light chain backbones as well as specific mouse residues outside of the CDRs that were structurally determined to be required for maintenance of ZIKV binding/neutralization.

A matrix of transfections was performed in Expi293 cells that represented all possible combinations of the 5 humanized heavy chains and 3 humanized light chains. Also, the mouse heavy and light chain of ZV54 were included in the matrix to yield 20 different transfection events. Briefly, Expi293 cells in defined culture medium were transfected in 96 well plates with ExpiFectamine (ThermoFisher) and heavy and light chain DNA as described by the manufacturer. Transfected cells were incubated for 5-7 days at 37° C. in air supplemented with 5% CO₂. Supernatants were harvested and quantified using biolayer interferometry (Octet) and protein A biosensors.

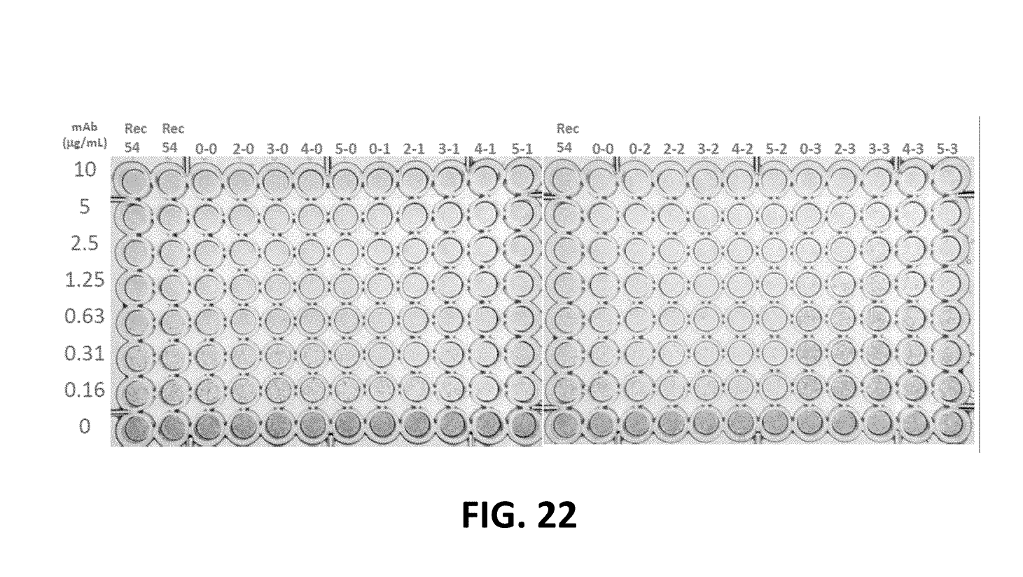

Vero cells were plated in 96 well plates and grown overnight at 37° C. in air supplemented with 5% CO₂. ZIKV strain MR766 (1:2500 dilution of the Visterra stock) was incubated with varying dilutions of each antibody-containing supernatant (10-0.16 μg/mL) and incubated for 1 hour at 37° C. Culture media on the Vero cells was removed and the antibody:virus mixture applied an incubated for 1 hour at 37° C. After incubation, media containing 1% carboxymethyl cellulose was carefully added to each well to create a viscous environment that prevents virus from diffusing in the culture. Cultures were incubated for 40 hours at 37° C. in air supplemented with 5% CO₂. Overlay was removed from all wells, cells fixed with formalin and the monolayer was stained with 4G2 (fusion loop antibody, raised against dengue, but cross-reactive with ZIKV). 4G2 binding was detected using a goat anti-mouse IgG-HRP conjugate followed by TrueBlue peroxidase substrate. Infected foci of cells appear as blue spots in the well that can be visualized. In FIG. 22, an input concentration of virus (the 1:2500 described above) was used that was too high to count the number of foci and generate a curve. The experiment was done in a qualitative way in which infected wells could easily be differentiated from protected wells by quick visual inspection.

FIG. 22 shows the results of this neutralization experiment. The first digit in the antibody name represents the heavy chain, with 0 being the ZV-54 mouse heavy chain and 1 through 5 being the various humanized heavy chains. The second digit represents the light chain, with 0 being the ZV-54 mouse light chain and 1 through 5 being the various humanized light chains. For example, 1-4 represents humanized heavy chain 1 combined with humanized light chain 5. Rec54 represents purified recombinant ZV-54. Visualizing the plates for infection allowed for the selection of 3-2 and 5-1 as the best candidates of the humanization campaign. These were renamed A-3/2 and A-5/1 which represent the leads of the A series campaign of humanization and affinity maturation (no affinity here, just this is the A series of engineering). FIG. 22 also shows that the light chain number 3 performed quite poorly, demonstrating that the approach of multiple heavy and light chain germline genes for assessment as described herein is a suitable approach.

### Example 10: Affinity Maturation of Humanized Antibodies

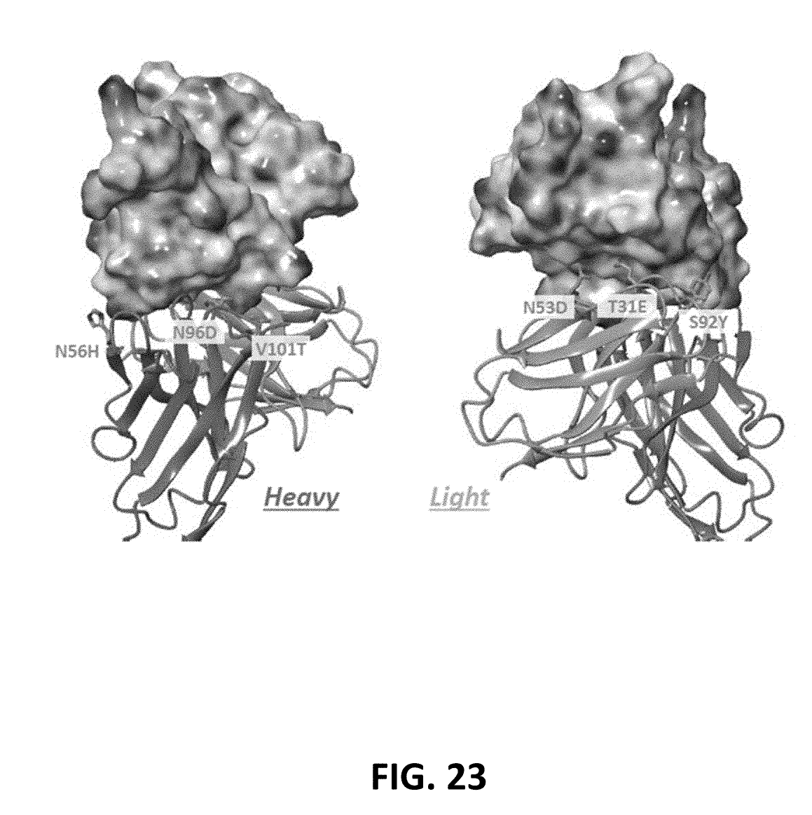

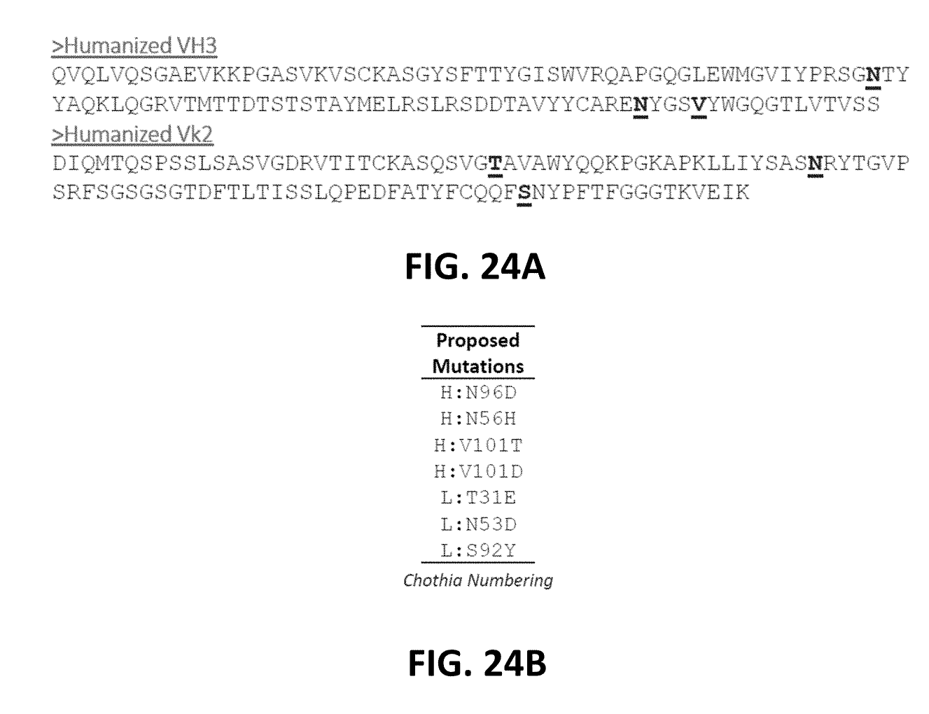

FIG. 23 represents a model of the humanized antibody (in this case A-3/2) docked to the ZIKV EDIII protein. This model was created using epitope mapping data as well as homology modeling of other antibodies in database. Three light chain and three heavy chain residues are noted in FIG. 23 as sites for mutation that are predicted to have an impact on the affinity of A-3/2 for EDIII. FIG. 24A illustrates the sequence of A-3/2 and the sites for affinity maturation, and FIG. 24B lists the specific mutations that were engineered at each selected position.

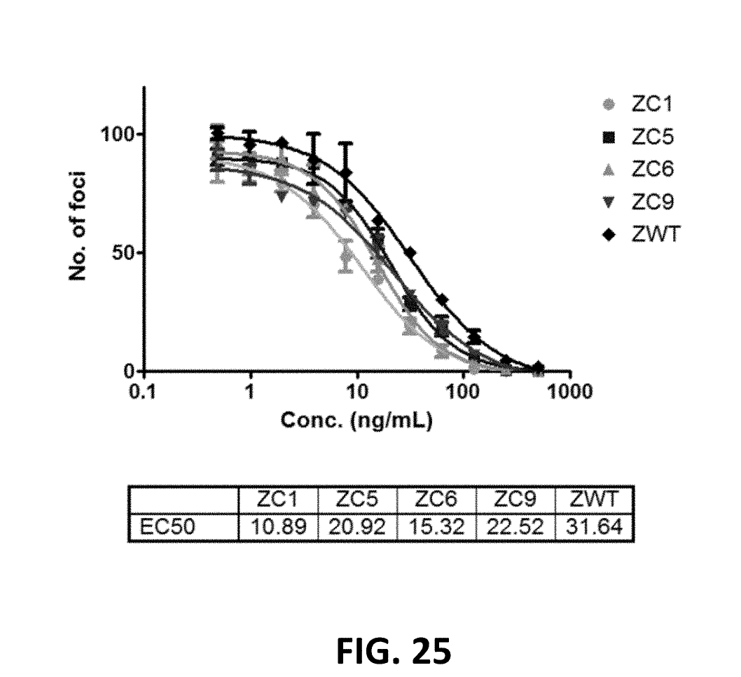

For affinity maturation, Expi293 cells were first transfected with various combinations of the A series heavy chains 3 and 5 as well as light chains 2 and 1. Various combinations of affinity enhancing mutations were also included in this matrix. Transfections were performed in 96 well plates as described above. Supernatants were harvested and antibody quantified. A neutralization assay was performed as described above with the exception that the virus input was reduced so as to visualize and count the foci. The goal was to achieve 50-100 foci per well which was successful. Foci were counted for each dilution of antibody and the results plotted. A subset of the results is shown in FIG. 25. ZWT represents the starting ZV-54 mouse antibody. ZC1, ZC5, ZC6 and ZC9 are four clones obtained from the affinity maturation process. FIG. 25 shows that affinity maturation resulted in enhanced neutralization potency (<20 ng/ml EC₅₀). These data show that humanization was successful and many antibodies have improved neutralization potency as compared to ZV-54.

### Example 11: Biophysical Assessment of Antibody Candidates

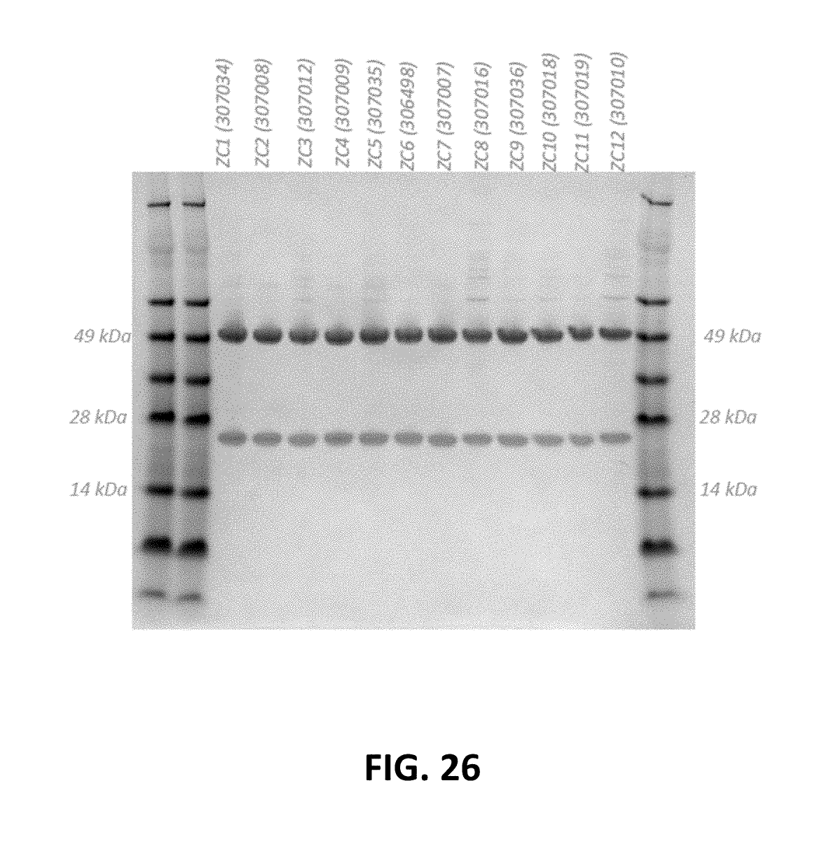

FIG. 26 shows the assessment of lead candidate antibodies (lot number and C series engineering number listed) by reducing SDS PAGE. Two micrograms of each antibody was resolved on 4-12% Bis-Tris gels and visualized with Coomassie Blue. All antibodies migrated at the appropriate size (50 kD heavy chain, 25 kD light chain).

FIG. 27 shows the assessment of lead candidate antibodies (lot number and C series engineering number listed) by non-reducing SDS PAGE. Two micrograms of each antibody was resolved on 4-12% Bis-Tris gels and visualized with Coomassie Blue. All antibodies migrated at the appropriate size (150 kD).

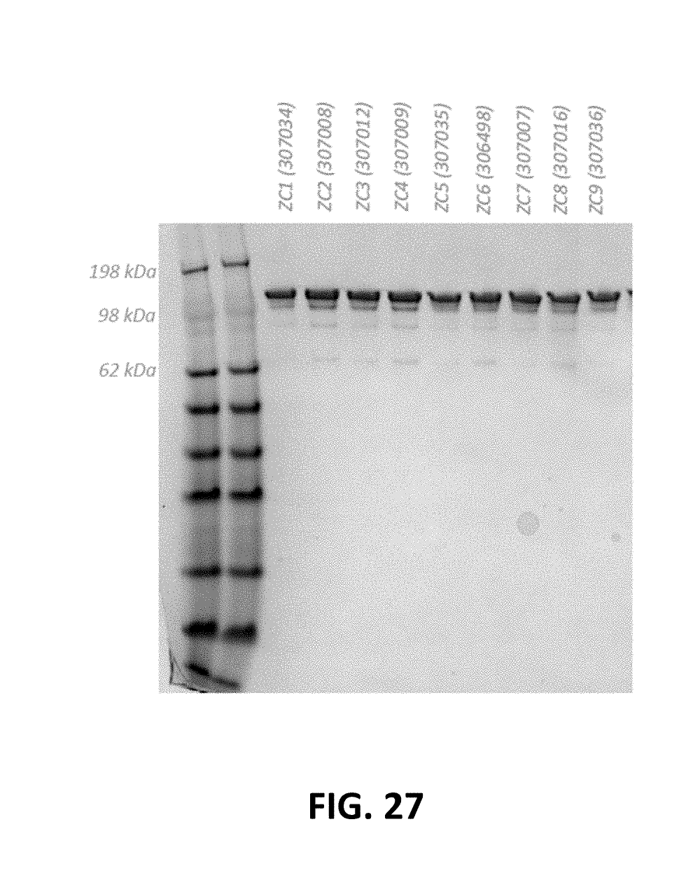

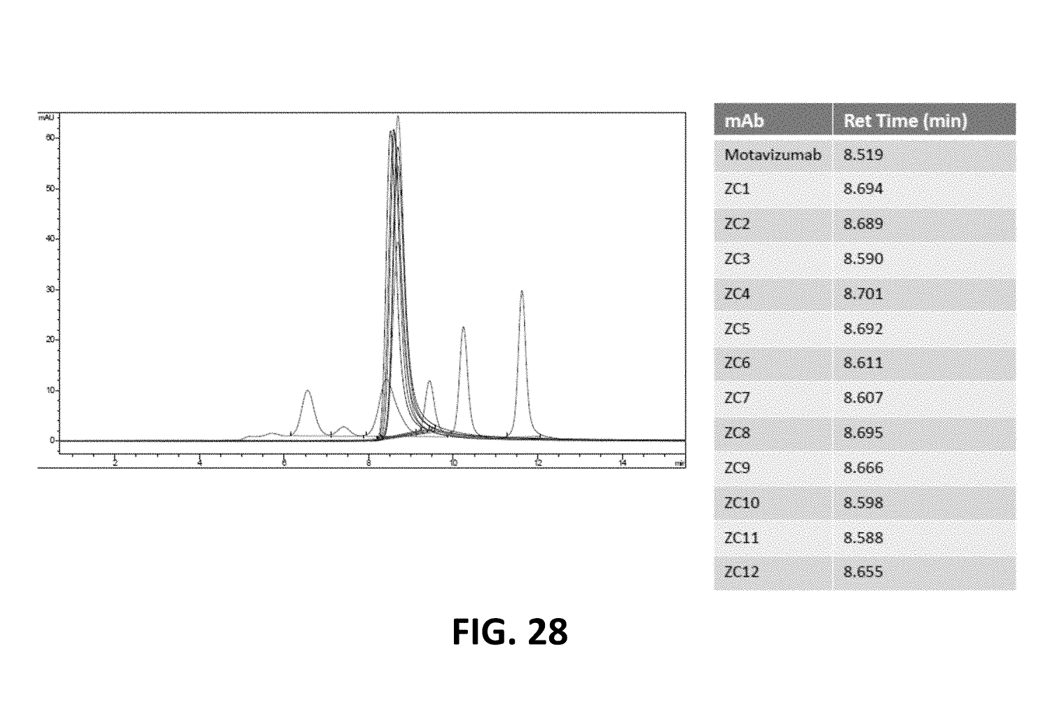

FIG. 28 shows the analysis of lead candidate C series antibodies using size exclusion chromatography (SEC-HPLC). This assay was performed to determine if the purified antibodies were monomeric and did not contain any degraded fragments or multimeric aggregates. This is a criterion for the “developability” of antibodies. Twenty micrograms of each antibody was loaded onto a Phenomenex 3000 size exclusion column with PBS as the mobile phase. Protein elution was detected by A280. Motavizumab was included as an antibody control and the red trace represents the various sizing standards included in the run. All antibodies were retained for an appropriate time for a 150 kD protein. Also, no aggregates or fragments were observed for any antibody and each protein A-purified mAb was determined to be >95% monomer.

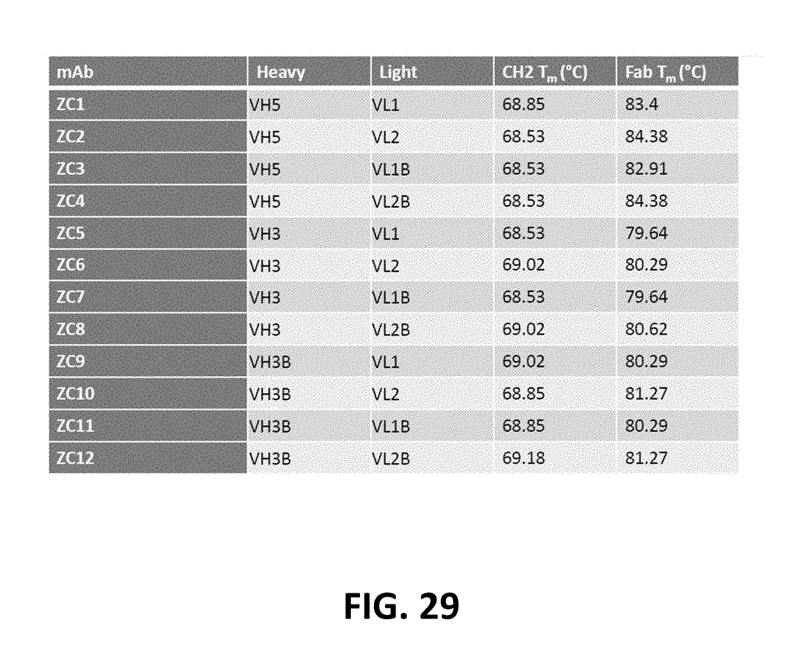

Next, lead candidate C series antibodies were assessed for thermal stability (FIG. 29). Briefly, antibody was mixed with dye Sypro Orange in a 48 well plate. Plates were subjected to increasing temperature using a qPCR-capable thermal cycler and fluorescence was measured at each single degree increase in temperature. Sypro Orange only fluoresces when bound to the hydrophobic parts of a protein. As the temperature increases, the protein eventually unfolds allowing Sypro Orange to bind and fluorescence is detected. This is called differential scanning fluorimetry. The results of the fluorescence are plotted and the first derivative of the graph is used to determine the melting temperature of each antibody. The results for each antibody are depicted in FIG. 29. All antibodies had typical CH2 (constant region domain 2 of the heavy chain) melting temperatures for antibodies (>60° C.). The Fab region was more stable than the CH2 region which again is typical for antibodies. For each C series antibody, the source of the heavy and light chains are listed and represent the A series nomenclature for each chain, e.g., Heavy VH5 is the heavy chain from A series A-5/1. When chains are noted with a B, this represents the A series nomenclature for the germline of the chain but the chain also contains affinity enhancing mutations.

### Example 12: Analysis of Non-Specific Binding and Cross-Reactivity of Antibody Candidates

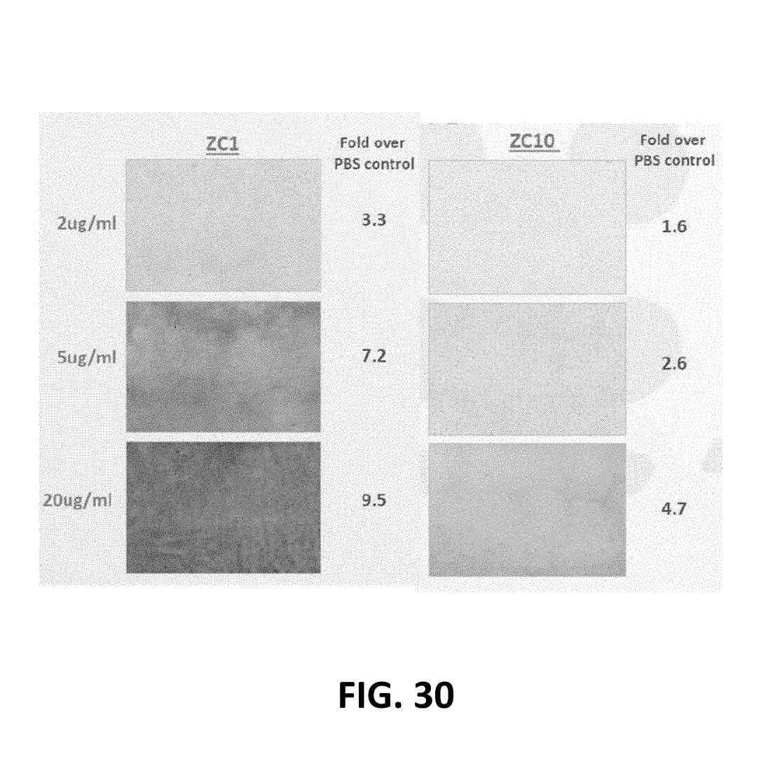

To determine whether the lead candidate antibodies demonstrate off-target binding to any human membrane proteins, a technology which identifies interactions with cell surface receptors by screening test ligands for binding against over 4,500 full length proteins that are individually over-expressed in human 293T cells was used. This physiologically-relevant system, coupled with a broad coverage of the plasma membrane proteome, allows even low affinity interactions to be detected with a high degree of sensitivity and specificity. As a first step, each antibody was assessed for non-specific binding in their assay by applying varying concentrations of antibody to their platform of 293T cells that do not express any transfected membrane proteins at this stage. FIG. 30 shows the results of two candidates, ZC1 and ZC10. All 12 C series antibodies were assessed in the background binding initial test and it was shown that some of the antibodies demonstrated higher than typical background (ZC1 as an example). Others demonstrated very low, typical background (ZC10 as an example). Upon analysis of the data, it was clear that all antibodies that contained the A series VH5 humanized heavy chain demonstrated higher than expected background. Antibodies containing the A series VH3 humanized heavy chain performed typically. Also, affinity enhancing mutations did not increase the background in this assay (ZC10 contains an affinity enhancing mutation). Given that the VH3 chain did not demonstrate unexpected non-specific binding, we chose it for further analysis as a conservative approach.

### Example 13: Fe Engineering of Antibody Candidates

FcRn interaction with IgG is believed to be mediated through Fc. The binding of Fc to FcRn is generally pH specific (typically little or no binding at pH7.4 and strong binding in acidic environment). Structure of FcRn in complex with Fc domain of IgG1 is known and each FcRn molecule binds to an Fc-monomer. Fab domains can also influence binding of IgG to FcRn.

Network analysis of Fc-FcRn complex highlights the centrality of H310 in engagement with FcRn. H310 is highly interconnected to multiple other highly networked residues. Mutations in the H310 cluster, and neighboring (connected nodes) may strengthen the H310 network. Analysis of sub-networks informs introduction of synergistic mutations for favorable FcRn interaction, with minimal impact on other Fc residues.

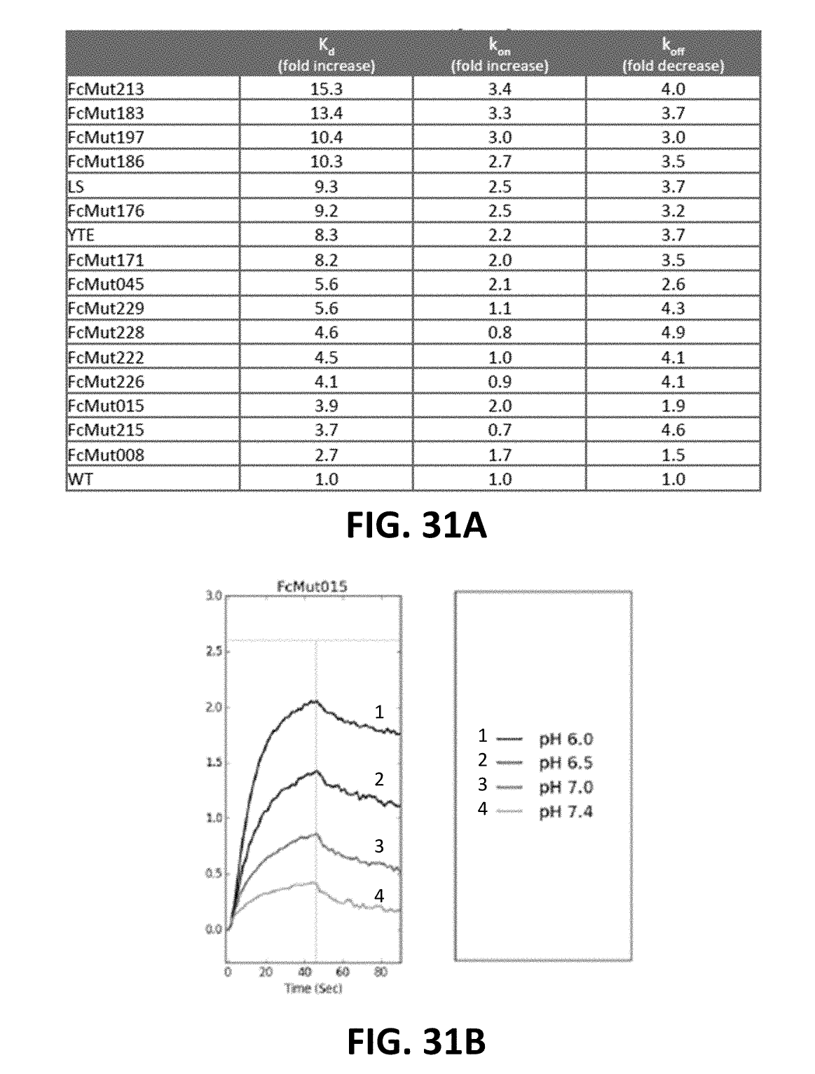

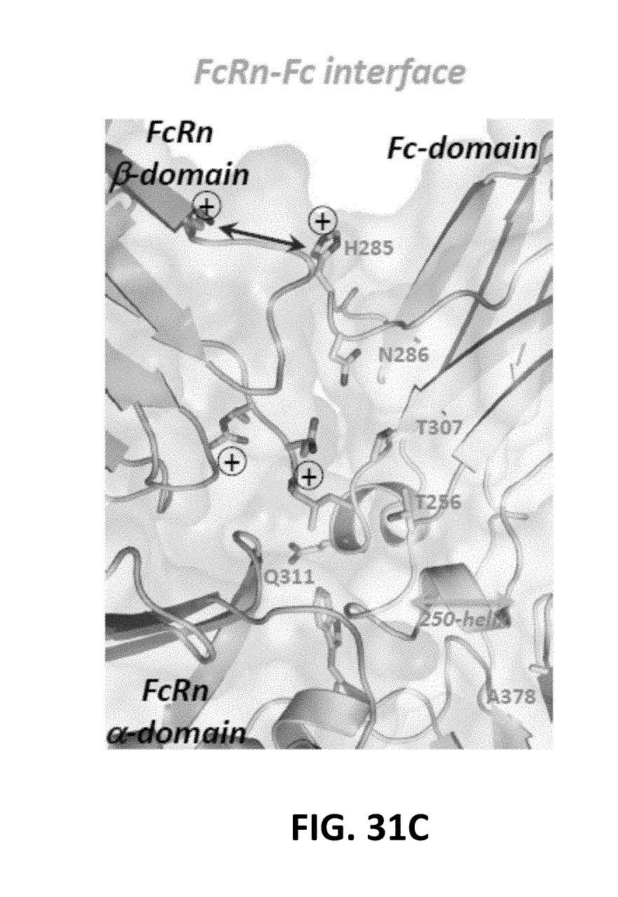

To identify Fc variants with improved binding to FcRn at pH 6.0, various Fc mutations were engineered into IgG1 Motazivumab (WT). All antibodies were assessed for binding to FcRn using biolayer interferometry. Briefly, anti-CH1 biosensors (ForteBio) were loaded with each antibody of interest. Loaded biosensors were exposed to recombinant FcRn protein at pH 6.0 to detect binding. After saturation, biosensors were exposed to buffer alone at pH 6.0 to measure dissociation of the FcRn from each antibody. The result is a response curve representing on rate and off rate of the antibody. These rates were calculated using the ForteBio software and compared to the rate of wild type Motavizumab. The fold increase in on-rate and the fold decrease in off-rate as compared to wild type are listed in the table. Numerous antibody mutations significantly increased and decreased on and off rate, respectively (FIG. 31A). Also shown is the binding of a representative Fc variant to FcRn at a pH range of 6.0 to 7.4. It is important that the affinity of the Fc mutant antibodies for FcRn is improved at pH 6.0 but not significantly enhanced at a higher pH such as pH 7.4. FIG. 31B demonstrates that this representative antibody still shows poor binding to FcRn at pH 7.4, a desirable feature.

FIG. 31C shows the interaction of the CH2 domain of the antibody Fc with the FcRn molecule. The engineering efforts described herein attempted to improve shape complementarity, charge complementarity and hydrophobic complementarity. A few positions on the Fc are noted as important for the interaction. Also noted is the 250 helix. This helix is dynamic and moves depending on the pH of the environment. This is critical in the binding of the Fc domain to FcRn at pH 6.0 but not pH 7.4.

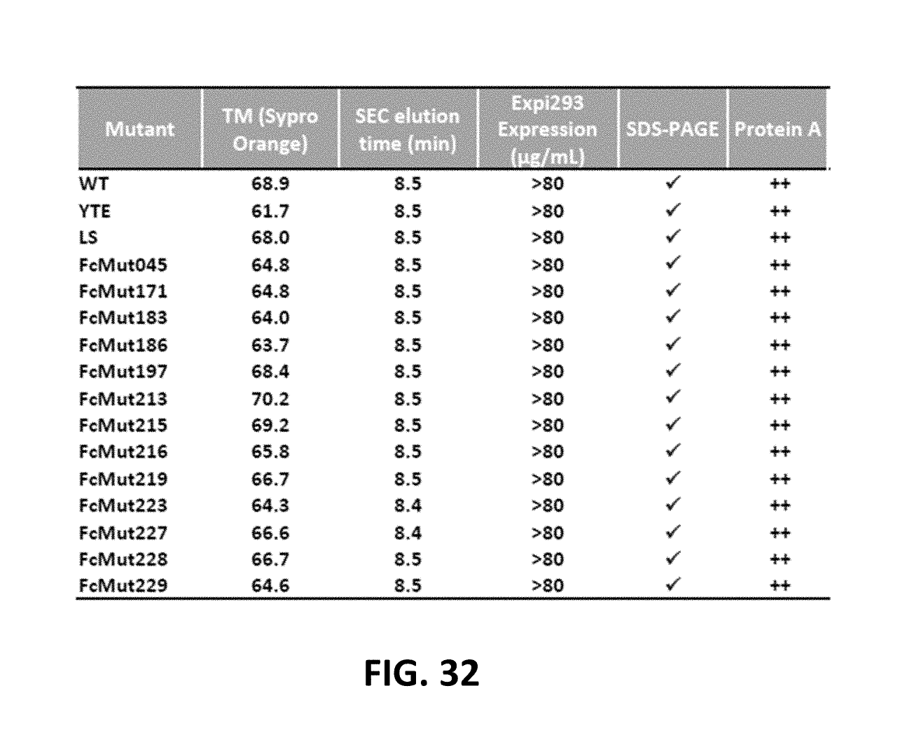

All lead Fc variants were tested in developability assays, the results of which are summarized in FIG. 32. For Sypro orange, SDS PAGE and SEC-HPLC, the methods used are similar to those used in Example 12. For expression determination, constructs were transfected into Expi293 cells in 96 well culture dishes using ExpiFectamine as described by the manufacturer. After 5-7 days, supernatants were quantified using biolayer interferometry (Octet) equipped with anti-human CH1 biosensors using a motavizumab standard curve. For protein A binding assessment the following was performed. Constructs were transfected into Expi293 cells in 30 mL cultures using ExpiFectamine as described by the manufacturer. After 5-7 days, supernatants were harvested and antibodies purified. Using biolayer interferometry with protein A biosensors, Fc-modified antibody affinity to protein A was measured and compared to antibody containing a wild type Fc domain. As shown in FIG. 32, all Fc variants performed comparably to the WT antibody in all of these experiments.

### Example 14: In Vivo Assessment of Half-Life of Engineered Antibodies

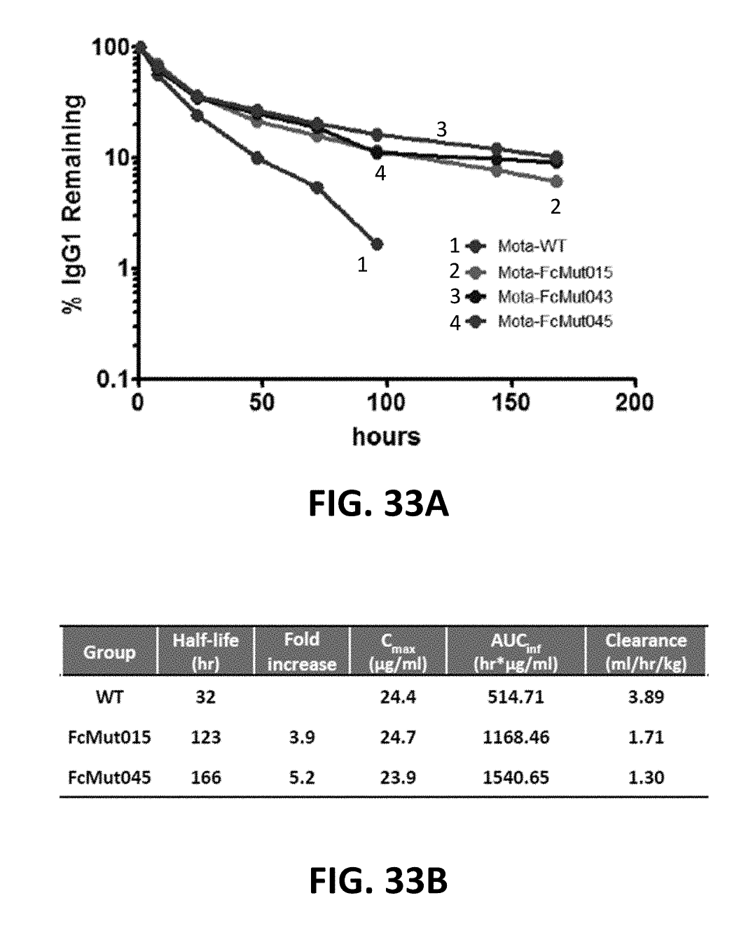

Motavizumab wildtype was compared to Motavizumab containing three of Fc engineering mutations in the in vivo assessment of engineered antibody half-life. Antibodies (2-5 mg/kg) were administered to mice transgenic for human FcRn and daily samples of mouse serum were obtained (day 0-day 4). ELISA was performed on serum to quantify the amount of Motazimunab in the serum. ELISA results were converted to percent of antibody remaining based on the day 0 timepoint representing 100%. As shown in FIGS. 33A-33B, all three antibody variants demonstrated extended half-life in this well-established mouse model of antibody half-life. Motavizumab wild-type has a half-life of 32 hours. FcMut043 and FcMut045 mutants built on FcMut008 show significant half-life improvement. FcMut045 mutant enhanced half-life 5.2 fold (about 166 hours half-life). The half-life as well as the other parameters in FIG. 33B were calculated using the Winonlin software.

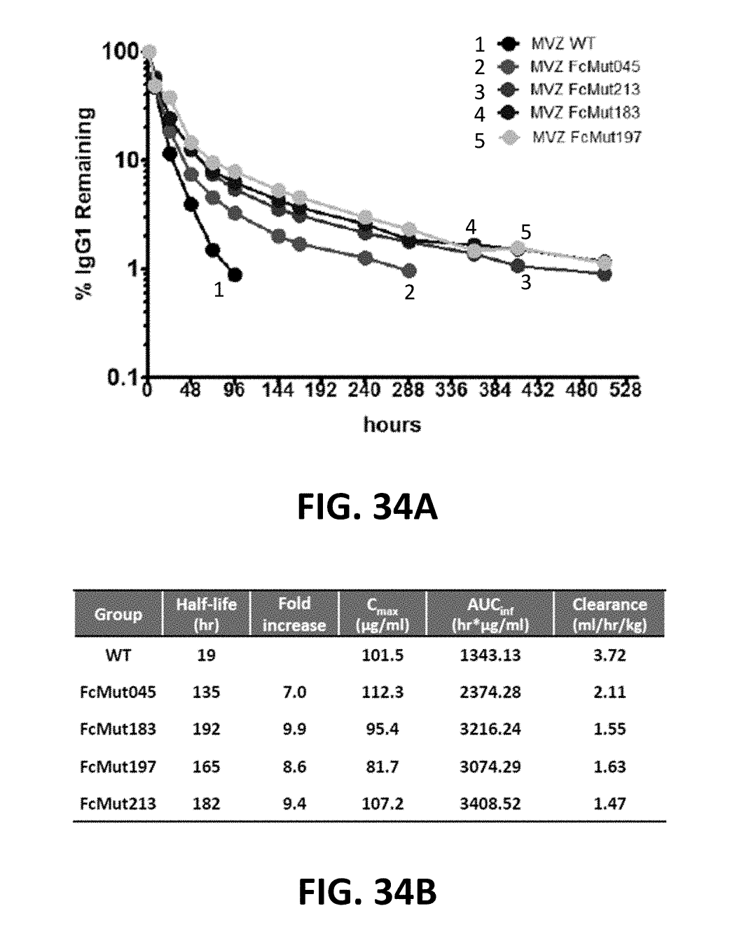

A similar experiment was conducted with later stage Fc variants in the context of Motazivumab (FIGS. 34A-34B). Next generation variants (FcMut171, FcMut183, FcMut186, and FcMut197) demonstrated further enhanced half-life with more than nine fold increase in half-life observed with FcMut213. One of the early variants (FcMut045) was included to demonstrate the improved half-life seen with the later stage designs as compared to the early stage designs.

### Example 15: In Vivo Assessment of Half-Life of Engineered Antibodies

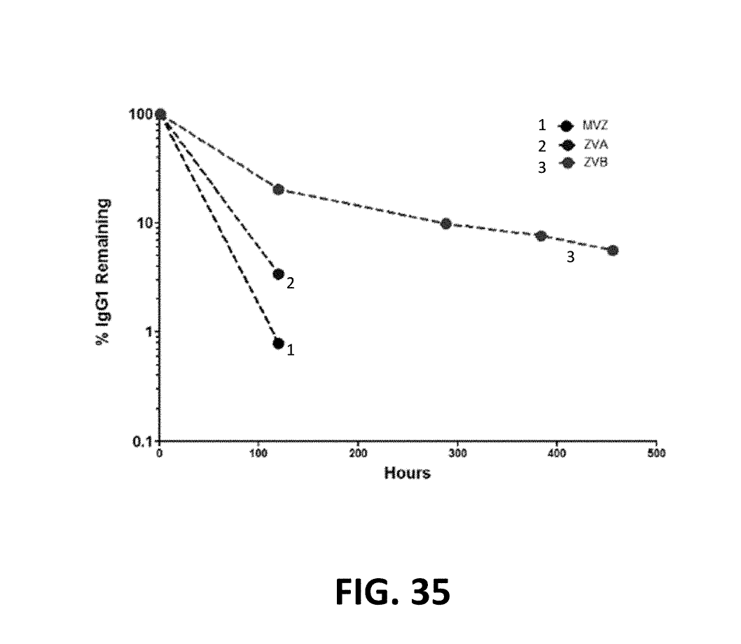

Two Zika antibodies (ZVA and ZVB) as well as wild type Motavizumab (MVZ) were administered to mice transgenic for human FcRn at a dose of 2-5 mg/kg, and daily samples of mouse serum were obtained (day 0-day 4). ELISA was performed on serum to quantify the amount of Motazimunab. ELISA results were converted to percent of antibody remaining based on the day 0 timepoint representing 100%. ZVA antibody represents the A series antibody A-3/2. The ZVB antibody represents the A series antibody A-5/1 containing the affinity enhancing light chain modification S92Y. As shown in FIG. 35, ZVB had a much longer half-life than either Motavizumab or ZVA. These antibodies contained wild-type Fc regions so the extended half-life of the ZVB antibody is a property of the antibody itself, and not any Fc engineering.

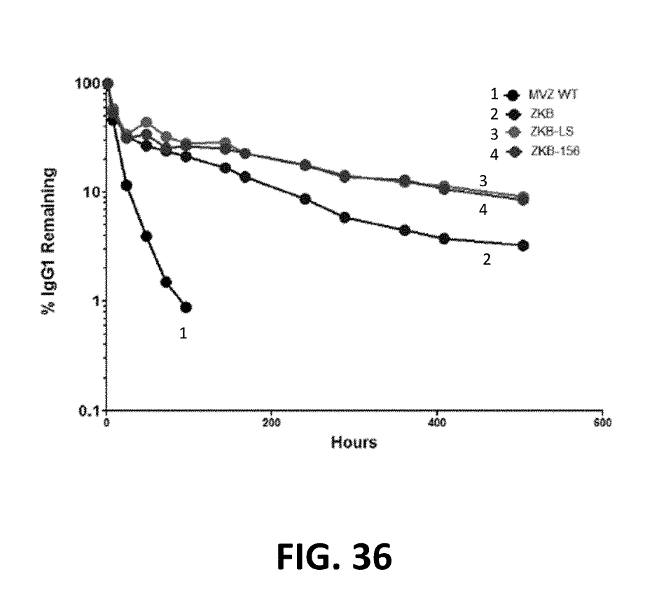

In the next experiment, Motavizumab wild type (MVZ WT) was tested against the ZVB antibody (ZB-1/4, ZKB in the graph) and the ZVB antibody containing Fc modification 156 (L234A/L235A (“LALA”) and T256D/T307R/Q311V (half-life extension), ZKB-156 in the graph) as well as the “LS” half-life extension Fc mutation (ZKB-LS). Both LS and Fc modification 156 extended half-life significantly compared to the wild-type ZVB (FIG. 36). These data demonstrate that the Visterra mutation is comparable to the literature derived LS mutation, and also demonstrates that the FC engineering efforts described herein can extend the half-life of an antibody that already has a very long half-life in this transgenic mouse model. “ZVB” is indicated as “ZKB” in FIG. 36.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An anti-Zika virus (ZIKV) antibody molecule, comprising a heavy chain variable region (VH) and a light chain variable region (VL), wherein the VH comprises three heavy chain complementarity determining regions (HCDR1, HCDR2, and HCDR3), and the VL comprises three light chain complementarity determining regions (LCDR1, LCDR2, and LCDR3), and wherein:
(a) the VH comprises: (i) an HCDR1 comprising the amino acid sequence G-Y-S/T-T-T/S-Y (SEQ ID NO: 351); (ii) an HCDR2 comprising the amino acid sequence Y-P-R-S-N/G-N/H (SEQ ID NO: 352); and (iii) an HCDR3 comprising the amino acid sequence E-N/D-Y-G-S-V/T/D-Y (SEQ ID NO: 353); and the VL comprises: (i) an LCDR1 comprising the amino acid sequence K/R-A-S-Q-S/N-V-G-T/E-A-V-A (SEQ ID NO: 354); (ii) an LCDR2 comprising the amino acid sequence S-A-S-N/D-R/L-Y-T (SEQ ID NO: 355); and (iii) an LCDR3 comprising the amino acid sequence Q-Q-F-S/Y-N/S-Y-P-F/Y-T (SEQ ID NO: 356); or
(b) the VH comprises: (i) an HCDR1 comprising the amino acid sequence S/T-Y-G-I-S(SEQ ID NO: 357); (ii) an HCDR2 comprising the amino acid sequence V-I-Y-P-R-S-N/G-N/H-T-Y-Y-N/A-E/Q-R/K-F/L-R/Q-G (SEQ ID NO: 358); and (iii) an HCDR3 comprising the amino acid sequence E-N/D-Y-G-S-V/T/D-Y (SEQ ID NO: 353); and the VL comprises: (i) an LCDR1 comprising the amino acid sequence K/R-A-S-Q-S/N-V-G-T/E-A-V-A (SEQ ID NO: 354); (ii) an LCDR2 comprising the amino acid sequence S-A-S-N/D-R/L-Y-T (SEQ ID NO: 355); and (iii) an LCDR3 comprising the amino acid sequence Q-Q-F-S/Y-N/S-Y-P-F/Y-T (SEQ ID NO: 356).

2. The antibody molecule of claim 1, wherein:
(a) the VH comprises: (i) an HCDR1 comprising the amino acid sequence of SEQ ID NO: 101 or 107; (ii) an HCDR2 comprising the amino acid sequence of any of SEQ ID NOs: 102, 108, or 114; or (iii) an HCDR3 comprising the amino acid sequence of any of SEQ ID NOs: 103, 113, 115, or 116; and the VL comprises: (i) an LCDR1 comprising the amino acid of any of SEQ ID NOs: 104, 109, 111, or 117; (ii) an LCDR2 comprising the amino acid of any of SEQ ID NOs: 105, 112, or 118; or (iii) an LCDR3 comprising the amino acid sequence of any of SEQ ID NOs: 106, 110, or 119, or
(b) the VH comprises: (i) an HCDR1 comprising the amino acid sequence of SEQ ID NO: 201 or 203; (ii) an HCDR2 comprising the amino acid sequence of any of SEQ ID NOs: 202, 204, 205, 206, 207, 208, or 209; or (iii) an HCDR3 comprising the amino acid sequence of any of SEQ ID NOs: 103, 113, 115, or 116; and the VL comprises: (i) an LCDR1 comprising the amino acid of any of SEQ ID NOs: 104, 109, 111, or 117; (ii) an LCDR2 comprising the amino acid of any of SEQ ID NOs: 105, 112, or 118; or (iii) an LCDR3 comprising the amino acid sequence of any of SEQ ID NOs: 106, 110, or 119.

3. The antibody molecule of claim 1, comprising the HCDR1, HCDR2, HCDR3, LCDR1, LCDR2, and LCDR3 of any of monoclonal antibodies ZV54, ZV67, ZA-1/1, ZA-1/2, ZA-1/3, ZA-2/1, ZA-2/2, ZA-2/3, ZA-3/1, ZA-3/2, ZA-3/3, ZA-4/1, ZA-4/2, ZA-4/3, ZA-5/1, ZA-5/2, ZA-5/3, ZB-1/1, ZB-1/2, ZB-1/3, ZB-1/4, ZB-2/1, ZB-2/2, ZB-2/3, ZB-2/4, ZB-3/1, ZB-3/2, ZB-3/3, ZB-3/4, ZB-4/1, ZB-4/2, ZB-4/3, ZB-4/4, ZB-5/1, ZB-5/2, ZB-5/3, ZB-5/4, ZC1, ZC2, ZC3, ZC4, ZC5, ZC6, ZC7, ZC8, ZC9, ZC10, ZC11, or ZC12.

4. The antibody molecule of claim 1, which comprises one or both of (a) a VH comprising the amino acid sequence of any of SEQ ID NOs: 1, 3, 5, 9, 10, 11, 12, 13, 14, 15, 16, 17, or 22, or an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith, or (b) a VL comprising the amino acid sequence of any of SEQ ID NOs: 2, 4, 6, 7, 8, 18, 19, 20, or 21, or an amino acid sequence that differs by no more than 1, 2, 3, 4, 5, 6, 7, 8, 9, 10, 11, 12, 13, 14, or 15 amino acid residues therefrom, or has at least 85, 90, 95, 96, 97, 98, 99, or 100% homology therewith.

5. The antibody molecule of claim 1, which comprises:
(a) a VH comprising the amino acid sequence of SEQ ID NO: 1 and a VL comprising the amino acid sequence of SEQ ID NO: 2;
(b) a VH comprising the amino acid sequence of SEQ ID NO: 3 and a VL comprising the amino acid sequence of SEQ ID NO: 4;
(c) a VH comprising the amino acid sequence of SEQ ID NO: 5 and a VL comprising the amino acid sequence of SEQ ID NO: 6;
(d) a VH comprising the amino acid sequence of SEQ ID NO: 5 and a VL comprising the amino acid sequence of SEQ ID NO: 7;
(e) a VH comprising the amino acid sequence of SEQ ID NO: 5 and a VL comprising the amino acid sequence of SEQ ID NO: 8;
(f) a VH comprising the amino acid sequence of SEQ ID NO: 9 and a VL comprising the amino acid sequence of SEQ ID NO: 6;
(g) a VH comprising the amino acid sequence of SEQ ID NO: 9 and a VL comprising the amino acid sequence of SEQ ID NO: 7;
(h) a VH comprising the amino acid sequence of SEQ ID NO: 9 and a VL comprising the amino acid sequence of SEQ ID NO: 8;
(i) a VH comprising the amino acid sequence of SEQ ID NO: 10 and a VL comprising the amino acid sequence of SEQ ID NO: 6;
(j) a VH comprising the amino acid sequence of SEQ ID NO: 10 and a VL comprising the amino acid sequence of SEQ ID NO: 7;
(k) a VH comprising the amino acid sequence of SEQ ID NO: 10 and a VL comprising the amino acid sequence of SEQ ID NO: 8;
(l) a VH comprising the amino acid sequence of SEQ ID NO: 11 and a VL comprising the amino acid sequence of SEQ ID NO: 6;
(m) a VH comprising the amino acid sequence of SEQ ID NO: 11 and a VL comprising the amino acid sequence of SEQ ID NO: 7;
(n) a VH comprising the amino acid sequence of SEQ ID NO: 11 and a VL comprising the amino acid sequence of SEQ ID NO: 8;
(o) a VH comprising the amino acid sequence of SEQ ID NO: 12 and a VL comprising the amino acid sequence of SEQ ID NO: 6;
(p) a VH comprising the amino acid sequence of SEQ ID NO: 12 and a VL comprising the amino acid sequence of SEQ ID NO: 7;
(q) a VH comprising the amino acid sequence of SEQ ID NO: 12 and a VL comprising the amino acid sequence of SEQ ID NO: 8;
(r) a VH comprising the amino acid sequence of SEQ ID NO: 13 and a VL comprising the amino acid sequence of SEQ ID NO: 6;
(s) a VH comprising the amino acid sequence of SEQ ID NO: 13 and a VL comprising the amino acid sequence of SEQ ID NO: 18;
(t) a VH comprising the amino acid sequence of SEQ ID NO: 13 and a VL comprising the amino acid sequence of SEQ ID NO: 19;
(u) a VH comprising the amino acid sequence of SEQ ID NO: 13 and a VL comprising the amino acid sequence of SEQ ID NO: 20;
(v) a VH comprising the amino acid sequence of SEQ ID NO: 14 and a VL comprising the amino acid sequence of SEQ ID NO: 6;
(w) a VH comprising the amino acid sequence of SEQ ID NO: 14 and a VL comprising the amino acid sequence of SEQ ID NO: 18;
(x) a VH comprising the amino acid sequence of SEQ ID NO: 14 and a VL comprising the amino acid sequence of SEQ ID NO: 19;
(y) a VH comprising the amino acid sequence of SEQ ID NO: 14 and a VL comprising the amino acid sequence of SEQ ID NO: 20;
(z) a VH comprising the amino acid sequence of SEQ ID NO: 15 and a VL comprising the amino acid sequence of SEQ ID NO: 6;
(aa) a VH comprising the amino acid sequence of SEQ ID NO: 15 and a VL comprising the amino acid sequence of SEQ ID NO: 18;
(bb) a VH comprising the amino acid sequence of SEQ ID NO: 15 and a VL comprising the amino acid sequence of SEQ ID NO: 19;
(cc) a VH comprising the amino acid sequence of SEQ ID NO: 15 and a VL comprising the amino acid sequence of SEQ ID NO: 20;
(dd) a VH comprising the amino acid sequence of SEQ ID NO: 16 and a VL comprising the amino acid sequence of SEQ ID NO: 6;
(ee) a VH comprising the amino acid sequence of SEQ ID NO: 16 and a VL comprising the amino acid sequence of SEQ ID NO: 18;
(ff) a VH comprising the amino acid sequence of SEQ ID NO: 16 and a VL comprising the amino acid sequence of SEQ ID NO: 19;
(gg) a VH comprising the amino acid sequence of SEQ ID NO: 16 and a VL comprising the amino acid sequence of SEQ ID NO: 20;
(hh) a VH comprising the amino acid sequence of SEQ ID NO: 17 and a VL comprising the amino acid sequence of SEQ ID NO: 6;
(ii) a VH comprising the amino acid sequence of SEQ ID NO: 17 and a VL comprising the amino acid sequence of SEQ ID NO: 18;
(jj) a VH comprising the amino acid sequence of SEQ ID NO: 17 and a VL comprising the amino acid sequence of SEQ ID NO: 19;
(kk) a VH comprising the amino acid sequence of SEQ ID NO: 17 and a VL comprising the amino acid sequence of SEQ ID NO: 20;
(ll) a VH comprising the amino acid sequence of SEQ ID NO: 13 and a VL comprising the amino acid sequence of SEQ ID NO: 20;
(mm) a VH comprising the amino acid sequence of SEQ ID NO: 13 and a VL comprising the amino acid sequence of SEQ ID NO: 7;
(nn) a VH comprising the amino acid sequence of SEQ ID NO: 13 and a VL comprising the amino acid sequence of SEQ ID NO: 6;
(oo) a VH comprising the amino acid sequence of SEQ ID NO: 13 and a VL comprising the amino acid sequence of SEQ ID NO: 21;
(pp) a VH comprising the amino acid sequence of SEQ ID NO: 10 and a VL comprising the amino acid sequence of SEQ ID NO: 20;
(qq) a VH comprising the amino acid sequence of SEQ ID NO: 10 and a VL comprising the amino acid sequence of SEQ ID NO: 7;
(rr) a VH comprising the amino acid sequence of SEQ ID NO: 10 and a VL comprising the amino acid sequence of SEQ ID NO: 6;
(ss) a VH comprising the amino acid sequence of SEQ ID NO: 10 and a VL comprising the amino acid sequence of SEQ ID NO: 21;
(tt) a VH comprising the amino acid sequence of SEQ ID NO: 22 and a VL comprising the amino acid sequence of SEQ ID NO: 20;
(uu) a VH comprising the amino acid sequence of SEQ ID NO: 22 and a VL comprising the amino acid sequence of SEQ ID NO: 7;
(vv) a VH comprising the amino acid sequence of SEQ ID NO: 22 and a VL comprising the amino acid sequence of SEQ ID NO: 6; or
(ww) a VH comprising the amino acid sequence of SEQ ID NO: 22 and a VL comprising the amino acid sequence of SEQ ID NO: 21.

6. The antibody molecule of claim 1, which comprises an Fc region.

7. The antibody molecule of claim 6, wherein the Fc region comprises:
(i) one or more mutations or one or more combinations of mutations chosen from M252W, V308F/N434Y, R255Y, P257L/N434Y, V308F, P257N/M252Y, G385N, P257N/V308Y, N434Y, M252Y/S254T/T256E (“YTE”), M428L/N434S (“LS”), or any combination thereof.
(ii) (a) one or more (e.g., 2, 3, 4, 5, or all) combinations of mutations chosen from: T256D/Q311V/A378V, H285N/T307Q/N315D, H285D/T307Q/A378V, T307Q/Q311V/A378V, T256D/N286D/T307R/Q311V/A378V, or T256D/T307R/Q311V; (b) a mutation or a combination of mutations capable of disrupting an Fc effector function, e.g., L234A/L235A (also known as “LALA” mutation), or (c) both (a) and (b); or
(iii) both (i) and (ii)

8. The antibody molecule of claim 1, which binds to one, two, or all of:
(a) the E protein of Zika virus, domain III of the E protein, or the lateral ridge of domain III of the E protein);
(b) an epitope on Zika virus or an E protein of Zika virus, that is not cross-reactive to one or more different flaviviruses chosen from dengue virus, Japanese encephalitis virus, West Nile virus, or a combination thereof; or
(c) an epitope on Zika virus comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acid residues in domain III of the E protein.

9. The antibody molecule of claim 1, which binds to Zika virus, the E protein of Zika virus, or domain III of the E protein, with a dissociation constant (K_{D}) of about 100 nM or less, about 10 nM or less, about 5 nM or less, about 1 nM or less, about 0.5 nM or less, about 0.1 nM or less, about 0.05 nM or less, or about 0.01 nM or less, about 10 nM to about 0.01 nM, about 5 nM to about 0.01 nM, about 3 to about 0.05 nM, about 1 nM and about 0.1 nM, about 50 nM or less, about 40 nM or less, about 30 nM or less, about 20 nM or less, about 10 nM or less, about 8 nM or less, about 6 nM or less, about 4 nM or less, about 2 nM or less, about 1 nM or less, about 0.5 nM or less, about 0.2 nM or less, about 0.1 nM or less, about 0.05 nM or less, about 0.02 nM or less, or about 0.01 nM or less.

10. The antibody molecule of claim 1, which has one, two, three, four, five or all of the following properties:
(a) reduces one or more biological activities of Zika virus, in vitro, ex vivo, or in vivo;
(b) reduces binding of Zika virus to a cell surface, fusion between a Zika virus E protein and an endosomal membrane, or both;
(c) inhibits Zika virus in a microneutralization assay;
(d) inhibits Zika virus with an EC50 of about 10 μg/mL or less, about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less;
(e) inhibits Zika virus with an EC90 of about 20 μg/mL or less, about 10 μg/mL or less, about 5 μg/mL or less, about 2 μg/mL or less, about 1 μg/mL or less, about 0.8 μg/mL or less, about 0.6 μg/mL or less, about 0.4 μg/mL or less, about 0.2 μg/mL or less, or about 0.1 μg/mL or less; or
(f) does not bind to domain II of the E protein, or binds to domain II of the E protein with a dissociation constant (K_{D}) of about 500 nM or more, about 750 nM or more, about 1 μM or more, about 2 μM or more, about 3 μM or more, about 4 μM or more, or about 5 μM or more.

11. The antibody molecule of claim 1, which binds to one, two, or all of:
(a) one or more ZIKV strains chosen from Zika virus isolate Brazil-ZKV2015 (KU497555.1), Zika virus isolate SSABR1 (KU707826.1), Zika virus strain Natal RGN (KU527068.1), Zika virus strain BeH815744 (KU365780.1), Zika virus strain BeH819966 (KU365779.1), Zika virus strain BeH819015 (KU365778.1), Zika virus strain BeH818995 (KU365777.1), Zika virus strain ZikaSPH2015 (KU321639.1), Zika virus isolate Si322 (KU646828.1), Zika virus isolate Si323 (KU646827.1), Zika virus strain 103344 (KU501216.1), Zika virus strain 8375 (KU501217.1), Zika virus strain MRS_OPY_Martinique_PaRi_2015 (KU647676.1), Zika virus isolate Z1106027 (KU312315.1), Zika virus strain PRVABC59 (KU501215.1), Zika virus isolate Z1106032 (KU312313.1), Zika virus isolate Z1106031 (KU312314.1), Zika virus isolate Z1106033 (KU312312), Zika virus strain Haiti/1225/2014 (KU509998.1), Zika virus strain CK-ISL 2014 (KJ634273.1), Zika virus isolate Zika virus/H.sapiens-tc/THA/2014/SV0127-14 (KU681081.3), Zika virus strain H/PF/2013 (KJ776791.1), Zika virus strain PLCal_ZV (KF993678.1), Zika virus isolate Zika virus/H.sapiens-tc/PHL/2012/CPC-0740 (KU681082.3), Zika virus associated with an epidemic, Yap State, Micronesia, 2007 (EU545988.1), Zika virus isolate ARB13565 (KF268948.1), Zika virus isolate ArD_41519 (HQ234501.1), Zika virus isolate IbH_30656 (HQ234500.1), Zika virus isolate P6-740 (HQ234499.1), Zika virus strain (YP002790881.1), Zika virus strain (NC_012532.1), Zika virus isolate MR766-NIID (LC002520.1), Zika virus isolate MR_766 (HQ234498.1), Zika virus strain MR 766 (AY632535.2; DQ859059.1), Zika virus strain ArD158095 (KF383121.1), Zika virus isolate ARB15076 (KF268949.1), Zika virus isolate ARB7701 (KF268950.1), Zika virus strain ArD158084 (KF383119.1), Zika virus strain ArD157995 (KF383118.1), or Zika virus strain ArD7117 (KF383116.1);
(b) binds to a Zika virus strain comprising a glycosylation site at N154; or
(c) binds to a Zika virus strain that confers neurovirulence.

12. The antibody molecule of claim 1, which is a Fab, F(ab′)2, Fv, or a single chain Fv fragment (scFv), or comprises a heavy chain constant region chosen from IgG1, IgG2, IgG3, or IgG4, and/or a light chain constant region chosen from the light chain constant region of kappa or lambda.

13. The antibody molecule of claim 1, which is an isolated antibody molecule, a synthetic antibody molecule, and/or a humanized antibody molecule, or comprises one or more framework regions derived from a human framework germline sequence.

14. An antibody molecule that competes with the antibody molecule of claim 1 for binding to Zika virus, the E protein of Zika virus, or domain III of the E protein.

15. An antibody molecule that binds to the same epitope as, or an epitope that overlaps with, the epitope of the antibody molecule of claim 1.

16. A method of treating or preventing a Zika virus infection, the method comprising administering to a subject in need thereof the anti-ZIKV antibody molecule of claim 1, in an amount effective to treat or prevent the Zika virus infection, thereby treating or preventing the Zika virus infection.

17. The method of claim 16, wherein the antibody molecule is administered to the subject at dose of about 1 mg/kg to about 50 mg/kg, about 5 mg/kg to about 25 mg/kg, about 5 mg/kg to about 20 mg/kg, about 5 mg/kg to about 15 mg/kg, about 5 mg/kg to about 10 mg/kg, about 20 mg/kg to about 25 mg/kg, about 15 mg/kg to about 25 mg/kg, about 10 mg/kg to about 25 mg/kg, about 10 mg/kg to about 40 mg/kg, about 20 mg/kg to about 30 mg/kg, about 25 mg/kg or less, about 20 mg/kg or less, about 15 mg/kg or less, about 10 mg/kg or less, about 5 mg/kg or less, about 2 mg/kg or less, or about 1 mg/kg or less.

18. The method of claim 16, wherein the antibody molecule is administered to the subject
(a) after the subject is exposed to Zika virus, or is known or suspected to be exposed to Zika virus;
(b) less than about 96 hours, less than about 72 hours, less than about 60 hours, less than about 48 hours, less than about 36 hours, less than about 24 hours, less than about 12 hours, or less than about 6 hours, after the subject is exposed to Zika virus, or is known or suspected to be exposed to Zika virus;
(c) prior to the subject being exposed to Zika virus, or known or suspected to be exposed to Zika virus;
(d) after, or in response to, one or more manifestations of a Zika virus infection chosen from fever, rash, joint pain, headache, vomiting, conjunctivitis, or a combination thereof;
(e) less than about 96 hours, less than about 72 hours, less than about 60 hours, less than about 48 hours, less than about 36 hours, less than about 24 hours, less than about 12 hours, or less than about 6 hours, after the subject exhibits one or more manifestations of Zika virus chosen from fever, rash, joint pain, headache, vomiting, conjunctivitis, or a combination thereof; or
(f) prior to onset of one or more manifestations of a Zika virus infection chosen from fever, rash, joint pain, headache, vomiting, conjunctivitis, or a combination thereof.

19. The method of claim 16, wherein the subject:
(a) is pregnant human female, or a pregnant human female in the first, second or third trimester of pregnancy;
(b) is about 24 months of age or younger, about 18 months of age or younger, about 12 months of age or younger, about 6 months of age or younger, about 3 months of age or younger, about 2 months of age or younger, about 1 months of age or younger, or about 2 weeks of age or younger;
(c) has, or is at risk of having, Zika fever;
(d) has, or is at risk of having, microcephaly, or is at risk of giving birth to a child having microcephaly;
(e) has, or is at risk of having, Guillain-Barrd syndrome (GBS);
(f) exhibits, or is at risk of exhibiting, one or more neurological abnormalities;
(g) is at risk of being infected, or has been infected, with dengue virus (DENV), Japanese encephalitis virus (JEV), West Nile virus (WNV), chikungunya virus (CHIKV), or a combination thereof;
(h) has developed, or is detected to have, an anti-DENV antibody, an anti-JEV antibody, an anti-WNV antibody, an anti-CHIKV antibody, or a combination thereof.

20. The method of claim 16, wherein the antibody molecule is administered to the subject by parenteral, intravenous, intramuscular, or subcutaneous administration.

21. The method of claim 16, wherein the antibody molecule is administered in combination with a second therapeutic agent or different therapeutic modality.

22. The method of any of claim 21, wherein the second therapeutic agent or different therapeutic modality is chosen from a 4-aminoquinoline derivative, a nucleoside inhibitor, an interferon, an RNA-dependent RNA polymerase (RdRp) inhibitor, an adenosine analogue, a vaccine, a second antibody molecule, or a combination thereof.

23. The method of claim 22, wherein the Zika vaccine is chosen from an inactivated purified vaccine, a virus-like particle (VLP), a YF17DD chimeric vaccine, a DNA vaccine, a live dengue recombinant vaccine, a DNA vaccine, a live recombinant adenovirus, a recombinant Zika viral protein, a lentivirus-vectored vaccine, a measles-vectored vaccine, a Zika targeted mutation live attenuated vaccine, a live VSV recombinant vaccine, an E protein, a synthetic replilink peptide, ChimeriVax (YF17D), or a combination thereof.

24. The method of claim 16, wherein the antibody molecule is administered to reduce a risk of enhancing a flavivirus infection in the subject.

25. The method of claim 16, which treats the Zika virus infection.

26. The method of claim 16, which prevents the Zika virus infection.

27. A method of inhibiting Zika virus, the method comprising contacting a cell or a subject infected with Zika virus with the anti-ZIKV antibody molecule of claim 1, in an amount effective to inhibit Zika virus, thereby inhibiting Zika virus.

28. The method of claim 27, wherein the antibody molecule is contacted with the cell in vitro, ex vivo, or in vivo.

29. The method of claim 27, comprising administering to the subject the antibody molecule.

30. A method of reducing a risk of enhancing a flavivirus infection in a subject, the method comprising:
(a) identify a subject infected, or is at risk of being infected, with Zika virus; and
(b) administering to the subject an anti-ZIKV antibody molecule that is not cross-reactive with the flavivirus,
thereby reducing the risk of enhancing the flavivirus infection.

31. The method of claim 30, wherein the flavivirus is a dengue virus.

32. The method of claim 30, wherein the anti-ZIKV antibody molecule comprises a VH and a VL, wherein the VH comprises three heavy chain complementarity determining regions (HCDR1, HCDR2, and HCDR3), and the VL comprises three light chain complementarity determining regions (LCDR1, LCDR2, and LCDR3), and wherein:
(a) the VH comprises: (i) an HCDR1 comprising the amino acid sequence G-Y-S/T-T-T/S-Y (SEQ ID NO: 351); (ii) an HCDR2 comprising the amino acid sequence Y-P-R-S-N/G-N/H (SEQ ID NO: 352); and (iii) an HCDR3 comprising the amino acid sequence E-N/D-Y-G-S-V/T/D-Y (SEQ ID NO: 353); and the VL comprises: (i) an LCDR1 comprising the amino acid sequence K/R-A-S-Q-S/N-V-G-T/E-A-V-A (SEQ ID NO: 354); (ii) an LCDR2 comprising the amino acid sequence S-A-S-N/D-R/L-Y-T (SEQ ID NO: 355); and (iii) an LCDR3 comprising the amino acid sequence Q-Q-F-S/Y-N/S-Y-P-F/Y-T (SEQ ID NO: 356); or
(b) the VH comprises: (i) an HCDR1 comprising the amino acid sequence S/T-Y-G-I-S(SEQ ID NO: 357); (ii) an HCDR2 comprising the amino acid sequence V-I-Y-P-R-S-N/G-N/H-T-Y-Y-N/A-E/Q-R/K-F/L-R/Q-G (SEQ ID NO: 358); and (iii) an HCDR3 comprising the amino acid sequence E-N/D-Y-G-S-V/T/D-Y (SEQ ID NO: 353); and the VL comprises: (i) an LCDR1 comprising the amino acid sequence K/R-A-S-Q-S/N-V-G-T/E-A-V-A (SEQ ID NO: 354); (ii) an LCDR2 comprising the amino acid sequence S-A-S-N/D-R/L-Y-T (SEQ ID NO: 355); and (iii) an LCDR3 comprising the amino acid sequence Q-Q-F-S/Y-N/S-Y-P-F/Y-T (SEQ ID NO: 356).

33. A method of detecting Zika virus in a sample from a subject, the method comprising:
(i) contacting the sample with the anti-ZIKV antibody molecule of claim 1, under conditions that allow interaction of the antibody molecule and Zika virus or a polypeptide from Zika virus to occur, and
(ii) detecting formation of a complex between the antibody molecule and the sample,
thereby detecting Zika virus.

34. A pharmaceutical composition comprising the anti-ZIKV antibody molecule of claim 1 and a pharmaceutical acceptable carrier.

35. A nucleic acid molecule encoding a heavy chain variable region (VH), a light chain variable region (VL), or both, of the antibody molecule of claim 1.

36. A vector comprising the nucleic acid molecule of claim 35.

37. A cell comprising a nucleic acid molecule of claim 35.

38. A kit comprising the antibody molecule of claim 1 and instructions to use of the antibody molecule.

39. A container comprising the antibody molecule of claim 1.

40. A method of producing an anti-ZIKV antibody molecule, the method comprising culturing a cell of claim 37 under conditions that allow production of an antibody molecule described herein, thereby producing the antibody molecule.

41. A vaccine comprising an immunogen comprising an epitope bound by the antibody molecule of claim 1.


SEQUENCE LISTING (470 sequences)

SEQ ID NO: 1 - protein, 116 aa, Artificial Sequence
  Description of Artificial Sequence: Synthetic polypeptide
QVQLQQSGVGLARPGTSVKLSCKASGYSFTTYGISWVTQRPGQGLEWIGVIYPRSNNTYYNERFRGKATLTADKSSSSAYLELRGLTAEDSAVYFCARENYGSVYWGQGTTLTVSS

SEQ ID NO: 2 - protein, 107 aa, Artificial Sequence
  Description of Artificial Sequence: Synthetic polypeptide
DIVMTQSQKFMSTSVGDRVTITCKASQSVGTAVAWYHQKPGQSPKLLIYSASNRYTGVPDRFTGSGSGTDFTLTITYMQSEDLADYFCQQFSNYPFTFGGGTKLEIK

SEQ ID NO: 3 - protein, 116 aa, Artificial Sequence
  Description of Artificial Sequence: Synthetic polypeptide
QVQLQQSGTGLARPGASVKLSCKASGYTFTSYGISWVTQRAGQGLEWIGVIYPRSGNTYYNEKFRGKATLTADKSSSSAYMELRGLTAEDSAVYFCARENYGSVYWGQGTTLTVSS

SEQ ID NO: 4 - protein, 107 aa, Artificial Sequence
  Description of Artificial Sequence: Synthetic polypeptide
DIVMTQSQKFMSTSVGDRVSITCKASQNVGTAVAWYQQKPGQSPKLLIYSASNRYTGVPDRFTGSGSGTDFTLTISNMQSEDLADYFCQQFSSYPYTFGGGTKLEIK

SEQ ID NO: 5 - protein, 116 aa, Artificial Sequence
  Description of Artificial Sequence: Synthetic polypeptide
QVQLVQSGAEVKKPGSSVKVSCKASGYSFTTYGISWVRQAPGQGLEWMGVIYPRSGNTYYAQKFQGRVTITADKSTSTAYMELSSLRSEDTAVYFCARENYGSVYWGQGTLVTVSS

SEQ ID NO: 6 - protein, 107 aa, Artificial Sequence
  Description of Artificial Sequence: Synthetic polypeptide
EIVMTQSPATLSVSPGERATLSCRASQSVGTAVAWYQQKPGQAPRLLIYSASNRYTGIPARFSGSGSGTEFTLTISSLQSEDFADYFCQQFSNYPFTFGGGTKVEIK

SEQ ID NO: 7 - protein, 107 aa, Artificial Sequence
  Description of Artificial Sequence: Synthetic polypeptide
DIQMTQSPSSLSASVGDRVTITCKASQSVGTAVAWYQQKPGKAPKLLIYSASNRYTGVPSRFSGSGSGTDFTLTISSLQPEDFATYFCQQFSNYPFTFGGGTKVEIK

SEQ ID NO: 8 - protein, 107 aa, Artificial Sequence
  Description of Artificial Sequence: Synthetic polypeptide
DIRMTQSPSSFSASTGDRVTITCRASQSVGTAVAWYQQKPGKAPKLLIYSASNLYTGVPSRFSGSGSGTDFTLTISCLQSEDFATYFCQQFSNYPFTFGGGTKVEIK

SEQ ID NO: 9 - protein, 116 aa, Artificial Sequence
  Description of Artificial Sequence: Synthetic polypeptide
QVQLVQSGAEVKKPGSSVKVSCKASGYSFTTYGISWVRQAPGQGLEWMGVIYPRSGNTYYAEKFRGRVTITADKSTSTAYMELSSLRSEDTAVYYCARENYGSVYWGQGTLVTVSS

SEQ ID NO: 10 - protein, 116 aa, Artificial Sequence
  Description of Artificial Sequence: Synthetic polypeptide
QVQLVQSGAEVKKPGASVKVSCKASGYSFTTYGISWVRQAPGQGLEWMGVIYPRSGNTYYAQKLQGRVTMTTDTSTSTAYMELRSLRSDDTAVYYCARENYGSVYWGQGTLVTVSS

SEQ ID NO: 11 - protein, 116 aa, Artificial Sequence
  Description of Artificial Sequence: Synthetic polypeptide
QVQLVQSGAEVKKPGASVKVSCKASGYSFTTYGISWVRQAPGQGLEWMGVIYPRSGNTYYAEKLRGRVTMTADKSTSTAYMELRSLRSDDTAVYYCARENYGSVYWGQGTLVTVSS

SEQ ID NO: 12 - protein, 116 aa, Artificial Sequence
  Description of Artificial Sequence: Synthetic polypeptide
QVQLVQSGAEVKKPGASVKVSCKASGYSFTTYGISWVRQATGQGLEWMGVIYPRSGNTYYAEKFRGRVTMTANKSISTAYMELSSLRSEDTAVYYCARENYGSVYWGQGTLVTVSS

SEQ ID NO: 13 - protein, 116 aa, Artificial Sequence
  Description of Artificial Sequence: Synthetic polypeptide
QVQLVQSGAEVKKPGASVKVSCKASGYTFTSYGISWVRQATGQGLEWMGVIYPRSGNTYYAEKFRGRVTMTADKSISTAYMELSSLRSEDTAVYYCARENYGSVYWGQGTLVTVSS

SEQ ID NO: 14 - protein, 116 aa, Artificial Sequence
  Description of Artificial Sequence: Synthetic polypeptide
QVQLVQSGAEVKKPGASVKVSCKASGYTFTSYGISWVRQATGQGLEWMGVIYPRSGNTYYAEKFRGRVTMTADKSISTAYMELSSLRSEDTAVYYCAREDYGSVYWGQGTLVTVSS

SEQ ID NO: 15 - protein, 116 aa, Artificial Sequence
  Description of Artificial Sequence: Synthetic polypeptide
QVQLVQSGAEVKKPGASVKVSCKASGYTFTSYGISWVRQATGQGLEWMGVIYPRSGHTYYAEKFRGRVTMTADKSISTAYMELSSLRSEDTAVYYCARENYGSVYWGQGTLVTVSS

SEQ ID NO: 16 - protein, 116 aa, Artificial Sequence
  Description of Artificial Sequence: Synthetic polypeptide
QVQLVQSGAEVKKPGASVKVSCKASGYTFTSYGISWVRQATGQGLEWMGVIYPRSGNTYYAEKFRGRVTMTADKSISTAYMELSSLRSEDTAVYYCARENYGSTYWGQGTLVTVSS

SEQ ID NO: 17 - protein, 116 aa, Artificial Sequence
  Description of Artificial Sequence: Synthetic polypeptide
QVQLVQSGAEVKKPGASVKVSCKASGYTFTSYGISWVRQATGQGLEWMGVIYPRSGNTYYAEKFRGRVTMTADKSISTAYMELSSLRSEDTAVYYCARENYGSDYWGQGTLVTVSS

SEQ ID NO: 18 - protein, 107 aa, Artificial Sequence
  Description of Artificial Sequence: Synthetic polypeptide
EIVMTQSPATLSVSPGERATLSCRASQSVGEAVAWYQQKPGQAPRLLIYSASNRYTGIPARFSGSGSGTEFTLTISSLQSEDFADYFCQQFSNYPFTFGGGTKVEIK

SEQ ID NO: 19 - protein, 107 aa, Artificial Sequence
  Description of Artificial Sequence: Synthetic polypeptide
EIVMTQSPATLSVSPGERATLSCRASQSVGTAVAWYQQKPGQAPRLLIYSASDRYTGIPARFSGSGSGTEFTLTISSLQSEDFADYFCQQFSNYPFTFGGGTKVEIK

SEQ ID NO: 20 - protein, 107 aa, Artificial Sequence
  Description of Artificial Sequence: Synthetic polypeptide
EIVMTQSPATLSVSPGERATLSCRASQSVGTAVAWYQQKPGQAPRLLIYSASNRYTGIPARFSGSGSGTEFTLTISSLQSEDFADYFCQQFYNYPFTFGGGTKVEIK

SEQ ID NO: 21 - protein, 107 aa, Artificial Sequence
  Description of Artificial Sequence: Synthetic polypeptide
DIQMTQSPSSLSASVGDRVTITCKASQSVGTAVAWYQQKPGKAPKLLIYSASNRYTGVPSRFSGSGSGTDFTLTISSLQPEDFATYFCQQFYNYPFTFGGGTKVEIK

SEQ ID NO: 22 - protein, 116 aa, Artificial Sequence
  Description of Artificial Sequence: Synthetic polypeptide
QVQLVQSGAEVKKPGASVKVSCKASGYTFTSYGISWVRQAPGQGLEWMGVIYPRSGNTYYAQKLQGRVTMTTDTSTSTAYMELRSLRSDDTAVYYCARENYGSVYWGQGTLVTVSS

SEQ ID NO: 23-100 - skipped (78 entries)

SEQ ID NO: 101 - protein, 7 aa, Artificial Sequence
  Description of Artificial Sequence: Synthetic peptide
GYSFTTY

SEQ ID NO: 102 - protein, 6 aa, Artificial Sequence
  Description of Artificial Sequence: Synthetic peptide
YPRSNN

SEQ ID NO: 103 - protein, 7 aa, Artificial Sequence
  Description of Artificial Sequence: Synthetic peptide
ENYGSVY

SEQ ID NO: 104 - protein, 11 aa, Artificial Sequence
  Description of Artificial Sequence: Synthetic peptide
KASQSVGTAVA

SEQ ID NO: 105 - protein, 7 aa, Artificial Sequence
  Description of Artificial Sequence: Synthetic peptide
SASNRYT

SEQ ID NO: 106 - protein, 9 aa, Artificial Sequence
  Description of Artificial Sequence: Synthetic peptide
QQFSNYPFT

SEQ ID NO: 107 - protein, 7 aa, Artificial Sequence
  Description of Artificial Sequence: Synthetic peptide
GYTFTSY

SEQ ID NO: 108 - protein, 6 aa, Artificial Sequence
  Description of Artificial Sequence: Synthetic peptide
YPRSGN

SEQ ID NO: 109 - protein, 11 aa, Artificial Sequence
  Description of Artificial Sequence: Synthetic peptide
KASQNVGTAVA

SEQ ID NO: 110 - protein, 9 aa, Artificial Sequence
  Description of Artificial Sequence: Synthetic peptide
QQFSSYPYT

SEQ ID NO: 111 - protein, 11 aa, Artificial Sequence
  Description of Artificial Sequence: Synthetic peptide
RASQSVGTAVA

SEQ ID NO: 112 - protein, 7 aa, Artificial Sequence
  Description of Artificial Sequence: Synthetic peptide
SASNLYT

SEQ ID NO: 113 - protein, 7 aa, Artificial Sequence
  Description of Artificial Sequence: Synthetic peptide
EDYGSVY

SEQ ID NO: 114 - protein, 6 aa, Artificial Sequence
  Description of Artificial Sequence: Synthetic peptide
YPRSGH

SEQ ID NO: 115 - protein, 7 aa, Artificial Sequence
  Description of Artificial Sequence: Synthetic peptide
ENYGSTY

SEQ ID NO: 116 - protein, 7 aa, Artificial Sequence
  Description of Artificial Sequence: Synthetic peptide
ENYGSDY

SEQ ID NO: 117 - protein, 11 aa, Artificial Sequence
  Description of Artificial Sequence: Synthetic peptide
RASQSVGEAVA

SEQ ID NO: 118 - protein, 7 aa, Artificial Sequence
  Description of Artificial Sequence: Synthetic peptide
SASDRYT

SEQ ID NO: 119 - protein, 9 aa, Artificial Sequence
  Description of Artificial Sequence: Synthetic peptide
QQFYNYPFT

SEQ ID NO: 120-200 - skipped (81 entries)

SEQ ID NO: 201 - protein, 5 aa, Artificial Sequence
  Description of Artificial Sequence: Synthetic peptide
TYGIS

SEQ ID NO: 202 - protein, 17 aa, Artificial Sequence
  Description of Artificial Sequence: Synthetic peptide
VIYPRSNNTYYNERFRG

SEQ ID NO: 203 - protein, 5 aa, Artificial Sequence
  Description of Artificial Sequence: Synthetic peptide
SYGIS

SEQ ID NO: 204 - protein, 17 aa, Artificial Sequence
  Description of Artificial Sequence: Synthetic peptide
VIYPRSGNTYYNEKFRG

SEQ ID NO: 205 - protein, 17 aa, Artificial Sequence
  Description of Artificial Sequence: Synthetic peptide
VIYPRSGNTYYAQKFQG

SEQ ID NO: 206 - protein, 17 aa, Artificial Sequence
  Description of Artificial Sequence: Synthetic peptide
VIYPRSGNTYYAEKFRG

SEQ ID NO: 207 - protein, 17 aa, Artificial Sequence
  Description of Artificial Sequence: Synthetic peptide
VIYPRSGNTYYAQKLQG

SEQ ID NO: 208 - protein, 17 aa, Artificial Sequence
  Description of Artificial Sequence: Synthetic peptide
VIYPRSGNTYYAEKLRG

SEQ ID NO: 209 - protein, 17 aa, Artificial Sequence
  Description of Artificial Sequence: Synthetic peptide
VIYPRSGHTYYAEKFRG

SEQ ID NO: 210-250 - skipped (41 entries)

SEQ ID NO: 251 - protein, 111 aa, Artificial Sequence
  Description of Artificial Sequence: Synthetic polypeptide
RLKGVSYSLCTAAFTFTKIPADTGHGTVTVEVQYAGTDGPCKVPASSVASLNDLTPVGRLVTVNPVITESTENSKMMLELEPPFGDSYIVIGVGEKKITHHWHKSGSSIGK

SEQ ID NO: 252 - protein, 111 aa, Artificial Sequence
  Description of Artificial Sequence: Synthetic polypeptide
QLKGTTYGVCSKAFKFLGTPAETLHGTVVLELQYTGTDGPCKVPIQMAVDMQTLTPVGRLITANPFVSVATANAKVLIELDPPFGDSYIVVGRGEQQINHHWHRSGSTIGK

SEQ ID NO: 253 - protein, 110 aa, Artificial Sequence
  Description of Artificial Sequence: Synthetic polypeptide
RLKGVSYSLCTAAFTFTKIPAETQHGTVTVEVQYAGTDGPCKVPASTQDEKGATQNGRLITANPVITESTENSKMMLEAEPPFGESYIVIGVGEKKITHSWFKKGSSIGK

SEQ ID NO: 254 - protein, 111 aa, Artificial Sequence
  Description of Artificial Sequence: Synthetic polypeptide
RLKGVSYSVCTAAFTFTKIPADTGHGTVTVEVQYAGTDGPCKVPASSVASLNDLTPVGRLVTVNPVITESTENSKMMLELEPPFGDSYIVIGRGEQQINHHWHKSGSSIGK

SEQ ID NO: 255 - protein, 111 aa, Artificial Sequence
  Description of Artificial Sequence: Synthetic polypeptide
QLKGTTYGVCSKAFTFTKIPADTGHGTVTVEVQYAGTDGPCKVPASSVASLNDLTPVGRLVTVNPVITESTENSKMMLELEPPFGDSYIVIGRGEQQINHHWHKSGSSIGK

SEQ ID NO: 256 - protein, 111 aa, Artificial Sequence
  Description of Artificial Sequence: Synthetic polypeptide
QLKGTTYGVCSKAFTFLGTPADTGHGTVVVEVQYAGTDGPCKVPASSVASLNDLTPVGRLVTVNPVITESTENSKMLLELEPPFGDSYIVIGRGEQQINHHWHKSGSSIGK

SEQ ID NO: 257 - protein, 111 aa, Artificial Sequence
  Description of Artificial Sequence: Synthetic polypeptide
QLKGTTYGLCTAAFTFTGIPADTGHGTVVLELQYAGTDGPCKVPIQSVASLNDLTPVGRLVTVNPFVSVATANAKVLIELEPPFGDSYIVVGVGEKKITHHWHKSGSSIGK

SEQ ID NO: 258 - protein, 111 aa, Artificial Sequence
  Description of Artificial Sequence: Synthetic polypeptide
QLKGTTYGVCSKAFKFTKTPADTGHGTVTLELQYTGTDGPCKVPISSVASLNDLTPVGRLVTVNPVVTESTENSKMMIELEPPFGDSYIVVGRGEQQINHHWHKSGSSIGK

SEQ ID NO: 259 - protein, 111 aa, Artificial Sequence
  Description of Artificial Sequence: Synthetic polypeptide
QLKGTTYGVCTAAFTFTKTPADTGHGTVVLELQYAGTDGPCKVPISSVASLNDLTPVGRLVTVNPFVSESTENAKVLIELEPPFGDSYIVVGRGEKQINHHWHKSGSSIGK

SEQ ID NO: 260 - protein, 111 aa, Artificial Sequence
  Description of Artificial Sequence: Synthetic polypeptide
RLKGVSYSLCSKAFKFTKIPAETLHGTVTVEVQYTGTDGPCKVPAQMAVDMQTLTPVGRLITANPVITVATANSKMMLELDPPFGDSYIVIGRGEQKITHHWHRSGSTIGK

SEQ ID NO: 261 - protein, 111 aa, Artificial Sequence
  Description of Artificial Sequence: Synthetic polypeptide
QLKGTTYGVCTAAFTFLGTPADTGHGTVVLELQYAGTDGPCKVPISSVASLNDLTPVGRLVTVNPFVSESTENAKVLIELEPPFGDSYIVVGVGEKQINHHWHKSGSSIGK

SEQ ID NO: 262 - protein, 111 aa, Artificial Sequence
  Description of Artificial Sequence: Synthetic polypeptide
RLKGVSYSLCTAAFTFTKIPAETLHGTVTVEVQYAGTDGPCKVPAQMAVDMQTLTPVGRLITANPVIENKTENSKMMLELDPPFGDSYIVIGVGEKKITHHWHRSGSTIGK

SEQ ID NO: 263 - protein, 111 aa, Artificial Sequence
  Description of Artificial Sequence: Synthetic polypeptide
RLKGVSYSLCTEKFSFTKIPAETLHGTVTVEVQYEGTDGPCKVPAQMAVDMQTLTPVGRLITANPVITESTANSKMMLELDPPFGDSYIVIGVGEKKITHHWHRSGSTIGK

SEQ ID NO: 264 - protein, 111 aa, Artificial Sequence
  Description of Artificial Sequence: Synthetic polypeptide
RLKGVSYSLCTKAFTFTKIPAETLHGTVTVEVQYYGTDGPCKVPAQMAVDMQTLTPVGRLITANPVITESTENSKMMLELDPPFGDSYIVIGVGEKKITHHWHRSGSTIGK

SEQ ID NO: 265 - protein, 111 aa, Artificial Sequence
  Description of Artificial Sequence: Synthetic polypeptide
RLKGVSYSLCTAAFTFDKIPAETLHGTVTVEVKYAGTDGPCKVPAQMAVDMQTLTPVGRLITANPVITESTEESSMMLELDPPFGDSYIVIGVGEKKITHHWHRSGSTIGK

SEQ ID NO: 266-299 - skipped (34 entries)

SEQ ID NO: 300 - DNA, 348 bp, Artificial Sequence
  Description of Artificial Sequence: Synthetic polynucleotide
caagtccaactcgtgcaatccggggcagaagtgaagaaaccaggtagctcagtgaaggtatcatgtaaagcaagcgggtacagtttcacgacctacggtatcagttgggtacggcaggctccgggacagggtttggaatggatgggtgtaatttaccctagaagcggtaatacatactatgctcagaagtttcaagggagggtcacgattacagccgacaaaagtacaagcaccgcctacatggagctgtcatccctgcgatcagaagatacggctgtctacttttgcgcccgagagaattatggctccgtctactggggtcagggcacgcttgtcacagtaagttcc

SEQ ID NO: 301 - DNA, 348 bp, Artificial Sequence
  Description of Artificial Sequence: Synthetic polynucleotide
caggttcagttggtgcagtctggagcggaagtaaagaagcctggttccagtgtgaaggtaagttgtaaggctagcggttacagcttcacaacttacggcataagttgggtcagacaagcgccggggcaagggctcgaatggatgggagtcatatatccccggtctggcaacacctattacgctgagaagtttcgaggtcgagtcacaataacggcggataaaagtacctccactgcctacatggagttgtcatctcttcgatctgaggatacggctgtctactactgcgcacgagaaaattatggctccgtctactggggacaggggacattggtcacggtcagtagt

SEQ ID NO: 302 - DNA, 348 bp, Artificial Sequence
  Description of Artificial Sequence: Synthetic polynucleotide
caagtacaactcgtacaaagtggcgcagaggtgaaaaaacccggagcttcagtgaaggtcagttgtaaggcatctggttactccttcaccacttacggaataagttgggtacggcaggcaccgggccaggggcttgagtggatgggagtgatttacccccgcagcggtaatacctattatgcacaaaaactccaggggcgggtaaccatgactaccgacacttctacttctactgcgtacatggagttgagatcattgaggtctgatgatacagcggtttactactgcgctcgagagaactatggttccgtgtattggggccaaggcacattggtaacagtttcttcc

SEQ ID NO: 303 - DNA, 348 bp, Artificial Sequence
  Description of Artificial Sequence: Synthetic polynucleotide
caggttcaactggttcaatctggggctgaagtcaagaaaccaggcgcgtcagtgaaagtctcttgcaaagcgtctggttattctttcacgacatacggtatatcatgggttcgacaagcccctggacagggtttggaatggatgggggtcatttatcccagaagtggcaatacctattatgcggagaagctcaggggccgggtaacgatgaccgcggacaaaagtaccagcaccgcttatatggaacttcgatctctcagatcagatgacacggcagtctactactgcgcccgcgaaaactacgggtcagtgtactgggggcagggcactctggtcactgtatctagt

SEQ ID NO: 304 - DNA, 348 bp, Artificial Sequence
  Description of Artificial Sequence: Synthetic polynucleotide
caggtacaactcgtgcagagcggggccgaagtcaagaagcccggcgcatccgttaaagtatcatgcaaggcgtcaggatattctttcacaacttatggcatttcttgggttcggcaggctaccggccaaggtttggaatggatgggcgttatctaccctaggtccggaaatacctattatgctgaaaagtttagaggtcgcgttactatgaccgcaaacaagtcaatttctaccgcctacatggaactctcaagcctgaggagtgaggacactgctgtatattactgtgcgcgggaaaattatgggagtgtgtactggggccaggggactttggttacggtttcatcc

SEQ ID NO: 305 - DNA, 321 bp, Artificial Sequence
  Description of Artificial Sequence: Synthetic polynucleotide
gagatagttatgacccagagcccagctacattgtccgtgtcccccggtgaaagagccacgctgagttgtagagcgtcacagagtgtagggacggcggttgcgtggtatcaacagaagccagggcaggcaccaaggctcttgatttattcagcatccaatcgatacactggcattccagctagattctcaggttctggttcaggcacggaatttacattgacaatctcatccctgcaatcagaagatttcgccgattatttctgtcaacaattctccaattacccattcacgtttggaggagggacaaaagtggaaatcaaa

SEQ ID NO: 306 - DNA, 321 bp, Artificial Sequence
  Description of Artificial Sequence: Synthetic polynucleotide
gacattcaaatgacacagagtccttctagccttagcgcctcagtaggtgacagggttacgatcacctgcaaagcgagccagagcgttggaactgcagttgcgtggtatcagcaaaagcctgggaaggctcccaagctgctcatatattccgcctcaaaccgctatacaggagttcccagcagattttcaggatctggatctggcacggatttcacgctgaccatcagttcactgcaaccggaagacttcgctacctatttttgtcagcagttttcaaactacccttttactttcgggggtggaaccaaagtggaaattaaa

SEQ ID NO: 307 - DNA, 321 bp, Artificial Sequence
  Description of Artificial Sequence: Synthetic polynucleotide
gacatcagaatgactcaaagtccctctagtttttcagcctctacaggcgatcgagttactataacgtgtcgggctagccagtccgttggaaccgccgtcgcctggtatcagcaaaaacctggaaaggccccgaaattgctgatatactctgcttctaatctttacactggcgttccctccaggttttctggctcagggagcggtactgattttacgctgacaatttcttgtcttcaatcagaggacttcgccacctatttttgtcaacagttttcaaattacccgtttacgttcggcggcgggaccaaggtcgaaattaag

SEQ ID NO: 308 - DNA, 348 bp, Artificial Sequence
  Description of Artificial Sequence: Synthetic polynucleotide
caagtgcaactcgttcagtcaggcgcagaggtcaagaagcctggtgcatccgtcaaagtttcatgcaaggcctccggatatacattcacctcctatggaataagctgggtccggcaggcaaccggccagggtcttgaatggatgggggtgatatatccaagatccggaaacacatactacgccgagaaatttcgtgggcgcgtcaccatgacagccgacaaaagcatatctaccgcctatatggaactttctagccttcgttctgaggatacagcagtgtattattgtgccagggagaactacggatctgtctattggggccagggtacactcgtaacagttagcagc

SEQ ID NO: 309 - DNA, 348 bp, Artificial Sequence
  Description of Artificial Sequence: Synthetic polynucleotide
caagtgcaactcgttcagtcaggcgcagaggtcaagaagcctggtgcatccgtcaaagtttcatgcaaggcctccggatatacattcacctcctatggaataagctgggtccggcaggcaaccggccagggtcttgaatggatgggggtgatatatccaagatccggaaacacatactacgccgagaaatttcgtgggcgcgtcaccatgacagccgacaaaagcatatctaccgcctatatggaactttctagccttcgttctgaggatacagcagtgtattattgtgccagggaggactacggatctgtctattggggccagggtacactcgtaacagttagcagc

SEQ ID NO: 310 - DNA, 348 bp, Artificial Sequence
  Description of Artificial Sequence: Synthetic polynucleotide
caagtgcaactcgttcagtcaggcgcagaggtcaagaagcctggtgcatccgtcaaagtttcatgcaaggcctccggatatacattcacctcctatggaataagctgggtccggcaggcaaccggccagggtcttgaatggatgggggtgatatatccaagatccggacacacatactacgccgagaaatttcgtgggcgcgtcaccatgacagccgacaaaagcatatctaccgcctatatggaactttctagccttcgttctgaggatacagcagtgtattattgtgccagggagaactacggatctgtctattggggccagggtacactcgtaacagttagcagc

SEQ ID NO: 311 - DNA, 348 bp, Artificial Sequence
  Description of Artificial Sequence: Synthetic polynucleotide
caagtgcaactcgttcagtcaggcgcagaggtcaagaagcctggtgcatccgtcaaagtttcatgcaaggcctccggatatacattcacctcctatggaataagctgggtccggcaggcaaccggccagggtcttgaatggatgggggtgatatatccaagatccggaaacacatactacgccgagaaatttcgtgggcgcgtcaccatgacagccgacaaaagcatatctaccgcctatatggaactttctagccttcgttctgaggatacagcagtgtattattgtgccagggagaactacggatctacctattggggccagggtacactcgtaacagttagcagc

SEQ ID NO: 312 - DNA, 348 bp, Artificial Sequence
  Description of Artificial Sequence: Synthetic polynucleotide
caagtgcaactcgttcagtcaggcgcagaggtcaagaagcctggtgcatccgtcaaagtttcatgcaaggcctccggatatacattcacctcctatggaataagctgggtccggcaggcaaccggccagggtcttgaatggatgggggtgatatatccaagatccggaaacacatactacgccgagaaatttcgtgggcgcgtcaccatgacagccgacaaaagcatatctaccgcctatatggaactttctagccttcgttctgaggatacagcagtgtattattgtgccagggagaactacggatctgactattggggccagggtacactcgtaacagttagcagc

SEQ ID NO: 313 - DNA, 321 bp, Artificial Sequence
  Description of Artificial Sequence: Synthetic polynucleotide
gaaatcgtaatgacccagagtcctgccactctgtcagtgtctccaggtgagcgtgcaactctctcttgccgcgcctctcaatctgtggggaccgctgtggcatggtaccagcaaaaacccggacaggcccctcgtcttctcatctattctgcatctaaccgatacactgggatccctgccaggttttctgggagtgggtcaggaaccgaatttaccctcacaatttcttctttgcaaagcgaagattttgccgattacttctgccaacaattctccaactacccatttacctttggtggaggtactaaagtggagataaaa

SEQ ID NO: 314 - DNA, 321 bp, Artificial Sequence
  Description of Artificial Sequence: Synthetic polynucleotide
gaaatcgtaatgacccagagtcctgccactctgtcagtgtctccaggtgagcgtgcaactctctcttgccgcgcctctcaatctgtgggggaggctgtggcatggtaccagcaaaaacccggacaggcccctcgtcttctcatctattctgcatctaaccgatacactgggatccctgccaggttttctgggagtgggtcaggaaccgaatttaccctcacaatttcttctttgcaaagcgaagattttgccgattacttctgccaacaattctccaactacccatttacctttggtggaggtactaaagtggagataaaa

SEQ ID NO: 315 - DNA, 321 bp, Artificial Sequence
  Description of Artificial Sequence: Synthetic polynucleotide
gaaatcgtaatgacccagagtcctgccactctgtcagtgtctccaggtgagcgtgcaactctctcttgccgcgcctctcaatctgtggggaccgctgtggcatggtaccagcaaaaacccggacaggcccctcgtcttctcatctattctgcatctgaccgatacactgggatccctgccaggttttctgggagtgggtcaggaaccgaatttaccctcacaatttcttctttgcaaagcgaagattttgccgattacttctgccaacaattctccaactacccatttacctttggtggaggtactaaagtggagataaaa

SEQ ID NO: 316 - DNA, 321 bp, Artificial Sequence
  Description of Artificial Sequence: Synthetic polynucleotide
gaaatcgtaatgacccagagtcctgccactctgtcagtgtctccaggtgagcgtgcaactctctcttgccgcgcctctcaatctgtggggaccgctgtggcatggtaccagcaaaaacccggacaggcccctcgtcttctcatctattctgcatctaaccgatacactgggatccctgccaggttttctgggagtgggtcaggaaccgaatttaccctcacaatttcttctttgcaaagcgaagattttgccgattacttctgccaacaattctacaactacccatttacctttggtggaggtactaaagtggagataaaa

SEQ ID NO: 317 - DNA, 348 bp, Artificial Sequence
  Description of Artificial Sequence: Synthetic polynucleotide
caagtacaactcgtacaaagtggcgcagaggtgaaaaaacccggagcttcagtgaaggtcagttgtaaggcatctggttacaccttcacctcttacggaataagttgggtacggcaggcaccgggccaggggcttgagtggatgggagtgatttacccccgcagcggtaatacctattatgcacaaaaactccaggggcgggtaaccatgactaccgacacttctacttctactgcgtacatggagttgagatcattgaggtctgatgatacagcggtttactactgcgctcgagagaactatggttccgtgtattggggccaaggcacattggtaacagtttcttcc

SEQ ID NO: 318 - DNA, 321 bp, Artificial Sequence
  Description of Artificial Sequence: Synthetic polynucleotide
gacattcaaatgacacagagtccttctagccttagcgcctcagtaggtgacagggttacgatcacctgcaaagcgagccagagcgttggaactgcagttgcgtggtatcagcaaaagcctgggaaggctcccaagctgctcatatattccgcctcaaaccgctatacaggagttcccagcagattttcaggatctggatctggcacggatttcacgctgaccatcagttcactgcaaccggaagacttcgctacctatttttgtcagcagttttacaactacccttttactttcgggggtggaaccaaagtggaaattaaa

SEQ ID NO: 319-350 - skipped (32 entries)

SEQ ID NO: 351 - protein, 6 aa, Artificial Sequence
  Description of Artificial Sequence: Synthetic peptide
  MOD_RES (3)..(3): Ser or Thr
  MOD_RES (5)..(5): Thr or Ser
GYXTXY

SEQ ID NO: 352 - protein, 6 aa, Artificial Sequence
  Description of Artificial Sequence: Synthetic peptide
  MOD_RES (5)..(5): Asn or Gly
  MOD_RES (6)..(6): Asn or His
YPRSXX

SEQ ID NO: 353 - protein, 7 aa, Artificial Sequence
  Description of Artificial Sequence: Synthetic peptide
  MOD_RES (2)..(2): Asn or Asp
  MOD_RES (6)..(6): Val, Thr or Asp
EXYGSXY

SEQ ID NO: 354 - protein, 11 aa, Artificial Sequence
  Description of Artificial Sequence: Synthetic peptide
  MOD_RES (1)..(1): Lys or Arg
  MOD_RES (5)..(5): Ser or Asn
  MOD_RES (8)..(8): Thr or Glu
XASQXVGXAVA

SEQ ID NO: 355 - protein, 7 aa, Artificial Sequence
  Description of Artificial Sequence: Synthetic peptide
  MOD_RES (4)..(4): Asn or Asp
  MOD_RES (5)..(5): Arg or Leu
SASXXYT

SEQ ID NO: 356 - protein, 9 aa, Artificial Sequence
  Description of Artificial Sequence: Synthetic peptide
  MOD_RES (4)..(4): Ser or Tyr
  MOD_RES (5)..(5): Asn or Ser
  MOD_RES (8)..(8): Phe or Tyr
QQFXXYPXT

SEQ ID NO: 357 - protein, 5 aa, Artificial Sequence
  Description of Artificial Sequence: Synthetic peptide
  MOD_RES (1)..(1): Ser or Thr
XYGIS

SEQ ID NO: 358 - protein, 17 aa, Artificial Sequence
  Description of Artificial Sequence: Synthetic peptide
  MOD_RES (7)..(7): Asn or Gly
  MOD_RES (8)..(8): Asn or His
  MOD_RES (12)..(12): Asn or Ala
  MOD_RES (13)..(13): Glu or Gln
  MOD_RES (14)..(14): Arg or Lys
  MOD_RES (15)..(15): Phe or Leu
  MOD_RES (16)..(16): Arg or Gln
VIYPRSXXTYYXXXXXG

SEQ ID NO: 359-400 - skipped (42 entries)

SEQ ID NO: 401 - protein, 393 aa, Dengue virus
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFK

SEQ ID NO: 402 - protein, 393 aa, Dengue virus
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKVVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFK

SEQ ID NO: 403 - protein, 391 aa, Dengue virus
MRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKIVQHENLKYTVIITVHTGDQHQVGNETQGVTAEITSQASTAEAILPEYGTLGLECSPRTGLDFNEMILLTMKDKAWMVHRQWFFDLPLPWTSGATTKTPTWNRKELLVTFKNAHAKKQEVVVLGSQEGAMHTALTGATEIQTSGGTSIFAGHLKCRLKMDKLKLKGMSYAMCLNTFVLKKEVSETQHGTILIKVEYKGEDAPCKIPFSTEDGQGKAHNGRLITANPVVTKKEEPVNIEAEPPFGESNIVIGIGDKALKINWYR

SEQ ID NO: 404 - protein, 393 aa, Dengue virus
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTAMITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFR

SEQ ID NO: 405 - protein, 398 aa, Japanese encephalitis virus
FNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGFFGKGSIDTCAKFSCTSKAIGRTIQPENIKYKVGIFVHGTTTSENHGNYSAQVGASQAAKFTVTPNAPSVTLKLGDYGEVTLDCEPRSGLNTEAFYVMTVGSKSFLVHREWFHDLALPWTSPSSTAWRNRELLMEFEGAHATKQSVVALGSQEGGLHQALAGAIVVEYSSSVMLTSGHLKCRLKMDKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHK

SEQ ID NO: 406 - protein, 399 aa, West Nile virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQAGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHK

SEQ ID NO: 407 - protein, 402 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHR

SEQ ID NO: 408 - protein, 332 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - protein, 24 aa, Dengue virus
TTTDSRVDRWGNGLGNETEAKKQE

SEQ ID NO: 410 - protein, 24 aa, Dengue virus
TTTESRVDRWGNGIGNDTKAKKQD

SEQ ID NO: 411 - protein, 23 aa, Dengue virus
ITTDSRVDRWGNGLGNETAKKQE

SEQ ID NO: 412 - protein, 24 aa, Dengue virus
ITTATRVDRWGNGVGNDTNAKRQD

SEQ ID NO: 413 - protein, 24 aa, Zika virus
MASDSRVDRWGNGLVNDTHAKRQT

SEQ ID NO: 414 - protein, 86 aa, Dengue virus
TRGGEPHMIVSKQERGKSLLFKTSAGVNMCTLIAMDLGELCEDTMTYKCPRITEAEPDDVDCWCNATDTWVTYGTCSQTGEHRRDK

SEQ ID NO: 415 - protein, 86 aa, Dengue virus
TRNGEPHMIVGRQEKGKSLLFKTEDGVNMCTLMAIDLGELCEDTITYKCPLLRQNEPEDIDCWCNSTSTWVTYGTCTTTGEHRREK

SEQ ID NO: 416 - protein, 86 aa, Dengue virus
SRDGEPRMIVGKNERGKSLLFKTASGINMCTLIAMDLGEMCDDTVTYKCPLIAEVEPEDIDCWCNLTSTWVTYGTCNQAGEHRRDK

SEQ ID NO: 417 - protein, 86 aa, Dengue virus
TRDGEPLMIVAKHERGRPLLFKTTEGINKCTLIAMDLGEMCEDTVMYKCPLLVNTEPEDIDCWCNLTSTWVMYGTCTQSGERRREK

SEQ ID NO: 418 - protein, 87 aa, Japanese encephalitis virus
NFQGKLLMTINNTDIADVIAIPTSKGENRCWVRAIDVGYMCEDTITYECPKLAMGNDPEDVDCWCDNQEVYVQYGRCTRTRHSKRSR

SEQ ID NO: 419 - protein, 80 aa, West Nile virus
NFQGKVMMTVNATDVTDVITIPTAAGKNLCIVRAMDVGYMCDDTITYECPVLSAGNDPEDIDCWCTKSAVYVRYGRCTKT

SEQ ID NO: 420 - protein, 89 aa, Zika virus
TRRGSAYYMYLDRNDAGEAISFPTTLGMNKCYIQIMDLGHTCDATMSYECPMLDEGVEPDDVDCWCNTTSTWVVYGTCHHKKGEARRSR

SEQ ID NO: 421 - protein, 36 aa, Dengue virus
IVTVHTGDQHQVGNETTEHGTTATITPQAPTSEIQL

SEQ ID NO: 422 - protein, 36 aa, Dengue virus
VVTPHSGEEHAVGNDTGKHGKEIKVTPQSSITEAEL

SEQ ID NO: 423 - protein, 34 aa, Dengue virus
IITVHTGDQHQVGNETQGVTAEITSQASTAEAIL

SEQ ID NO: 424 - protein, 36 aa, Dengue virus
VVTVHNGDTHAVGNDTSNHGVTAMITPRSPSVEVKL

SEQ ID NO: 425 - protein, 41 aa, Japanese encephalitis virus
GIFVHGTTTSENHGNYSAQVGASQAAKFTVTPNAPSVTLKL

SEQ ID NO: 426 - protein, 41 aa, West Nile virus
AIFVHGPTTVESHGNYSTQAGATQAGRFSITPAAPSYTLKL

SEQ ID NO: 427 - protein, 41 aa, Zika virus
MLSVHGSQHSGMIVNDTGHETDENRAKVEITPNSPRAEATL

SEQ ID NO: 428 - protein, 41 aa, Zika virus; identical to SEQ ID NO: 427

SEQ ID NO: 429 - protein, 41 aa, Zika virus; identical to SEQ ID NO: 427

SEQ ID NO: 430 - protein, 41 aa, Zika virus; identical to SEQ ID NO: 427

SEQ ID NO: 431 - protein, 41 aa, Zika virus; identical to SEQ ID NO: 427

SEQ ID NO: 432 - protein, 41 aa, Zika virus; identical to SEQ ID NO: 427

SEQ ID NO: 433 - protein, 41 aa, Zika virus; identical to SEQ ID NO: 427

SEQ ID NO: 434 - protein, 41 aa, Zika virus; identical to SEQ ID NO: 427

SEQ ID NO: 435 - protein, 41 aa, Zika virus; identical to SEQ ID NO: 427

SEQ ID NO: 436 - protein, 41 aa, Zika virus; identical to SEQ ID NO: 427

SEQ ID NO: 437 - protein, 41 aa, Zika virus; identical to SEQ ID NO: 427

SEQ ID NO: 438 - protein, 41 aa, Zika virus; identical to SEQ ID NO: 427

SEQ ID NO: 439 - protein, 41 aa, Zika virus; identical to SEQ ID NO: 427

SEQ ID NO: 440 - protein, 41 aa, Zika virus; identical to SEQ ID NO: 427

SEQ ID NO: 441 - protein, 41 aa, Zika virus; identical to SEQ ID NO: 427

SEQ ID NO: 442 - protein, 41 aa, Zika virus; identical to SEQ ID NO: 427

SEQ ID NO: 443 - protein, 41 aa, Zika virus; identical to SEQ ID NO: 427

SEQ ID NO: 444 - protein, 41 aa, Zika virus; identical to SEQ ID NO: 427

SEQ ID NO: 445 - protein, 41 aa, Zika virus; identical to SEQ ID NO: 427

SEQ ID NO: 446 - protein, 41 aa, Zika virus; identical to SEQ ID NO: 427

SEQ ID NO: 447 - protein, 41 aa, Zika virus; identical to SEQ ID NO: 427

SEQ ID NO: 448 - protein, 41 aa, Zika virus; identical to SEQ ID NO: 427

SEQ ID NO: 449 - protein, 41 aa, Zika virus; identical to SEQ ID NO: 427

SEQ ID NO: 450 - protein, 41 aa, Zika virus; identical to SEQ ID NO: 427

SEQ ID NO: 451 - protein, 41 aa, Zika virus; identical to SEQ ID NO: 427

SEQ ID NO: 452 - protein, 41 aa, Zika virus; identical to SEQ ID NO: 427

SEQ ID NO: 453 - protein, 41 aa, Zika virus
MLSVHGSQHSGMIVNDIGHETDENRAKVEVTPNSPRAEATL

SEQ ID NO: 454 - protein, 41 aa, Zika virus
MLSVHGSQHSGMIVNDTGHETDENRAKVEVTPNSPRAEATL

SEQ ID NO: 455 - protein, 35 aa, Zika virus
MLSVHGSQHSGMIVNDENRAKVEVTPNSPRAEATL

SEQ ID NO: 456 - protein, 41 aa, Zika virus
  MOD_RES (17)..(17): Any amino acid
MLSVHGSQHSGMIVNDXGHETDENRAKVEITPNSPRAEATL

SEQ ID NO: 457 - protein, 37 aa, Zika virus
MLSVHGSQHSGMIGYETDEDRAKVEVTPNSPRAEATL

SEQ ID NO: 458 - protein, 37 aa, Zika virus; identical to SEQ ID NO: 457

SEQ ID NO: 459 - protein, 41 aa, Zika virus
MLSVHGSQHSGMTVNDIGYETDENRAKVEVTPNSPRAEATL

SEQ ID NO: 460 - protein, 41 aa, Zika virus
MLSVHGSQHSGMIVNDTGYETDENRAKVEVTPNSPRAEATL

SEQ ID NO: 461 - protein, 37 aa, Zika virus; identical to SEQ ID NO: 457

SEQ ID NO: 462 - protein, 35 aa, Zika virus; identical to SEQ ID NO: 455

SEQ ID NO: 463 - protein, 41 aa, Zika virus; identical to SEQ ID NO: 453

SEQ ID NO: 464 - protein, 35 aa, Zika virus; identical to SEQ ID NO: 455

SEQ ID NO: 465 - protein, 41 aa, Zika virus; identical to SEQ ID NO: 453

SEQ ID NO: 466 - protein, 41 aa, Zika virus; identical to SEQ ID NO: 453

SEQ ID NO: 467 - protein, 41 aa, Zika virus
MLPVHGSQHSGMIVNDIGHETDENRAKVEVTPNSPRAEATL

SEQ ID NO: 468 - protein, 41 aa, Zika virus; identical to SEQ ID NO: 453

SEQ ID NO: 469 - protein, 111 aa, West Nile virus
QLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGK

SEQ ID NO: 470 - protein, 111 aa, Zika virus
RLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGK